SEQUENCE LISTING (108 sequences)

SEQ ID NO: 1 - DNA, 1023 bp, Chlamydia trachomatis
atgagtagcaagctagtgaactatctccgtttgactttcctatcttttttagggatcgcatctacttcattagacgctatgcctgcggggaatccggcgtttccagtcatcccggggattaatattgaacagaaaaatgcctgttctttcgatttatgtaattcttatgatgtactatccgcactgtccggtaacctgaagctctgcttctgcggagattatatcttttcagaagaagctcaggtaaaagatgtccctgtcgttacctctgtgacaacagctggggttggtccttctcctgatattacttcgacaaccaaaacgcgaaatttcgatctcgtgaactgtaatctcaatacaaactgtgtagctgtagctttttcccttcctgatcgttcgctgagcgcgattcctctgtttgatgtgagtttcgaagtgaaagtaggaggactgaaacaatactaccgccttcccatgaatgcctatcgagacttcacctcggaacctctcaattctgaatcagaagttacggacgggatgattgaagtacagtccaattacggatttgtttgggatgttagcttgaaaaaagtcatatggaaagatggcgtttcctttgtaggcgtcggtgcagactatcgccatgcttcttgccctattgactacatcattgcaaacagtcaagctaatccagaagtattcatcgctgactcggatgggaaactgaacttcaaggagtggagtgtctgcgtaggtcttactacctatgtgaatgactacgttcttccttacttagcgttttctatagggagtgtttctcgccaagctccggacgacagcttcaaaaaattagaagatcgcttcactaacctcaaatttaaagttcgtaaaattaccagctctcatcgtggaaacatctgcatcggagcgacaaactatgtcgccgataacttcttctacaacgtagaaggaagatggggaagccagcgcgctgtgaacgtctccggaggattccaattctaa

SEQ ID NO: 2 - protein, 340 aa, Chlamydia trachomatis
MSSKLVNYLRLTFLSFLGIASTSLDAMPAGNPAFPVIPGINIEQKNACSFDLCNSYDVLSALSGNLKLCFCGDYIFSEEAQVKDVPVVTSVTTAGVGPSPDITSTTKTRNFDLVNCNLNTNCVAVAFSLPDRSLSAIPLFDVSFEVKVGGLKQYYRLPMNAYRDFTSEPLNSESEVTDGMIEVQSNYGFVWDVSLKKVIWKDGVSFVGVGADYRHASCPIDYIIANSQANPEVFIADSDGKLNFKEWSVCVGLTTYVNDYVLPYLAFSIGSVSRQAPDDSFKKLEDRFTNLKFKVRKITSSHRGNICIGATNYVADNFFYNVEGRWGSQRAVNVSGGFQF

SEQ ID NO: 3 - protein, 601 aa, Chlamydia trachomatis
MKMNRIWLLLLTFSSAIHSPVQGESLVCKNALQDLSFLEHLLQVKYAPKTWKEQYLGWDLVQSSVSAQQKLRTQENPSTSFCQQVLADFIGGLNDFHAGVTFFAIESAYLPYTVQKSSDGRFYFVDIMTFSSEIRVGDELLEVDGAPVQDVLATLYGSNHKGTAAEESAALRTLFSRMASLGHKVPSGRTTLKIRRPFGTTREVRVKWRYVPEGVGDLATIAPSIRAPQLQKSMRSFFPKKDDAFHRSSSLFYSPMVPHFWAELRNHYATSGLKSGYNIGSTDGFLPVIGPVIWESEGLFRAYISSVTDGDGKSHKVGFLRIPTYSWQDMEDFDPSGPPPWEEFAKIIQVFSSNTEALIIDQTNNPGGSVLYLYALLSMLTDRPLELPKHRMILTQDEVVDALDWLTLLENVDTNVESRLALGDNMEGYTVDLQVAEYLKSFGRQVLNCWSKGDIELSTPIPLFGFEKIHPHPRVQYSKPICVLINEQDFSCADFFPVVLKDNDRALIVGTRTAGAGGFVFNVQFPNRTGIKTCSLTGSLAVREHGAFIENIGVEPHIDLPFTANDIRYKGYSEYLDKVKKLVCQLINNDGTIILAEDGSF

SEQ ID NO: 4 - DNA, 1806 bp, Chlamydia trachomatis
atgaaaatgaataggatttggctattactgcttaccttttcttctgccatacattctcctgtacaaggagaaagcttggtttgcaagaatgctcttcaagatttgagttttttagagcatttattacaggttaaatatgctcctaaaacatggaaagagcaatacttaggatgggatcttgttcaaagctccgtttctgcacagcagaagcttcgtacacaagaaaatccatcaacaagtttttgccagcaggtccttgctgattttatcggaggattaaatgactttcacgctggagtaactttctttgcgatagaaagtgcttaccttccttataccgtacaaaaaagtagtgacggccgtttctactttgtagatatcatgactttttcttcagagatccgtgttggagatgagttgctagaggtggatggggcgcctgtccaagatgtactcgctactctatatggaagcaatcacaaagggactgcagctgaagagtcggctgctttaagaacactattttctcgcatggcctctttagggcacaaagtaccttctgggcgcactactttaaagattcgtcgtccttttggtactacgagagaagttcgtgtgaaatggcgttatgttcctgaaggtgtaggagatttggctaccatagctccttctatcagggctccacagttacagaaatcgatgagaagctttttccctaagaaagatgatgcgtttcatcggtctagttcgctattctactctccaatggttccgcatttttgggcagagcttcgcaatcattatgcaacgagtggtttgaaaagcgggtacaatattgggagtaccgatgggtttctccctgtcattgggcctgttatatgggagtcggagggtcttttccgcgcttatatttcttcggtgactgatggggatggtaagagccataaagtaggatttctaagaattcctacatatagttggcaggacatggaagattttgatccttcaggaccgcctccttgggaagaatttgctaagattattcaagtattttcttctaatacagaagctttgattatcgaccaaacgaacaacccaggtggtagtgtcctttatctttatgcactgctttccatgttgacagaccgtcctttagaacttcctaaacatagaatgattctgactcaggatgaagtggttgatgctttagattggttaaccctgttggaaaacgtagacacaaacgtggagtctcgccttgctctgggagacaacatggaaggatatactgtggatctacaggttgccgagtatttaaaaagctttggacgtcaagtattgaattgttggagtaaaggggatatcgagttatcaacgcctattcctctttttggttttgagaagattcatccacatcctcgagttcaatactctaaaccgatttgtgttttgatcaatgagcaagacttttcttgtgctgacttcttccctgtagttttgaaagacaatgatcgagctcttattgttggtactcgaacagctggagctggaggatttgtctttaatgtgcagttcccaaatagaactggaataaaaacttgttctttaacaggatcattagctgttagagagcatggtgccttcattgagaacatcggagtcgaaccgcatatcgatctgccttttacagcgaatgatattcgctataaaggctattccgagtatcttgataaggtcaaaaaattggtttgtcagctgatcaataacgacggtaccattattcttgcggaagatggtagtttttaa

SEQ ID NO: 5 - protein, 432 aa, Chlamydia trachomatis
MKKYFYKGFVGALLLACGSTNLAFAQASSMDSQLWSVEDLDSYLSSKGFVETRKRDGVLRLAGDVRARWIYAKEDLETTQTPAKPMLPTNRYRSEFNLYVDYTAANSWMTSKMNWVTIAGGESSAAGLDINRAFLGYRFYKNPETQAEVFAEIGRSGLGDIFDSDVQFNSNFDGIHLYAARRISEKLPFTMIVHGGPFVVNMAEKEYAWVVEAILNKLPGNFVVKTSVVDWNTLTAKTNDPADASAAQPAKPNTKYDYLVWQWLVGKSTAMPWFNGQTKNLYTYGAYLFNPLAEIPENWKQSTTPTTKITNGKENHAWFIGCSLGGVRRAGDWSATVRYEYVEALAIPEIDVAGIGRGNQMKYWFAQAIKQGLDPKESNGFTNYKGVSYQFVMGLTDSVSFRAYAAYSKPANDNLGSDFTYRKYDLGLISSF

SEQ ID NO: 6 - DNA, 1299 bp, Chlamydia trachomatis
atgaaaaaatacttttataaagggtttgtaggcgcgcttttattagcttgtgggtctacaaacttggcttttgcgcaggctagttcgatggatagccagctatggtctgttgaagatttagattcttatttgagttccaaaggttttgtcgagactcgtaagcgcgatggagttctacgtttagctggagatgtccgcgctcgatggatttatgcaaaagaggatcttgagacaactcagactcctgctaaacctatgttacctaccaatcggtatcgtagtgaattcaatttgtatgtggattacaccgctgctaatagttggatgacttcgaaaatgaattgggtaacgattgctggcggagaatcttctgcagcagggttagatattaatcgtgccttcttaggataccgattctacaaaaacccagaaacgcaagcagaagtatttgcagagattggtcgctctggattgggagatatttttgattccgacgttcagtttaatagtaatttcgacggaattcatttatacgctgcgcgacgtattagtgagaaacttcctttcaccatgattgttcatggtggtccttttgtcgtgaatatggcagagaaagagtatgcttgggtcgtggaagctattttgaataaactcccaggaaatttcgttgtgaaaacgagtgttgttgactggaatacgttaacagcaaaaacgaatgatccagcagacgcaagcgctgcacaaccagctaaacctaataccaagtacgattatttagtatggcaatggttggttgggaagagcacagctatgccatggtttaatggacaaacaaaaaatctttacacttacggagcctatctctttaatccattagcggaaataccagagaactggaaacaatcaacaactcctacaaccaaaattacaaatggtaaggaaaaccatgcttggttcatcggctgctctctaggcggtgttcgacgagctggagactggtctgcaacagttcgttatgagtatgttgaagctttagcgattccagaaattgatgtcgcgggtattggtcgcggaaaccaaatgaaatattggtttgctcaagctatcaaacaaggattggatcctaaagaatctaacggctttactaactataaaggagtttcctatcagtttgttatgggtctgacagattcggtttctttccgagcttatgctgcttattctaagcctgctaacgataaccttggtagcgacttcacctatcgtaagtatgacctaggtttaatttcttcattctaa

SEQ ID NO: 7 - protein, 878 aa, Chlamydia trachomatis
MRPDHMNFCCLCAAILSSTAVLFGQDPLGETALLTKNPNHVVCTFFEDCTMESLFPALCAHASQDDPLYVLGNSYCWFVSKLHITDPKEALFKEKGDLSIQNFRFLSFTDCSSKESSPSIIHQKNGQLSLRNNGSMSFCRNHAEGSGGAISADAFSLQHNYLFTAFEENSSKGNGGAIQAQTFSLSRNVSPISFARNRADLNGGAICCSNLICSGNVNPLFFTGNSATNGGAICCISDLNTSEKGSLSLACNQETLFASNSAKEKGGAIYAKHMVLRYNGPVSFINNSAKIGGAIAIQSGGSLSILAGEGSVLFQNNSQRTSDQGLVRNAIYLEKDAILSSLEARNGDILFFDPIVQESSSKESPLPSSLQASVTSPTPATASPLVIQTSANRSVIFSSERLSEEEKTPDNLTSQLQQPIELKSGRLVLKDRAVLSAPSLSQDPQALLIMEAGTSLKTSSDLKLATLSIPLHSLDTEKSVTIHAPNLSIQKIFLSNSGDENFYENVELLSKEQNNIPLLTLSKEQSHLHLPDGNLSSHFGYQGDWTFSWKDSDEGHSLIANWTPKNYVPHPERQSTLVANTLWNTYSDMQAVQSMINTIAHGGAYLFGTWGSAVSNLFYAHDSSGKPIDNWHHRSLGYLFGISTHSLDDHSFCLAAGQLLGKSSDSFITSTETTSYIATVQAQLATPLMKISAQACYNESIHELKTKYRSFSKEGFGSWHSVAVSGEVCASIPIVSNGSGLFSSFSIFSKLQGFSGTQDGFEESSGEIRSFSASSFRNISLPMGITFEKKSQKTRNYYYFLGAYIQDLKRDVESGPVVLLKNAVSWDAPMANLDSRAYMFRLTNQRALHRLQTLLNVSYVLRGQSHSYSLDLGTTYRF

SEQ ID NO: 8 - DNA, 2637 bp, Chlamydia trachomatis
atgcgacctgatcatatgaacttctgttgtctatgtgctgctattttgtcatccacagcggtcctctttggccaggatcccttaggtgaaaccgccctcctcactaaaaatcctaatcatgtcgtctgtacattttttgaggactgtaccatggagagcctctttcctgctctttgtgctcatgcatcacaagatgatcctttgtatgtacttggaaattcctactgttggttcgtatctaaactccatatcacggaccccaaagaggctctttttaaagaaaaaggagatctttccattcaaaattttcgcttcctttccttcacagattgctcttccaaggaaagctctccttctattattcatcaaaagaatggtcagttatccttgcgcaataatggtagcatgagtttctgtcgaaatcatgctgaaggctctggaggagccatctctgcggatgccttttctctacaacacaactatcttttcacagcttttgaagagaattcttctaaaggaaatggcggagccattcaggctcaaaccttctctttatctagaaatgtgtcgcctatttctttcgcccgtaatcgtgcggatttaaatggcggcgctatttgctgtagtaatcttatttgttcagggaatgtaaaccctctctttttcactggaaactccgccacgaatggaggcgctatttgttgtatcagcgatctaaacacctcagaaaaaggctctctctctcttgcttgtaaccaagaaacgctatttgcaagcaattctgctaaagaaaaaggcggggctatttatgccaagcacatggtattgcgttataacggtcctgtttccttcattaacaacagcgctaaaataggtggagctatcgccatccagtccggagggagtctctctatccttgcaggtgaaggatctgttctgttccagaataactcccaacgcacctccgaccaaggtctagtaagaaacgccatctacttagagaaagatgcgattctttcttccttagaagctcgcaacggagatattcttttctttgatcctattgtacaagaaagtagcagcaaagaatcgcctcttccctcctctttgcaagccagcgtgacttctcccaccccagccaccgcatctcctttagttattcagacaagtgcaaaccgttcagtgattttctcgagcgaacgtctttctgaagaagaaaaaactcctgataacctcacttcccaactacagcagcctatcgaactgaaatccggacgcttagttttaaaagatcgcgctgtcctttccgcgccttctctctctcaggatcctcaagctctcctcattatggaagcgggaacttctttaaaaacttcctctgatttgaagttagctacgctaagtattccccttcattccttagatactgaaaaaagcgtaactatccacgcccctaacctttctatccaaaagatcttcctctctaattctggagatgagaatttttatgaaaatgtagagcttctcagtaaagagcaaaacaatattcctctccttactctctctaaagagcaatctcatttacatcttcctgatgggaacctctcttctcactttggatatcaaggagattggactttttcttggaaagattctgatgaagggcattctctgattgctaattggacgcctaaaaactatgtgcctcatccagaacgtcaatctacactcgttgcgaacactctttggaacacctattccgatatgcaagctgtgcagtcgatgattaatacaatagcgcacggaggagcctatctatttggaacgtggggatctgctgtttctaatttattctatgctcacgacagctctgggaaacctatcgataattggcatcatagaagccttggctacctattcggtatcagtactcacagtttagatgaccattctttctgcttggctgcaggacaattactcgggaaatcgtccgattcctttattacgtctacagaaacgacctcctatatagctactgtacaagcgcaactcgctacccctctaatgaaaatctctgcacaggcatgctataatgaaagtatccatgagctaaaaacaaaatatcgctccttctctaaagaaggattcggatcctggcatagcgttgcagtatccggagaagtgtgcgcatcgattcctattgtatccaatggttccggactgttcagctccttctctattttctctaaactgcaaggattttcaggaacacaggacggttttgaggagagttcgggagagattcggtccttttctgccagctctttcagaaatatttcacttcctatgggaataacatttgaaaaaaaatcccaaaaaacacgaaactactattactttctgggagcctacatccaagacctaaaacgtgatgtggaatcgggacctgtagtgttactcaaaaatgccgtctcctgggatgctcctatggcgaacttggattcgcgagcctacatgttcaggcttacgaatcaaagagctctgcatagacttcagacgctgttaaatgtgtcttacgtactgcgcgggcaaagccatagttactccctggatctggggaccacttacaggttctag

SEQ ID NO: 9 - protein, 1013 aa, Chlamydia trachomatis
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 10 - DNA, 3042 bp, Chlamydia trachomatis
atgcaaacgtctttccataagttctttctttcaatgattctagcttattcttgctgctctttaagtgggggggggtatgcagcagaaatcatgattcctcaaggaatttacgatggggagacgttaactgtatcatttccctatactgttataggagatccgagtgggactactgttttttctgcaggagagttaacgttaaaaaatcttgacaattctattgcagctttgcctttaagttgttttgggaacttattagggagttttactgttttagggagaggacactcgttgactttcgagaacatacggacttctacaaatggagctgcactaagtgacagcgctaatagcgggttatttactattgagggttttaaagaattatctttttccaattgcaactcattacttgccgtactgcctgctgcaacgactaataatggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttatagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctgagcaaccaacaaatggacaggcttctaatacgagtgataattacggagatggaggagctatcttctgtaagaatggtgcgcaagcagcaggatccaataactctggatcagtttcctttgatggagagggagtagttttctttagtagcaatgtagctgctgggaaagggggagctatttatgccaaaaagctctcggttgctaactgtggccctgtacaattcttagggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccgaacctctaaaaattaacgatggtgaaggatacacaggggatattgtttttgctaatggaaacagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcaatcattggtagcacaactgctggttctgttacaattagtgggcctatcttttttgaggatttggatgatacagcttatgataggtatgattggctaggttctaatcaaaaaatcgatgtcctgaaattacagttagggactcagccctcagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattcagcaattcaagcaagtgtggatgggcgctcttattgtcgaggattatgggtttctggagtttcgaatttcttctatcatgaccgcgatgctttaggtcagggatatcggtatattagtgggggttattccttaggagcaaactcctactttggatcatcgatgtttggtctagcatttaccgaagtatttggtagatctaaagattatgtagtgtgtcgttccaatcatcatgcttgcataggatccgtttatctatctaccaaacaagctttatgtggatcctatttgttcggagatgcgtttatccgtgctagctacgggtttgggaaccagcatatgaaaacctcatacacatttgcagaggagagcgatgttcgttgggataataactgtctggttggagagattggagtgggattaccgattgtgattactccatctaagctctatttgaatgagttgcgtcctttcgtgcaagctgagttttcttatgccgatcatgaatcttttacagaggaaggcgatcaagctcgggcattcaggagtggacatctcatgaatctatcagttcctgttggagtaaaatttgatcgatgttctagtacacaccctaataaatatagctttatgggggcttatatctgtgatgcttatcgcaccatctctgggactcagacaacactcctatcccatcaagagacatggacaacagatgcctttcatttggcaagacatggagtcatagttagagggtctatgtatgcttctctaacaagcaatatagaagtatatggccatggaagatatgagtatcgagatacttctcgaggttatggtttgagtgcaggaagtaaagtccggttctaa

SEQ ID NO: 11 - protein, 1034 aa, Chlamydia trachomatis
MIKRTSLSFACLSFFYLSTISILQANETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGRLSIVDENYESVYDSMDLSRGKAEQLILSIETTNDGQLDSNWQSSLNTSLLSPPHYGYQGLWTPNWITTTYTITLNNNSSAPTSATSIAEQKKTSETFTPSNTTTASIPNIKASAGSGSGSASNSGEVTITKHTLVVNWAPVGYIVDPIRRGDLIANSLVHSGRNMTMGLRSLLPDNSWFALQGAATTLFTKQQKRLSYHGYSSASKGYTVSSQASGAHGHKFLLSFSQSSDKMKEKETNNRLSSRYYLSALCFEHPMFDRIALIGAAACNYGTHNMRSFYGTKKSSKGKFHSTTLGASLRCELRDSMPLRSIMLTPFAQALFSRTEPASIRESGDLARLFTLEQAHTAVVSPIGIKGAYSSDTWPTLSWEMELAYQPTLYWKRPLLNTLLIQNNGSWVTTNTPLAKHSFYGRGSHSLKFSHLKLFANYQAEVATSTVSHYINAGGALVF

SEQ ID NO: 12 - DNA, 3105 bp, Chlamydia trachomatis
atgattaaaagaacttctctatcctttgcttgcctcagttttttttatctttcaactatatccattttgcaagctaatgaaacggatacgctacagttccggcgatttactttttcggatagagagattcagttcgtcctagatcccgcctctttaattaccgcccaaaacatcgttttatctaatttacagtcaaacggaaccggagcctgtaccatttcaggcaatacgcaaactcaaatcttttctaattccgttaacaccaccgcagattctggtggagcctttgatatggttactacctcattcacggcctctgataatgctaatctactcttctgcaacaactactgcacacataataaaggcggaggagctattcgttccggaggacctattcgattcttaaataatcaagacgtgcttttttataataacatatcggcaggggctaaatatgttggaacaggagatcacaacgaaaaaaataggggcggtgcgctttatgcaactactatcactttgacagggaatcgaactcttgcctttattaacaatatgtctggagactgcggtggagccatctctgctgacactcaaatatcaataactgataccgttaaaggaattttatttgaaaacaatcacacgctcaatcatataccgtacacgcaagctgaaaatatggcacgaggaggagcaatctgtagtagaagagacttgtgctcaatcagcaataattctggtcccatagtttttaactataaccaaggcgggaaaggtggagctattagcgctacccgatgtgttattgacaataacaaagaaagaatcatcttttcaaacaatagttccctgggatggagccaatcttcttctgcaagtaacggaggagccattcaaacgacacaaggatttactttacgaaataataaaggctctatctacttcgacagcaacactgctacacacgccgggggagccattaactgtggttacattgacatccgagataacggacccgtctattttctaaataactctgctgcctggggagcggcctttaatttatcgaaaccacgttcagcgacaaattatatccatacagggacaggcgatattgtttttaataataacgttgtctttactcttgacggtaatttattagggaaacggaaactttttcatattaataataatgagataacaccatatacattgtctctcggcgctaaaaaagatactcgtatctatttttatgatcttttccaatgggagcgtgttaaagaaaatactagcaataacccaccatctcctaccagtagaaacaccattaccgttaacccggaaacagagttttctggagctgttgtgttctcctacaatcaaatgtctagtgacatacgaactctgatgggtaaagaacacaattacattaaagaagccccaactactttaaaattcggaacgctagccatagaagatgatgcagaattagaaatcttcaatatcccgtttacccaaaatccgactagccttcttgctttaggaagcggcgctacgctgactgttggaaagcacggtaagctcaatattacaaatcttggtgttattttacccattattctcaaagaggggaagagtccgccttgtattcgcgtcaacccacaagatatgacccaaaatactggtaccggccaaactccatcaagcacaagtagtataagcactccaatgattatctttaatgggcgcctctcaattgtagacgaaaattatgaatcagtctacgacagtatggacctctccagagggaaagcagaacaactaattctatccatagaaaccactaatgatgggcaattagactccaattggcaaagttctctgaatacttctctactctctcctccacactatggctatcaaggtctatggactcctaattggataacaacaacctataccatcacgcttaataataattcttcagctccaacatctgctacctccatcgctgagcagaaaaaaactagtgaaacttttactcctagtaacacaactacagctagtatccctaatattaaagcttccgcaggatcaggctctggatcggcttccaattcaggagaagttacgattaccaaacatacccttgttgtaaactgggcaccagtcggctacatagtagatcctattcgtagaggagatctgatagccaatagcttagtacattcaggaagaaacatgaccatgggcttacgatcattactcccggataactcttggtttgctttgcaaggagctgcaacaacattatttacaaaacaacaaaaacgtttgagttatcatggctactcttctgcatcaaaggggtataccgtctcttctcaagcatcaggagctcatggtcataagtttcttctttccttctcccagtcatctgataagatgaaagaaaaagaaacaaataaccgcctttcttctcgttactatctttctgctttatgtttcgaacatcctatgtttgatcgcattgctcttatcggagcagcagcttgcaattatggaacacataacatgcggagtttctatggaactaaaaaatcttctaaagggaaatttcactctacaaccttaggagcttctcttcgctgtgaactacgcgatagtatgcctttacgatcaataatgctcaccccatttgctcaggctttattctctcgaacagaaccagcttctatccgagaaagcggtgatctagctagattatttacattagagcaagcccatactgccgttgtctctccaataggaatcaaaggagcttattcttctgatacatggccaacactctcttgggaaatggaactagcttaccaacccaccctctactggaaacgtcctctactcaacacactattaatccaaaataacggttcttgggtcaccacaaataccccattagctaaacattccttttatgggagaggttctcactccctcaaattttctcatctgaaactatttgctaactatcaagcagaagtggctacttccactgtctcacactacatcaatgcaggaggagctctggtcttttaa

SEQ ID NO: 13 - protein, 964 aa, Chlamydia trachomatis
MKKAFFFFLIGNSLSGLAREVPSRIFLMPNSVPDPTKESLSNKISLTGDTHNLTNCYLDNLRYILAILQKTPNEGAAVTITDYLSFFDTQKEGIYFAKNLTPESGGAIGYASPNSPTVEIRDTIGPVIFENNTCCRLFTWRNPYAADKIREGGAIHAQNLYINHNHDVVGFMKNFSYVQGGAISTANTFVVSENQSCFLFMDNICIQTNTAGKGGAIYAGTSNSFESNNCDLFFINNACCAGGAIFSPICSLTGNRGNIVFYNNRCFKNVETASSEASDGGAIKVTTRLDVTGNRGRIFFSDNITKNYGGAIYAPVVTLVDNGPTYFINNIANNKGGAIYIDGTSNSKISADRHAIIFNENIVTNVTNANGTSTSANPPRRNAITVASSSGEILLGAGSSQNLIFYDPIEVSNAGVSVSFNKEADQTGSVVFSGATVNSADFHQRNLQTKTPAPLTLSNGFLCIEDHAQLTVNRFTQTGGVVSLGNGAVLSCYKNGTGDSASNASITLKHIGLNLSSILKSGAEIPLLWVEPTNNSNNYTADTAATFSLSDVKLSLIDDYGNSPYESTDLTHALSSQPMLSISEASDNQLQSENIDFSGLNVPHYGWQGLWTWGWAKTQDPEPASSATITDPQKANRFHRTLLLTWLPAGYVPSPKHRSPLIANTLWGNMLLATESLKNSAELTPSGHPFWGITGGGLGMMVYQDPRENHPGFHMRSSGYSAGMIAGQTHTFSLKFSQTYTKLNERYAKNNVSSKNYSCQGEMLFSLQEGFLLTKLVGLYSYGDHNCHHFYTQGENLTSQGTFRSQTMGGAVFFDLPMKPFGSTHILTAPFLGALGIYSSLSHFTEVGAYPRSFSTKTPLINVLVPIGVKGSFMNATHRPQAWTVELAYQPVLYRQEPGIAAQLLASKGIWFGSGSPSSRHAMSYKISQQTQPLSWLTLHFQYHGFYSSSTFCNYLNGEIALRF

SEQ ID NO: 14 - DNA, 2895 bp, Chlamydia trachomatis
atgaaaaaagcgtttttctttttccttatcggaaactccctatcaggactagctagagaggttccttctagaatctttcttatgcccaactcagttccagatcctacgaaagagtcgctatcaaataaaattagtttgacaggagacactcacaatctcactaactgctatctcgataacctacgctacatactggctattctacaaaaaactcccaatgaaggagctgctgtcacaataacagattacctaagcttttttgatacacaaaaagaaggtatttattttgcaaaaaatctcacccctgaaagtggtggtgcgattggttatgcgagtcccaattctcctaccgtggagattcgtgatacaataggtcctgtaatctttgaaaataatacttgttgcagactatttacatggagaaatccttatgctgctgataaaataagagaaggcggagccattcatgctcaaaatctttacataaatcataatcatgatgtggtcggatttatgaagaacttttcttatgtccaaggaggagccattagtaccgctaatacctttgttgtgagcgagaatcagtcttgttttctctttatggacaacatctgtattcaaactaatacagcaggaaaaggtggcgctatctatgctggaacgagcaattcttttgagagtaataactgcgatctcttcttcatcaataacgcctgttgtgcaggaggagcgatcttctcccctatctgttctctaacaggaaatcgtggtaacatcgttttctataacaatcgctgctttaaaaatgtagaaacagcttcttcagaagcttctgatggaggagcaattaaagtaactactcgcctagatgttacaggcaatcgtggtaggatcttttttagtgacaatatcacaaaaaattatggcggagctatttacgctcctgtagttaccctagtggataatggccctacctactttataaacaatatcgccaataataaggggggcgctatctatatagacggaaccagtaactccaaaatttctgccgaccgccatgctattatttttaatgaaaatattgtgactaatgtaactaatgcaaatggtaccagtacgtcagctaatcctcctagaagaaatgcaataacagtagcaagctcctctggtgaaattctattaggagcagggagtagccaaaatttaattttttatgatcctattgaagttagcaatgcaggggtctctgtgtccttcaataaggaagctgatcaaacaggctctgtagtattttcaggagctactgttaattctgcagattttcatcaacgcaatttacaaacaaaaacacctgcaccccttactctcagtaatggttttctatgtatcgaagatcatgctcagcttacagtgaatcgattcacacaaactgggggtgttgtttctcttgggaatggagcagttctgagttgctataaaaatggtacaggagattctgctagcaatgcctctataacactgaagcatattggattgaatctttcttccattctgaaaagtggtgctgagattcctttattgtgggtagagcctacaaataacagcaataactatacagcagatactgcagctaccttttcattaagtgatgtaaaactctcactcattgatgactacgggaactctccttatgaatccacagatctgacccatgctctgtcatcacagcctatgctatctatttctgaagctagcgataaccagctacaatcagaaaatatagatttttcgggactaaatgtccctcattatggatggcaaggactttggacttggggctgggcaaaaactcaagatccagaaccagcatcttcagcaacaatcactgatccacaaaaagccaatagatttcatagaaccttactactaacatggcttcctgccgggtatgttcctagcccaaaacacagaagtcccctcatagctaacaccttatgggggaatatgctgcttgcaacagaaagcttaaaaaatagtgcagagctgacacctagtggtcatcctttctggggaattacaggaggaggactaggcatgatggtttaccaagatcctcgagaaaatcatcctggattccatatgcgctcttccggatactctgcggggatgatagcagggcagacacacaccttctcattgaaattcagtcagacctacaccaaactcaatgagcgttacgcaaaaaacaacgtatcttctaaaaattactcatgccaaggagaaatgctcttctcattgcaagaaggtttcttgctgactaaattagttgggctttacagctatggagaccataactgtcaccatttctatactcaaggagaaaatctaacatctcaagggacgttccgcagtcaaacgatgggaggtgctgtcttttttgatctccctatgaaaccctttggatcaacgcatatactgacagctccctttttaggtgctcttggtatttattctagcctgtctcactttactgaggtgggagcctatccgcgaagcttttctacaaagactcctttgatcaatgtcctagtccctattggagttaaaggtagctttatgaatgctacccacagacctcaagcctggactgtagaattggcataccaacccgttctgtatagacaagaaccagggatcgcagcccagctcctagccagtaagggtatttggttcggtagtggaagcccctcatcgcgtcatgccatgtcctataaaatctcacagcaaacacaacctttgagttggttaactctccatttccagtatcatggattctactcctcttcaaccttctgtaattatctcaatggggaaattgctctgcgattctag

SEQ ID NO: 15 - protein, 1531 aa, Chlamydia trachomatis
MSSEKDIKSTCSKFSLSVVAAILASVSGLASCVDLHAGGQSVNELVYVGPQAVLLLDQIRDLFVGSKDSQAEGQYRLIVGDPSSFQEKDADTLPGKVEQSTLFSVTNPVVFQGVDQQDQVSSQGLICSFTSSNLDSPRDGESFLGIAFVGDSSKAGITLTDVKASLSGAALYSTEDLIFEKIKGGLEFASCSSLEQGGACAAQSILIHDCQGLQVKHCTTAVNAEGSSANDHLGFGGGAFFVTGSLSGEKSLYMPAGDMVVANCDGAISFEGNSANFANGGAIAASGKVLFVANDKKTSFIENRALSGGAIAASSDIAFQNCAELVFKGNCAIGTEDKGSLGGGAISSLGTVLLQGNHGITCDKNESASQGGAIFGKNCQISDNEGPVVFRDSTACLGGGAIAAQEIVSIQNNQAGISFEGGKASFGGGIACGSFSSAGGASVLGTIDISKNLGAISFSRTLCTTSDLGQMEYQGGGALFGENISLSENAGVLTFKDNIVKTFASNGKILGGGAILATGKVEITNNSEGISFTGNARAPQALPTQEEFPLFSKKEGRPLSSGYSGGGAILGREVAILHNAAVVFEQNRLQCSEEEATLLGCCGGGAVHGMDSTSIVGNSSVRFGNNYAMGQGVSGGALLSKTVQLAGNGSVDFSRNIASLGGGALQASEGNCELVDNGYVLFRDNRGRVYGGAISCLRGDVVISGNKGRVEFKDNIATRLYVEETVEKVEEVEPAPEQKDNNELSFLGRAEQSFITAANQALFASEDGDLSPESSISSEELAKRRECAGGAIFAKRVRIVDNQEAVVFSNNFSDIYGGAIFTGSLREEDKLDGQIPEVLISGNAGDVVFSGNSSKRDEHLPHTGGGAICTQNLTISQNTGNVLFYNNVACSGGAVRIEDHGNVLLEAFGGDIVFKGNSSFRAQGSDAIYFAGKESHITALNATEGHAIVFHDALVFENLEERKSAEVLLINSRENPGYTGSIRFLEAESKVPQCIHVQQGSLELLNGATLCSYGFKQDAGAKLVLAAGAKLKILDSGTPVQQGHAISKPEAEIESSSEPEGAHSLWIAKNAQTTVPMVDIHTISVDLASFSSSQQEGTVEAPQVIVPGGSYVRSGELNLELVNTTGTGYENHALLKNEAKVPLMSFVASGDEASAEISNLSVSDLQIHVVTPEIEEDTYGHMGDWSEAKIQDGTLVISWNPTGYRLDPQKAGALVFNALWEEGAVLSALKNARFAHNLTAQRMEFDYSTNVWGFAFGGFRTLSAENLVAIDGYKGAYGGASAGVDIQLMEDFVLGVSGAAFLGKMDSQKFDAEVSRKGVVGSVYTGFLAGSWFFKGQYSLGETQNDMKTRYGVLGESSASWTSRGVLADALVEYRSLVGPVRPTFYALHFNPYVEVSYASMKFPGFTEQGREARSFEDASLTNITIPLGMKFELAFIKGQFSEVNSLGISYAWEAYRKVEGGAVQLLEAGFDWEGAPMDLPRQELRVALENNTEWSSYFSTVLGLTAFCGGFTSTDSKLGYEANTGLRLIF

SEQ ID NO: 16 - DNA, 4596 bp, Chlamydia trachomatis
atgagttccgagaaagatataaaaagcacctgttctaagttttctttgtctgtagtagcagctatccttgcctctgttagcgggttagctagttgcgtagatcttcatgctggaggacagtctgtaaatgagctggtatatgtaggccctcaagcggttttattgttagaccaaattcgagatctattcgttgggtctaaagatagtcaggctgaaggacagtataggttaattgtaggagatccaagttctttccaagagaaagatgcggatactcttcccgggaaggtagagcaaagtactttgttctcagtaaccaatcccgtggttttccaaggtgtggaccaacaggatcaagtctcttcccaagggttaatttgtagttttacgagcagcaaccttgattctcctcgtgacggagaatcttttttaggtattgcttttgttggggatagtagtaaggctggaatcacattaactgacgtgaaagcttctttgtctggagcggctttatattctacagaagatcttatctttgaaaagattaagggtggattggaatttgcatcatgttcttctctagaacaggggggagcttgtgcagctcaaagtattttgattcatgattgtcaaggattgcaggttaaacactgtactacagccgtgaatgctgaggggtctagtgcgaatgatcatcttggatttggaggaggcgctttctttgttacgggttctctttctggagagaaaagtctctatatgcctgcaggagatatggtagttgcgaattgtgatggggctatatcttttgaaggaaacagcgcgaactttgctaatggaggagcgattgctgcctctgggaaagtgctttttgtcgctaatgataaaaagacttcttttatagagaaccgagctttgtctggaggagcgattgcagcctcttctgatattgcctttcaaaactgcgcagaactagttttcaaaggcaattgtgcaattggaacagaggataaaggttctttaggtggaggggctatatcttctctaggcaccgttcttttgcaagggaatcacgggataacttgtgataagaatgagtctgcttcgcaaggaggcgccatttttggcaaaaattgtcagatttctgacaacgaggggccagtggttttcagagatagtacagcttgcttaggaggaggcgctattgcagctcaagaaattgtttctattcagaacaatcaggctgggatttccttcgagggaggtaaggctagtttcggaggaggtattgcgtgtggatctttttcttccgcaggtggtgcttctgttttagggaccattgatatttcgaagaatttaggcgcgatttcgttctctcgtactttatgtacgacctcagatttaggacaaatggagtaccagggaggaggagctctatttggtgaaaatatttctctttctgagaatgctggtgtgctcacctttaaagacaacattgtgaagacttttgcttcgaatgggaaaattctgggaggaggagcgattttagctactggtaaggtggaaattactaataattccgaaggaatttcttttacaggaaatgcgagagctccacaagctcttccaactcaagaggagtttcctttattcagcaaaaaagaagggcgaccactctcttcaggatattctgggggaggagcgattttaggaagagaagtagctattctccacaacgctgcagtagtatttgagcaaaatcgtttgcagtgcagcgaagaagaagcgacattattaggttgttgtggaggaggcgctgttcatgggatggatagcacttcgattgttggcaactcttcagtaagatttggtaataattacgcaatgggacaaggagtctcaggaggagctcttttatctaaaacagtgcagttagctgggaatggaagcgtcgatttttctcgaaatattgctagtttgggaggaggagctcttcaagcttctgaaggaaattgtgagctagttgataacggctatgtgctattcagagataatcgagggagggtttatgggggtgctatttcttgcttacgtggagatgtagtcatttctggaaacaagggtagagttgaatttaaagacaacatagcaacacgtctttatgtggaagaaactgtagaaaaggttgaagaggtagagccagctcctgagcaaaaagacaataatgagctttctttcttagggagagcagaacagagttttattactgcagctaatcaagctcttttcgcatctgaagatggggatttatcacctgagtcatccatttcttctgaagaacttgcgaaaagaagagagtgtgctggaggagctatttttgcaaaacgggttcgtattgtagataaccaagaggccgttgtattctcgaataacttctctgatatttatggcggcgccatttttacaggttctcttcgagaagaggataagttagatgggcaaatccctgaagtcttgatctcaggcaatgcaggggatgttgttttttccggaaattcctcgaagcgtgatgagcatcttcctcatacaggtgggggagccatttgtactcaaaatttgacgatttctcagaatacagggaatgttctgttttataacaacgtggcctgttcgggaggagctgttcgtatagaggatcatggtaatgttcttttagaagcttttggaggagatattgtttttaaaggaaattcttctttcagagcacaaggatccgatgctatctattttgcaggtaaagaatcgcatattacagccctgaatgctacggaaggacatgctattgttttccacgacgcattagtttttgaaaatctagaagaaaggaaatctgctgaagtattgttaatcaatagtcgagaaaatccaggttacactggatctattcgatttttagaagcagaaagtaaagttcctcaatgtattcatgtacaacaaggaagccttgagttgctaaatggagccacattatgtagttatggttttaaacaagatgctggagctaagttggtattggctgctggagctaaactgaagattttagattcaggaactcctgtacaacaagggcatgctatcagtaaacctgaagcagaaatcgagtcatcttctgaaccagagggtgcacattctctttggattgcgaagaatgctcaaacaacagttcctatggttgatatccatactatttctgtagatttagcctccttctcttctagtcaacaggaggggacagtagaagctcctcaggttattgttcctggaggaagttatgttcgatctggagagcttaatttggagttagttaacacaacaggtactggttatgaaaatcatgctttattgaagaatgaggctaaagttccattgatgtctttcgttgcttctggtgatgaagcttcagccgaaatcagtaacttgtcggtttctgatttacagattcatgtagtaactccagagattgaagaagacacatacggccatatgggagattggtctgaggctaaaattcaagatggaactcttgtcattagttggaatcctactggatatcgattagatcctcaaaaagcaggggctttagtatttaatgcattatgggaagaaggggctgtcttgtctgctctgaaaaatgcacgctttgctcataatctcactgctcagcgtatggaattcgattattctacaaatgtgtggggattcgcctttggtggtttccgaactctatctgcagagaatctggttgctattgatggatacaaaggagcttatggtggtgcttctgctggagtcgatattcaattgatggaagattttgttctaggagttagtggagctgctttcctaggtaaaatggatagtcagaagtttgatgcggaggtttctcggaagggagttgttggttctgtatatacaggatttttagctggatcctggttcttcaaaggacaatatagccttggagaaacacagaacgatatgaaaacgcgttatggagtactaggagagtcgagtgcttcttggacatctcgaggagtactggcagatgctttagttgaataccgaagtttagttggtcctgtgagacctactttttatgctttgcatttcaatccttatgtcgaagtatcttatgcttctatgaaattccctggctttacagaacaaggaagagaagcgcgttcttttgaagacgcttcccttaccaatatcaccattcctttagggatgaagtttgaattggcgttcataaaaggacagttttcagaggtgaactctttgggaataagttatgcatgggaagcttatcgaaaagtagaaggaggcgcggtgcagcttttagaagctgggtttgattgggagggagctccaatggatcttcctagacaggagctgcgtgtcgctctggaaaataatacggaatggagttcttacttcagcacagtcttaggattaacagctttttgtggaggatttacttctacagatagtaaactaggatatgaggcgaatactggattgcgattgatcttttaa

SEQ ID NO: 17 - protein, 340 aa, Chlamydia trachomatis; identical to SEQ ID NO: 2

SEQ ID NO: 18 - DNA, 1023 bp, Chlamydia trachomatis; identical to SEQ ID NO: 1

SEQ ID NO: 19 - protein, 350 aa, Chlamydia trachomatis
MSLFSKFKAQWMFLHSRELCSSTSDIGNTCSDPVFQVLCNPVRSEISYKVGDSLGVFPTNPSILVDSVLDALQYGPRSPVVSRHADSVLPLHEFLTSYVDLDKIPKSLRPFFPGDLDDTWSLAEAILVYQPRIPFEEFIRSAMPLLPRFYSIASSPTCSHGKLELLVRCVSFQGKTQLRYGLCSAFLCKDLQEGESFRGFIQPTRHFTLEQKNFGKPLIMIGAGTGIAPYKGFLQHRIYHQDVGSNILFFGERFEKSNFYYRDFLQELIVSGKLQLFTAFSRDSESKLYVQNVIEQQKELIQEVYEQEAFFFVCGKKILGTEVKRALEQILGPKAVRELIAQKRLVSDVY

SEQ ID NO: 20 - DNA, 1053 bp, Chlamydia trachomatis
atgtctttattttctaaattcaaagctcagtggatgtttttacattcacgtgagctttgttcttccacatcggatattgggaatacttgttcggatcctgtttttcaggttttatgtaatccggttcgttctgagatttcctataaagttggggattctttgggggtattcccaacaaatccttccatattagtcgattcagttctagatgctttacagtatggccccaggtctcctgtcgtatctcggcatgcagattctgttctccctcttcacgaatttcttactagttacgtagacttagataaaattccaaaatcgttaagaccttttttcccaggggatttagacgatacctggtctttagctgaagctattttggtttaccagccgcgtattccttttgaagagtttattcggagtgcgatgcctttattgcctcgattttattctatagcttcttctccaacatgttctcatgggaagctagagttgctcgtgcgctgtgttagtttccaaggtaaaacgcagctgcgctatggattatgttcggcttttttatgtaaggacttacaagagggagagtcttttcgtgggtttatacaaccgacgcggcattttactttggagcagaaaaattttgggaaacctttaattatgatcggagcagggacaggtatcgctccgtacaaagggttcttacaacatcgaatataccatcaggacgtaggctccaatattctattctttggagagcgttttgagaaaagtaacttctattaccgggattttctccaggagctgatcgtttcaggaaaactccagttattcacagccttttccagagattccgagtctaaattgtatgttcagaatgttatagagcaacaaaaagaacttatacaagaagtctacgaacaagaagctttcttttttgtttgtgggaaaaaaatccttggtacggaagttaaacgtgctttagagcagatattaggtcctaaggcggtacgagagctgattgcacagaagagactagtttcagacgtatactaa

SEQ ID NO: 21 - protein, 1770 aa, Chlamydia trachomatis
MKFMSATAVFAAALSSVTEASSIQDQIKNTDCNVSKLGYSTSQAFTDMMLADNTEYRAADSVSFYDFSTSSRLPRKHLSSSSEASPTTEGVSSSSSGETDEKTEEELDNGGIIYAREKLTISESQDSLSNQSIELHDNSIFFGEGEVIFDHRVALKNGGAIYGEKEVVFENIKSLLVEVNIAVEKGGSVYAKERVSLENVTEATFSSNGGEQGGGGIYSEQDMLISDCNNVHFQGNAAGATAVKQCLDEEMIVLLAECVDSLSEDTLDSTPETEQTESNGNQDGSSETEDTQVSESPESTPSPDDVLGKGGGIYTEKSLTITGITGTIDFVSNIATDSGAGVFTKENLSCTNTNSLQFLKNSAGQHGGGAYVTQTMSVTNTTSESITTPPLIGEVIFSENTAKGHGGGICTNKLSLSNLKTVTLTKNSAKESGGAIFTDLASIPITDTPESSTPSSSSPASTPEVVASAKINRFFASTAKPAAPSLTEAESDQTDQTETSDTNSDIDVSIENILNVAINQNTSAKKGGAIYGKKAKLSRINNLELSGNSSQDVGGGLCLTESVEFDAIGSLLSHYNSAAKEGGAIHSKTVTLSNLKSTFTFADNTVKAIVESTPEAPEEIPPVEGEESTATEDPNSNTEGSSANTNLEGSQGDTADTGTGDVNNESQDTSDTGNAESEEQLQDSTQSNEENTLPNSNIDQSNENTDESSDSHTEEITDESVSSSSESGSSTPQDGGAASSGAPSGDQSISANACLAKSYAASTDSSPVSNSSGSEEPVTSSSDSDVTASSDNPDSSSSGDSAGDSEEPTEPEAGSTTETLTLIGGGAIYGETVKIENFSGQGIFSGNKAIDNTTEGSSSKSDVLGGAVYAKTLFNLDSGSSRRTVTFSGNTVSSQSTTGQVAGGAIYSPTVTIATPVVFSKNSATNNANNTTDTQRKDTFGGAIGATSAVSLSGGAHFLENVADLGSAIGLVPGTQNTETVKLESGSYYFEKNKALKRATIYAPVVSIKAYTATFNQNRSLEEGSAIYFTKEASIESLGSVLFTGNLVTLTLSTTTEGTPATTSGDVTKYGAAIFGQIASSNGSQTDNLPLKLIASGGNICFRNNEYRPTSSDTGTSTFCSIAGDVKLTMQAAKGKTISFFDAIRTSTKKTGTQATAYDTLDINKSEDSETVNSAFTGTILFSSELHENKSYIPQNVVLHSGSLVLKPNTELHVISFEQKEGSSLVMTPGSVLSNQTVADGALVINNMTIDLSSVEKNGIAEGNIFTPPELRIIDTTTGGSGGTPSTDSESNQNSDDTEEQNNNDASNQGESANGSSSPAVAAAHTSRTRNFAAAATATPTTTPTATTTTSNQVILGGEIKLIDPNGTFFQNPALRSDQQISLLVLPTDSSKMQAQKIVLTGDIAPQKGYTGTLTLDPDQLQNGTISVLWKFDSYRQWAYVPRDNHFYANSILGSQMLMVTVKQGLLNDKMNLARFEEVSYNNLWISGLGTMLSQVGTPTSEEFTYYSRGASVALDAKPAHDVIVGAAFSKMIGKTKSLKRENNYTHKGSEYSYQASVYGGKPFHFVINKKTEKSLPLLLQGVISYGYIKHDTVTHYPTIRERNKGEWEDLGWLTALRVSSVLRTPAQGDTKRITVYGELEYSSIRQKQFTETEYDPRYFDNCTYRNLAIPMGLAFEGELSGNDILMYNRFSVAYMLSIYRNSPTCKYQVLSSGEGGEIICGVPTRNSARGEYSTQLYLGPLWTLYGSYTIEADAHTLAHMMNCGARMTF

SEQ ID NO: 22 - DNA, 5313 bp, Chlamydia trachomatis
atgaaatttatgtcagctactgctgtatttgctgcagcactctcctccgttactgaggcgagctcgatccaagatcaaataaagaataccgactgcaatgttagcaaattaggatattcaacttctcaagcatttactgatatgatgctagcagacaacacagagtatcgagctgctgatagtgtttcattctatgacttttcgacatcttccagattacctagaaaacatcttagtagtagtagtgaagcttctccaacgacagaaggagtgtcttcatcttcatctggagaaactgatgagaaaacagaagaagaactagacaatggcggaatcatttatgctagagagaaactaactatctcagaatctcaggactctctctctaatcaaagcatagaactccatgacaatagtattttcttcggagaaggtgaagttatctttgatcacagagttgccctcaaaaacggaggagctatttatggagagaaagaggtagtctttgaaaacataaaatctctactagtagaagtaaatatcgcggtcgagaaagggggtagcgtctatgcaaaagaacgagtatctttagaaaatgttaccgaagcaaccttctcctccaatggtggggaacaaggtggtggtggaatctattcagaacaggatatgttaatcagtgattgcaacaatgtacatttccaagggaatgctgcaggagcaacagcagtaaaacaatgtctggatgaagaaatgatcgtattgctcgcagaatgcgttgatagcttatccgaagatacactggatagcactccagaaacggaacagactgagtcaaatggaaatcaagacggttcgtctgaaacagaagatacacaagtatcagaatcaccagaatcaactcctagccccgacgatgttttaggtaaaggtggtggtatctatacagaaaaatctttgaccatcactggaattacagggactatagattttgtcagtaacatagctaccgattctggagcaggtgtattcactaaagaaaacttgtcttgcaccaacacgaatagcctacagtttttgaaaaactcggcaggtcaacatggaggaggagcctacgttactcaaaccatgtctgttactaatacaactagtgaaagtataactactccccctctcataggagaagtgattttctctgaaaatacagctaaagggcacggtggtggtatctgcactaacaaactttctttatctaatttaaaaacggtgactctcactaaaaactctgcaaaggagtctggaggagctatttttacagatctggcgtctataccaataacagataccccagaatcttctaccccctcttcctcctcgcctgcaagcactcctgaagtagttgcttctgctaaaataaatcgattctttgcctctacggcaaaaccggcagccccttctctaacagaggctgagtctgatcaaacggatcaaacagaaacttctgatactaatagcgatatagacgtgtcgattgagaacattttgaatgtcgctatcaatcaaaacacttctgcgaaaaaaggaggggctatttacgggaaaaaagctaaactttcccgtattaacaatcttgaactttcagggaattcatcccaggatgtaggaggaggtctctgtttaactgaaagcgtagaatttgatgcaattggatcgctcttatcccactataactctgctgctaaagaaggtggggctattcattctaaaacggttactctatctaacctcaagtctaccttcacttttgcagataacactgttaaagcaatagtagaaagcactcctgaagctccagaagagattcctccagtagaaggagaagagtctacagcaacagaagatccaaattctaatacagaaggaagttcggctaacactaaccttgaaggatctcaaggggatactgctgatacagggactggtgatgttaacaatgagtctcaagacacatcagatactggaaacgctgaatctgaagaacaactacaagattctacacaatctaatgaagaaaatacccttcccaatagtaatattgatcaatctaacgaaaacacagacgaatcatctgatagccacactgaggaaataactgacgagagtgtctcatcgtcctctgaaagtggatcatctactcctcaagatggaggagcagcttcttcaggggctccctcaggagatcaatctatctctgcaaacgcttgtttagctaaaagctatgctgcgagtactgatagctcccccgtatctaattcttcaggttcagaagagcctgtcacttcttcttcagattcagacgttactgcatcttctgataatccagactcttcctcatctggagatagcgctggagactctgaagaaccgactgagccagaagctggttctacaacagaaactcttactttaataggaggaggtgctatctatggagaaactgttaagattgagaacttctctggccaaggaatattttctggaaacaaagctatcgataacaccacagaaggctcctcttccaaatctgacgtcctcggaggtgcggtctatgctaaaacattgtttaatctcgatagcgggagctctagacgaactgtcaccttctccgggaatactgtctcttctcaatctacaacaggtcaggttgctggaggagctatctactctcctactgtaaccattgctactcctgtagtattttctaaaaactctgcaacaaacaatgctaataacactacagatactcagagaaaagacacctttggaggagctatcggagctacttctgctgtttctctatcaggaggggctcatttcttagaaaacgttgctgacctcggatctgctattgggttggtgccaggcacacaaaatacagaaacagtgaaattagagtctggctcctactactttgaaaaaaataaagctttaaaacgagctactatttacgcacctgtcgtttccattaaagcctatactgcgacatttaaccaaaacagatctctagaagaaggaagcgcgatttactttacaaaagaagcatctattgagtctttaggctctgttctcttcacaggaaacttagtaaccctaacgctaagcacaactacagaaggcacaccagccacaacctcaggagatgtaacaaaatatggtgctgctatctttggacaaatagcaagctcaaacggatctcagacggataaccttcccctgaaactcattgcttcaggaggaaatatttgtttccgaaacaatgaataccgtcctacttcttctgataccggaacctctactttctgtagtattgcgggagatgttaaattaaccatgcaagctgcaaaagggaaaacgatcagtttctttgatgcaatccggacctctactaagaaaacaggtacacaggcaactgcctacgatactctcgatattaataaatctgaggattcagaaactgtaaactctgcgtttacaggaacgattctgttctcctctgaattacatgaaaataaatcctatattccacaaaacgtagttctacacagtggatctcttgtattgaagccaaataccgagcttcatgttatttcttttgagcagaaagaaggctcttctctcgttatgacacctggatctgttctttcgaaccagactgttgctgatggagctttggtcataaataacatgaccattgatttatccagcgtagagaaaaatggtattgctgaaggaaatatctttactcctccagaattgagaatcatagacactactacaggtggaagcggtggaaccccatctacagatagtgaaagtaaccagaatagtgatgataccgaggagcaaaataataatgacgcctcgaatcaaggagaaagcgcgaatggatcgtcttctcctgcagtagctgctgcacacacatctcgtacaagaaactttgccgctgcagctacagccacacctacgacaacaccaacggctacaactacaacaagcaaccaagtaatcctaggaggagaaattaaactcatcgatcctaatgggaccttcttccagaaccctgcattaagatccgaccaacaaatctccttgttagtgctccctacagactcatcaaaaatgcaagctcagaaaatagtactgacgggtgatattgctcctcagaaaggatatacaggaacactcactctggatcctgatcaactacaaaatggaacgatctcagtgctctggaaatttgactcttatagacaatgggcttatgtacctagagacaatcatttctatgcgaactcgattctgggatctcaaatgttaatggtcacagtcaaacaaggcttgctcaacgataaaatgaatctagctcgctttgaggaagttagctataacaacctgtggatatcaggactaggaacgatgctatcgcaagtaggaacacctacttctgaagaattcacttattacagcagaggagcttctgttgccttagatgctaaaccagcccatgatgtgattgttggagctgcatttagtaagatgatcgggaaaacaaaatccttgaaaagagagaataactacactcacaaaggatccgaatattcttaccaagcatcggtatacggaggcaaaccattccactttgtaatcaataaaaaaacggaaaaatcgctaccgctattgttacaaggagtcatctcttacggatatatcaaacatgatacagtgactcactatccaacgatccgtgaacgaaacaaaggagaatgggaagacttaggatggctgacagctctccgtgtctcctctgtcttaagaactcctgcacaaggggatactaaacgtatcactgtttacggagaattggaatactccagtatccgtcagaaacaattcacagaaacagaatacgatcctcgttacttcgacaactgcacctatagaaacttagcaattcctatggggttagcattcgaaggagagctctctggtaacgatattttgatgtacaacagattctctgtagcatacatgctatcaatctatcgaaattctccaacatgcaaataccaagtgctctcttcaggagaaggcggagaaattatttgtggagtaccgacaagaaactcagctcgcggagaatacagcacgcagctgtacctgggacctttgtggactctgtatggatcctacacgatagaagcagacgcacatacactagctcatatgatgaactgcggtgctcgtatgacattctaa

SEQ ID NO: 23 - protein, 408 aa, Chlamydia trachomatis
MMKPLRFGYFFCAIYFTLLQAAFAKEPNSCPDCQNNWKEVTHTDQLPENIIHADDACYHSGYVQALIDMHFLDSCCQVIVENQTAYLFSLPTDDVTRNAIINLIKDLPFIHSVEICQASYQTCHHQGPHGKTSLPEQRSFCTKVCGKEAIWLPQNTILFSPLVADPRQATNSAGIRFNDEVLGKRVGSATFGGDFIFLRLFDISRFHGDMDIGLQGAVFSVFDLDHPEACMVNSDFFVAALCNFAVNKWSYRFRLWHLSSHLGDEFILANQLPPKKRYNRSDEAVDFFASFRYTPQIRVYGGIGYIISRDLTFPEDPLYFEGGIELRPFGLREDNLHAQPVFAMHFRFWEEHDFSIDQTYIVGMEWSKFQDVGRKVRAVLEYHQGFSHEGQFVREECDYYGFRLSYGF

SEQ ID NO: 24 - DNA, 1227 bp, Chlamydia trachomatis
atgatgaaacctctacgtttcggttatttcttttgcgcaatctattttactttgttacaggcagcgtttgctaaagaaccgaattcttgtcccgactgccagaataattggaaagaagtcacccacacggatcaactcccagaaaacatcattcatgctgatgatgcttgttatcactctggttatgtacaggctctcattgatatgcatttcttagatagctgctgccaggtcatcgttgaaaaccaaactgcttacttattttctcttcctacagatgatgttacgcgcaacgccattatcaacctaattaaagaccttccattcattcactccgtagaaatctgccaagcatcctatcaaacctgtcatcatcaaggccctcatggaaagacttctcttccagaacaacgttctttctgtacaaaggtctgtggaaaagaagctatttggttaccacagaataccatcctattctcgcctcttgtagcagatcctagacaagcaactaatagtgcaggtatccgttttaacgacgaagtcttaggaaaacgtgttggctctgctaccttcggtggagatttcatcttcttacgattatttgatatctcccgattccatggagacatggatattggtctccaaggagctgtattctctgttttcgacctggatcatccagaagcttgcatggtcaactctgacttttttgtcgccgctttgtgcaactttgcagtgaacaaatggagctaccgcttcagactatggcatctttcttctcatcttggcgacgaatttattcttgccaaccagttacctcctaaaaaacgttataatcgaagcgatgaagccgtcgatttctttgcttcttttcgttacactccacagatccgtgtttatggaggtattgggtatatcattagtcgagatttaacattccctgaagatcctctttactttgaaggaggtatcgaactacgtcctttcggattacgggaagacaaccttcatgcccaacccgtctttgctatgcattttcgcttttgggaagagcatgacttttctatagaccaaacttatatagtaggcatggagtggtccaaattccaggatgtagggagaaaagtgcgcgctgtattggaataccaccaaggtttctcccacgaaggacaatttgtccgagaagaatgcgattattatggctttcgattaagttatggcttctag

SEQ ID NO: 25 - protein, 792 aa, Chlamydia trachomatis
MLGIRKKTILQLAVLLLLTFSRSSFCSTSEGRMVVESITITTQGENTQNKRAIPKIKTKQGTLFSQADFDEDLRTLSKDFDRVEPIVEFRNGQAVISLILTAKPVIREINISGNEAIPTHKILKTLELYKNDLFDRELFFKNFDALRTLYLKRGYYDSQLSYSHNHNEKEGFIDISIEIKEGRHGRIKKLTISGITRTEASDLGDIVLTKQYSTTTSWFTGAGVYHPDMVEQDLFAITNYFQNKGYADAKVSKEVSTDAKGNITLLIVVDKGPLYTLGHVHIEGFTALSKRLLDKQLLVGPNSLYCPDKIWTGAQKIRSAYARYGYVNTNVDVSFSAHPTLPVYDVTYRVSEGSPYKIGLIKIKGNTHTKHDVILHETSLFPGDTFDRLKLEGTETRLRNTGYFKSVSVYTVRSQLDPLDSNDLYRDVFIEVKETETGNLGLFLGFSSIDHLFGGAEIAESNFDLFGARNFLKKGFKSLRGGGEYLFLKANLGDKVTDYTVKWTKPHFLNTPWILGVELDKSINKALSKDYSVDTYGGNISTTYILNDKLKYGMYYRGSQTSLSLRKKTSSSNRPGPDLDSNKGFVSAAGLNVLYDSIDNPRKPTMGIRSSLNFELSGLGGTYQFTKLTASGSIYRLLTKKGVLKVRAEAKFIKPFGTTTAQGIPVSERFFLGGETTVRGYKPFIIGPKFSPTEPQGGLSSLLLTEEFQYPLISQPCINAFVFLDSGFIGIEEYTIRLKDLCSSAGFGLRFDMMNNVPIMLGWGWPFRPTEILNNEKIDVSQRFFFALGGVF

SEQ ID NO: 26 - DNA, 2379 bp, Chlamydia trachomatis
atgcttggaatacgcaaaaaaacgattctgcaactcgctgttttactgttgctcaccttttcacgaagttctttctgttcaacttcagaaggacgtatggtcgtagagtctatcaccattacgactcaaggagagaatactcaaaataaacgagctattcctaaaataaaaacaaagcaggggacgttgttctctcaagcagattttgatgaagatctaagaacactttcgaaagattttgatcgagtagagcctatcgtagagtttcgtaatggacaagctgtgatctctctgattctgacggcaaaacctgttatcagagagatcaatatttcaggaaatgaagctatccccactcataaaattctgaaaactttagagctttataaaaatgatctttttgatcgggaattattctttaaaaattttgatgcgctaagaactctttatttgaaacgagggtactacgattctcaactctcctattctcataatcataatgagaaagagggctttatcgatatttccatcgagattaaagaaggacgtcacggtcgcataaaaaaattaacgatttcgggaattacgcgaacagaagcatcagacttaggtgacattgttttaactaaacaatactccacaacaacgagctggttcactggtgccggagtgtatcatccggacatggtagagcaagacttatttgctatcacaaattacttccaaaataaaggatatgctgatgctaaagtaagcaaagaggtctctacagatgctaaaggaaacattactttgcttatcgttgtagacaaaggacctttatacacattaggtcacgtacatatagaaggattcacagcgttatccaaaagactgctcgataaacaactattggttggacctaactccttatattgcccagataaaatttggactggagcacaaaagattcgtagcgcatacgctagatatggctacgtgaacactaacgttgatgtctccttctcagcgcaccccactctacctgtttacgatgttacctatcgagtgagtgaaggatctccctacaaaatcgggttaattaaaatcaaagggaacactcatactaagcatgatgtgattttgcatgagactagtcttttccctggagacacttttgatagattaaaactggaaggtacagagactcgtttacgcaacaccggctactttaaaagtgtaagtgtctatacggttcgttcccaattagatcctcttgattctaacgacctttatcgagatgtttttattgaagtcaaagagactgaaacaggaaatcttgggctattcttaggattcagctccattgaccatttatttggaggggcagaaattgcagaaagcaactttgatttatttggagcccgaaactttctcaaaaaaggattcaaatctttaagaggtggtggagaatacctcttcctaaaagctaatttaggagataaggtcaccgattacactgttaaatggacgaaaccacacttcttaaataccccttggattcttggagtagaattagataaatcaattaataaagctttatcaaaagactactctgtggatacctatggagggaatatcagtaccacctacattcttaacgataagttaaaatatgggatgtattaccgtggtagccaaacaagcttaagtttgcgcaaaaaaacgtccagctctaatagacctggaccagatttagatagtaataaaggatttgtttccgcagcgggactcaatgttctctatgattctattgataatcctagaaaacctactatgggaatccgcagctccttaaactttgaattatctggtttaggcggaacttaccaatttactaaactaacagctagtggttctatctatcgcttattaactaaaaaaggtgttttgaaagtccgtgcagaagctaagtttatcaaacctttcggaacaacaactgcacaaggcattcctgtcagcgaacggttcttcttaggaggtgaaaccactgttcgcggttacaaaccttttattattggaccgaaattttctcctactgaaccacaaggaggcttgtcttccctactattaacagaagaatttcaatatcctttgatttctcaaccttgcattaatgcctttgtatttctagattccggattcattgggatagaagagtacactattcgcctgaaagacctttgcagtagcgccggatttggtctacgctttgatatgatgaataatgtgccaattatgctaggctggggttggccgttccgcccaacagaaatcctcaataatgaaaaaattgatgtatctcaaagattcttttttgccttgggaggagtattctag

SEQ ID NO: 27 - protein, 254 aa, Chlamydia trachomatis
MFAFLTSKKVGILPSRWGSSRFPGKPLAKILGKTLVQRSYENALSSQSLDCVVVATDDQRIFDHVVEFGGLCVMTSTSCANGTERVEEVVSRHFPQAEIVVNIQGDEPCLSPTVIDGLVSTLENNPAADMVTSVTETTDPEAILTDHKVKCVFDKNGKALYFSRSAIPHNFKHPTPIYLHIGVYAFRKAFLSEYVKIPPSSLSLAEDLEQLRVLEIGRSIYVHVVQNATGPSVDYPEDITKVEQYLLCLSKASF

SEQ ID NO: 28 - DNA, 765 bp, Chlamydia trachomatis
gtgtttgcgtttttaaccagcaaaaaagtcggcattctcccctctagatggggaagctcccgcttccccggaaaacctctagcaaaaattctagggaaaacccttgttcaaagatcctatgaaaatgccttaagcagtcaatctctagattgcgttgttgtggcaacagatgatcaacgaatttttgaccatgtcgttgaatttggggggctctgtgtcatgactagcacatcttgcgctaacggaactgagcgagtagaagaggttgtgtctcgacattttcctcaagcagagattgttgtgaacatccaaggagacgagccctgtttatctcctaccgtcatagatgggcttgtgagcacgctagagaacaatcctgctgcagatatggtcacatctgttacagaaacaacagaccccgaagcgatattgacagatcacaaagtgaagtgtgttttcgataagaatggcaaagctctttactttagcagaagcgctattcctcacaactttaaacacccaacgcctatttatctgcatattggtgtttatgcttttagaaaagcttttctaagtgaatatgttaaaattcctccttcctcgttaagcctagccgaagatcttgaacaattacgagtattagaaataggtcgttctatctacgttcatgtcgttcagaatgcaacgggcccttctgttgattatcccgaagatataaccaaagtggagcagtatttattatgtctttcaaaagcatctttttga

SEQ ID NO: 29 - protein, 692 aa, Chlamydia trachomatis
MIRQWYGFFLCLLFSYTSCFGVEENSGRATPTVELVSESEQAVEGEVLRIGVLIAIPEGEHIYWKNPGKLGMPLRISWDLPSGCRLLEEHWPTPEIFEEDGVVYFGYKHSTMVVADIRVSKEIETRPLEIKAQVEWLSCGASCLPGSSSRVLVIPIDQGPLIPNSKETFTFSRALAAQPRPLDAAIKISYQPDGLDVFVPAGKADRATQAWFIAENTRDFAYAQEVPLEQATTYIWKLKHPEGNMPKGIGLSGILIFKDDAGKVVASYQVEENQVEQLSALSWRFLSILLMAFIGGILLNIMPCVLPLITLKVFSLIKSAADHHSSSVIGGIGFTLGAIVSFWGLAFCAFLLKVLGQNIGWGFQLQEPMFVAVLIIVFFLFALSSLGVFEMGIICLSLGKKLQEEGGASVRKNQIWGAFFNGMLTTLVTTPCTGPFLGSVFGLVMAVSFVKQLAIFTAIGLGMASPYLLFASFPKMLAILPKPGPWMSTFKQLTGFMLLATATWLIWIFGVETSATAVTILLVGLWLAAVGAWILGRWGTLVSPRNQRLLASVVFIFCILSSLVITSIGVRYFDENVPPAHSSDWQSFSPEKLADLREKGIPVFVNFTAKWCLTCQLNKPLLHANMQAFAAKGVVTLEADWTKKDPKITEELARLGRASVPSYVYYPAGNKAPLILPERLSQSALEEMVFSQ

SEQ ID NO: 30 - DNA, 2079 bp, Chlamydia trachomatis
atgattcggcaatggtatggattttttctttgcttgctgttcagctatacgtcttgttttggtgtagaagaaaatagtggaagagctacgcctacagtagaacttgttagtgaaagcgaacaagctgttgaaggagaagtgcttcgtatcggagtattgattgctattccagaaggagagcatatctactggaaaaatccagggaagcttggaatgcctttgcgcatttcttgggatttgccatcaggatgtaggttgctggaggaacattggccgactccagagattttcgaagaggatggggttgtttattttggttataaacattctacaatggtggttgcggatattcgcgtttctaaagagatagaaacgcgtccattggagataaaagcgcaagttgaatggttgtcttgcggtgcatcttgtctcccaggttcttcgtcaagggttcttgtgattcctatagatcaggggccgttaattcctaatagtaaagagacattcactttttcccgtgcgttagcggctcaacctcgacctttggatgctgccataaagatttcttatcagcctgatggcttagatgtttttgtgccagcagggaaagcggatcgggcaacccaggcatggttcattgctgaaaacacgcgagattttgcttatgctcaagaggttcctcttgagcaagcgactacgtacatatggaagttgaaacatcctgaaggaaatatgcctaagggtattgggttgtcggggattcttatattcaaggatgatgcagggaaagtagtcgcttcgtatcaagtagaagagaatcaagtcgaacagctttcggcattgagctggaggtttctctctattcttcttatggctttcattggtggaatcttattaaacatcatgccctgtgtattgcctctgattactttgaaagtatttagtttaattaaatcggcggcagatcaccattcttcctctgtgattggagggattgggtttactttaggggctattgtaagcttttggggactcgctttttgtgcgtttttgttaaaggttttagggcaaaatattggatggggattccagcttcaagaacccatgtttgttgccgttttaattattgtcttcttcttatttgctctgagttcgttaggcgtttttgagatgggaataatttgtctgagcctagggaaaaaattgcaagaagagggaggggcatcggtaaggaagaatcagatctggggagcttttttcaatgggatgttgactaccctggttacaactccttgcactgggccttttcttggctctgtatttggattagttatggcagtgtcttttgttaagcagctggcaatttttactgctataggattaggaatggcaagtccctatctattatttgcttcttttccgaagatgctagccattttacctaaacctggtccttggatgagtacgtttaaacagttgactgggtttatgttgcttgctactgcaacttggcttatctggatttttggggtagagacgagtgcaaccgctgtaactattcttcttgtaggattgtggttggctgctgtaggtgcatggattctagggagatggggaacccttgtatctccgcgtaatcagcggcttcttgcttccgttgtattcattttctgtattttaagttccttagtgattacctctataggtgtccgttattttgatgagaacgtccctcctgcacatagctctgattggcaatctttttctcccgaaaagctagctgatttacgcgaaaaagggattccagtttttgtaaatttcactgcaaagtggtgtttaacgtgtcaactcaataagcctcttcttcatgccaatatgcaagcttttgctgctaagggcgtagttactttagaagcagattggacgaaaaaagatccaaaaattacagaagaactcgctcgtttaggccgagccagtgtaccttcttatgtgtattaccctgcggggaacaaagctccgcttattcttccagaaagattatcgcaatctgctttggaagagatggttttttctcagtag

SEQ ID NO: 31 - protein, 326 aa, Chlamydia trachomatis
MFRYTLSRSLFFILALFFCSACDSRSMITHGLSGRDANEIVVLLVSKGVAAQKVPQAASSTGGSGEQLWDISVPAAQITEALAILNQAGLPRMKGTSLLDLFAKQGLVPSEMQEKIRYQEGLSEQMATTIRKMDGIVDASVQISFSPEEEDQRPLTASVYIKHRGVLDNPNSIMVSKIKRLVASAVPGLCPENVSVVSDRASYSDITINGPWGLSDEMNYVSVWGIILAKHSLTKFRLVFYFLILLLFILSCGLLWVIWKTHTLISALGGTKGFFDPAPYSQLSFTQNKPAPKETPGAAEGAEAQTASEQPSKENAEKQEENNEDA

SEQ ID NO: 32 - DNA, 981 bp, Chlamydia trachomatis
atgtttcgttatactctttctcgatccttatttttcattttggctcttttcttctgctcggcttgtgatagtcgttccatgattacacacggcttgtcaggacgtgatgctaatgaaatcgtagtgcttctagtcagtaaaggggtcgctgcacagaaagttccccaagcagcgtcctcaacaggaggatctggagaacaactctgggatatttcggttcctgcagcacaaattacagaggctctagctattctgaaccaagctgggcttccaagaatgaaaggaaccagccttcttgatctattcgctaaacaagggctggtcccttctgaaatgcaagaaaaaatccgctaccaagaaggtctttcagaacaaatggctacgaccattagaaagatggacggtatcgtcgatgcgagcgtacagatttccttttctcctgaagaagaagatcaacggccgctaacagcctctgtatatatcaaacacagaggggtattagacaaccctaacagtattatggtgtctaagattaaacgtttagttgcgagtgctgtcccaggactatgtcccgagaacgtttccgtagtcagtgaccgagcttcttatagtgacattactattaatggcccttggggactctccgatgaaatgaattatgtttctgtatgggggatcattctagctaagcattcccttactaaattccgccttgttttctatttcttaattctccttctcttcattctttcctgtgggctactctgggtcatttggaaaacacacacactgatttctgctctgggtggaacaaaaggattctttgatcctgctccttactcacagctctctttcactcagaataagccagctccaaaagaaactcctggagcagcagaaggtgcagaagcgcaaaccgcttcggaacaaccctctaaagaaaacgcagaaaaacaagaagagaataacgaggacgcttaa

SEQ ID NO: 33 - protein, 200 aa, Chlamydia trachomatis
MEKRGVIVHILVCLLTIFGTFSLPAFGAHFLAEEEQFYMDRFVFSGQYPDMETMEIHAERKKRVQFDVTGSFPKLESVVYKGSFGLLRSKIKGECPELSSVNLSCTSCRMDLDFRGEWKKNASIYIRNEQEPITIMLPKDIGVVVYTQVDMNSKVVAEGSLIKRGRGFWKKTFRNSLVGESPVTLTFHVETRNGGVIFLR

SEQ ID NO: 34 - DNA, 603 bp, Chlamydia trachomatis
atggagaagagaggcgttattgtgcatatactagtttgtttgttgacaatcttcggaacgttcagtttacccgctttcggcgcgcattttctcgcggaagaagagcagttttatatggatcggtttgttttctctgggcagtatccagatatggaaactatggaaatccatgcagaaagaaaaaaacgtgtacaatttgatgtgacgggaagcttccctaagttggagagcgtggtttataagggatcttttggattgctgcgttcgaaaataaagggagagtgtccagaactgtcttctgtaaatctttcttgtacctcctgcagaatggatttagattttcgaggggagtggaaaaagaatgcgtctatttatattcgtaatgagcaagagccaattacaattatgttgcctaaagacattggtgtagttgtctatacgcaggttgatatgaatagtaaagtagttgcagagggatcactaatcaagagaggaagaggtttttggaagaaaacttttcggaattctttggtaggagaatcccctgtgacgctaacttttcatgtagagactcgtaatggaggagttatttttctccgttag

SEQ ID NO: 35 - protein, 305 aa, Chlamydia trachomatis
MFDSDHNSIFQSDLCQRLVVHSILLTFLVILLCTSLYPSSAFIVGLLSSACAALGTYEMGAMVRIKFPFSFTRYSALGSAIFIALTCLTARCKMCFPEHIDLLPWFFLFFWTIRLVFKSRHYKLGPIGSTGLALFCMLYVSVPIRLFLHILYGFVHTDTPFVGIWWAIFLIATTKSSDIFGYFFGKAFGKKRIAPVISPNKTVVGFIAGCCGSILVSLLFYSHLPKAFADQIAVPWILIALGTVLGVSGFFGDIIESTFKRDAQIKNSSDLESIGGMLDVLDSLLLSTPIVYAILLITQNRTFLG

SEQ ID NO: 36 - DNA, 918 bp, Chlamydia trachomatis
atgttcgattcggatcataattccatttttcaaagcgatttgtgtcagcgtctggtcgtccactcgattcttcttactttccttgtcattcttctctgtacatctttatatcccagctcagcctttattgtagggcttctttcctccgcttgcgcagctctaggaacatatgagatgggggctatggttagaatcaagtttccattttctttcacacgctatagtgcattaggatccgctattttcattgctctgacctgtcttacagctcgttgtaaaatgtgttttccagagcatatagacctacttccttggttctttctcttcttttggacgattcgcttagtatttaaaagtcgccattataaacttggtcccataggctcaactgggctcgcgttgttttgtatgctttatgtatcagtccctatccgcttgttcctccacattttgtatgggtttgtgcataccgatactccatttgtaggaatttggtgggcgatttttcttatcgctacaacaaaaagctctgatatttttggttacttctttggaaaagcttttgggaaaaaacgcattgcaccagtcattagcccgaacaaaacagtagtaggcttcattgctggttgctgtggatctatcttggttagccttcttttctactcccatcttcctaaagcctttgctgatcagattgcggtgccttggatcctcattgctttaggtactgtgttgggcgttagtggattctttggagacattatcgaatctacgttcaaacgggatgcacagatcaagaacagcagtgatctggagtctatcggaggaatgctagatgtgctagactccttgcttctttcgactcctatcgtttacgctatcctccttatcactcaaaataggacatttttaggatga

SEQ ID NO: 37 - protein, 621 aa, Chlamydia trachomatis
MRRSVCYVTPSVARAGQISTWRFEYSSANFLPEGTLLKFDLGIDGRPIDWEIPSTDLSQPCNTIYLETPSEDIVAAKAVYAPGGYIPTFEFTLPCDVEAGDTFSIILGSSPNFPQEDSSGNGAQLFTQRRKPFSLYVDPSGKGSFEDPDIFTMDIRGNVLKNIRIFAPSYVIKNKRFDITVRFEDEFGNLTNFSPEETHIELSYEHLRENLNWQLFIPETGFVILPNLYFNEPGIYRIQLRNQATKEVFTSAPIKCFAETSSHLLWGLLHGESERVDSEGNIESCLRYFRDDCALNFFATSSFEIQDGLTPETIKTINQTVADFNEEDRFIALSGAQYLSEEPGEGIREVLLMKEPKSPGKHKECKLFPLSKLYKQSTSHELISIPSFTASKKFGYNFNNFHPEFERVVEIYNAWGCSERTEAEGNPFPIKGSIDSENPEGTVLSALKRNLRFGFVAGGLDDRNLYNHFFDSDQQQYSPGLTAVICNKYSRDSLLEALYQRQCYATTGQRIIVNFQITSAPMGSELSTAIKPGLVINRHISGYVAGTAKIASIEIIRNEDILHTFHPDGNNFEYEYDDLSPFAQVTLKDPQNGAPFAFYYLRVTQENGAMAWSSPIWIDLN

SEQ ID NO: 38 - DNA, 1866 bp, Chlamydia trachomatis
atgcgcagatctgtttgttacgttactccttcagttgctagggctggtcaaatttctacctggcgtttcgaatattcttcagctaatttccttcccgaaggcacattgctaaaatttgacctgggaatagacggacgccctatagactgggagattccttctacagatctttctcaaccatgtaatacaatttatttagaaacgccttccgaggatattgtggctgcaaaagctgtgtatgctcccggaggctatatccctactttcgaatttactctcccttgtgatgtggaagctggggacactttctctattattcttggctcctctcccaacttccctcaagaggactcttcaggtaatggtgctcaattatttactcaacgccgtaaacctttctctctttatgttgacccatcagggaaaggatcttttgaagatcccgatatcttcacaatggatatcagaggaaatgtattaaaaaatatccggatttttgctccttcttatgtgatcaaaaacaaacgctttgatattacagtgcgcttcgaagatgaatttgggaacttaaccaatttctccccagaagagacccatatcgagctttcgtacgaacatcttcgcgaaaacctcaattggcaattgttcatccctgaaacaggctttgtgatccttccaaacctgtatttcaatgaaccaggtatttatcgtattcaactacgcaatcaagcaacaaaagaggtctttacatcagcgcctatcaaatgttttgcagaaacctcctctcatcttttgtgggggcttctacatggagaatctgaacgtgtcgactctgaaggtaatatcgagtcttgcttgcgttattttcgtgatgactgcgcgttaaacttttttgcaacatcctctttcgaaattcaagatggcctgaccccagaaaccattaaaaccattaaccaaaccgttgctgattttaatgaagaagatcgtttcattgccttatccggagcacagtacctttctgaagagcctggcgagggaattcgtgaagtattgctgatgaaggaacccaaatccccagggaaacataaagaatgcaaactatttcctttatctaagctatataagcaatcaactagtcatgagttaatctcaatccccagcttcactgcttcaaagaaatttggatacaattttaataatttccatcctgaatttgaaagagttgttgaaatttataatgcctggggatgctctgaaagaactgaagctgaaggaaaccctttccctattaaaggttctatcgactcagaaaatccagagggaactgttctatctgctttaaagagaaacctgcgttttggattcgtagccggtggtcttgatgatagaaatctatacaatcacttttttgattccgatcaacagcaatactcccctggattaacagctgtgatctgcaataaatattctcgggattccttactcgaggcattataccaacgacaatgctatgctacaaccggccaaagaattatcgtgaatttccagattacatctgctcctatgggctccgaactctccacagccattaaaccagggctcgtgatcaatagacatatttcgggatatgtagcaggaactgccaagattgcgtcgatcgaaatcatccgcaatgaggatattctccataccttccacccagatggaaataactttgagtatgagtacgacgatctctctccttttgcacaagtcactctaaaagatcctcaaaatggagctccttttgctttttactacttacgagtcactcaagagaatggagctatggcttggagctctcctatttggatagatcttaactaa

SEQ ID NO: 39 - protein, 697 aa, Chlamydia trachomatis
MKRFFPLFIGVLLAHTLPSEGLSHQQAVQKKISYLSHFKGITGIMDVEDGVLHIHDDLRLQANKAYVENRTDCGIKIVAHGNVMVNYRGKILICDYLEYYEDTDSCLLTNGRCSLYPWFIGGSTITISPSSIIIHKGYISTSEGPQKHICLSGDYLKYSSDSVLSMGPSRLSICNTPVLLLPQISIMPMEIPKPPITFRGGSGGFLGSYLGVSYSPISKKHCSTTLFLDGFFKHGIGLGYNMRFSSQENPSNAINIKSYYAHRLAIDSSGAKDRYRLHGDFTFSKERAHLAGEFHLSDSWETVVDIFPNNFSLKNTGPTEVSLSWRDNNLFGKMTSSVKVNSFQNVKQELPQAILHHRPVRIRRSRIFLENRLEAGFLDFHFSSNIPGSNFSSWRFSSAHKVYRGLVLPIGTLTPSLSGTAIYYTRMLSPNAAHCQLSGSLSFDYRVALQKEYRHARHIVEPFCSFLKTTRPVLSSDEPHIFSIKDAFHSINLLHVGLESKVLNKHSTPSHLKLWTTYIFDEPHAKDTFPKTACWFSLPLTLQNTLSLDAEWIWKKSRWDHLNVIWEWILNDNLGLTLEFLHRSKYGFIKCAKDNYTLDVSRSLDTLLASPLSDRRNLITGKLFVRPHPHWNYNLNLRYGWHRPDSPSYLEYQMILGHKIFEHWQLFSVYEKREADKRCFFYLKLDKRKQKHRHPFG

SEQ ID NO: 40 - DNA, 2094 bp, Chlamydia trachomatis
gtgaaacgatttttcccactttttattggagtgctgctcgcgcacactttgccgtcagaaggtctttctcatcaacaagctgtccaaaaaaaaatttcttatctgagccattttaaaggcattacaggaattatggatgttgaggatggggtattacatatccatgatgatctacgtcttcaagccaataaagcctatgttgaaaatcgcacggattgtgggatcaaaatcgttgctcatggcaacgttatggtcaattatcgcgggaaaattttaatctgtgattatcttgagtactatgaagatacagattcttgtttactcaccaatggccgctgttcgttatacccatggttcattggaggatccactataacgatctcaccatcttctattatcattcataaagggtatatctcgacttctgaaggtcctcagaaacatatttgtttatccggagattatttaaaatactcttcagacagcgtattatctatgggaccctcacgtctttctatctgtaatacgcctgtgttattgcttcctcaaatctccattatgcctatggagattcctaagcctccgattacttttcgaggtgggagtggaggatttctgggatcctacttaggtgttagttattcccctatatctaaaaagcattgttctacgactttgttcttggatggtttttttaaacatggaataggtctcggctataacatgcgcttttcctctcaggaaaatccaagcaatgccataaatattaaaagctattacgcacatcgattagctattgattcatcaggagcaaaagatcgctatcgattacatggagacttcactttttccaaagaacgagcccatcttgctggtgaattccatttaagtgatagctgggaaacagttgtggatatcttcccaaataacttctctttaaaaaatacaggccctacagaagttagcctatcatggcgcgataacaatttatttgggaaaatgacttcctctgtcaaagtcaactcctttcaaaatgttaaacaagaattgcctcaagcaattcttcatcaccgaccagtacgtatcaggcgctctcgcattttcctagagaatcgcttagaagctggttttttagattttcatttcagtagtaatattccaggctctaacttctcatcatggaggttctcatccgctcacaaagtctaccgtgggcttgttcttcctataggaacgttaaccccttcgctatctggaactgctatctactatacccgcatgctctccccaaatgcagcccattgtcaattatctggatcgctatcttttgattatcgcgttgctttacaaaaagaatatcggcatgcaagacatattgtagagcctttttgctcctttttaaaaaccactcgtcctgtattatcctctgatgagcctcatattttctcgattaaagatgcttttcactctatcaaccttctacatgtaggattggagtcaaaagtcttaaacaaacattccactccttcgcatctgaaactatggacgacctatatctttgatgaacctcacgctaaggacactttccctaaaactgcttgctggttctctcttcctcttacactccaaaatactttatccttagatgcggaatggatttggaaaaaaagccgatgggatcatctcaatgtaatctgggaatggattttgaatgataatctcggtcttactttagaatttttacatagaagtaagtatggctttattaagtgcgctaaagataactacacactcgatgtaagccgatctttagacacattactagcctctcctctttccgatcgaagaaatttgattactggcaaactttttgttcgtccacatcctcattggaattataatcttaatcttcgttatggatggcatcgtccagactctccatcctatttagaataccagatgattctgggtcataaaatctttgagcactggcagctattctctgtctacgaaaaacgtgaagctgataagcgctgcttcttttatctaaaattagataaacgaaaacagaaacaccgccatccttttggataa

SEQ ID NO: 41 - protein, 1016 aa, Chlamydia trachomatis
MPFSLRSTSFCFLACLCSYSYGFASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVSRDVGFVASLHALGDYILNYTQDDRDGFLARYGGFQATAASHYENGSIFGVAFGQLYGQTKSRMYYSKDAGNMTMLSCFGRSYVDIKGTETVMYWETAYGYSVHRMHTQYFNDKTQKFDHSKCHWHNNNYYAFVGAEHNFLEYCIPTRQFARDYELTGFMRFEMAGGWSSSTRETGSLTRYFARGSGHNMSLPIGIVAHAVSHVRRSPPSKLTLNMGYRPDIWRVTPHCNMEIIANGVKTPIQGSPLARHAFFLEVHDTLYIHHFGRAYMNYSLDARRRQTAHFVSMGLNRIF

SEQ ID NO: 42 - DNA, 3051 bp, Chlamydia trachomatis
atgcctttttctttgagatctacatcattttgttttttagcttgtttgtgttcctattcgtatggattcgcgagctctcctcaagtgttaacacctaatgtaaccactccttttaagggggacgatgtttacttgaatggagactgcgcttttgtcaatgtctatgcaggggcagagaacggctcaattatctcagctaatggcgacaatttaacgattaccggacaaaaccatacattatcatttacagattctcaagggccagttcttcaaaattatgccttcatttcagcaggagagacacttactctgaaagatttttcgagtttgatgttctcgaaaaatgtttcttgcggagaaaagggaatgatctcagggaaaaccgtgagtatttccggagcaggcgaagtgattttttgggataactctgtggggtattctcctttgtctattgtgccagcatcgactccaactcctccagcaccagcaccagctcctgctgcttcaagctctttatctccaacagttagtgatgctcggaaagggtctattttttctgtagagactagtttggagatctcaggcgtcaaaaaaggggtcatgttcgataataatgccgggaattttggaacagtttttcgaggtaatagtaataataatgctggtagtgggggtagtgggtctgctacaacaccaagttttacagttaaaaactgtaaagggaaagtttctttcacagataacgtagcctcctgtggaggcggagtagtctacaaaggaactgtgcttttcaaagacaatgaaggaggcatattcttccgagggaacacagcatacgatgatttagggattcttgctgctactagtcgggatcagaatacggagacaggaggcggtggaggagttatttgctctccagatgattctgtaaagtttgaaggcaataaaggttctattgtttttgattacaactttgcaaaaggcagaggcggaagcatcctaacgaaagaattctctcttgtagcagatgattcggttgtctttagtaacaatacagcagaaaaaggcggtggagctatttatgctcctactatcgatataagcacgaatggaggatcgattctatttgaaagaaaccgagctgcagaaggaggcgccatctgcgtgagtgaagcaagctctggttcaactggaaatcttactttaagcgcttctgatggggatattgttttttctgggaatatgacgagtgatcgtcctggagagcgcagcgcagcaagaatcttaagtgatggaacgactgtttctttaaatgcttccggactatcgaagctgatcttttatgatcctgtagtacaaaataattcagcagcgggtgcatcgacaccatcaccatcttcttcttctatgcctggtgctgtcacgattaatcagtccggtaatggatctgtgatttttaccgccgagtcattgactccttcagaaaaacttcaagttcttaactctacttctaacttcccaggagctctgactgtgtcaggaggggagttggttgtgacggaaggagctaccttaactactgggaccattacagccacctctggacgagtgactttaggatccggagcttcgttgtctgccgttgcaggtgctgcaaataataattatacttgtacagtatctaagttggggattgatttagaatcctttttaactcctaactataagacggccatactgggtgcggatggaacagttactgttaacagcggctctactttagacctagtgatggagagtgaggcagaggtatatgataatccgctttttgtgggatcgctgacaattccttttgttactctatcttctagtagtgctagtaacggagttacaaaaaattctgtcactattaatgatgcagacgctgcgcactatgggtatcaaggctcttggtctgcagattggacgaaaccgcctctggctcctgatgctaaggggatggtacctcctaataccaataacactctgtatctgacatggagacctgcttcgaattacggtgaatatcgactggatcctcagagaaagggagaactagtacccaactctctttgggtagcgggatctgcattaagaacctttactaatggtttgaaagaacactatgtttctagagatgttggatttgtagcatctctgcatgctctcggggattatattttgaattatacgcaagatgatcgggatggctttttagctagatatgggggattccaggcgaccgcagcctcccattatgaaaatgggtcaatatttggagtggcttttggacaactctatggtcagacaaagagcagaatgtattactctaaagatgctgggaacatgacgatgttgtcctgtttcggaagaagttacgtagatattaaaggaacagaaactgttatgtattgggagacggcttatggctattctgtgcacagaatgcatacgcagtattttaatgacaaaacgcagaagttcgatcattcgaaatgtcattggcacaacaataactattatgcgtttgtgggtgccgagcataatttcttagagtactgcattcctactcgtcagttcgctagagattatgagcttacagggtttatgcgttttgaaatggccggaggatggtccagttctacacgagaaactggctccctaactagatatttcgctcgcgggtcagggcataatatgtcgcttccaataggaattgtagctcatgcagtttctcatgtgcgaagatctcctccttctaaactgacactaaatatgggatatagaccagacatttggcgtgtcactccacattgcaatatggaaattattgctaacggagtgaagacacctatacaaggatctccgctggcacggcatgccttcttcttagaagtgcatgatactttgtatattcatcattttggaagagcctatatgaactattcgctggatgctcgtcgtcgacaaacggcacattttgtatccatgggcttgaatagaatcttttaa

SEQ ID NO: 43 - protein, 553 aa, Chlamydia trachomatis
MRIGDPMNKLIRRAVTIFAVTSVASLFASGVLETSMAESLSTNVISLADTKAKDNTSHKSKKARKNHSKETPVDRKEVAPVHESKATGPKQDSCFGRMYTVKVNDDRNVEITQAVPEYATVGSPYPIEITATGKRDCVDVIITQQLPCEAEFVRSDPATTPTADGKLVWKIDRLGQGEKSKITVWVKPLKEGCCFTAATVCACPEIRSVTKCGQPAICVKQEGPENACLRCPVVYKINIVNQGTATARNVVVENPVPDGYAHSSGQRVLTFTLGDMQPGEHRTITVEFCPLKRGRATNIATVSYCGGHKNTASVTTVINEPCVQVSIAGADWSYVCKPVEYVISVSNPGDLVLRDVVVEDTLSPGVTVLEAAGAQISCNKVVWTVKELNPGESLQYKVLVRAQTPGQFTNNVVVKSCSDCGTCTSCAEATTYWKGVAATHMCVVDTCDPVCVGENTVYRICVTNRGSAEDTNVSLMLKFSKELQPVSFSGPTKGTITGNTVVFDSLPRLGSKETVEFSVTLKAVSAGDARGEAILSSDTLTVPVSDTENTHIY

SEQ ID NO: 44 - DNA, 1662 bp, Chlamydia trachomatis
atgcgaataggagatcctatgaacaaactcatcagacgagcagtgacgatcttcgcggtgactagtgtggcgagtttatttgctagcggggtgttagagacctctatggcagagtctctctctacaaacgttattagcttagctgacaccaaagcgaaagacaacacttctcataaaagcaaaaaagcaagaaaaaaccacagcaaagagactcccgtagaccgtaaagaggttgctccggttcatgagtctaaagctacaggacctaaacaggattcttgctttggcagaatgtatacagtcaaagttaatgatgatcgcaatgttgaaatcacacaagctgttcctgaatatgctacggtaggatctccctatcctattgaaattactgctacaggtaaaagggattgtgttgatgttatcattactcagcaattaccatgtgaagcagagttcgtacgcagtgatccagcgacaactcctactgctgatggtaagctagtttggaaaattgaccgcttaggacaaggcgaaaagagtaaaattactgtatgggtaaaacctcttaaagaaggttgctgctttacagctgcaacagtatgcgcttgtccagagatccgttcggttacaaaatgtggacaacctgctatctgtgttaaacaagaaggcccagagaatgcttgtttgcgttgcccagtagtttacaaaattaatatagtgaaccaaggaacagcaacagctcgtaacgttgttgttgaaaatcctgttccagatggttacgctcattcttctggacagcgtgtactgacgtttactcttggagatatgcaacctggagagcacagaacaattactgtagagttttgtccgcttaaacgtggtcgtgctaccaatatagcaacggtttcttactgtggaggacataaaaatacagcaagcgtaacaactgtgatcaacgagccttgcgtacaagtaagtattgcaggagcagattggtcttatgtttgtaagcctgtagaatatgtgatctccgtttccaatcctggagatcttgtgttgcgagatgtcgtcgttgaagacactctttctcccggagtcacagttcttgaagctgcaggagctcaaatttcttgtaataaagtagtttggactgtgaaagaactgaatcctggagagtctctacagtataaagttctagtaagagcacaaactcctggacaattcacaaataatgttgttgtgaagagctgctctgactgtggtacttgtacttcttgcgcagaagcgacaacttactggaaaggagttgctgctactcatatgtgcgtagtagatacttgtgaccctgtttgtgtaggagaaaatactgtttaccgtatttgtgtcaccaacagaggttctgcagaagatacaaatgtttctttaatgcttaaattctctaaagaactgcaacctgtatccttctctggaccaactaaaggaacgattacaggcaatacagtagtattcgattcgttacctagattaggttctaaagaaactgtagagttttctgtaacattgaaagcagtatcagctggagatgctcgtggggaagcgattctttcttccgatacattgactgttccagtttctgatacagagaatacacacatctattaa

SEQ ID NO: 45 - protein, 1751 aa, Chlamydia trachomatis
MKWLSATAVFAAVLPSVSGFCFPEPKELNFSRVGTSSSTTFTETVGEAGAEYIVSGNASFTKFTNIPTTDTTTPTNSNSSSSNGETASVSEDSDSTTTTPDPKGGGAFYNAHSGVLSFMTRSGTEGSLTLSEIKITGEGGAIFSQGELLFTDLTGLTIQNNLSQLSGGAIFGESTISLSGITKATFSSNSAEVPAPVKKPTEPKAQTASETSGSSSSSGNDSVSSPSSSRAEPAAANLQSHFICATATPAAQTDTETSTPSHKPGSGGAIYAKGDLTIADSQEVLFSINKATKDGGAIFAEKDVSFENITSLKVQTNGAEEKGGAIYAKGDLSIQSSKQSLFNSNYSKQGGGALYVEGDINFQDLEEIRIKYNKAGTFETKKITLPKAQASAGNADAWASSSPQSGSGATTVSNSGDSSSGSDSDTSETVPATAKGGGLYTDKNLSITNITGIIEIANNKATDVGGGAYVKGTLTCENSHRLQFLKNSSDKQGGGIYGEDNITLSNLTGKTLFQENTAKEEGGGLFIKGTDKALTMTGLDSFCLINNTSEKHGGGAFVTKEISQTYTSDVETIPGITPVHGETVITGNKSTGGNGGGVCTKRLALSNLQSISISGNSAAENGGGAHTCPDSFPTADTAEQPAAASAATSTPESAPVVSTALSTPSSSTVSSLTLLAASSQASPATSNKETQDPNADTDLLIDYVVDTTISKNTAKKGGGIYAKKAKMSRIDQLNISENSATEIGGGICCKESLELDALVSLSVTENLVGKEGGGLHAKTVNISNLKSGFSFSNNKANSSSTGVATTASAPAAAAASLQAAAAAVPSSPATPTYSGVVGGAIYGEKVTFSQCSGTCQFSGNQAIDNNPSQSSLNVQGGAIYAKTSLSIGSSDAGTSYIFSGNSVSTGKSQTTGQIAGGAIYSPTVTLNCPATFSNNTASMATPKTSSEDGSSGNSIKDTIGGAIAGTAITLSGVSRFSGNTADLGAAIGTLANANTPSATSGSQNSITEKITLENGSFIFERNQANKRGAIYSPSVSIKGNNITFNQNTSTHDGSAIYFTKDATIESLGSVLFTGNNVTATQASSATSGQNTNTANYGAAIFGDPGTTQSSQTDAILTLLASSGNITFSNNSLQNNQGDTPASKFCSIAGYVKLSLQAAKGKTISFFDCVHTSTKKIGSTQNVYETLDINKEENSNPYTGTIVFSSELHENKSYIPQNAILHNGTLVLKEKTELHVVSFEQKEGSKLIMKPGAVLSNQNIANGALVINGLTIDLSSMGTPQAGEIFSPPELRIVATTSSASGGSGVSSSIPTNPKRISAAAPSGSAATTPTMSENKVFLTGDLTLIDPNGNFYQNPMLGSDLDVPLIKLPTNTSDVQVYDLTLSGDLFPQKGYMGTWTLDSNPQTGKLQARWTFDTYRRWVYIPRDNHFYANSILGSQNSMIVVKQGLINNMLNNARFDDIAYNNFWVSGVGTFLAQQGTPLSEEFSYYSRGTSVAIDAKPRQDFILGAAFSKMVGKTKAIKKMHNYFHKGSEYSYQASVYGGKFLYFLLNKQHGWALPFLIQGVVSYGHIKHDTTTLYPSIHERNKGDWEDLGWLADLRISMDLKEPSKDSSKRITVYGELEYSSIRQKQFTEIDYDPRHFDDCAYRNLSLPVGCAVEGAIMNCNILMYNKLALAYMPSIYRNNPVCKYRVLSSNEAGQVICGVPTRTSARAEYSTQLYLGPFWTLYGNYTIDVGMYTLSQMTSCGARMIF

SEQ ID NO: 46 - DNA, 5256 bp, Chlamydia trachomatis
atgaaatggctgtcagctactgcggtgtttgctgctgttctcccctcagtttcagggttttgcttcccagaacctaaagaattaaatttctctcgcgtaggaacttcttcctctaccacttttactgaaacagttggagaagctggggcagaatatatcgtctctggtaacgcatctttcacaaaatttaccaacattcctactaccgatacaacaactcccacgaactcaaactcctctagctctaacggagagactgcttccgtttctgaggatagtgactctacaacaacgactcctgatcctaaaggtggcggcgccttttataacgcgcactccggagttttatcctttatgacacgatcaggaacagaaggttccttaactctgtctgagataaaaataactggtgaaggcggtgctatcttctctcaaggagagctgctatttacagatctgacaggtctaaccatccaaaataacttatcccagctatccggaggagcgatttttggagaatctacaatctccctatcagggattactaaagcgactttctcctccaactctgcagaagttcctgctcctgttaagaaacctacagaacctaaagctcaaacagcaagcgaaacgtcgggttctagtagttctagcggaaatgattcggtgtcttcccccagttccagtagagctgaacccgcagcagctaatcttcaaagtcactttatttgtgctacagctactcctgctgctcaaaccgatacagaaacatcaactccctctcataagccaggatctgggggagctatctatgctaaaggcgaccttactatcgcagactctcaagaggtactattctcaataaataaagctactaaagatggaggagcgatctttgctgagaaagatgtttctttcgagaatattacatcattaaaagtacaaactaacggtgctgaagaaaagggaggagctatctatgctaaaggtgacctctcaattcaatcttctaaacagagtctttttaattctaactacagtaaacaaggtggtggggctctatatgttgaaggagatataaacttccaagatcttgaagaaattcgcattaagtacaataaagctggaacgttcgaaacaaaaaaaatcactttaccaaaagctcaagcatctgcaggaaatgcagatgcttgggcctcttcctctcctcaatctggttctggagcaactacagtctccaactcaggagactctagctctggctcagactcggatacctcagaaacagttccagccacagctaaaggcggtgggctttatactgataagaatctttcgattactaacatcacaggaattatcgaaattgcaaataacaaagcgacagatgttggaggtggtgcttacgtaaaaggaacccttacttgtgaaaactctcaccgtctacaatttttgaaaaactcttccgataaacaaggtggaggaatctacggagaagacaacatcaccctatctaatttgacagggaagactctattccaagagaatactgccaaagaagagggcggtggactcttcataaaaggtacagataaagctcttacaatgacaggactggatagtttctgtttaattaataacacatcagaaaaacatggtggtggagcctttgttaccaaagaaatctctcagacttacacctctgatgtggaaacaattccaggaatcacgcctgtacatggtgaaacagtcattactggcaataaatctacaggaggtaatggtggaggcgtgtgtacaaaacgtcttgccttatctaaccttcaaagcatttctatatccgggaattctgcagctgaaaatggtggtggagcccacacatgcccagatagcttcccaacggcggatactgcagaacagcccgcagcagcttctgccgcgacgtctactcccgagtctgccccagtggtctcaactgctctaagcacaccttcatcttctaccgtctcttcattaaccttactagcagcctcttcacaagcctctcctgcaacctctaataaggaaactcaagatcctaatgctgatacagacttattgatcgattatgtagttgatacgactatcagcaaaaacactgctaagaaaggcggtggaatctatgctaaaaaagccaagatgtcccgcatagaccaactgaatatctctgagaactccgctacagagataggtggaggtatctgctgtaaagaatctttagaactagatgccctagtctccttatctgtaacagagaaccttgttgggaaagaaggtggaggcttacatgctaaaactgtaaatatttctaatctgaaatcaggcttctctttctcgaacaacaaagcaaactcctcatccacaggagtcgcaacaacagcttcagcacctgctgcagctgctgcttccctacaagcagccgcagcagccgtaccatcatctccagcaacaccaacttattcaggtgtagtaggaggagctatctatggagaaaaggttacattctctcaatgtagcgggacttgtcagttctctgggaaccaagctatcgataacaatccctcccaatcatcgttgaacgtacaaggaggagccatctatgccaaaacctctttgtctattggatcttccgatgctggaacctcctatattttctcggggaacagtgtctccactgggaaatctcaaacaacagggcaaatagcgggaggagcgatctactcccctactgttacattgaattgtcctgcgacattctctaacaatacagcctctatggctacaccaaagacttcttctgaagatggatcctcaggaaattctattaaagataccattggaggagccattgcagggacagccattaccctatctggagtctctcgattttcagggaatacggctgatttaggagctgcaataggaactctagctaatgcaaatacacccagtgcaactagcggatctcaaaatagcattacagaaaaaattactttagaaaacggttcttttatttttgaaagaaaccaagctaataaacgtggagcgatttactctcctagcgtttccattaaagggaataatattaccttcaatcaaaatacatccactcatgatggaagtgctatctactttacaaaagatgctacgattgagtctttaggatctgttctttttacaggaaataacgttacagctacacaagctagttctgcaacatctggacaaaatacaaatactgccaactatggggcagccatctttggagatccaggaaccactcaatcgtctcaaacagatgccattttaacccttcttgcttcttctggaaacattacttttagcaacaacagtttacagaataaccaaggtgatactcccgctagcaagttttgtagtattgcaggatacgtcaaactctctctacaagccgctaaagggaagactattagctttttcgattgtgtgcacacctctaccaaaaaaataggttcaacacaaaacgtttatgaaactttagatattaataaagaagagaacagtaatccatatacaggaactattgtgttctcttctgaattacatgaaaacaaatcttacatcccacagaatgcaatccttcacaacggaactttagttcttaaagagaaaacagaactccacgtagtctcttttgagcagaaagaagggtctaaattaattatgaaacccggagctgtgttatctaaccaaaacatagctaacggagctctagttatcaatgggttaacgattgatctttccagtatggggactcctcaagcaggggaaatcttctctcctccagaattacgtatcgttgccacgacctctagtgcatccggaggaagcggggtcagcagtagtataccaacaaatcctaaaaggatttctgcagcagcgccttcaggttctgccgcaactactccaactatgagcgagaacaaagttttcctaacaggagaccttactttaatagatcctaatggaaacttttaccaaaaccctatgttaggaagcgatctagatgtaccactaattaagcttccgactaacacaagtgacgtccaagtctatgatttaactttatctggggatcttttccctcagaaagggtacatgggaacctggacattagattctaatccacaaacagggaaacttcaagccagatggacattcgatacctatcgtcgctgggtatacatacctagggataatcatttttatgcgaactctatcttaggctcccaaaactcaatgattgttgtgaagcaagggcttatcaacaacatgttgaataatgcccgcttcgatgatatcgcttacaataacttctgggtttcaggagtaggaactttcttagctcaacaaggaactcctctttccgaagaattcagttactacagccgcggaacttcagttgccatcgatgccaaacctagacaagattttatcctaggagctgcatttagtaagatggtggggaaaaccaaagccatcaaaaaaatgcataattacttccataagggctctgagtactcttaccaagcttctgtctatggaggtaaattcctgtatttcttgctcaataagcaacatggttgggcacttcctttcctaatacaaggagtcgtgtcctatggacatattaaacatgatacaacaacactttacccttctatccatgaaagaaataaaggagattgggaagatttaggatggttagcggatcttcgtatctctatggatcttaaagaaccttctaaagattcttctaaacggatcactgtctatggggaacttgagtattccagcattcgccagaaacagttcacagaaatcgattacgatccaagacacttcgatgattgtgcttacagaaatctgtcgcttcctgtgggatgcgctgtcgaaggagctatcatgaactgtaatattcttatgtataataagcttgcattagcctacatgccttctatctacagaaataatcctgtctgtaaatatcgggtattgtcttcgaatgaagctggtcaagttatctgcggagtgccaactagaacctctgctagagcagaatacagtactcaactatatcttggtcccttctggactctctacggaaactatactatcgatgtaggcatgtatacgctatcgcaaatgactagctgcggtgctcgcatgatcttctaa

SEQ ID NO: 47 - protein, 975 aa, Chlamydia trachomatis
MNRVIEIHAHYDQRQLSQSPNTNFLVHHPYLTLIPKFLLGALIVYAPYSFAEMELAISGHKQGKDRDTFTMISSCPEGTNYIINRKLILSDFSLLNKVSSGGAFRNLAGKISFLGKNSSASIHFKHININGFGAGVFSESSIEFTDLRKLVAFGSESTGGIFTAKEDISFKNNHHIAFRNNITKGNGGVIQLQGDMKGSVSFVDQRGAIIFTNNQAVTSSSMKHSGRGGAISGDFAGSRILFLNNQQITFEGNSAVHGGAIYNKNGLVEFLGNAGPLAFKENTTIANGGAIYTSNFKANQQTSPILFSQNHANKKGGAIYAQYVNLEQNQDTIRFEKNTAKEGGGAITSSQCSITAHNTIIFSDNAAGDLGGGAILLEGKKPSLTLIAHSGNIAFSGNTMLHITKKASLDRHNSILIKEAPYKIQLAANKNHSIHFFDPVMALSASSSPIQINAPEYETPFFSPKGMIVFSGANLLDDAREDVANRTSIFNQPVHLYNGTLSIENGAHLIVQSFKQTGGRISLSPGSSLALYTMNSFFHGNISSKEPLEINGLSFGVDISPSNLQAEIRAGNAPLRLSGSPSIHDPEGLFYENRDTAASPYQMEILLTSDKIVDISKFTTDSLVTNKQSGFQGAWHFSWQPNTINNTKQKILRASWLPTGEYVLESNRVGRAVPNSLWSTFLLLQTASHNLGDHLCNNRSLIPTSYFGVLIGGTGAEMSTHSSEEESFISRLGATGTSIIRLTPSLTLSGGGSHMFGDSFVADLPEHITSEGIVQNVGLTHVWGPLTVNSTLCAALDHNAMVRICSKKDHTYGKWDTFGMRGTLGASYTFLEYDQTMRVFSFANIEATNILQRAFTETGYNPRSFSKTKLLNIAIPIGIGYEFCLGNSSFALLGKGSIGYSRDIKRENPSTLAHLAMNDFAWTTNGCSVPTSAHTLANQLILRYKACSLYITAYTINREGKNLSNSLSCGGYVGF

SEQ ID NO: 48 - DNA, 2928 bp, Chlamydia trachomatis
atgaatcgagttatagaaatccatgctcactacgatcaaagacaactttctcaatctccaaatacaaacttcttagtacatcatccttatcttactcttattcccaagtttctactaggagctctaatcgtctatgctccttattcgtttgcagaaatggaattagctatttctggacataaacaaggtaaagatcgagatacctttaccatgatctcttcctgtcctgaaggcactaattacatcatcaatcgcaaactcatactcagtgatttctcgttactaaataaagtttcatcagggggagcctttcggaatctagcagggaaaatttccttcttaggaaaaaattcttctgcgtccattcattttaaacacattaatatcaatggttttggagccggagtcttttctgaatcctctattgaatttactgatttacgaaaacttgttgcttttggatctgaaagcacaggaggaatttttactgcgaaagaggacatctcttttaaaaacaaccaccacattgccttccgcaataatatcaccaaagggaatggtggcgttatccagctccaaggagatatgaaaggaagcgtatcctttgtagatcaacgtggagctatcatctttaccaataaccaagctgtaacttcttcatcaatgaaacatagtggtcgtggaggagcaattagcggtgacttcgcaggatccagaattctttttcttaataaccaacaaattactttcgaaggcaatagcgctgtgcatggaggtgctatctacaataagaatggccttgtcgagttcttaggaaatgcaggacctcttgcctttaaagagaacacaacaatagctaacgggggagctatatacacaagtaatttcaaagcgaatcaacaaacatcccccattctattctctcaaaatcatgcgaataagaaaggcggagcgatttacgcgcaatatgtgaacttagaacagaatcaagatactattcgctttgaaaaaaataccgctaaagaaggcggtggagccatcacctcttctcaatgctcaattactgctcataataccatcattttttccgataatgctgccggagatcttggaggaggagcaattcttctagaagggaaaaaaccttctctaaccttgattgctcatagtggtaatattgcatttagcggcaataccatgcttcatatcaccaaaaaagcttccctagatcgacacaattctatcttaatcaaagaagctccctataaaatccaacttgcagcgaacaaaaaccattctattcatttctttgatcctgtcatggcattgtcagcatcatcttcccctatacaaatcaatgctcctgagtatgaaactcccttcttctcacctaagggtatgatcgttttctcgggtgcgaatcttttagatgatgctagggaagatgttgcaaatagaacatcgatttttaaccaacccgttcatctatataatggcaccctatctatcgaaaatggagcccatctgattgtccaaagcttcaaacagaccggaggacgtatcagtttatctccaggatcctccttggctctatacacgatgaactcgttcttccatggcaacatatccagcaaagaacccctagaaattaatggtttaagctttggagtagatatctctccttctaatcttcaagcagagatccgtgccggcaacgctcctttacgattatccggatccccatctatccatgatcctgaaggattattctacgaaaatcgcgatactgcagcatcaccataccaaatggaaatcttgctcacctctgataaaattgtagatatctccaaatttactactgattctctagttacgaacaaacaatcaggattccaaggagcctggcattttagctggcagccaaatactataaacaatactaaacaaaaaatattaagagcttcttggctcccaacaggagaatatgtccttgaatccaatcgagtggggcgtgccgttcctaattccttatggagcacatttttacttttacagacagcctctcataacttaggcgatcatctatgtaataatcgatctcttattcctacttcatacttcggagttttaattggaggaactggagcagaaatgtctacccactcctcagaagaagaaagctttatatctcgtttaggagctacaggaacctctatcatacgcttaactccctccctgacactctctggaggaggctcacatatgttcggagattcgttcgttgcagacttaccagaacacatcacttcagaaggaattgttcagaatgtcggtttaacccatgtctggggaccccttactgtcaattctacattatgtgcagccttagatcacaacgcgatggtccgcatatgctccaaaaaagatcacacctatgggaaatgggatacattcggtatgcgaggaacattaggagcctcttatacattcctagaatatgatcaaactatgcgcgtattctcattcgccaacatcgaagccacaaatatcttgcaaagagcttttactgaaacaggctataacccaagaagtttttccaagacaaaacttctaaacatcgccatccccatagggattggttatgaattctgcttagggaatagctcttttgctctactaggtaagggatccatcggttactctcgagatattaaacgagaaaacccatccactcttgctcacctggctatgaatgattttgcttggactaccaatggctgttcagttccaacctctgcacacacattggcaaatcaattgattcttcgctataaagcatgttccttatacatcacggcatatactatcaaccgtgaagggaagaacctctccaatagcttatcctgcggaggctatgttggcttctaa

SEQ ID NO: 49 - protein, 210 aa, Chlamydia trachomatis
MYKYFIVDTSGSQPFLAYVDCRDVLEVWSLPTGPDQGVVLNFIFNSLDLPFQGIGVSVGPGGFSATRVGVAFAQGLSLAKNVPLVGYSSLEGYLSLGQEEEALLLPLGKKGGIVALNSELSLDGFLLTDTTSTPGILLSYSEALEYCLDKGCCHVISPDPTYFVELFSSRISVRKVVPCIDRIRKYVVSQFVLSQNLPLCLDYRSISSFF

SEQ ID NO: 50 - DNA, 633 bp, Chlamydia trachomatis
gtgtacaaatattttattgtagacacttccggttctcagccgtttttggcctatgtcgattgtcgagatgttttagaagtatggtctttgcctacagggccagatcaaggagttgtgttaaatttcattttcaacagcctggatttgcctttccaaggtattggagtctctgttggtcctgggggattttctgcaactagagtgggagttgcttttgctcaagggctctctctggctaaaaatgtccctttagttggctatagctctttagaaggatatctttctttgggtcaagaagaggaggctttgcttttgcctctagggaaaaagggtgggattgtagctttaaactcagagctttctcttgatggttttctgcttacagatactacttctactccgggaattttattgtcttattctgaagctctagagtattgtttagataagggatgttgtcatgtgatctctccggatccaacgtatttcgtagaactattttcttcgcgtatttcggtaaggaaggtggttccttgtatcgatcgaatccgtaagtacgttgtttcgcagtttgttctgtctcaaaatcttccgttgtgtttagattaccggagcatctcttcctttttttag

SEQ ID NO: 51 - protein, 141 aa, Chlamydia trachomatis
MIDVSVVGPVLAMALAMIGSAVGCGMAGVASHAVMSRIDEGHGKIIGLSAMPSSQSIYGLIFMLLLNDAIKDGKVSAVSGIVMGIAVGSALLLSAFMQGKCCVSAIQAYARSSAIYGKSFASIGIVESFALFAFVFALLLF

SEQ ID NO: 52 - DNA, 426 bp, Chlamydia trachomatis
atgatagatgtatcagtagtggggcctgtattggctatggctttggcaatgattggtagcgctgttggatgtggaatggctggagtcgcttctcacgcagtgatgtctcgaatcgatgaaggacacgggaagattattggtctgtctgctatgccctcatcacaatccatttacgggttgattttcatgttactgctgaatgatgcaattaaggatggaaaagtctctgcagtcagtggtatcgtaatgggtatagctgtaggatctgcgttattgctttctgcttttatgcaagggaagtgctgtgtgagtgctattcaagcctatgcgcgttcctctgcaatatatggtaaatcatttgcttcgattgggattgttgagtcttttgcgttatttgctttcgtttttgcgctattgttattctaa

SEQ ID NO: 53 - protein, 101 aa, Chlamydia trachomatis
MAKKSAVAREVKRRKLVEANFQKRAELRKLAKSLSVSEEERERAREALNKMRRDTSPSRLHNRCLLTGRPRGYLRKFAISRICFRQMASMGDIPGVVKASW

SEQ ID NO: 54 - DNA, 306 bp, Chlamydia trachomatis
atggcgaagaagtcagcagtagctagagaagttaaacgtcgaaagttagtagaagctaattttcagaagagagcagagcttcgaaaacttgcgaagagtttatctgttagcgaggaagaaagagaaagagctcgggaagctctcaataaaatgagaagagatacttctccttctcgtttacataatagatgcctattaacaggccgtcctcgtggataccttagaaagtttgctatctcaagaatttgttttagacaaatggcttctatgggagatatcccaggcgttgtgaaagcaagttggtag

SEQ ID NO: 55 - protein, 393 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLALSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFRF

SEQ ID NO: 56 - DNA, 1182 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcgccacttggtgtgacgctatcagcatgcgtgttggttactacggagactttgttttcgaccgtgttttgaaaactgatgtgaataaagaatttcagatgggtgccaagcctacaactgatacaggcaatagtgcagctccatccactcttacagcaagagagaatcctgcttacggccgacatatgcaggatgctgagatgtttacaaatgccgcttgcatggcattgaatatttgggatcgttttgatgtattctgtacattaggagccaccagtggatatcttaaaggaaactctgcttctttcaatttagttggattgtttggagataatgaaaatcaaaaaacggtcaaagcggagtctgtaccaaatatgagctttgatcaatctgttgttgagttgtatacagatactacttttgcgtggagcgtcggcgctcgcgcagctttgtgggaatgtggatgtgcaactttaggagcttcattccaatatgctcaatctaaacctaaagtagaagaattaaacgttctctgcaatgcagcagagtttactattaataaacctaaagggtatgtaggtaaggagtttcctcttgatcttacagcaggaacagatgctgcgacaggaactaaggatgcctctattgattaccatgaatggcaagcaagtttagctctctcttacagactgaatatgttcactccctacattggagttaaatggtctcgagcaagctttgatgccgatacgattcgtatagcccagccaaaatcagctacagctatttttgatactaccacgcttaacccaactattgctggagctggcgatgtgaaaactggcgcagagggtcagctcggagacacaatgcaaatcgtttccttgcaattgaacaagatgaaatctagaaaatcttgcggtattgcagtaggaacaactattgtggatgcagacaaatacgcagttacagttgagactcgcttgatcgatgagagagcagctcacgtaaatgcacaattccgcttctaa

SEQ ID NO: 57 - protein, 194 aa, Chlamydia trachomatis
MLKMFWLNSLVFFSLLLSACGYTVLSPHYVEKKFSLSEGIYVCPIEGDSLGDLVSSLSYELEKRGLHTRSQGTSSGYVLKVSLFNETDENIGFAYTPQKPDEKPVKHFIVSNEGRLALSAKVQLIKNRTQEILVEKCLRKSVTFDFQPDLGTANAHQLALGQFEMHNEAIKSASRILYSQLAETIVQQVYYDLF

SEQ ID NO: 58 - DNA, 585 bp, Chlamydia trachomatis
atgctgaaaatgttttggttgaatagcctcgttttcttctcgttactactatcagcctgcggctatacagtgctctccccccactatgtagaaaagaaattctcgctttccgaaggcatctatgtctgccctatcgaaggagattcattaggagatctcgtatcctctctttcttacgaattagaaaagcgaggactccacacacgatctcaaggaacctcttctggttatgtactcaaagtctctcttttcaatgagactgatgaaaatattggattcgcatacactccccaaaaacctgatgaaaaacctgtaaaacacttcattgtctctaatgaagggcgcttagcgttatcagcaaaagtccaactaatcaaaaaccgcacacaagaaatattagtggagaaatgcctgagaaaatcggttacttttgattttcaacctgacctcggaaccgcgaatgctcatcagctagctctcggacaatttgaaatgcataatgaagcaataaaaagcgcttctcgtatattgtattcgcaattagcagagactattgtacaacaggtatactatgaccttttctga

SEQ ID NO: 59 - protein, 542 aa, Chlamydia trachomatis
MFKLVSYIILSWVLVCLAQPDVSVVASVVSCICGYSLLWAGLFALVEQLSWKKVWCIAFIWTWTVEGAHFSWMLEDLYVGTSIYFVWGILLSYLATLFASFSCLVVWCCRKQYRGALVWLPGVWVAIEAIRYYGLLSGVSFDFIGWPLTATAYGRQFGSFFGWAGQSFLVIAANICCFAVCLLKHSFSKGLWLTLCAFPYLLGGAHYEYLKKHFSDSEVLRVAIVQPGYSPHMHAGRTASAIWRGLVSLCQTIQTPVDVIVFPEVSVPFGLHRQAYTLHENQPVLESLLPNKSWGEFFTNLDWIQAIAERYQCTVIMGMERWENKGGILHLYNAAECVSREGEITSYDKRILVPGGEYIPGGKIGFSLCQTFFPEFALPFQRLPGEFSGVVNITERIKAGISICYEETFGYAIRPYKRQQADILVNLTNDGWYPRSRLPLVHFYHGMLRNQELGIPCIRACRTGVSAAVDSLGRIVGILPWESRTCPVSTGVLQVSVPLYSYHTVYARLGDAPLLLIAVCSVIGAIAYFYRKKKETPPQTFF

SEQ ID NO: 60 - DNA, 1629 bp, Chlamydia trachomatis
gtgtttaaacttgtgtcatacatcatcctttcttgggtgctggtctgtttggctcagccggatgtaagtgttgtagcttctgttgttagttgtatttgcggttacagcttactttgggctgggctttttgctttagtagagcaattatcttggaagaaagtttggtgcatcgcttttatttggacttggactgtcgaaggcgctcatttctcttggatgcttgaagatctttatgtagggacaagcatctattttgtttggggtatactgctttcttatctcgccaccctatttgctagtttttcttgtttggttgtgtggtgttgtcgcaagcaatataggggagctcttgtttggcttccaggggtttgggtggcgatagaagcaatacgctattatgggttgctttcaggagtttcttttgattttattggctggcctcttacagcgacagcctatggccggcaattcggcagcttttttggatgggctggacaaagctttctagttattgctgccaatatatgctgttttgcagtatgtttattaaaacactctttttccaaaggtttgtggttgacgttgtgcgcgttcccttatctgttaggcggagcgcattacgaatacctaaagaagcatttttccgactctgaagtgcttcgagttgccatcgtgcagcctggatatagtcctcatatgcatgcagggaggacggctagtgctatttggagaggtttggtttctttgtgccagactattcaaactcctgtagatgtgatcgttttcccagaagtaagtgttccttttggcttacatagacaagcctatactcttcatgaaaatcagcctgtattagaaagtttgcttcctaacaaatcttggggcgagtttttcacaaatttggattggatccaagcgatagctgaacgttatcaatgcaccgttatcatgggaatggaacgatgggaaaataaagggggaatactgcatttgtataatgctgctgaatgcgtatcgcgagaaggggaaataactagctatgataagcggattcttgttcctggaggtgagtacatccctggagggaaaataggtttttccttgtgtcaaacctttttcccagaatttgctcttccctttcaacgtttgccaggagagttttctggagttgtgaatataacagagcgaataaaagctgggatctctatttgttatgaggagacatttgggtatgcaattcgcccttacaaaaggcaacaagccgatattttagtaaatcttactaatgacggttggtatccgcgttcaaggctgcctctagtacatttttatcatggcatgttacgtaatcaagagttgggtataccttgtattcgcgcctgtcgcacaggagtttctgctgcagtggattctttgggtagaattgtcggcatacttccctgggaatcgagaacttgcccagtttctacaggagtactccaagtttccgtccctctttacagttatcatactgtatatgcaaggctgggtgatgctcctctgttactgattgcagtttgttcggttatcggagcgattgcctatttttataggaaaaagaaagagaccccaccacaaacatttttttga

SEQ ID NO: 61 - protein, 553 aa, Chlamydia trachomatis
MSSNKHASLCQKTPSLCRELQKAPALLLTEDIRFKALLNERIDSVAELFPCTYNSPYYKFISKSDLSAETSPLKVGVMLSGGPAPGGHNVILGLLHSIKKLHPNSQLLGFIRNGEGLLNNNTVEITDEFIEEFRNSGGFNCIGTGRTNIITEENKARCLQTANELDLDGLVIIGGDGSNTATAILAEYFAKHQAKTVLVGVPKTIDGDLQHLFLDLTFGFDTATKFYSSIISNISRDALSCKGHYHFIKLMGRSSSHITLECALQTHPNIALIGEEIAEKSISLETLIHDICETIADRAAMGKYHGVILIPEGVIEFIPEIQSLVKEIESIPEQENLYQALSLSSQQLLCQFPEDICHQLLYNRDAHGNVYVSKISVDKLLIHLVRQHLETHFRQVPFNAISHFLGYEGRSGTPTHFDNVYSYNLGYGAGVLVFNRCNGYLSTIEGLTSPIEKWRLRALPIVRMLTTKQGKDSKHYPLIKKRLVDIASPVFNKFSLYRKIWALEDSYRFVGPLQIHSPEDAHSDDFPPLILFLNHNEWQKRCSICLEIPDQDY

SEQ ID NO: 62 - DNA, 1662 bp, Chlamydia trachomatis
atgtcgtcgaataaacatgcttctctttgtcaaaagacgccttctttgtgtcgggagcttcaaaaagctcctgctcttctattaacagaagacataaggtttaaagctcttcttaatgaacgcattgactctgttgcagaactatttccatgcacttataactctccctactacaaatttatttcgaagtccgatctttccgctgagacctctccccttaaagtgggcgttatgctttctggaggcccagctcctggtgggcacaatgtcatcttaggattgctacacagtattaaaaagctccatccgaatagtcagcttttaggatttattcgcaatggagaaggacttctcaataataatactgtagaaatcacagatgaattcattgaagagtttcgtaactctggaggctttaattgcataggaacaggtcgcactaatatcataaccgaagaaaataaagcgcgctgtttacaaacagcaaatgaactcgatttagatggattagtgattattggaggcgatggttcgaatacagccacggcgattcttgctgaatattttgctaagcatcaagcaaaaacggtattagttggtgttcccaaaactattgatggagatttgcagcacctatttttagacctcacatttgggtttgatactgctactaaattttattcatccatcatcagcaacatttctagagacgcattatcgtgtaaaggccactatcattttattaaactaatgggccggtcttcttctcatatcacgctagaatgcgcactacagactcacccaaatattgctcttataggcgaagagattgcagaaaaaagcatctccttagaaacattaatccatgatatttgtgaaacaatagcagatcgagctgctatggggaaataccatggcgttattctcatccctgaaggagtcattgagtttattcctgaaatacagtctctggttaaagaaattgaatccattccagagcaggagaatctttaccaagctttatccttatcttctcagcaacttttatgccaatttccggaagatatttgccatcagctcttgtataatagagatgctcatggcaacgtctatgtatcaaaaattagtgttgataaacttctgattcatctagttcgtcaacatttagaaacacattttagacaagttcccttcaatgcaatctcccattttttaggttatgaagggcgttcaggaactcctacacattttgataatgtgtatagctataacttaggatatggtgctggggttctcgtttttaaccgctgtaatgggtatttatccacgatcgaaggtctaactagccctattgaaaaatggcgattgcgcgctttacccattgttcgaatgttgacgaccaagcaggggaaagacagtaaacattatcctctgataaaaaaaagattggtagatattgctagtcctgtttttaataagttctcactgtatcggaaaatctgggctttagaagactcctatcgctttgtagggccattacaaatacattctccggaggatgctcattctgatgattttcctcctttaattttgtttttgaatcataatgaatggcaaaaacgctgttctatttgtttagaaatccccgatcaggattattaa

SEQ ID NO: 63 - protein, 167 aa, Chlamydia trachomatis
MICCDKVLSSVQSMPVIDKCSVTKCLQTAKQAAVLALSLFAVFASGSLSILSAAVLFSGTAAVLPYLLILTTALLGFVCAVIVLLRNLSAVVQSCKKRSPEEIEGAARPSDQQESGGRLSEESASPQASPTSSTFGLESALRSIGDSVSGAFDDINKDNSRSRSHSF

SEQ ID NO: 64 - DNA, 439 bp, Chlamydia trachomatis
atgatctgctgtgacaaagtcttgtcgagcgtacaatcaatgcctgttatagataaatgctctgtaacgaaatgcttacaaacggctaagcaagcagctgttcttgcgttgtctttgtttgcggtgtttgcttcaggaagtttatccatattatcagcggcggtactgtttagtggcactgctgctgttcttccatatctgctgatattaacaacagctcttctaggatttgtttgtgctgttattgtgcttttaagaaatttatcagcagttgttcagagttgtaaaaagagatcacctgaagaaattgaaggggctgctcgtccctctgatcagcaggaatcaggaggacgtttgtccgaggagagcgcttcaccacaagcatctcctacttcgtctacttttggtcttgaatccgctttgcgctcaataggagata

SEQ ID NO: 65 - protein, 89 aa, Chlamydia trachomatis
MSKKCALTGRKPRRGYSYAIRGISKKKKGIGLKVTGRTKRRFFPNMMTKRLWSTEENRFLKLKISAAALRLVDKLGLDQVVARAKSKGF

SEQ ID NO: 66 - DNA, 270 bp, Chlamydia trachomatis
atgtcgaaaaaatgtgcgcttacaggaagaaagcctcgtcgcggttatagctatgctatccgagggatttctaaaaagaaaaaagggatcggtttgaaagttacaggaagaacaaaacgtcgattcttccctaatatgatgactaagagactatggtctactgaggaaaatcgcttcctcaaactcaaaatttctgcagcagctttacgccttgttgataaactagggttagatcaggttgttgctagagctaaaagcaagggtttttag

SEQ ID NO: 67 - protein, 157 aa, Chlamydia trachomatis
MLFGYLVGFLAADPEERMTSGGKRVVVLRLGVKSRVGSKDETVWCRCNIWNNRYDKMLPYLKKGSSVIVAGELSLESYVGRDGSPQASISVSVDTLKFNSGSSRPDARGSDEGRQRANDNVSIGFDGESLDTDSALDKEVYAGFGEDQQYASEDVPF

SEQ ID NO: 68 - DNA, 474 bp, Chlamydia trachomatis
atgttgttcggatatttggtaggatttctagctgccgatcctgaagaaagaatgacatccggaggtaaacgggttgttgttttacgtttgggtgtaaaatctcgtgtaggatctaaagatgaaacagtgtggtgcagatgcaatatctggaacaaccgttatgataagatgcttccttatttgaagaaaggttcttcagtcattgttgctggagagctttctttagaaagctatgtaggtagagacggttctccacaagcttctatttctgtaagcgtagatacattaaaatttaattccggatcttctcgtcctgatgctagaggttcagatgaaggtcgtcagagagctaatgataatgtctctattggatttgatggagaaagtttagatacagactctgcgcttgataaggaagtctatgcagggtttggagaagaccaacagtatgctagtgaggatgttcctttttag

SEQ ID NO: 69 - protein, 247 aa, Chlamydia trachomatis
MKKQEKMHPQNLLKVFIFFLAFFISYPSAEAHSPLQSSIQEKILTARPGDYAVLSRGSQKFFFLIRQSSSEATWVEMSEFASLTQQEKKLVEQSSWKNAFHQLQSSKKVYLLRISKNPLMIFVLKNAQWMPLSEKDPLPFFVKILRLPLSPAPSHLIKYKGKERTPWSPRTSLNGELITLPSSAWISVWPKDSSPLSEKNILIYFSNNERLAFPLWTSIDTPTGTVIIKTIEMGHQAASSYPALPNF

SEQ ID NO: 70 - DNA, 744 bp, Chlamydia trachomatis
atgaaaaagcaagaaaaaatgcaccctcaaaaccttcttaaagtttttatttttttcttggcatttttcatatcctatccctcggctgaagcccattctcctctccaatcatcaatccaagaaaaaattctaactgcccgccccggagactatgccgtcttaagccgaggatctcaaaaatttttctttttaattcgccaaagttcttcggaagcgacttgggtcgaaatgtctgaatttgcctccctaacacagcaagaaaaaaaattagtagaacagtcttcctggaagaatgccttccatcaactccaatcttcaaaaaaagtgtacttgttacgaatttccaaaaatcctcttatgatttttgttctcaaaaatgcgcaatggatgcctctctcagaaaaagatcctttgcctttctttgtaaaaatccttcgactccctttatctccagccccctctcacttaattaaatacaaagggaaagaacgcaccccctggtctccgcgaacatctttgaatggagaactcataacccttccttccagtgcttggatttctgtttggccaaaagattcttctcctctatcagaaaaaaatattctcatatatttttctaacaatgaacgtttagcgtttcctctatggactagtattgatactcctacagggacagtgattattaagactattgaaatggggcaccaagccgcctcctcctatccagctcttcccaatttctag

SEQ ID NO: 71 - DNA, 456 bp, Chlamydia trachomatis
aatatgagcactgtacccgttgttcaaggagctggatcttccaattcggcacaggatatttccactagaccattaacactgaaagagcgtatatcgaatcttctatcttccactgcatttaaggtgggattagtggtgataggactacttttagtgattgctactttgatattcctagtttcggcagcttcgtttgtaaatgccatctatctagtagctattcctgctattttgggatgcgtgaatatctgcgtaggaattttatccatggaaggacactgttctccggagagatggatcttatgtaagaaggtattaaagacttcagaagatatcatcgatgatgggcagataaacaactctaataaagtgtttactgatgagaggttgaatgccataggtggggtagtggaatctctatctagaagaaatagtctggtggatcagacccaatga

SEQ ID NO: 72 - protein, 151 aa, Chlamydia trachomatis
NMSTVPVVQGAGSSNSAQDISTRPLTLKERISNLLSSTAFKVGLVVIGLLLVIATLIFLVSAASFVNAIYLVAIPAILGCVNICVGILSMEGHCSPERWILCKKVLKTSEDIIDDGQINNSNKVFTDERLNAIGGVVESLSRRNSLVDQTQ

SEQ ID NO: 73 - DNA, 354 bp, Chlamydia trachomatis
gggctagtttcttttattgttaaaagaattgcttttatcgataaaagaaacttcaagagcccttttctagaaaggagtctggaagttatgaaaaaaactgctttactcgctgctttatgtagtgttgtttctttaagtagttgttgtcgtatcgttgactgttgcttcgaagatccatgcgcacctatccaatgttcaccttgtgaatctaagaagaaagacgtagacggtggttgcaactcttgtaacgggtatgtcccagcttgcaaaccttgcggaggggatacgcaccaagatgctaaacatggccctcaagctagaggaattccagttgacggcaaatgcagacaatag

SEQ ID NO: 74 - protein, 117 aa, Chlamydia trachomatis
GLVSFIVKRIAFIDKRNFKSPFLERSLEVMKKTALLAALCSVVSLSSCCRIVDCCFEDPCAPIQCSPCESKKKDVDGGCNSCNGYVPACKPCGGDTHQDAKHGPQARGIPVDGKCRQ

SEQ ID NO: 75 - DNA, 1650 bp, Chlamydia trachomatis
gctagtaagggagcccctttagtgtttaaacttgtgtcatacatcatcctttcttgggtgctggtctgtttggctcagccggatgtaagtgttgtagcttctgttgttagttgtatttgcggttacagcttactttgggctgggctttttgctttagtagagcaattatcttggaagaaagtttggtgcatcgcttttatttggacttggactgtcgaaggcgctcatttctcttggatgcttgaagatctttatgtagggacaagcatctattttgtttggggtatactgctttcttatctcgccaccctatttgctagtttttcttgtttggttgtgtggtgttgtcgcaagcaatataggggagctcttgtttggcttccaggggtttgggtggcgatagaagcaatacgctattatgggttgctttcaggagtttcttttgattttattggctggcctcttacagcgacagcctatggccggcaattcggcagcttttttggatgggctggacaaagctttctagttattgctgccaatatatgctgttttgcagtatgtttattaaaacactctttttccaaaggtttgtggttgacgttgtgcgcgttcccttatctgttaggcggagcgcattacgaatacctaaagaagcatttttccgactctgaagtgcttcgagttgccatcgtgcagcctggatatagtcctcatatgcatgcagggaggacggctagtgctatttggagaggtttggtttctttgtgccagactattcaaactcctgtagatgtgatcgttttcccagaagtaagtgttccttttggcttacatagacaagcctatactcttcatgaaaatcagcctgtattagaaagtttgcttcctaacaaatcttggggcgagtttttcacaaatttggattggatccaagcgatagctgaacgttatcaatgcaccgttatcatgggaatggaacgatgggaaaataaagggggaatactgcatttgtataatgctgctgaatgcgtatcgcgagaaggggaaataactagctatgataagcggattcttgttcctggaggtgagtacatccctggagggaaaataggtttttccttgtgtcaaacctttttcccagaatttgctcttccctttcaacgtttgccaggagagttttctggagttgtgaatataacagagcgaataaaagctgggatctctatttgttatgaggagacatttgggtatgcaattcgcccttacaaaaggcaacaagccgatattttagtaaatcttactaatgacggttggtatccgcgttcaaggctgcctctagtacatttttatcatggcatgttacgtaatcaagagttgggtataccttgtattcgcgcctgtcgcacaggagtttctgctgcagtggattctttgggtagaattgtcggcatacttccctgggaatcgagaacttgcccagtttctacaggagtactccaagtttccgtccctctttacagttatcatactgtatatgcaaggctgggtgatgctcctctgttactgattgcagtttgttcggttatcggagcgattgcctatttttataggaaaaagaaagagaccccaccacaaacatttttttga

SEQ ID NO: 76 - protein, 549 aa, Chlamydia trachomatis
ASKGAPLVFKLVSYIILSWVLVCLAQPDVSVVASVVSCICGYSLLWAGLFALVEQLSWKKVWCIAFIWTWTVEGAHFSWMLEDLYVGTSIYFVWGILLSYLATLFASFSCLVVWCCRKQYRGALVWLPGVWVAIEAIRYYGLLSGVSFDFIGWPLTATAYGRQFGSFFGWAGQSFLVIAANICCFAVCLLKHSFSKGLWLTLCAFPYLLGGAHYEYLKKHFSDSEVLRVAIVQPGYSPHMHAGRTASAIWRGLVSLCQTIQTPVDVIVFPEVSVPFGLHRQAYTLHENQPVLESLLPNKSWGEFFTNLDWIQAIAERYQCTVIMGMERWENKGGILHLYNAAECVSREGEITSYDKRILVPGGEYIPGGKIGFSLCQTFFPEFALPFQRLPGEFSGVVNITERIKAGISICYEETFGYAIRPYKRQQADILVNLTNDGWYPRSRLPLVHFYHGMLRNQELGIPCIRACRTGVSAAVDSLGRIVGILPWESRTCPVSTGVLQVSVPLYSYHTVYARLGDAPLLLIAVCSVIGAIAYFYRKKKETPPQTFF

SEQ ID NO: 77 - DNA, 645 bp, Chlamydia trachomatis
gaaaattgttataggatcaggagagaaacgtttcccatgctggggagcatttccttcacaacatataaagaaaacctcatgagaaagactatttttaaagcgtttaatttattattctcccttctttttctttcttcatgctcttatccttgcagagattgggaatgccatggttgcgactccgcaagacctcgtaaatcctcttttggattcgtacctttctactccgatgaagaaattcaacaagcttttgttgaagattttgattccaaagaagagcagctgtacaaaacgagcgcacagagtacctctttccgaaatatcactttcgctacagatagttattctattaaaggagaggataacctcacgattcttgcaagcttagttcgtcatttgcataaatctcctaaagctacgctatatatagagggccatacagatgaacgtggagctgcagcttataacctagctttaggagctcgtcgtgcgaatgctgtaaaacaatacctcatcaaacagggaatcgctgcagaccgcttattcactatttcttacggaaaagaacatcctgttcatccaggccataatgaattagcttggcaacaaaatcgtcgtactgaatttaagatccatgctcgctaa

SEQ ID NO: 78 - protein, 214 aa, Chlamydia trachomatis
ENCYRIRRETFPMLGSISFTTYKENLMRKTIFKAFNLLFSLLFLSSCSYPCRDWECHGCDSARPRKSSFGFVPFYSDEEIQQAFVEDFDSKEEQLYKTSAQSTSFRNITFATDSYSIKGEDNLTILASLVRHLHKSPKATLYIEGHTDERGAAAYNLALGARRANAVKQYLIKQGIAADRLFTISYGKEHPVHPGHNELAWQQNRRTEFKIHAR

SEQ ID NO: 79 - protein, 943 aa, Neisseria gonorrhoeae
MNKKHGFPLTLTALAIATAFPAYAAQAGAAALDAAQSQSLKEVTVRAAKVGRRSKEATGLGKIVKTSETLNKEQVLGIRDLTRYDPGVAVVEQGNGASGGYSIRGVDKNRVAVSVDGVAQIQAFTVQGSLSGYGGRGGSGAINEIEYENISTVEIDKGAGSSDHGSGALGGAVAFRTKEAADLISDGKSWGIQAKTAYGSKNRQFMKSLGAGFSKDGWEGLLIRTERQGRETRPHGDIADGVEYGIDRLDAFRQTYDIKRKTTEPFFLVEGENTLKPVAKLAGYGIYLNRQLNRWVKERIEQNQPLSAEEEAQVREAQARHENLSAQAYTGGGRILPDPMDYRSGSWLAKLGYRFGGRHYVGGVFEDTKQRYDIRDMTEKQYYGTDEAEKFRDKSGVYDGDDFRDGLYFVPNIEEWKGDKNLVKGIGLKYSRTKFIDEHHRRRRMGLLYRYENEKYSDNWADKAVLSFDKQGVATDNNTLKLNCAVYPAVDKSCRASADKPYSYDSSDRFHYREQHNVLNASFEKSLKNKWTKHHLTLGFGYDASKAVSRPEQLSHNAARISESTGFDEKNQDKYRLGKPEVVEGSVCGYIETLRSRKCVPRKINGSNIHISLNDRFSIGKYFDFSLGGRYDRKNFTTSEELVRSGRYADRSWNSGIVFKPNRHFSVSYRASSGFRTPSFQELFGIDIYHDYPKGWQRPALKSEKAANREIGLQWKGDFGFLEISSFRNRYTDMIAVADQKTKLPDSAGRLTEIDIRDYYNAQNMSLQGINILGKIDWNGVYGKLPEGLYTTLAYNRIKPKSVSNRPDLSLRSYALDAVQPSRYVLGFGYDQPEGKWGANIMLTYSKGKNPDELAYLAGDQKRYSAGRVTSSWKTADVSAYLNLKKRLTLRAAIYNIGNYRYVTWESLRQTAESTANRHGGDSNYGRYAAPGRNFSLALEMKF

SEQ ID NO: 80 - DNA, 3300 bp, Neisseria gonorrhoeae
ctcgggataacggcatcaatctttcgggaaatggttcgactaatcctcaaagtttcaaagccgacaatcttcttgtaacgggcggcttttacggcccgcaggcggcggaattgggcggcactattttcaataaggatgggaaatctcttggtataactgaagatattgaaaatgaagttgaaaatgaagctgatgttggcgaacagttagaacctgaagttaaaccccaattcggcgtggtattcggtgcgaagaaagataataaagaggtggaaaaatgaataagaaacacggttttccgctgactttgacggcgttggccattgcaaccgcttttccggcttatgctgcccaagcgggggcggcggcacttgatgcggcgcaaagtcaatcattgaaagaggttaccgtccgtgccgccaaagtgggacggcgatcgaaagaggcgacaggtttgggcaaaatcgtcaaaacgtcggaaacgttgaacaaagaacaggtactcggtatccgcgacctgacgcgctacgatccgggcgtggcggttgtcgaacagggcaacggcgcgagcggcggctactcgatacgcggcgtagataaaaaccgtgtggcggtttcggttgacggcgttgcccaaatacaggcgtttaccgtgcagggatcgttgagcggatacggcggacgcggcggcagcggcgcaatcaacgaaatcgaatatgaaaacatcagcacggtggaaatcgacaaaggcgccggttcgtccgatcacggcagcggcgcactcggcggcgcggtcgccttccgcaccaaagaggcggcagacctgatttcagacggcaaaagctgggggatacaggcaaaaaccgcttacggcagtaaaaaccgccaatttatgaagtcgctcggcgcggggttcagcaaagacggttgggaagggctgctaatccgaaccgaacgccaagggcgggaaacgcgcccgcacggcgatattgcggacggggtggaatacggcatagaccgtttggacgcgttccgccagacatacgatattaaacgcaagacaacagagccatttttcttagtagagggcgagaatacactcaagcccgtggcaaaattggcgggctacgggatatatttgaaccgccagctcaaccgctgggtaaaagaacgtattgaacaaaatcagcctttaagtgctgaagaagaggcgcaggtgcgggaggcgcaggcgcgccacgaaaacctgtccgcccaagcctacacgggcggcggcaggatattgcccgatccgatggattaccgcagcggctcttggcttgccaagctgggctaccgcttcggcggcaggcattatgtcggcggcgtgtttgaggataccaaacagcgttacgacatccgcgatatgacggaaaaacagtattacggtacggacgaggcggaaaagtttagagacaagagcggggtgtacgacggcgacgatttccgcgacggcttgtattttgtgccgaatatagaagagtggaagggcgataaaaatttggtcaagggcataggtttgaaatattcccgcaccaaatttattgacgaacatcaccgccgccgccgtatgggtttgctgtaccgttatgaaaatgagaaatactcggacaactgggcggataaggcggtgttgtcgtttgacaaacagggcgtggcaaccgacaacaacacgctgaagctgaattgcgccgtgtatcctgccgtggacaaatcctgccgcgcgtcggcggacaaaccgtattcctacgacagcagcgaccgtttccactaccgcgaacagcacaatgttttgaatgcctcgtttgagaagtcgctgaaaaacaaatggacgaaacaccatctgactttgggcttcggttacgatgcttccaaagcagtatcccgcccagaacagctttcccacaatgcggcaaggatttcggaatccacgggattcgatgaaaagaatcaagataagtaccgtttgggtaagcccgaagtcgtcgaagggtcggtctgcggctatatcgaaaccctgcgttcccgcaaatgcgtgccaagaaaaatcaacggcagcaatatccacatttctttgaacgaccgtttttcaatcggcaaatattttgatttcagcttgggcggcaggtacgaccggaaaaacttcaccacgtcggaagaactcgtccgcagcgggcggtatgccgaccgttcgtggaacagcggcatcgtgttcaaaccgaaccggcatttttccgtgtcttaccgcgcctccagcggcttcagaacgccttccttccaagaacttttcgggatagacatttatcacgattatccgaaaggctggcagcgtcccgccctgaaatcggaaaaggcagccaaccgggaaatcggtttgcagtggaagggcgatttcggctttttggaaatcagcagtttccgcaaccgttataccgatatgattgccgttgccgatcaaaaaaccaaattgccggattcagcaggacgattgacagagattgatatacgcgattattacaatgcccaaaatatgtcgcttcaaggcatcaacatcttggggaaaatcgactggaacggcgtatacggcaaactgcccgaaggcctgtacaccacattggcgtacaaccgtatcaaaccgaaatcggtatccaaccggccggacttgtccctccgcagctatgctttggatgcggtacagccgtcgcgttatgttttggggttcggatacgaccagcccgaggggaaatggggcgcaaacattatgctgacctattccaaagggaaaaaccctgacgagcttgcttatctggcaggcgatcaaaaacgatattcggcaggaagggttacgtcttcttggaaaacggcagatgtttccgcttatctgaatctgaaaaaacggctgaccttgagggcggctatctacaatatcggcaactaccgctacgttacttgggaatccttgcgccagactgcggaaagcacggcaaaccggcacggcggcgacagcaactatggaaggtatgccgcaccgggcaggaacttcagcctcgcgctcgaaatgaagttttaaaggaaatgccgtctggaagcttgatctgcaccccaaaagtcggactaaaccgccaactgattaaggtgcaggtttttttgattcaatataaacaagatttccgccgtcattcccgcgcaggcgggaatccggacattcaatgctaaggcaatttatcggaaatgactgaaactcaaaaaaccggattccca

SEQ ID NO: 81 - protein, 728 aa, Neisseria gonorrhoeae
MRKLNYYGIALLPLMLASCGGNFGVQPVVESTPTAYPVTFKSKDVPTSPPPAEPSVETTPVNRPAVGAAMRLLRRNTAFHREDGTAIPDSKQAEEKLSFKEGDVLFLYGSKGNKLQQLKSEIHKRDSDVEIRTSEKENKKYGYEFVDAGYVYTKNGKDEIEQNSGGKRFTHRFGYDGFVYYSGERPSQSLPSAGTVKYFGNWQYMTDAKRHRTGKAVASDDLGYITFYGNDIGATSYAAKDADDREKHPAEYTVDFDKKILKGELIKNQYVQKKNDPKKPLTIYNITADLNGNRFTGSAKVNTEVKTRHADKEYLFFHTDADQRLEGGFFGDNGEELAGRFISNDNGVFGVFAGKQKTNASGTNPAMPFGKHTKILDSLKISVDEATDENPRPFEVSTMPDFGHPDKLLVEGREIPLVSKEKTIDLADGRKMTVSACCDFLTYVKLGRIKTERPAVKPKAQDEEDSGINNGEESEDEEEIAEESEDEVSEDDNGEDEDEIVEEEADEAEEIEEEAEEEEPEEESPEEGNGVSDGIPPAPEALKGRDIDLFLKGIRTAEADIPKTGTAHYTGTWEARIGEPIQWDNKADKAAKAEFDVDFGNKSISGTLTEQNGVEPAFRIENGVIEGNGFHPTARTRDNGINLSGNGSTNPQSFKADNLLVTGGFYGPQAAELGGTIFNKDGKSLGITEDIENEVENEADVGEQLEPEVKPQFGVVFGAKKDNKEVEK

SEQ ID NO: 82 - protein, 907 aa, Neisseria gonorrhoeae
MQQQHLFRFNILCLSLMTALPAYAENVQAGQAQEKQLDTIQVKAKKQKTRRDNEVTGLGKLVKTADTLSKEQVLDIRDLTRYDPGIAVVEQGRGASSGYSIRGMDKNRVSLTVDGLAQIQSYTAQAALGGTRTAGSSGAINEIEYENVKAVEISKGSNSVEQGSGALAGSVAFQTKTADDVIGEGRQWGIQSKTAYSGKNRGLTQSIALAGRIGGAEALLIRTGRHAGEIRAHEAAGRGVQSFNRLAPVEDGSDYAYFVVEGECPDGYAACKDKPKKDVVGEDKRQTVSTRDYTGPNRFLADPLSYESRSWLFRPGFRFENKRHYIGGILERTQQTFDTRDMTVPAFLTKAVFDANSKQAGSLRGNGKYAGNHKYGGLFTNGENNAPVGAEYGTGVFYDETHTKSRYGLEYVYTNADKDTWADYARLSYDRQGIGLDNHFQQTHCSADGSDKYCRPSADKPFSYYKSDRVIYGESHRLLQAAFKKSFDTAKIRHNLSVNLGYDRFGSNLRHQDYYYQSANRAYSLKTPPQNNGKKTSPYWVSIGRGNVVTGQICRSGNNTYTDCTPRSINGKSYYAAVRDNVRLGRWADVGAGLRYDYRSTHSDDGSVSTGTHRTLSWNTGIVLKPADWLDLTYRTSTGFRLPSFAEMYGWRSGDKIKAVKIDPEKSFNKEAGIVFKGDFGNLEASWFDNAYRDLIVRGYEAEIKNGKEQAKGAPAYLNAQSARITGINILGKIDWNGVWDKLPEGWYSTFAYNRVRVRDIKKRADRTDIQSHLFDAIQPSRYVVGSGYDQPEGKWGVNGMLTYSKAKEITELLGSRALLNGNSRNTKATARRTRPWYIVDVSGYYTVKKHFTLRAGVYNLLNHRYVTWENVRQTAAGAVNQHKNVGVYNRYAAPGRNYTFSLEMKF

SEQ ID NO: 83 - DNA, 3035 bp, Neisseria gonorrhoeae
cgaagagttgggcggatggtttgcctatccgggcaatgaacaaacgaaaaatgcgcaagcttcatccggcaatggaaattcagcaggcagcgcgaccgtggtattcggtgcgaaacgccaaaagcttgtgcaataagcacggctgccgaacaatcgagaataaggcttcagacggcatcgttcctgccgattccgtctgaaagcgaagattagggaaacactatgcaacagcaacatttgttccgattcaatattttatgcctgtctttaatgactgcgctgcccgcttatgcagaaaatgtgcaagccggacaagcacaggaaaaacagttggacaccatacaggtaaaagccaaaaaacagaaaacccgccgcgataacgaagtaaccggtttgggcaaattggtcaaaaccgccgacacactcagcaaagaacaggtactcgacatccgcgacctgacgcgttacgaccccggcatcgccgtcgtcgaacaggggcgcggcgcaagctcgggctactcgatacgcggtatggacaaaaaccgcgtctccttgacggtggacggcttggcgcaaatacagtcctacaccgcgcaggcggcattgggcgggacgaggacggcgggcagcagcggcgcaatcaatgaaatcgagtatgaaaacgttaaggctgtcgaaatcagcaaaggctcaaactcggtcgaacaaggcagcggcgcattggcgggttcggtcgcatttcaaaccaaaaccgcagacgatgttatcggggaaggcaggcagtggggcattcagagtaaaaccgcctattccggcaaaaaccgggggcttacccaatccatcgcgctggcggggcgcatcggcggtgcggaggctttgctgatccgcaccggccggcacgcgggggaaatccgcgcccacgaagccgccggacgcggcgttcagagctttaacaggctggcgccggttgaagacggcagtgactatgcctattttgtggtcgaaggagaatgccctgatggatatgcggcttgtaaagacaaaccgaaaaaagatgttgtcggcgaagacaaacgtcaaacggtttccacccgagactacacgggccccaaccgcttccttgccgatccgctttcatacgaaagccggtcgtggctgttccgcccgggttttcgttttgagaataagcggcactacatcggcggcatactcgaacgcacgcaacaaactttcgacacgcgcgatatgacggttccggcattcctgaccaaggcggtttttgatgcaaattcaaaacaggcgggttctttgcgcggcaacggcaaatacgcgggcaaccacaaatacggcgggctgtttaccaacggcgaaaacaatgcgccggtgggcgcggaatacggtacgggcgtgttttacgacgagacgcacaccaaaagccgctacggtttggaatatgtctataccaatgccgataaagacacttgggcggattatgcccgcctctcttacgaccggcagggcatcggtttggacaaccattttcagcagacgcactgttctgccgacggttcggacaaatattgccgcccgagtgccgacaagccgttttcctattacaaatccgaccgcgtgatttacggggaaagccacaggctcttgcaggcggcattcaaaaaatccttcgataccgccaaaatccgccacaacctgagcgtgaatctcggttacgaccgcttcggctctaatctgcgccatcaggattattattatcaaagtgccaaccgcgcctattcgttgaaaacgccccctcaaaacaacggcaaaaaaaccagcccctattgggtcagcataggcaggggaaatgtcgttacggggcaaatctgccgctcgggcaacaatacttatacggactgcacgccgcgcagcatcaacggcaaaagctattacgcggcggtccgggacaatgtccgtttgggcaggtgggcggatgtcggcgcgggcttgcgctacgactaccgcagcacgcattcggacgacggcagcgtttccaccggcacgcaccgcaccctgtcctggaacaccggcatcgtcctcaaacctgccgactggctggatttgacttaccgcacttcaaccggcttccgcctgccctcgtttgcggaaatgtacggctggcggtcgggcgataaaataaaagccgtcaaaatcgatccggaaaaatcgttcaacaaagaagccggcatcgtgtttaaaggcgatttcggcaacttggaggcaagttggttcgacaatgcctaccgcgatttgattgtccggggttatgaagcggaaattaaaaacggcaaagaacaagccaaaggcgccccggcttacctcaatgcccaaagcgcgcggattaccggcatcaatattttgggcaaaatcgattggaacggcgtatgggataaattgcccgaaggttggtattctacatttgcctataatcgtgtccgtgtccgcgacatcaaaaaacgcgcagaccgcaccgatattcaatcacacctgtttgatgccatccaaccctcgcgctatgtcgtcggctcgggctatgaccaaccggaaggcaaatggggcgtgaacggtatgctgacttattccaaagccaaggaaatcacagagttgttgggcagccgggctttgctcaacggcaacagccgcaatacaaaagccaccgcgcgccgtacccgcccttggtatattgtggatgtgtccggttattacacggttaaaaaacacttcaccctccgtgcgggcgtgtacaacctcctcaaccaccgctatgttacttgggaaaatgtgcggcaaactgccgccggcgcagtcaaccaacacaaaaatgtcggcgtttacaaccgatatgccgcccccggccgcaactacacatttagcttggaaatgaagttctaaacgtccgaacgccgcaaatgccgtctgaaaggcttcagacggcgttttttacacaatccccaccgtttcccatccttcccgatacaccg

SEQ ID NO: 84 - protein, 683 aa, Neisseria gonorrhoeae
MNNPLVNQAAMVLPVFLLSACLGGGGSFDLDSVDTEAPRAAPKYQDVPSKKPEARKDQGGYGFAMRFKRRNWYRAANENEVKLKESDWEQTDDDEIKNPFKQKNIINALPGNEGELLQDSSQQGKGTSKVRDHHDFKYVWSGFFYKRIKITTKKDESHKIIEARSGPDGYIFYKGRNPSRKLPVSGEVTYKGTWDFLTDVKANQKFTDLGNASTKSGDQYSAFSGELDYIVKKEEDKKEKHKGLGLTTEITVDFEKKTLIGKLIKNNMLINNNTKPTTQYYSLEAQVTGNRFSGKAMATEKGENKQHPFVSDSSSLSGGFFGPQGEELGFRFLSDDGKVAVVGSAKTKDETASSGGTSGGASVSTSNGAAGTSSENKLTTVLDAVELTPNGKKIKDLDNFSNAAQLVVDGIMIPLLPKDSESGGSHTDKGENGKTAFIYETTYTPESDKEDAQTGMATNGVQTVSNTAGGTSGKTKTHYEVQACCSNLNYLKYGLLTRKNSSQADAKMGQVEQSMFLQGERTDEKEIPQEQNVVYSGTWYGHIATNGTSWTREASDQENGNRANFDVNFKDKRITGTLTAENRSEATFTIEAMIEGNGFKGTAKTGNGGFAPDQNSSTGTHKVHITNAAVQGGFYGPNAEELGGWFAYPGNGQTKNAQTSSGNGNSAGSATVVFGAKRQQLVK

SEQ ID NO: 85 - DNA, 2052 bp, Neisseria gonorrhoeae
atgaacaatccattggtgaatcaggctgctatggtgctgcccgtgtttttgttgagcgcttgtctgggcggaggcggcagtttcgatcttgattctgtcgataccgaagccccgcgtgccgcgccaaagtatcaagatgttccttccaaaaaaccggaagcccgaaaagaccaaggcggatacggtttcgcgatgcgcttcaagcggcggaattggtatcgggcggcaaacgaaaacgaggttaaactgaaagagagtgattgggaacaaacggatgatgatgagatcaaaaaccctttcaaacaaaaaaatattattaatgccttacctggaaatgagggggaattattgcaagattccagtcaacaaggtaagggtacatctaaggttagggaccatcacgattttaaatacgtatggtcgggttttttttataaacggattaagattacaactaaaaaagacgaatctcataaaataatcgaagccagaagcggtcctgacggttatattttttataaaggcagaaatccctcgagaaaacttcctgtttcaggggaggttacgtacaaaggtacttgggattttttaactgatgtgaaagcaaatcagaaatttacagatttaggaaatgcttctacgaaatccggagaccaatatagtgctttttccggggagttggattatatagtcaaaaaagaggaggataaaaaagaaaagcacaaaggtttgggattaacaacggaaataacggttgattttgagaaaaaaaccctgatcggaaaattaattaaaaacaacatgttaatcaataataacactaaacccaccacccaatattacagccttgaggctcaagtaacaggcaaccgcttcagcggcaaggcgatggcaaccgaaaaaggcgaaaacaaacaacatccctttgtttccgactcgtcttctctgagcggcggctttttcggcccgcagggtgaggaattgggtttccgctttttgagcgacgatggaaaagttgccgttgtcggcagcgcgaaaaccaaagacgaaaccgcaagcagtggcggcacttcgggcggtgcaagcgtttccacatcaaacggtgcggcaggcacgtcgtctgaaaacaagctgaccacggttttggatgcggttgaattgacaccaaacggcaagaaaatcaaagatctcgacaacttcagcaacgccgcccaactggttgtcgacggcattatgattccgctcctgcccaaggattccgaaagcgggggcagtcatacagataaaggtgaaaacggcaaaacagcctttatctacgaaacaacctacacgccggaaagtgataaagaagacgctcaaacaggtatggcgaccaatggcgtgcaaaccgtttcaaatacggcaggcggcacaagtggcaaaacaaaaacccattatgaagtccaagcctgctgttccaacctcaattatctgaaatacgggttgctgacgcgtaaaaacagtagtcaagctgacgctaaaatgggacaagttgaacaaagtatgttcctccaaggcgagcgcaccgatgaaaaagaaattccacaagaacaaaatgtcgtttattcaggcacttggtacgggcatattgccaccaacggcacaagttggacccgcgaagcctccgatcaggaaaatggtaatcgggcaaattttgacgtgaatttcaaagacaaaagaattaccggcacgttaaccgctgaaaacaggtcggaggcaacctttaccattgaagccatgattgagggcaacggctttaaaggtacggcgaaaaccggtaatggcggctttgcgccggatcaaaacagcagcaccggtacacataaagtgcacatcacaaatgccgcggtgcagggcggtttttacgggcctaacgccgaagagttgggcggttggtttgcctatccgggcaatggacaaacgaaaaatgcgcaaacttcatccggcaatggaaattcagcaggcagcgcgaccgtggtattcggtgcgaaacgccaacagcttgtgaaataa

SEQ ID NO: 86 - protein, 215 aa, Neisseria gonorrhoeae
MASKSSKTNLDLNNLHLLNLPARLFIALLVVAAVLGLGYAGLFKSQMESLEEYEAKETELKNTYKQKSIDAASLNNLRDELASIRSAFDIMLKQLPTDAEIPNLVQELHQAGSSNGLRLDSVMPQPPVDDGPIKKLPYSISITGNYEQISQFTRDVGSLSRIITLESLKIAQSPENGGNPDGKSSILNLSAIATTYQAKSIEELAAEAAQNAEQK

SEQ ID NO: 87 - protein, 180 aa, Neisseria gonorrhoeae
MKHYALLISFLALSACSQSSEDLNEWMAQTRREAKAEIIPFQAPTLPVAPVYSPPQLTGPNAFDFRRMETAKKGENAPDTKRIKETLEKFSLENMRYVGILKSGQKVSGFIEAEGYVYTVGVGNYLGQNYGRIESITDDSIILNELIEDSTGNWVSRKAELLLNSSDKNTEQAAQPEEQN

SEQ ID NO: 88 - protein, 720 aa, Neisseria gonorrhoeae
MNTKLTKIISGLFVATAAFQTASAGNITDIKVSSLPNKQKIVKVSFDKEIVNPTGFVTSSPARIALDFEQTGISMDQQVLEYADPLLSKISAAQNSSRARLVLNLNKPGQYNTEVRGNKVWIFINESDDTVSAPARPAVKAAPAAPAKQQAAAPFTESVVSVSAPFSPAKQQAAASAKQQAATPAKQTNIDFRKDGKNAGIIELAALGFAGQPDISQQHDHIIVTLKNHTLPTALQRSLDVADFKTPVQKVTLKRLNNDTQLIITTTGNWELVNKSAAPGYFTFQVLPKKQNLESGGVNNAPKTFTGRKISLDFQDVEIRTILQILAKESGMNIVASDSVSGKMTLSLKDVPWDQALDLVMQARNLDMRQQGNIVNMAPRRAACQRQSLLTSGKRHCRSGRAVFPKLPIEIQKCGRIPQHPALDNADTTGNRNTLVSGRGSVLIDPATNTLIVTDTRSVIEKFRKLIDELDVPAQQVMIEARIVEAADGFSRDLGVKFGATGRKKLKNETSAFGWGVNSGFGGGDKWEAKPKSTCRLPCRKQHFAGARDFSGALNLELSASESLSKTKTLANPRVLTQNRKEAKIESGYEIPFTVTTRSGGGNSTNTELKKAVLGLTVTANITPDGQIIMTVKINKDSPRQCASGNNTILCISTKSLNTQAMVENGGTLIVGGIYEENNGNTLTKVPLLATSPLSATSLKHSGKNRPPRTADFQLPPREL

SEQ ID NO: 89 - DNA, 3829 bp, Neisseria gonorrhoeae
aaacgcacaggaggaaactgaatggcttctaaatcatctaaaaccaacttggatctcaacaaccttcacctgctcaaccttcctgccaggctttttatcgccctgctggtcgttgccgccgtgctggggctcggttatgccggattgttcaaaagccagatggaatcccttgaggaatatgaagcaaaagaaaccgaactgaaaaacacctacaaacagaaaagtatcgacgcggccagcctgaacaaccttagggacgaacttgcctcaatccgctctgccttcgatatcatgttgaaacagctgccgacagatgcagaaattcccaatttggttcaagagcttcatcaggcgggttcgagcaacggtctgcgcttggacagcgttatgccccaacctcccgtagatgacggtcccatcaaaaaattaccctattccatttccattaccggaaattacgaacagatcagccaatttacccgcgatgtcggcagtctctcccgaatcattacccttgagtcgctgaaaatcgcccaatctccggaaaacggcggcaatcctgacggcaagagcagtatcctgaacctcagcgccattgccaccacctaccaagcaaaatccatagaagagcttgccgcagaagcggcacaaaatgccgagcaaaaataacttacgttagggaaaccatgaaacactatgccttactcatcagctttctggctctctccgcgtgttcccaaagttctgaagacctaaacgaatggatggcacaaacgcgacgcgaagccaaagcagaaatcatacctttccaagcacctaccctgccggttgcgccggtatacagcccgccgcagcttacagggccgaacgcattcgacttccgccgcatggaaaccgccaaaaaaggggaaaatgcccccgacaccaagcgtattaaagaaacgctggaaaaattcagtttggaaaatatgcgttatgtcggcattttgaagtccggacagaaagtctccggcttcatcgaggctgaaggttatgtctacactgtcggtgtcggcaactatttgggacaaaactacggtagaatcgaaagcattaccgacgacagcatcatcctgaacgagctgatagaagacagcacgggcaactgggtttcccgtaaagcagaactgctgttgaattcttccgacaaaaacaccgaacaagcggcacagcctgaggaacaaaattaagaagaggattactccattatgaataccaaactgacaaaaatcatttccggtctctttgtcgcaaccgccgcctttcagacggcatcggcaggaaacattacagacatcaaagtttcctccctgcccaacaaacagaaaatcgtcaaagtcagctttgacaaagagattgtcaacccgaccggcttcgtaacctcctcaccggcccgcatcgccttggactttgaacaaaccggcatttccatggatcaacaggtactcgaatatgccgatcctctgttgagcaaaatcagtgccgcacaaaacagcagccgtgcgcgtctggttctgaatttgaacaaaccgggccaatacaataccgaagtacgcgggaacaaagtttggatattcattaacgaatcggacgataccgtgtccgcccccgcccgcccagccgtaaaagccgcgcctgccgcaccggcaaaacaacaggctgccgcaccgtttaccgagtccgtagtatccgtatccgcaccgttcagcccggcaaaacaacaggcagcggcatcggcaaaacaacaggcggcgacaccggcaaaacaaaccaatatcgatttccgcaaagacggcaaaaatgccggcattatcgaattggcggcattgggctttgccgggcagcccgacatcagccaacagcacgaccacatcatcgttacgctgaaaaaccataccctgccgaccgcgctccaacgcagtttggatgtggcagacttcaaaacaccggttcaaaaggttacgctgaaacgcctcaataacgacacccagctgattatcacaacaaccggcaactgggaactcgtcaacaaatccgccgcgcccggatactttaccttccaagtcctgccgaaaaaacaaaacctcgagtcaggcggcgtgaacaatgcgcccaaaaccttcacaggccggaaaatctcccttgacttccaagatgtcgaaatccgcaccatcctgcagattttggcaaaagaatccgggatgaacattgttgccagcgactccgtcagcggcaaaatgaccctctccctcaaagacgtaccttgggatcaggctttggatttggttatgcaggcgcgcaacctcgatatgcgccagcaagggaacatcgtcaacatggccccgcgacgagctgcttgccaaagacaaagccttcttacaagcggaaaaagacattgccgatctgggcgcgctgtattcccaaaacttccaattgaaatacaaaaatgtggaagaattccgcagcatcctgctttggacaatgccgacacgaccggaaaccgcaacacgcttgtcagcggcaggggcagcgtgctgatcgatcccgccaccaacaccctgattgttaccgatacccgcagcgtcatcgaaaaattccgcaaactgattgacgaattggacgtacccgcgcaacaagtgatgattgaggcgcgtatcgtcgaagcggcagacggcttctcgcgcgatttgggcgttaagttcggcgcgacaggcaggaaaaaactgaaaaatgagacgagcgcattcggctggggcgtgaactccggcttcgggggcggcgataaatgggaggccaaaccaaaatcaacctgccggttgccgtgccgcaaacagcatttcgctggtgcgcgcgatttctccggcgcgttgaatttggaattgtccgcatccgagtcgctttcaaaaaccaaaacgcttgccaatccgcgcgtgctgacccaaaaccgcaaagaggccaaaatcgaatccggttacgaaattccttttaccgtaactacacgctcgggcggcggcaactctaccaacacggaactcaaaaaagccgtcttggggctgaccgttacggcgaacatcacgcccgacggacaaatcatcatgaccgtcaaaatcaacaaagactcgcctcgacaatgtgcttcaggcaacaacacaatcctatgtatttcgaccaaaagcctgaatacgcaggctatggttgaaaacggcggcactttgattgtcggcggtatttatgaagaaaacaacggcaatacgctgaccaaagtccccctgttggctacatccccgttatcggcaacctctttaaaacactcgggaaaaaaccgaccgccgcgaactgctgattttcaattacccccgagggaattatagatacggcgcaacagcctgcgctattgatgcgtcaaaataagggcatatgttttacagcatatgccctttctttatgctttttgccgcgaccgaaatgccgtcattcccgcgagcgaatccaacttgtccggtttcggttgtttttcgtctcgtaacttttgagccgtcattcccgcgaaatcggaaatccagtccgttcagtttcggtcatttccgataaattcctgttgcttttcatttctagattcccactttcgtggaataacggcggaagggataaatcctcgcaatccaaagcctgctcatttccacaaaaaacagcaacccgaaacaccccgtcattcccgagcaggcggaatctagaaccgcaacgccaggaatctgtcggatacggctgaaaccgaacgactggattcccg

SEQ ID NO: 90 - protein, 174 aa, Neisseria gonorrhoeae
MKKALAALIALALPAAALAEGASGFYVQADAAHAKASSSLGSAKGFSPRISAGYRINDLRFAVDYTRYKNYKAPSTDFKLYSIGASVIYDFDTQSPVKPYFGARLSLNRASAHLGGSDSFSKTSAGLGVLAGVSYAVTPNVDLDAGYRYNYVGKVNTVKNVRSGELSAGVRVKF

SEQ ID NO: 91 - DNA, 710 bp, Neisseria gonorrhoeae
cggcaaagcagccggatgccgccgcgtatcttgaggcattgaaaatattacgatgcaaaaagaaaatttcagtataatacggcaggattctttaacggattattaacaatttttctccctgaccataaaggaaccaaaatatgaaaaaagcacttgccgcactgattgccctcgcactcccggccgccgcactggcggaaggcgcatccggcttttacgtccaagccgatgccgcacacgccaaagcctcaagctctttaggttctgccaaaggcttcagcccgcgcatctccgcaggctaccgcatcaacgacctccgcttcgccgtcgattacacgcgctacaaaaactataaagccccatccaccgatttcaaactttacagcatcggcgcgtccgtcatttacgacttcgacacccaatcgcccgtcaaaccgtatttcggcgcgcgcttgagcctcaaccgcgcttccgcccacttgggcggcagcgacagcttcagcaaaacctccgccggcctcggcgtattggcgggcgtaagctatgccgttaccccgaatgtcgatttggatgccggctaccgctacaactacgtcggcaaagtcaacactgtcaaaaacgtccgttccggcgaactgtccgccggcgtgcgcgtcaaattctgatatacgcgttattccgcaaaccgccgagccttcggcggttttttg

SEQ ID NO: 92 - protein, 792 aa, Neisseria gonorrhoeae
MKLKQIASALMMLGISPLAFADFTIQDIRVEGLQRTEPSTVFNYLPVKVGDTYNDTHGSAIIKSLYATGFFDDVRVETADGLLLLTVIVCPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSDRFDRQKFAQDMEKVTDFYQNNGYFDFRILDTDIQTNEDKTRQTIKITVHEGGRFRWGKVSIEGDTNEVPKAELEKLLTMKPGKWYERQQMTAVLGEIQNRMGSAGYAYSEISVQPLPNAGTKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDIYGKAFDPRKASTSVKQYKTTTAGGGVRMGIPVTEYDRVNFGLAAEHLTVNTYNKAPKRYADFIKQYGKTDGADGSFKGLLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGRTYTAAENGNNKSVYSENAHKSTFTNELRYSAGGAVTWLSPLGPMKFIYAYPLKKKPEDEIQRFQFQLGTTF

SEQ ID NO: 93 - DNA, 2379 bp, Neisseria gonorrhoeae
atgaaactgaaacagattgcctccgcactgatgatgttgggcatatcgcctttggcatttgccgacttcaccatccaagacatccgtgtcgaaggcttgcagcgtaccgagccgagcaccgtattcaactacctgcccgtcaaagtcggcgacacctacaacgacacacacggcagtgccatcatcaaaagcctgtacgccaccggtttctttgacgacgtacgagtcgaaactgcggacgggctgcttctgctgaccgttatcgtatgccctaccatcggctcgctcaacatcaccggcgccaaaatgctgcagaacgacgccatcaagaaaaacctcgaatcgttcgggctggcgcagtcgcaatactttaatcaggcgacactcaaccaggcagtcgccggcctgaaagaagaatatctcgggcgcggcaaactcaatatccaaatcacgcccaaagtaaccaaactcgcccgcaaccgcgtcgacatcgacatcacgattgacgagggcaaatccgccaaaatcaccgacatcgaatttgaaggcaaccaagtctattccgaccgcaaactgatgcggcagatgtcgctgaccgaaggcggcatttggacatggctgacacgaagcgaccggttcgaccgccagaaattcgcccaagacatggaaaaagtaaccgacttctaccagaacaacggctacttcgatttccgtatcctcgataccgacatccaaaccaacgaagacaaaaccaggcagaccatcaaaatcaccgtccacgaaggcggacgtttccgctggggcaaagtgtcgattgaaggcgacaccaacgaagtccccaaggccgaactggaaaaactgctgaccatgaagcccggcaaatggtacgaacgccagcagatgaccgccgttttgggtgagattcagaaccgcatgggctcggcaggctacgcatacagcgaaatcagcgtacagccgctgccgaacgccggaaccaaaaccgtcgatttcgtcctgcacatcgaaccgggcagaaaaatctacgtcaacgaaatccacatcaccggcaacaacaaaacccgcgacgaagtcgtgcgccgcgaattgcgccaaatggaatccgcgccttacgacacctccaagctgcaacgctccaaagagcgcgtcgagcttttgggctacttcgacaacgtacagtttgatgccgtcccgcttgccggtacgcccgacaaagtcgatttgaacatgagcctgaccgaacgttccaccggctcgctcgacttgagcgcgggctgggttcaggataccggcttggtcatgtccgccggcgtatcgcaggacaacctgttcggtacgggcaagtcggccgccctgcgcgcctcgcgaagcaaaaccacgctcaacggctcgctgtcgtttaccgacccgtacttcacggcagacggggtcagcctgggctacgatatttacggaaaagccttcgacccgcgcaaagcatcgaccagcgtcaaacaatataaaaccaccaccgccggcggcggcgtaaggatgggtatccccgttaccgaatacgaccgcgtcaatttcgggctggcggcggaacacctgaccgtcaacacctacaacaaagcacccaaacgctatgccgactttatcaaacaatacggcaaaaccgacggcgcagacggcagcttcaaaggcctgctgtacaaaggcactgtcggctgggggcgcaacaagaccgacagcgccttatggccgacgcgcggctacctgaccggcgtaaatgccgaaatcgccctgcccggcagcaaactgcaatactactccgccacccacaaccaaacctggttcttccccttaagcaaaaccttcacgctgatgctcggcggcgaagtcggcattgcgggcggctacggcagaaccaaagaaatccccttctttgaaaacttctacggcggcggcctgggttcggtgcgcggctacgaaagcggcacgctcggcccgaaagtgtatgacgaatacggcgaaaaaatcagctacggcggcaacaaaaaagccaacgtctccgccgagctgctcttcccgatgcccggtgcgaaagacgcacgcaccgtccgcctgagcctgtttgccgacgcaggcagcgtgtgggacggcagaacctataccgccgccgaaaacggtaacaacaaatcggtttactcggaaaacgcgcataaatccacctttaccaacgaattgcgctattccgccggcggcgcggttacctggctctcgcctttgggcccgatgaaattcatctacgcctacccgctgaagaaaaaaccggaagacgaaatccaacgcttccaattccagctcggcacgacgttctaa

SEQ ID NO: 94 - DNA, 1128 bp, Neisseria gonorrhoeae
atgaatacacgaaatatgcgctatattcttttgacaggactgttgccgacggcatccgcttttggagagaccgcgctgcaatgcgccgctttgacggacaatgttacgcgtttggcgtgttacgacaggatttttgcggcacagcttccgtcttcggcagggcaggaagggcaggagtcgaaagccgtactcaatctgacggaaaccgtccgcagcagcttggataagggcgaggcggtcattgttgttgaaaaaggcggggatgcgcttcctgccgacagtgcgggcgaaaccgccgatatctatacgcctttgagcctgatgtacgacttggacaaaaacgatttgcgcgggctgttgggcgtacgcgaacacaatccgatgtaccttatgccgttttggtataacaattcgcccaactatgccccgagttcgccgacgcgcggtacgactgtacaggaaaaattcggacagcagaaacgtgcggaaaccaaattgcaggtttcgttcaaaagcaaaattgccgaaaatttgtttaaaacccgcgcggatctgtggttcggctacacccaaagatccgattggcagatttacaaccaaggcaggaaatccgcgccgttccgcaatacggattacaaacctgaaattttcctgacccagcctgtgaaggcggatttgccgttcggcggcaggctgcgtatgctcggtgcgggttttgtccaccagtccaacggacagagccgtcccgaatcgcgttcgtggaacaggatttatgccatggcaggcatggaatggggcaaattgacggtgattccgcgcgtgtgggtgcgtgcgttcgatcagagcggcgataaaaacgacaatcccgatattgccgactatatggggtatggcgacgtgaagctgcagtaccgcctgaacgacaggcagaatgtgtattccgtattgcgctacaaccccaaaacgggctacggcgcgattgaagccgcctacacgtttccgattaagggcaaactcaaaggcgtggtacgcggattccacggttacggcgagagcctgatcgactacaaccacaagcagaacggtatcggtatcgggttgatgttcaacgactgggacggcatctga

SEQ ID NO: 95 - protein, 375 aa, Neisseria gonorrhoeae
MNTRNMRYILLTGLLPTASAFGETALQCAALTDNVTRLACYDRIFAAQLPSSAGQEGQESKAVLNLTETVRSSLDKGEAVIVVEKGGDALPADSAGETADIYTPLSLMYDLDKNDLRGLLGVREHNPMYLMPFWYNNSPNYAPSSPTRGTTVQEKFGQQKRAETKLQVSFKSKIAENLFKTRADLWFGYTQRSDWQIYNQGRKSAPFRNTDYKPEIFLTQPVKADLPFGGRLRMLGAGFVHQSNGQSRPESRSWNRIYAMAGMEWGKLTVIPRVWVRAFDQSGDKNDNPDIADYMGYGDVKLQYRLNDRQNVYSVLRYNPKTGYGAIEAAYTFPIKGKLKGVVRGFHGYGESLIDYNHKQNGIGIGLMFNDWDGI

SEQ ID NO: 96 - DNA, 1000 bp, Neisseria gonorrhoeae
ttttggcttccagcgtttcgttgttttcgtacaagtcgtaagtcagcttcagattgttggcttttttaaagtcttcgaccgtactctcgtcaacataattcgaccagttgtagatgttcagagtatcggtggcagcggcttcggcattggcagcaggtgcgctgcctgcttgaggctgcacggcgtttttttcgctgccgccgcaggctgccagagacagcgcggccaaaacggctaatacggattttttcatacgggcagattcctgatgaaagaggttggaaaaaaagaaaaccccgcgccccataaacaccccggcgcaaggtttgggtattgtaaagtaaatttgtgcaaactcaaagcgatattggcctgattttcctaaaaaattaccctgtttccaaaaaagggggggaaacggccgcccgattttgccgtttttttgcgccgtcagggtgtccgacgggcggatagagaaaaaaggcttgcatataatgtaaaccccctttaaaattgcgcgtttacagaatttatttttctttcaggagattccaatatggcaaacagcgcacaagcacgcaaacgtgcccgccagtccgtcaaacaacgcgcccacaacgctagcctgcgtaccgcattccgcaccgcagtgaaaaaagtattgaaagcagtcgaagcaggcgataaagctgccgcacaagcggtttaccaagagtccgtcaaagtcatcgaccgcatcgccgacaaaggcgtgtttcataaaaacaaagcggctcgccacaaaagccgcctgtctgcaaaagtaaaagcactggcttgatttttgcaaaaccgccaaggcggttgatacgcgataagcggaaaaccctgaagcccgacggtttcggggttttctgtatttcgggggtaaagttcgaaatggcggaaagggtgcggttttttatccgaatccgctataaaatgccgtttgaaaaccaatatgccgacaatgggggcggag

SEQ ID NO: 97 - protein, 713 aa, Neisseria gonorrhoeae
MNAPFFRLSLLSLTLAAGFAHAAENNANVALDTVTVKGDRQGSKIRTNIVTLQQKDESTATDMRELLKEEPSIDFGGGNGTSQFLTLRGMGQNSVDIKVDNAYSDSQILYHQGRFIVDPALVKVVSVQKGAGSASAGIGATNGAIIAKTVDAQDLLKGLDKNWGVRLNSGFAGNNGASYGASVFGKEGNFDGLFSYNRNDEKDYEAGKGFRNDNGGKTVPYSALDKRSYLAKIGTTFGDGDHRIVLSHMKDQHRGIRTVREEFAVSEKNSRITIKRQAPSYRETTQSNTNLAYTGKDLGFVEKLDANAYVLEKKRYSADDKDNGYAGNVKGPNHTRIATRSMNFNFDSRLAEQTLLKYGINYRHQEIKPQAFLNSEFEIKDKEKATNEEKKKNRENEKIAKAYRLTNPTKTDTGAYIEAIHEIDGFTLTGGLRYDRFKVKTHDGKTVSSSSLNPSFGVIWQPREHWSFSASHNYAGRSPRLYDALQTHGKRGIISIADGTKAERARNTEIGFNYNDGTFAANGSYFRQTIKDALANPQNRHDSVAVREAVNAGYIKNHGYELGASYRTGGLTAKVGVSHSKPRFYDTHKDKLLSANPEFGAQVGRTWTASLAYRFKNPNLEIGWRGRYVQKAVGSILAAGQKDRDGKLENVVRQGFGVNDVFANWKPLGKDTLNVNLSVNNVFDKFYYPHSQRWTNTLPGVGRDVRLGVNYKF

SEQ ID NO: 98 - DNA, 2600 bp, Neisseria gonorrhoeae
aaaccggtacggcgttgccccgccttagctcaaagagaacgattccctaaggtgctgaagcaccgagtgaatcggttccgtactatttgtactgtctgcggcttcgccgccttgtcctgatttttgttagtccacatatacatttccgacaaaacctgtcaacaaaaaacaacgcttcgcaaataaaaacgataatcagctttacacaacccccccccgctaatataaacaaaaataattattattattttttcttatcctgccaaaccttaacggtttggcttaacttcccttcatacactcaaaaggacgaacaaatgaacgccccgtttttccgcctcagcctgctctcgctcacacttgccgccggctttgcccacgcggcagaaaataatgccaatgtcgcattggataccgttaccgtaaaaggcgaccgccaaggcagcaaaatccgtaccaacatcgttacgcttcaacaaaaagacgaaagcaccgcaaccgatatgcgcgaactcttaaaagaagagccctccatcgatttcggcggcggcaacggcacgtcccaattcctgacgctgcgcggtatgggtcagaactctgtcgacatcaaggtggacaacgcctattccgacagccaaatcctttaccaccaaggcagatttattgtcgatcccgctttggttaaagtcgtttccgtacagaaaggcgcgggttccgcctctgccggtatcggcgcgaccaacggcgcgatcatcgccaaaaccgtcgatgcccaagacctgctcaaaggcttggataaaaactggggcgtgcgcctcaacagcggctttgccggcaacaacggcgcaagctacggcgcaagcgtattcggaaaagagggcaacttcgacggtttgttctcttacaaccgcaacgatgaaaaagattacgaagccggcaaaggtttccgcaatgacaacggcggcaaaaccgtaccgtacagcgcgctggacaaacgcagctacctcgccaaaatcggaacaaccttcggcgacggcgaccaccgcatcgtgttgagccatatgaaagaccaacaccggggcatccgcactgtgcgtgaagagtttgccgtcagcgaaaaaaattcacggataactattaaacgccaagccccatcctaccgcgaaaccactcaatccaacaccaacttggcgtacaccggcaaagatttgggctttgtcgaaaaactggatgccaacgcctatgtgttggaaaagaaacgctattccgccgatgacaaagataacggctacgcaggcaatgtaaaaggccccaaccatacccgaatcgccactcggagtatgaacttcaacttcgacagccgccttgccgaacaaaccctgttgaaatacggcatcaactaccgccatcaggaaatcaaaccgcaagcgtttttgaactcggaatttgaaataaaagataaagaaaaagcaactaatgaagagaaaaagaagaaccgtgaaaatgaaaaaattgccaaagcctaccgcctgaccaacccgaccaaaaccgataccggcgcgtatatcgaagccattcacgagattgacggctttaccctgaccggcgggctgcgttacgaccgcttcaaggtgaaaacccacgacggcaaaaccgtttcaagcagcagcctcaacccgagtttcggcgtgatttggcagccgcgcgaacactggagcttcagcgcgagccacaactacgccggccgcagcccgcgcctgtatgacgctctgcaaacccacggcaagcgcggcatcatctcgattgccgacggcacgaaagccgaacgcgcgcgcaataccgaaatcggcttcaactacaacgacggcacgtttgccgcaaacggcagctacttccggcagaccatcaaagacgcgcttgccaatccgcaaaaccgccacgactccgtcgccgtccgcgaagccgtcaacgccggctacatcaaaaaccacggttacgaattgggcgcgtcctaccgcaccggcggcctgaccgccaaagtcggcgtaagccacagcaaaccgcgcttttacgatacgcacaaagacaagctgttgagcgcgaaccctgaatttggcgcacaagtcggccgcacttggacggcctcccttgcctaccgcttcaaaaacccgaatctggaaatcggctggcgcggtcgttatgttcaaaaagccgtgggttcgatattggcggcaggtcaaaaagaccgcgacggcaaattggaaaacgttgtacgccaaggtttcggtgtgaacgatgtcttcgccaactggaaaccgctgggcaaagacacgctcaatgttaatctttcggttaacaacgtgttcgacaagttctactatccgcacagccaacgctggaccaataccctgccgggcgtgggacgtgatgtacgcctgggcgtgaactacaagttctaaaacgcacatcccgaaaaaatgccgtctgaaagcctttcagacggcatctgtcctgataatttgatatatagtggattaacaaaaaccggtacggcgttgccccgccttagctcaaagggaacgattccctaaggtgctgaa

SEQ ID NO: 99 - protein, 236 aa, Neisseria gonorrhoeae
MTKQLKLSALFVALLASGTAVAGEASVQGYTVSGQSNEIVRNNYGECWKNAYFDKASQGRVECGDAVAVPEPEPAPVAVVEQAPQYVDETISLSAKTLFGFDKDSLRAEAQDNLKVLAQRLSRTNVQSVRVEGHTDFMGSEKYNQALSERRAYVVANNLVSNGVPASRISAVGLGESQAQMTQVCQAEVAKLGAKASKAKKREALIACIEPDRRVDVKIRSIVTRQVVPARNHHQH

SEQ ID NO: 100 - DNA, 1349 bp, Neisseria gonorrhoeae
gaattcctatccgatttgccgccatgtttctacagcggcctgtatgttggcaattcagcagttgcttctgtatctgctgtacaaatctaatgagggaataaaatgaccaaacagctgaaattaagcgcattattcgttgcattgctcgcttccggcactgctgttgcgggcgaggcgtccgttcagggttacaccgtaagcggccagtcgaacgaaatcgtacgcaacaactatggagaatgctggaaaaacgcctactttgataaagcaagccaaggtcgcgtagaatgcggcgatgcggttgccgtccccgagcccgaacccgcgcctgtcgccgttgtggagcaggctcctcaatatgttgatgaaaccatttccctgtctgccaaaaccctgttcggtttcgataaggattcattgcgcgccgaagctcaagacaacctgaaagtattggcgcaacgcctgagtcgaaccaatgtccaatctgtccgcgtcgaaggccataccgactttatgggttctgaaaaatacaatcaggctctgtccgaacgccgcgcatacgtagtggcaaacaacctggtcagcaacggcgtacctgcttctagaatttctgctgtcggcttgggcgaatctcaagcgcaaatgactcaagtttgtcaagccgaagttgccaaactgggtgcgaaagcctctaaagccaaaaaacgtgaggctctgattgcatgtatcgaacctgaccgccgcgtagatgtgaaaatccgcagcatcgtaacccgtcaggttgtgccggcacgcaatcatcaccaacactaaggctaggtaatatcttgccgatgcatgaggttagcggattttgtaccgggtactgttgcaatattcgtgaaacgtcggccggtatcgatgatgtgaaacaaaccccgcttttgcggggtttgtttttttgggtggttttctgaaacggctatcgtcagaatcggggtgcaggttcggattcggattcagattcatgtttgtgtcccattgccgcgctttatagtggattaacaaaaatcaggacaaggcgacgaagccgcagacagtacaatagtacggcaaggcgaggcaacgccgtaccggtttaaatttaatccactatatcggttgaaactctgattttaaggcggtaggatgtgggtttgcccatagcaagggaatcctttctgtatcaagccccgaaagggataattcatacaaattcacgcctttccccctcattgggaaatggatggaatcgtgcccgatgtgtgcggcactgtatgccggatatggttttatcatcatcccttttcggttgaaaccccgcggaattc

SEQ ID NO: 101 - DNA, 32 bp, Artificial Sequence
  Primer
gggaatccatatgaaaaaactcttgaaatcgg

SEQ ID NO: 102 - DNA, 29 bp, Artificial Sequence
  Primer
ggaagatctttagaagcggaattgtgcat

SEQ ID NO: 103 - DNA, 36 bp, Artificial Sequence
  Primer
ctgcagaatcgcgaatgaaaaaactcttgaaatcgg

SEQ ID NO: 104 - DNA, 35 bp, Artificial Sequence
  Primer
ctgcagaatcgcgaatgagtagcaagctagtgaac

SEQ ID NO: 105 - DNA, 29 bp, Artificial Sequence
  Primer
aggagatctttagaattggaatcctccgg

SEQ ID NO: 106 - DNA, 1194 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcaccacttggtgtgacgctatcagcatgcgcgttggttactacggagactttgttttcgaccgtgttttgaaaactgatgtgaataaagaatttcagatgggagcggcgcctactaccagcgatgtagaaggcttacaaaacgatccaacaacaaatgttgctcgtccaaatcccgcttatggcaaacacatgcaagatgctgaaatgtttacgaacgctgcttacatggcattaaatatctgggatcgttttgatgtattttgtacattgggagcaactaccggttatttaagaggaaactccgcttccttcaacttagttggattattcggaacaaaaacacaatattctaagtttaatacagcgaatcttgttcctaacactgctttggatcgagctgtggttgagctttatacagacaccacctttgcttggagcgtaggtgctcgtgcagctctctgggaatgtgggtgtgcaacgttaggagcttctttccaatatgctcaatctaaacctaaagtagaagagttaaatgttctttgtaatgcatccgaatttactattaataagccgaaaggatatgttggggtggaatttccacttgatattaccgcaggaacagaagctgcgacagggactaaggatgcctctattgactaccatgagtggcaagcaagtttagccctttcttacagattaaatatgttcactccttacattggagttaaatggtctagagtaagttttgatgccgacacgatccgtatcgctcagcctaaattggctgaagcaatcttggatgtcactactctaaacccgaccatcgctggtaaaggagctgtggtctcttccggaagcgataacgaactggctgatacaatgcaaatcgtttccttgcagttgaacaagctgaaatctagaaaatcttgcggtattgcagtaggaacgactattgtagatgcagataaatacgcagttacagttgagactcgcttgatcgatgagagagcagctcacgtaaatgcacaattccgcttctaa

SEQ ID NO: 107 - DNA, 1182 bp, Chlamydia trachomatis; identical to SEQ ID NO: 56

SEQ ID NO: 108 - DNA, 1023 bp, Chlamydia trachomatis; identical to SEQ ID NO: 1

(19) United States
(12) Patent Application Publication
(10) Pub. No.: US 2005/0281847 A1
(43) Pub. Date: Dec. 22, 2005

(54) Vaccine composition

(75) Inventors: Francois-Xavier Jacques Berthet, Rixensart (BE); Yves Lobet, Rixensart (BE); Jan Poolman, Rixensart (BE); Vincent Georges Christian Louis Verlant, Rixensart (BE)

Correspondence Address: GLAXOSMITHKLINE;Corporate Intellectual Property - UW2220, P.O. Box 1539, King of Prussia, PA, 19406-0939, US

(73) Assignee: GlaxoSmithKline Biologicals, s.a.

(21) Appl. No.: 11/103,957

(22) Filed: Apr. 12, 2005

Related U.S. Application Data
(63) Continuation of application No. 10/467,534, filed on Feb. 3, 2004, now abandoned.

(30) Foreign Application Priority Data
Feb. 8, 2001 (GB) 0103169.9

(51) Int. Cl. A61K 39/118; C07K 14/295; C12N 1/21

(52) U.S. Cl. 424/263.1; 435/252.3; 530/350

(57) ABSTRACT

The present invention relates to the field of Gram-negative bacterial vaccine compositions, their manufacture, and the use of such compositions in medicine. More particularly it relates to the field of useful Gram-negative bacterial outer membrane vesicle (or bleb) compositions comprising heterologously expressed Chlamydia antigens, and advantageous methods of rendering these compositions more effective and safer as a vaccine.

### FIELD OF THE INVENTION

The present invention relates to the field of Gram-negative bacterial vaccine compositions, their manufacture, and the use of such compositions in medicine. More particularly it relates to the field of useful Gram-negative bacterial outer membrane vesicle (or bleb) compositions comprising heterologously expressed Chlamydia antigens, and advantageous methods of rendering these compositions more effective and safer as a vaccine.

### BACKGROUND OF THE INVENTION

Chlamydiae are obligate intracellular Gram negative bacteria which replicate only in cytoplasmic inclusions of eukaryotic cells. They have a unique developmental cycle which is represented by two major forms, the spore-like elementary body (EB) which is the infectious form transmitted from cell to cell, and the non infectious, metabolically active reticulate body (RB) which replicates within the host-cell.

Of the four known chlamydial species, Chlamydia trachomatis and C. pneumoniae are the important human pathogens. The recently defined species C. pneumoniae (Grayston 1989) is now recognized as a major cause of respiratory tract infections (Grayston 1993) and data are now growing for an association with atherosclerosis. The association is supported by seroepidemiological studies, studies demonstrating the presence of the bacterium in the atherosclerotic lesions, studies showing C. pneumoniae capability to replicate in the different cell types present in the atheroclerotic lesions, interventional trials with antibiotics in patients with coronary artery disease and experimental respiratory tract infection in rabbits or apolipoprotein-E deficient mice which leads to inflammatory changes in the aorta (Danesh 1997, Fong 1997, Laitinen 1997, Moazed 1997). Overall, those data implicate C. pneumoniae as a causative and/or aggravating factor of atherosclerosis.

C. trachomatis is a major human pathogen; transmitted from human to human (there is no known animal reservoir), it causes ocular and genital infections which can result in long term sequelae. Trachoma, a chlamydial ocular infection, is endemic in several developing countries and is the world's leading cause of preventable blindness with millions people affected by the disease. Genital chlamydial infections constitute the most common bacterial sexually transmitted disease (STD) worldwide. In 1996, WHO generated a new set of global estimates for four major STDs drawing an extensive review of the published and unpublished prevalence data (Gerbase 1998). It has been estimated that in 1995, 4 and 5.2 million new cases of C. trachomatis infection occured in individuals aged 15-49 for North America and Western Europe, respectively; worlwide, C. trachomatis totalized an estimate of 89.1 million new cases. Collectively, data show higher infection rates in women as compared to men (Washington 1987, Peeling 1995, Cates 1991); higher incidence is found in adolescent and young adults, approximately 70% of the chlamydial infections being reported in the 15-24 years of age group (Peeling 1995).

There is a clear need for effective vaccines against Chlamydia trachomatis and Chlamydia pneumoniae.

Outer Membrane Vesicles (Blebs)

Gram-negative bacteria are separated from the external medium by two successive layers of membrane structures. These structures, referred to as the cytoplasmic membrane and the outer membrane (OM), differ both structurally and functionally. The outer membrane plays an important role in the interaction of pathogenic bacteria with their respective hosts. Consequently, the surface exposed bacterial molecules represent important targets for the host immune response, making outer-membrane components attractive candidates in providing vaccine, diagnostic and therapeutics reagents.

Whole cell bacterial vaccines (killed or attenuated) have the advantage of supplying multiple antigens in their natural micro-environment. Drawbacks around this approach are the side effects induced by bacterial components such as endotoxin and peptidoglycan fragments. On the other hand, acellular subunit vaccines containing purified components from the outer membrane may supply only limited protection and may not present the antigens properly to the immune system of the host.

Proteins, phospholipids and lipopolysaccharides are the three major constituents found in the outer-membrane of all Gram-negative bacteria. These molecules are distributed asymmetrically: membrane phospholipids (mostly in the inner leaflet), lipooligosaccharides (exclusively in the outer leaflet) and proteins (inner and outer leaflet lipoproteins, integral or polytopic membrane proteins). For many bacterial pathogens which impact on human health, lipopolysaccharide and outer-membrane proteins have been shown to be immunogenic and amenable to confer protection against the corresponding disease by way of immunization.

The OM of Gram-negative bacteria is dynamic and, depending on the environmental conditions, can undergo drastic morphological transformations. Among these manifestations, the formation of outer-membrane vesicles or “blebs” has been studied and documented in many Gram-negative bacteria (Zhou, L et al. 1998. FEMS Microbiol. Lett. 163: 223-228). Among these, a non-exhaustive list of bacterial pathogens reported to produce blebs include: Bordetella pertussis, Borrelia burgdorferi, Brucella melitensis, Brucella ovis, Chlamydia psittaci, Chlamydia trachomatis, Esherichia coli, Haemophilus influenzae, Legionella pneumophila, Neisseria gonorrhoeae, Neisseria meningitidis, Pseudomonas aeruginosa and Yersinia enterocolitica. Although the biochemical mechanism responsible for the production of OM blebs is not fully understood, these outer membrane vesicles have been extensively studied as they represent a powerful methodology in order to isolate outer-membrane protein preparations in their native conformation.

Examples of bacterial species from which bleb vaccines can be made have been reviewed in WO 01/09350 (incorporated by reference herein). For example, N. meningitidis serogroup B (menB) excretes outer membrane blebs in sufficient quantities to allow their manufacture on an industrial scale. Such multicomponent outer-membrane protein vaccines from naturally-occurring menB strains have been found to be efficacious in protecting teenagers from menB disease and have become registered in Latin America. An alternative method of preparing outer-membrane vesicles is via the process of detergent extraction of the bacterial cells (EP 11243).

### SUMMARY OF THE INVENTION

The present inventors have found that Gram-negative bacterial blebs are an ideal context to present Chlamydia outer membrane proteins. In particular gonococcal blebs are useful in the case of presenting C. trachomatis OMPs and meningococcal blebs are useful in the case of presenting C. pneumoniae OMPs. This is because a) these outer-membrane proteins can integrate into such blebs in a native (or near-native) conformation thus retaining a useful immunological effect; b) blebs (particularly from Neisseria strains) can be produced in industrial quantities, c) blebs may be mucosally administered, and d) the combination of Chlamydia antigens with native bleb antigens can have important interactions for certain conditions such as salpingitis.

The present invention thus provides advantageous Gram-negative bacterial bleb preparations (derived from bleb-producing bacterial strains listed above, and preferably not derived from Chlamydia) presenting on its surface one or more recombinant (and preferably heterologous) protein antigens from Chlamydia trachomatis or Chlamydia pneumoniae. Advantagous vaccine formulations and methods of administration are also provided.

### DESCRIPTION OF THE INVENTION

The present invention provides a Gram-negative bacterial bleb presenting on its surface one or more outer membrane protein from Chlamydia.

In the context of this application the term “presenting on its surface” indicates that the Chlamydia protein should be exposed to the outer surface of the bleb and tethered to the outer membrane (preferably by being integrated into the outer membrane). Most preferably it should take up its native fold within the heterologous bleb context.

An efficient strategy to modulate the composition of a Bleb preparation in this way is to deliver one or more copies of a DNA segment containing an expression cassette comprising a gene encoding said Chlamydia outer membrane protein into the genome of a Gram-negative bacterium.

A non exhaustive list of preferred bacterial species that could be used as a recipient for such a cassette includes: Bordetella pertussis, Borrelia burgdorferi, Brucella melitensis, Brucella ovis, Chlamydia psittaci, Chlamydia trachomatis, Esherichia coli, Haemophilus influenzae, Legionella pneumophila, Neisseria gonorrhoeae, Neisseria meningitidis, Pseudomonas aeruginosa and Yersinia enterocolitica. Neisseria meningitidis, Neisseiria gonorrhoeae, Moraxella catarrhalis, Haemophilus influenzae, Pseudomonas aeruginosa, Chlamydia trachomatis, Chlamydia pneumoniae are more preferred for this purpose, and Neisseria gonorrhoeae and Neisseria meningitidis are most preferred for making the blebs of this invention. Preferably the Chlamydia OMPs are expressed heterologously, and in such situations Chlamydia strains should not be used to make the blebs of the invention.

The gene(s) contained in the expression cassette may be homologous (or endogenous) (i.e. exist naturally in the genome of the manipulated bacterium) or, preferably, heterologous (i.e. do not exist naturally in the genome of the manipulated bacterium). The introduced expression cassette may consist of unmodified, “natural” promoter/gene/operon sequences or engineered expression cassettes in which the promoter region and/or the coding region or both have been altered. A non-exhaustive list of preferred promoters (preferably strong) that could be used for expression includes the promoters porA, porB, lbpB, tbpB, p110, lst, hpuAB from N. meningitidis or N. gonorroheae, the promoters p2, p5, p4, ompF, p1, ompH, p6, hin47 from H. influenzae, the promoters ompH, ompG, ompCD, ompE, ompB1, ompB2, ompA of M. catarrhalis, the promoter λpL, lac, tac, araB of Escherichia coli or promoters recognized specifically by bacteriophage RNA polymerase such as the E. coli bacteriophage T7.

In a preferred embodiment of the invention the expression cassette is delivered and integrated in the bacterial chromosome by means of homologous and/or site specific recombination (as discussed in WO 01/09350 incorporated by reference herein). Integrative vectors used to deliver such genes and/or operons can be conditionally replicative or suicide plasmids, bacteriophages, transposons or linear DNA fragments obtained by restriction hydrolysis or PCR amplification. Integration is preferably targeted to chromosomal regions dispensable for growth in vitro. A non exhaustive list of preferred loci that can be used to target DNA integration includes the porA, porB, opa, opc, rnp, omp26, lecA, cps, lgtB genes of Neisseiria meningitidis and Neisseria gonorrhoeae, the P1, P5, hmw1/2, IgA-protease, fimE genes of NTHi; the lecA1, lecA2, omp106, uspA1, uspA2 genes of Moraxella catarrhalis. Alternatively, the expression cassette used to modulate the expression of bleb component(s) can be delivered into a bacterium of choice by means of episomal vectors such as circular/linear replicative plasmids, cosmids, phasmids, lysogenic bacteriophages or bacterial artificial chromosomes. Selection of the recombination event can be selected by means of selectable genetic marker such as genes conferring resistance to antibiotics (for instance kanamycin, erythromycin, chloramphenicol, or gentamycin), genes conferring resistance to heavy metals and/or toxic compounds or genes complementing auxotrophic mutations (for instance pur, leu, met, aro). Blebs may be made from the resulting modified strain.

The expression of some heterologous proteins in bacterial blebs may require the addition of outer-membrane targeting signal(s). The preferred method to solve this problem is by creating a genetic fusion between a heterologous gene and a gene coding for a resident OMP as a specific approach to target recombinant proteins to blebs. Most preferably, the heterologous gene is fused to the signal peptides sequences of such an OMP.

A particularly preferred application of this invention is the introduction of Chlamydia (trachomatis or pneumoniae) protective antigens (preferably outer membrane proteins) into Gram-negative bacterial blebs (preferably not from Chlamydia strains). This has several advantages including the fact that such blebs (and vaccines comprising them) are extremely suitable for mucosal administration, which is beneficial as a mucosal (IgA) immune response against the Chlamydia antigens present in the bleb will be more protective against Chlamydia infections which manifest themselves in the mucosa. Recombinant bacteria capable of producing blebs of the invention, processes of making such bacteria, and processes of making bleb preparations are further aspects of this invention.

Chlamydia trachomatis Antigens Integrated into a Gram Negative Bacterial Bleb

A particularly preferred embodiment is in the field of the prophylaxis or treatment of sexually-transmitted diseaseses (STDs). It is often difficult for practitioners to determine whether the principal cause of a STD is due to gonococcus or Chlamydia trachomatis infection. These two organisms are major causes of salpingitis—a disease which can lead to sterility in the host. It would be useful if a STD could be vaccinated against or treated with a combined vaccine effective against disease caused by both organisms. The Major Outer Membrane Protein (MOMP or OMPI or OMPI) of C. trachomatis has been shown to be the target of protective antibodies. However, the structural integrity of this integral membrane protein is important for inducing such antibodies. In addition, the epitopes recognised by these antibodies are variable and define more than 10 serovars. The bleb context of the invention allows the proper folding of one or more MOMP or other Chlamydia membrane proteins for vaccine purposes. The engineering of (preferably) a gonococcal strain expressing one or more C. trachomatis MOMP serovars and/or one or more other protective Chlamydia OMPs in the outer membrane, and the production of blebs therefrom, produces a single solution to the multiple problems of correctly folded membrane proteins, the presentation of sufficient MOMP serovars and/or other Chlamydia OMPs to protect against a wide spectrum of serovars, and the simultaneous prophylaxis/treatment of gonococcal infection (and consequently the non-requirement of practitioners to initially decide which organism is causing particular clinical symptoms—both organisms can be vaccinated against simultaneously thus allowing the treatment of the STD at a very early stage). Preferred loci for gene insertion in the gonoccocal chromosome are give above. Other preferred, protective C. trachomatis genes that could be incorporated are HMWP, PmpG and those OMPs disclosed in WO 99/28475 (incorporated by reference herein).

A particularly preferrred embodiment of the invention provides a Gram-negative bacterial bleb (preferably gonococcal) presenting on its surface the PorB outer membrane protein (see below) from Chlamydia trachomatis. A bacterial strain capable of producing such a bleb is a further aspect of the invention.

PorB Chlamydia trachomatis Serovar D (D/UW-3/Cx) DNA Sequence

ATGAGTAGCAAGCTAGTGAACTATCTCCGTTTGACTTTCCTATCTTTTTT
AGGGATCGCATCTACTTCATTAGACGCTATGCCTGCGGGGAATCCGGCGT
TTCCAGTCATCCCGGGGATTAATATTGAACAGAAAAATGCCTGTTCTTTC
GATTTATGTAATTCTTATGATGTACTATCCGCACTGTCCGGTAACCTGAA
GCTCTGCTTCTGCGGAGATTATATCTTTTCAGAAGAAGCTCAGGTAAAAG
ATGTCCCGTCGTTACCTCTGTGACAACAGCTGGGGTTGGTCCTTCTCCTG
ATATTACTTCGACAACCAAAACGCGAAATTTCGATCTCGTGAACTGTAAT
CTCAATACAAACTGTGTAGCTGTAGCTTTTTCCCTTCCTGATCGTTCGCT
GAGCGCGATTCCTCTGTTTGATGTGAGTTTCGAAGTGAAAGTAGGAGGAC
TGAAACAATACTACCGCCTTCCCATGAATGCCTATCGAGACTTCACCTCG
GAACCTCTCAATTCTGAATCAGAAGTTACGGACGGGATGATTGAAGTACA
GTCCAATTACGGATTTGTTTGGGATGTTAGCTTGAAAAAAGTCATATGGA
AAGATGGCGTTTCCTTTGTAGGCGTCGGTGCAGACTATCGCCATGCTTCT
TGCCCTATTGACTACATCATTGCAAACAGTCAAGCTAATCCAGAAGTATT
CATCGCTGACTCGGATGGGAAACTGAACTCAAGGAGTGGAGTGTCTGCGT
AGGTCTTACTACCTATGTGAATGACTACGTTCTTCCTTACTTAGCGTTTT
CTATAGGGAGTGTTTCTCGCCAAGCTCCGGACGACAGCTTCAAAAAATTA
GAAGATCGCTTCACTAACCTCAAATTTAAAGTTCGTAAAATTACCAGCTC
TCATCGTGGAAACATCTGCATCGGAGCGACAAACTATGTCGCCGATAACT
TCTTCTACAACGTAGAAGGAAGATGGGGAAGCCAGCGCGGTGTGAACGTC
TCCGGAGGATTCCAATTCTAA

Translated Amino Acid Sequence

MSSKLVNYLR LTFLSFLGIA STSLDAMPAG NPAFPVIPGI
NIEQKNACSF DLCNSYDVLS ALSGNLKLCF CGDYIFSEEA QVKDVPVVTS
VTTAGVGPSP DITSTTKTRN FDLVNCNLNT NCVAVAFSLP DRSLSAIPLF
DVSFEVKVGG LKQYYRLPMN AYRDFTSEPL NSESEVTDGM IEVQSNYGFV
WDVSLKKVIW KDGVSFVGVG ADYRHASCPI DYIIANSQAN PEVFIADSDG
KLNFKEWSVC VGLTTYVNDY VLPYLAFSIG SVSRQAPDDS FKKLEDRFTN
LKFKVRKITS SHRGNICIGA TNYVADNFFY NVEGRWGSQR AVNVSGGFQF

The presence of PorB in the blebs means that the antigen can be mucosally administered more easily, and provides more effective protection than if administered alone.

The present invention additionally provides a Gram-negative bacterial bleb (preferably gonococcal) presenting on its surface one or more of the following proteins from Chlamydia trachomatis, or C. trachomatis PorB in combination with one or more of the following proteins. It will be clear to a skilled person that instead of the sequences below (and the PorB sequence above), the natural analogue of the sequences from other C. trachomatis serovars or serotypes could be used, as could genes encoding functional analogues of the proteins comprising insertions, deletions or substitutions from the recited sequences which unaffect the immunological properties of the encoded protein. Preferably a sequence from a serovar D strain should be selected. A bacterial strain capable of producing such a bleb is a further aspect of the invention.

>gi|6578118|gb|AAC68456.2|predicted Protease containing IRBP and DHR
domains [Chlamydia trachomatis]
MKMNRIWLLLLTFSSAIHSPVQGESLVCKNALQDLSFLEHLLQVKYAPKTWKEQYLGWDLVQSSVSAQQK
LRTQENPSTSFCQQVLADFIGGLNDFHAGVTFFAIESAYLPYTVQKSSDGRFYFVDIMTFSSEIRVGDEL
LEVDGAPVQDVLATLYGSNHKGTAAEESAALRTLFSRNASLGHKVPSGRTTLKIRRPFGTTREVRVKWRY
VPEGVGDLATIAPSIRAPQLQKSMRSFFPKKDDAFHRSSSLFYSPMVPHFWAELRNHYATSGLKSGYNIG
STDGFLPVIGPVIWESEGLFRAYISSVTDGDGKSHKVGFLRIPTYSWQDMEDFDPSGPPPWEEFAKIIQV
FSSNTEALIIDQTNNPGGSVLYLYALLSMLTDRPLELPKHRMILTQDEVVDALDWLTLLENVDTNVESRL
ALGDNMEGYTVDLQVAEYLKSFGRQVLNCWSKGDIELSTPIPLFGFEKIHPHPRVQYSKPICVLINEQDF
SCADFFPVVLKDNDRALIVGTRTAGAGGFVFNVQFPNRTGIKTCSLTGSLAVREHGAFIENIGVEPHIDL
PFTANDIRYKGYSEYLDKVKKLVCQLINNDGTIILAEDCSF
>gi|3329331|gb|AE001359.1:101 -1906,
ATGAAAATGAATAGGATTTGGCTATTACTGCTTACCTTTTCTTCTGCCATACATTCTCCTGTACAAGGAG
AAAGCTTGGTTTGCAAGAATGCTCTTCAAGATTTGAGTTTTTTAGAGCATTTATTACAGGTTAAATATGC
TCCTAAAACATGGAAAGAGCAATACTTAGGATGGGATCTTGTTCAAAGCTCCGTTTCTGCACAGCAGAAG
CTTCGTACACAAGAAAATCCATCAACAAGTTTTTGCCAGCAGGTCCTTGCTGATTTTATCGGAGGATTAA
ATGACTTTCACGCTGGAGTAACTTTCTTTGCGATAGAAAGTGCTTACCTTCCTTATACCGTACAAAAAAG
TAGTGACGGCCGTTTCTACTTTGTAGATATCATGACTTTTTCTTCAGAGATCCGTGTTGGAGATGAGTTG
CTAGAGGTGGATGGGGCGCCTGTCCAAGATGTACTCGCTACTCTATATCGAAGCAATCACAAAGGGACTG
CAGCTGAAGAGTCGGCTGCTTTAACAACACTATTTTCTCGCATCGCCTCTTTAGGGCACAAAGTACCTTC
TGGGCGCACTACTTTAAAGATTCGTCGTCCTTTTGGTACTACGAGAGAAGTTCGTGTGAAATGGCGTTAT
GTTCCTGAAGGTGTAGGAGATTTGGCTACCATAGCTCCTTCTATCAGGGCTCCACAGTTACAGAAATCGA
TGAGAAGCTTTTTCCCTAAGAAAGATGATGCGTTTCATCGGTCTAGTTCGCTATTCTACTCTCCAATGGT
TCCGCATTTTTGGGCAGAGCTTCGCAATCATTATGCAACGAGTGGTTTGAAAAGCGGGTACAATATTGGG
AGTACCGATGGGTTTCTCCCTGTCATTGGGCCTGTTATATGGGAGTCGGAGGGTCTTTTCCGCCCTTATA
TTTCTTCGGTGACTGATGGGGATGGTAAGAGCCATAAAGTAGGATTTCTAAGAATTCCTACATATAGTTG
GCAGGACATGGAAGATTTTGATCCTTCAGGACCGCCTCCTTGGGAAGAATTTGCTAAGATTATTCAAGTA
TTTTCTTCTAATACAGAAGCTTTGATTATCGACCAAACGAACAACCCAGGTGGTAGTGTCCTTTATCTTT
ATGCACTGCTTTCCATGTTGACAGACCGTCCTTTAGAACTTCCTAAACATAGAATGATTCTGACTCAGGA
TGAAGTGGTTGATGCTTTAGATTGGTTAACCCTGTTGGAAAACGTAGACACAAACGTGGAGTCTCGCCTT
GCTCTGGGAGACAACATGGAAGCATATACTGTGGATCTACAGGTTGCCGAGTATTTAAAAAGCTTTGGAC
GTCAAGTATTGAATTGTTGGAGTAAAGGGGATATCGAGTTATCAACGCCTATTCCTCTTTTTGGTTTTGA
GAAGATTCATCCACATCCTCGAGTTCAATACTCTAAACCGATTTGTGTTTTGATCAATGAGCAAGACTTT
TCTTGTGCTGACTTCTTCCCTGTAGTTTTGAAAGACAATGATCGAGCTCTTATTGTTGGTACTCGAACAG
CTGGAGCTGGAGGATTTGTCTTTAATGTGCAGTTCCCAAATAGAACTGGAATAAAAACTTGTTCTTTAAC
AGGATCATTAGCTGTTAGAGAGCATGGTGCCTTCATTGAGAACATCGGAGTCGAACCGCATATCGATCTG
CCTTTTACAGCGAATGATATTCGCTATAAAGGCTATTCCGAGTATCTTGATAAGGTCAAAAAATTGGTTT
GTCAGCTGATCAATAACGACGGTACCATTATTCTTGCGGAAGATGGTAGTTTTTAA
>gi|6578109|gb|AAC68227.2|CHLPN 76kDa Homolog [Chlamydia trachomatis]
MKKYFYKGFVGALLLACGSTNLAFAQASSMDSQLWSVEDLDSYLSSKGFVETRKRDGVLRLACDVRARWI
YAKEDLETTQTPAKPMLPTNRYRSEFNLYVDYTAANSWMTSKMNWVTIAGGESSAAGLDINRAFLGYRFY
KNPETQAEVFAEIGRSGLGDIFDSDVQFNSNFDGIHLYAARRISEKLPFTMIVHGGPFVVNMAEKEYAWV
VEAILNKLPGNFVVKTSVVDWNTLTAKTNDPADASAAQPAKPNTKYDYLVWQWLVGKSTAMPWFNGQTKN
LYTYGAYLFNPLAEIPENWKQSTTPTTKITNGKENHAWFIGCSLGGVRRAGDWSATVRYEYVEALAIPEI
DVAGIGRGNQMKYWFAQAIKQGLDPKESNGFTNYKGVSYQFVMGLTDSVSFRAYAAYSKPANDNLGSDFT
YRKYDLGLISSF
>gi|3329068|gb|AE001333.1:c3495-2197,
ATGAAAAAATACTTTTATAAAGGGTTTGTAGGCGCGCTTTTATTAGCTTGTGGGTCTACAAACTTGGCTT
TTGCGCAGGCTAGTTCGATGGATAGCCAGCTATGGTCTGTTGAAGATTTAGATTCTTATTTGAGTTCCAA
AGGTTTTGTCGAGACTCGTAAGCGCGATGGAGTTCTACGTTTAGCTGGAGATGTCCGCGCTCGATGGATT
TATGCAAAAOAGGATCTTGAGACAACTCAGACTCCTGCTAAACCTATGTTACCTACCAATCGGTATCGTA
GTGAATTCAATTTGTATGTGGATTACACCGCTGCTAATAGTTGGATGACTTCGAAAATGAATTGGGTAAC
GATTGCTGGCGGAGAATCTTCTGCAGCAGGGTTAGATATTAATCGTGCCTTCTTAGGATACCGATTCTAC
AAAAACCCAGAAACGCAAGCAGAAGTATTTGCAGAGATTGGTCGCTCTGGATTGGGAGATATTTTTGATT
CCGACGTTCAGTTTAATAGTAATTTCGACGGAATTCATTTATACGCTGCGCGACGTATTAGTGAGAAACT
TCCTTTCACCATGATTGTTCATGGTGGTCCTTTTCTCGTGAATATGGCAGAGAAAGAGTATGCTTGGGTC
GTGGAAGCTATTTTGAATAAACTCCCAGGAAATTTCGTTGTGAAAACCAGTGTTGTTGACTGGAATACGT
TAACAGCAAAAACGAATGATCCAGCAGACGCAAGCGCTGCACAACCAGCTAAACCTAATACCAAGTACGA
TTATTTAGTATGGCAATGGTTGGTTGGGAAGAGCACAGCTATGCCATGGTTTAATGGACAAACAAAAAAT
CTTTACACTTACGGAGCCTATCTCTTTAATCCATTAGCGGAAATACCACAGAACTGGAAACAATCAACAA
CTCCTACAACCAAAATTACAAATGGTAAGGAAAACCATGCTTGGTTCATCGGCTGCTCTCTAGGCGGTGT
TCGACGAGCTGGAGACTGGTCTGCAACAGTTCGTTATGAGTATGTTGAAGCTTTAGCGATTCCAGAAATT
GATGTCGCGGGTATTGGTCGCGGAAACCAAATGAAATATTGGTTTGCTCAAGCTATCAAACAAGGATTGG
ATCCTAAAGAATCTAACGGCTTTACTAACTATAAAGGAGTTTCCTATCAGTTTGTTATGGGTCTGACAGA
TTCGGTTTCTTTCCGAGCTTATGCTGCTTATTCTAAGCCTGCTAACGATAACCTTGGTAGCGACTTCACC
TATCGTAAGTATGACCTAGGTTTAATTTCTTCATTCTAA
>gi|3329350|gb|AAC68472.1|Putative Outer Membrane Protein
I [Chlamydia trachomatis]
MRPDHNNFCCLCAAILSSTHVLFGQDPLGETALLTKNPNHVVCTFFEDCTMESLFPALCAHASQDDPLYV
LGNSYCWFVSKLHITDPKEALFKEKGDLSIQNFRFLSFTDCSSKESSPSIIHQKNGQLSLRNNGSMSFCR
NHAEGSGGAISADAFSLQHNYLFTAFEENSSKGNGGAIQAQTFSLSRNVSPISFARNRADLNGGAICCSN
LICSGNVNPLFFTGNSATNGGAICCISDLNTSEKGSLSLACNQETLFASNSAKEKGGAIYAKHMVLRYNG
PVSFINNSAKIGGAIAIQSGGSLSILAGEGSVLFQNNSQRTSDQGLVRNAIYLEKDAILSSLEARNGDIL
FFDPIVQESSSKESPLPSSLQASVTSPTPATASPLVIQTSANRSVIFSSERLSEEEKTPDNLTSQLQQPI
ELKSGRLVLKDRAVLSAPSLSQDPQALLIMEAGTSLKTSSDLKLATLSIPLHSLDTEKSVTIHAPNLSIQ
KIFLSNSGDENFYENVELLSKEQNNIPLLTLSKEQSHLHLPDGNLSSHFGYQGDWTFSWKDSDEGHSLIA
NWTPKNYVPHPERQSTLVANTLWNTYSDMQAVQSMINTIAHGGAYLFGTWGSAVSNLFYAHDSSGKPIDN
WHHRSLGYLFGISTHSLDDHSFCLAAGQLLGKSSDSFITSTETTSYIATVQAQLATPLMKISAQACYNES
IHELKTKYRSFSKEGFGSWHSVAVSGEVCASIPIVSNGSGLFSSFSIFSKLQGFSGTQDGFEESSGEIRS
FSASSFRNISLPMGITFEKKSQKTRNYYYFLOAYIQDLKRDVESGPVVLLKNAVSWDAPMANLDSRAYMF
RLTNQRALHRLQTLLNVSYVLRGQSHSYSLDLGTTYRF
>gi|3329348|gb|AE001361.1:c3451-815,
ATGCGACCTGATCATATGAACTTCTGTTGTCTATGTGCTGCTATTTTGTCATCCACAGCGGTCCTCTTTG
GCCAGGATCCCTTAGGTGAAACCGCCCTCCTCACTAAAAATCCTAATCATGTCGTCTGTACATTTTTTGA
GGACTGTACCATGGAGAGCCTCTTTCCTGCTCTTTGTGCTCATGCATCACAAGATGATCCTTTGTATGTA
CTTGGAAATTCCTACTGTTGGTTCGTATCTAAACTCCATATCACGGACCCCAAAGAGGCTCTTTTTAAAG
AAAAAGGAGATCTTTCCATTCAAAATTTTCGCTTCCTTTCCTTCACAGATTGCTCTTCCAAGGAAAGCTC
TCCTTCTATTATTCATCAAAAGAATGGTCAGTTATCCTTGCGCAATAATGGTAGCATGAGTTTCTGTCGA
AATCATGCTGAAGGCTCTGGAGGAGCCATCTCTGCGGATGCCTTTTCTCTACAACACAACTATCTTTTCA
CACCTTTTGAAGAGAATTCTTCTAAAGGAAATGGCGGAGCCATTCAGGCTCAAACCTTCTCTTTATCTAG
AAATGTGTCGCCTATTTCTTTCGCCCGTAATCGTGCGGATTTAAATGGCGGCGCTATTTGCTGTAGTAAT
CTTATTTGTTCAGGGAATGTAAACCCTCTCTTTTTCACTGGAAACTCCGCCACGAATGGAGGCGCTATTT
GTTGTATCAGCGATCTAAACACCTCAGAAAAAGGCTCTCTCTCTCTTGCTTGTAACCAAGAAACGCTATT
TGCAAGCAATTCTGCTAAAGAAAAAGGCGGGGCTATTTATGCCAAGCACATGGTATTGCGTTATAACGGT
CCTGTTTCCTTCATTAACAACAGCGCTAAAATAGGTGGAGCTATCGCCATCCAGTCCGGAGGGAGTCTCT
CTATCCTTGCAGGTCAAGGATCTGTTCTGTTCCAGAATAACTCCCAACGCACCTCCGACCAAGGTCTAGT
AAGAAACGCCATCTACTTAGAGAAAGATGCGATTCTTTCTTCCTTAGAAGCTCGCAACCGAGATATTCTT
TTCTTTGATCCTATTGTACAAGAAAGTAGCAGCAAAGAATCGCCTCTTCCCTCCTCTTTGCAAGCCAGCG
TGACTTCTCCCACCCCAGCCACCGCATCTCCTTTAGTTATTCAGACAAGTGCAAACCCTTCAGTGATTTT
CTCGAGCGAACGTCTTTCTGAAGAAGAAAAAACTCCTGATAACCTCACTTCCCAACTACAGCAGCCTATC
GAACTGAAATCCGGACGCTTAGTTTTAAAAGATCGCGCTGTCCTTTCCGCGCCTTCTCTCTCTCAGGATC
CTCAAGCTCTCCTCATTATGGAAGCGGGAACTTCTTTAAAAACTTCCTCTGATTTGAAGTTAGCTACGCT
AAGTATTCCCCTTCATTCCTTAGATACTGAAAAAAGCGTAACTATCCACGCCCCTAACCTTTCTATCCAA
AAGATCTTCCTCTCTAATTCTGGAGATGAGAATTTTTATGAAAATGTAGAGCTTCTCAGTAAAGAGCAAA
ACAATATTCCTCTCCTTACTCTCTCTAAAGAGCAATCTCATTTACATCTTCCTGATGGGAACCTCTCTTC
TCACTTTGGATATCAAGGAGATTGGACTTTTTCTTGGAAAGATTCTGATGAAGGGCATTCTCTGATTGCT
AATTGGACGCCTAAAAACTATGTGCCTCATCCAGAACGTCAATCTACACTCGTTQCGAACACTCTTTGGA
ACACCTATTCCGATATGCAAGCTGTGCAGTCGATGATTAATACAATAGCGCACGGAGGAGCCTATCTATT
TGGAACGTGGGGATCTGCTGTTTCTAATTTATTCTATGCTCACGACAGCTCTGGGAAACCTATCGATAAT
TGGCATCATAGAAGCCTTGGCTACCTATTCGGTATCAGTACTCACAGTTTAGATGACCATTCTTTCTGCT
TGGCTGCAGGACAATTACTCGGGAAATCGTCCGATTCCTTTATTACGTCTACAGAAACGACCTCCTATAT
AGCTACTGTACAAGCGCAACTCGCTACCCCTCTAATGAAAATCTCTGCACAGCCATGCTATAATGAAAGT
ATCCATGAGCTAAAAACAAAATATCGCTCCTTCTCTAAAGAAGGATTCGGATCCTGGCATAGCGTTGCAG
TATCCGGAGAAGTGTGCGCATCGATTCCTATTGTATCCAATGGTTCCGGACTGTTCAGCTCCTTCTCTAT
TTTCTCTAAACTGCAAGGATTTTCAGGAACACAGGACGGTTTTGAGGAGAGTTCGGGAGAGATTCGGTCC
TTTTCTGCCAGCTCTTTCAGAAATATTTCACTTCCTATGGGAATAACATTTGAAAAAAAATCCCAAAAAA
CACGAAACTACTATTACTTTCTGGGAGCCTACATCCAAGACCTAAAACGTGATGTGGAATCGGGACCTGT
AGTGTTACTCAAAAATGCCGTCTCCTGGGATGCTCCTATGGCGAACTTGGATTCGCGAGCCTACATGTTC
AGGCTTACGAATCAAAGAGCTCTGCATAGACTTCAGACGCTGTTAAATGTGTCTTACGTACTGCGCGGGC
AAAGCCATAGTTACTCCCTGGATCTGGGGACCACTTACAGGTTCTAG
>gi|3329346|gb|AAC68469.1| Putative Outer Membrane Protein G
[Chlamydia trachomatis]
MQTSFHKFFLSMILAYSCCSLSGGGYAAEIMIPQGIYDGETLTVSFPYTVIGDPSGTTVFSAGELTLKNL
DNSIAALPLSCFGNLLGSFTVLGRGHSLTFENIRTSTNGAALSDSANSGLFTIEGFKELSFSNCNSLLAV
LPAATTNNGSQTPTTTSTPSNGTIYSKTDLLLLNNEKFSFYSNLVSGDGGAIDAKSLTVQGISKLCVFQE
NTAQADGGACQVVTSFSAMANEAPIAFIANVAGVRGGGIAAVQDGQQGVSSSTSTEDPVVSFSRNTAVEF
DGNVARVGGGIYSYGNVAFLNNCKTLFLNNVASPVYIAAEQPTNGQASNTSDNYGDGGAIFCKNGAQAAG
SNNSGSVSFDGEGVVFFSSNVAAGKGGAIYAKKLSVANCGPVQFLGNIANDGGAIYLGESGELSLSADYG
DIIFDGNLKRTAKENAADVNGVTVSSQAISMGSGGKITTLRAKAGHQILFNDPIEMANGNNQPAQSSEPL
KINDGEGYTGDIVFANGNSTLYQNVTIEQGRIVLREKAKLSVNSLSQTGGSLYMEAGSTLDFVTPQPPQQ
PPAANQLITLSNLHLSLSSLLANNAVTNPPTNPPAQDSHPAIIGSTTAGSVTISGPIFFEDLDDTAYDRY
DWLGSNQKIDVLKLQLGTQPSANAPSDLTLGNEMPKYGYQGSWKLAWDPNTANNGPYTLKATWTKTGYNP
GPERVASLVPNSLWGSILDIRSAHSAIQASVDGRSYCRGLWVSGVSNFFYHDRDALGQGYRYISGGYSLG
ANSYFGSSMFGLAFTEVFGRSKDYVVCRSNHHACIGSVYLSTKQALCGSYLFGDAFIRASYGFGNQHMKT
SYTFAEESDVRWDNNCLVGEIGVGLPIVITPSKLYLNELRPFVQAEFSYADHESFTEEGDQARAFRSCHL
MNLSVPVGVKFDRCSSTHPNKYSFMGAYICDAYRTISGTQTTLLSHQETWTTDAFHLARHGVIVRGSMYA
SLTSNIEVYGHGRYEYRDTSRGYGLSAGSKVRF
>gi|3329342|gb|AE001360.1:7736-10777,
ATGCAAACGTCTTTCCATAAGTTCTTTCTTTCAATGATTCTAGCTTATTCTTGCTGCTCTTTAACTGGGG
GGGGGTATGCAGCAGAAATCATGATTCCTCAAGGAATTTACGATGGGGAGACCTTAACTGTATCATTTCC
CTATACTGTTATACGAGATCCGAGTGGGACTACTGTTTTTTCTGCAGGAGAGTTAACGTTAAAAAATCTT
GACAATTCTATTGCAGCTTTGCCTTTAAGTTGTTTTCGGAACTTATTAGCGAGTTTTACTGTTTTAGGGA
GAGGACACTCGTTGACTTTCGAGAACATACGGACTTCTACAAATGCAGCTGCACTAAGTGACAGCGCTAA
TAGCGGGTTATTTACTATTGAGGGTTTTAAAGAATTATCTTTTTCCAATTGCAACTCATTACTTGCCGTA
CTGCCTGCTGCAACGACTAATAATGCTAGCCAGACTCCGACCACAACATCTACACCGTCTAATGGTACTA
TTTATTCTAAAACAGATCTTTTGTTACTCAATAATGAGAAGTTCTCATTCTATAGTAATTTAGTCTCTGG
AGATGGGGGAGCTATAGATGCTAAGAGCTTAACGGTTCAAGGAATTAGCAAGCTTTGTGTCTTCCAAGAA
AATACTGCTCAAGCTGATGGGGGAGCTTGTCAAGTAGTCACCAGTTTCTCTGCTATGGCTAACGAGGCTC
CTATTGCCTTTATAGCCAATGTTGCAGGAGTAAGAGGGGCAGGGATTGCTGCTGTTCAGGATGGGCAGCA
GGGAGTGTCATCATCTACTTCAACAGAAGATCCACTAGTAAGTTTTTCCAGAAATACTGCGGTAGAGTTT
GATGGGAACGTAGCCCGAGTAGGAGGAGGGATTTACTCCTACGGGAACGTTGCTTTCCTGAATAATGGAA
AAACCTTGTTTCTCAACAATCTTGCTTCTCCTGTTTACATTGCTCCTGAGCAACCAACAAATGGACAGGC
TTCTAATACGAGTGATAATTACGGAGATGGAGGAGCTATCTTCTGTAAGAATGGTGCGCAAGCAGCAGGA
TCCAATAACTCTGCATCAGTTTCCTTTGATGCAGAGGGAGTAGTTTTCTTTAGTAGCAATGTAGCTGCTG
GGAAAGGGCGAGCTATTTATGCCAAAAAGCTCTCGGTTGCTAACTGTGGCCCTGTACAATTCTTAGGGAA
TATCGCTAATGATGGTGGAGCGATTTATTTAGCAGAATCTCGAGAGCTCAGTTTATCTGCTGATTATGGA
GATATTATTTTCGATGGGAATCTTAAAAGAACAGCCAAAGAGAATGCTCCCGATGTTAATGGCGTAACTG
TGTCCTCACAAGCCATTTCGATGGGATCGGGAGGGAAAATAACGACATTAAGAGCTAAAGCAGGGCATCA
GATTCTCTTTAATGATCCCATCGAGATGGCAAACGGAAATAACCAGCCAGCGCAGTCTTCCGAACCTCTA
AAAATTAACGATGGTGAAGGATACACAGGGGATATTGTTTTTGCTAATGGAAACAGTACTTTGTACCAAA
ATGTTACGATACAGCAAGGAAGGATTGTTCTTCGTGAAAAGGCAAAATTATCAGTGAATTCTCTAAGTCA
GACAGGTGGGAGTCTGTATATGGAACCTGGGAGTACATTGGATTTTGTAACTCCACAACCACCACAACAG
CCTCCTGCCGCTAATCAGTTGATCACGCTTTCCAATCTGCATTTGTCTCTTTCTTCTTTGTTAGCAAACA
ATGCAGTTACGAATCCTCCTACCAATCCTCCAGCGCAAGATTCTCATCCTGCAATCATTGGTAGCACAAC
TGCTGGTTCTGTTACAATTAGTGGGCCTATCTTTTTTGAGGATTTGGATGATACAGCTTATGATAGGTAT
GATTGGCTAGGTTCTAATCAAAAAATCGATGTCCTGAAATTACAGTTAGGGACTCAGCCCTCAGCTAATG
CCCCATCACATTTGACTCTAGGGAATGAGATGCCTAAGTATGGCTATCAAGGAAGCTGGAAGCTTGCGTG
GGATCCTAATACAGCAAATAATGGTCCTTATACTCTGAAAGCTACATGGACTAAAACTGGGTATAATCCT
GGGCCTGAGCGAGTAGCTTCTTTGGTTCCAAATAGTTTATGGGGATCCATTTTAGATATACGATCTGCGC
ATTCAGCAATTCAAGCAAGTGTGGATGGGCGCTCTTATTGTCGAGGATTATGGCTTTCTGGAGTTTCGAA
TTTCTTCTATCATGACCGCGATGCTTTAGGTCAGGGATATCGGTATATTAGTGGGGGTTATTCCTTAGGA
GCAAACTCCTACTTTGGATCATCGATGTTTGGTCTAGCATTTACCGAAGTATTTGGTAGATCTAAAGATT
ATGTAGTGTGTCGTTCCAATCATCATGCTTGCATAGGATCCGTTTATCTATCTACCAAACAAGCTTTATG
TGGATCCTATTTGTTCGGAGATGCGTTTATCCGTGCTAGCTACGGGTTTGGGAACCAGCATATGAAAACC
TCATACACATTTGCAGAGGAGAGCGATGTTCGTTGGGATAATAACTGTCTGGTTGGAGAGATTGGAGTGG
GATTACCGATTGTGATTACTCCATCTAAGCTCTATTTGAATGAGTTGCGTCCTTTCGTGCAAGCTGAGTT
TTCTTATGCCGATCATGAATCTTTTACAGAGGAAGGCGATCAAGCTCGGGCATTCAGGAGTGGACATCTC
ATGAATCTATCAGTTCCTGTTGGAGTAAAATTTGATCGATGTTCTAGTACACACCCTAATAAATATAGCT
TTATGGGGGCTTATATCTGTGATGCTTATCGCACCATCTCTGGGACTCAGACAACACTCCTATCCCATCA
AGAGACATGGACAACAGATGCCTTTCATTTGGCAAGACATGGAGTCATAGTTAGAGGGTCTATGTATGCT
TCTCTAACAAGCAATATAGAAGTATATGGCCATGGAAGATATGAGTATCGAGATACTTCTCGAGGTTATG
GTTTGAGTGCAGGAAGTAAAGTCCGGTTCTAA
>gi|3329345|gb|AAC68468.1| Putative Outer Membrane Protein F [Chlamydia
trachomatis]
MIKRTSLSFACLSFFYLSTISILQANETDTLQFRRFTFSDREIQFVLDPASLITAQNIVLSNLQSNGTGA
CTISGNTQTQIFSNSVNTTADSGGAFDMVTTSFTASDNANLLFCNNYCTHNKGGGAIRSGGPIRFLNNQD
VLFYNNISAGAKYVGTGDHNEKNRGGALYATTITLTGNRTLAFINNMSGDCGGAISADTQISITDTVKGI
LFENNHTLNHIPYTQAENMARGGAICSRRDLCSISNNSGPIVFNYNQGGKGGAISATRCVIDNNKERIIF
SNNSSLGWSQSSSASNGGAIQTTQGFTLRNNKGSIYFDSNTATHAGGAINCGYIDIRDNGPVYFLNNSAA
WGAAFNLSKPRSATNYIHTGTGDIVFNNNVVFTLDGNLLGKRKLFHINNNEITPYTLSLGAKKDTRIYFY
DLFQWERVKENTSNNPPSPTSRNTITVNPETEFSGAVVFSYNQMSSDIRTLMGKEHNYIKEAPTTLKFGT
LAIEDDAELEIFNIPFTQNPTSLLALGSGATLTVGKHGKLNITNLGVILPIILKEGKSPPCIRVNPQDMT
QNTGTGQTPSSTSSISTPMIIFNGRLSIVDENYESVYDSMDLSRGKAEQLILSIETTNDGQLDSNWQSSL
NTSLLSPPHYGYQGLWTPNWITTTYTITLNNNSSAPTSATSIAEQKKTSETFTPSNTTTASIPNIKASAG
SGSGSASNSGEVTITKHTLVVNWAPVGYIVDPIRRGDLIANSLVHSGRNMTMGLRSLLPDNSWFALQGAA
TTLFTKQQKRLSYHGYSSASKGYTVSSQASGAHGHKFLLSFSQSSDKMKEKETNNRLSSRYYLSALCFEH
PMFDRIALIGAAACNYGTHNMRSFYGTKKSSKGKFHSTTLGASLRCELRDSMPLRSIMLTPFAQALFSRT
EPASIRESGDLARLFTLEQAHTAVVSPIGIKGAYSSDTWPTLSWEMELAYQPTLYWKRPLLNTLLIQNNG
SWVTTNTPLAKHSFYGRGSHSLKFSHLKLFANYQAEVATSTVSHYIISTAGGALVF
>gi|3329342|gb|AE001360.1:C7571-4467,
ATGATTAAAAGAACTTCTCTATCCTTTGCTTGCCTCAGTTTTTTTTATCTTTCAACTATATCCATTTTGC
AAGCTAATGAAACGGATACGCTACAGTTCCGGCGATTTACTTTTTCGGATAGAGAGATTCAGTTCGTCCT
AGATCCCGCCTCTTTAATTACCGCCCAAAACATCGTTTTATCTAATTTACACTCAAACCGAACCGGAGCC
TGTACCATTTCAGGCAATACGCAAACTCAAATCTTTTCTAATTCCGTTAACACCACCGCAGATTCTGGTG
GAGCCTTTGATATGGTTACTACCTCATTCACGGCCTCTGATAATGCTAATCTACTCTTCTGCAACAACTA
CTGCACACATAATAAAGGCGGAGGAGCTATTCGTTCCGGAGGACCTATTCGATTCTTAAATAATCAAGAC
GTGCTTTTTTATAATAACATATCGGCAGGGGCTAAATATGTTGGAACAGGAGATCACAACGAAAAAAATA
GGGGCGGTGCGCTTTATGCAACTACTATCACTTTGACAGGGAATCGAACTCTTGCCTTTATTAACAATAT
GTCTGGAGACTGCGGTGGAGCCATCTCTGCTGACACTCAAATATCAATAACTGATACCGTTAAAGGAATT
TTATTTGAAAACAATCACACGCTCAATCATATACCGTACACGCAAGCTGAAAATATGGCACGAGGAGGAG
CAATCTGTAGTAGAAGAGACTTGTCCTCAATCAGCAATAATTCTGGTCCCATAGTTTTTAACTATAACCA
AGGCGGGAAAGGTGGAGCTATTAGCGCTACCCGATGTGTTATTGACAATAACAAAGAAAGAATCATCTTT
TCAAACAATAGTTCCCTGGGATGGAGCCAATCTTCTTCTGCAAGTAACGGAGGAGCCATTCAAACCACAC
AAGGATTTACTTTACGAAATAATAAAGGCTCTATCTACTTCGACAGCAACACTGCTACACACGCCGGGGG
AGCCATTAACTGTGGTTACATTGACATCCGAGATAACGGACCCGTCTATTTTCTAAATAACTCTGCTGCC
TGGGGAGCGGCCTTTAATTTATCGAAACCACGTTCAGCGACAAATTATATCCATACAGGGACAGGCGATA
TTGTTTTTAATAATAACGTTGTCTTTACTCTTGACGGTAATTTATTAGGGAAACGGAAACTTTTTCATAT
TAATAATAATGAGATAACACCATATACATTGTCTCTCGGCGCTAAAAAAGATACTCGTATCTATTTTTAT
GATCTTTTCCAATGGGAGCGTGTTAAAGAAAATACTAGCAATAACCCACCATCTCCTACCAGTAGAAACA
CCATTACCGTTAACCCGGAAACAGAGTTTTCTGGAGCTGTTGTGTTCTCCTACAATCAAATGTCTAGTGA
CATACGAACTCTGATGGGTAAAGAACACAATTACATTAAAGAAGCCCCAACTACTTTAAAATTCGGAACG
CTAGCCATAGAAGATGATGCAGAATTAGAAATCTTCAATATCCCGTTTACCCAAAATCCGACTAGCCTTC
TTGCTTTAGGAAGCGGCGCTACGCTCACTGTTGGAAAGCACGGTAAGCTCAATATTACAAATCTTGGTGT
TATTTTACCCATTATTCTCAAAGAGCGGAAGAGTCCGCCTTGTATTCGCGTCAACCCACAAGATATGACC
CAAAATACTGGTACCGGCCAAACTCCATCAAGCACAAGTAGTATAAGCACTCCAATGATTATCTTTAATG
GGCGCCTCTCAATTGTAGACGAAAATTATGAATCAGTCTACGACAGTATGGACCTCTCCAGAGGGAAAGC
AGAACAACTAATTCTATCCATAGAAACCACTAATCATGGGCAATTAGACTCCAATTGGCAAAGTTCTCTG
AATACTTCTCTACTCTCTCCTCCACACTATGGCTATCAAGGTCTATGGACTCCTAATTGGATAACAACAA
CCTATACCATCACGCTTAATAATAATTCTTCAGCTCCAACATCTGCTACCTCCATCGCTGAGCAGAAAAA
AACTAGTGAAACTTTTACTCCTAGTAACACAACTACAGCTAGTATCCCTAATATTAAAGCTTCCGCAGGA
TCAGGCTCTGGATCGGCTTCCAATTCAGCAGAAGTTACGATTACCAAACATACCCTTGTTGTAAACTGGG
CACCAGTCGGCTACATAGTAGATCCTATTCGTAGAGGAGATCTGATAGCCAATAGCTTAGTACATTCAGG
AAGAAACATGACCATGGGCTTACGATCATTACTCCCGGATAACTCTTGGTTTGCTTTGCAAGGAGCTGCA
ACAACATTATTTACAAAACAACAAAAACGTTTGAGTTATCATGGCTACTCTTCTGCATCAAAGGGGTATA
CCGTCTCTTCTCAAGCATCAGGAGCTCATGGTCATAAGTTTCTTCTTTCCTTCTCCCAGTCATCTGATAA
GATGAAAGAAAAAGAAACAAATAACCGCCTTTCTTCTCGTTACTATCTTTCTGCTTTATGTTTCGAACAT
CCTATGTTTGATCGCATTGCTCTTATCGGAGCAGCAGCTTGCAATTATGGAACACATAACATGCGGAGTT
TCTATGGAACTAAAAAATCTTCTAAAGGGAAATTTCACTCTACAACCTTAGGAGCTTCTCTTCGCTGTGA
ACTACGCGATAGTATGCCTTTACGATCAATAATGCTCACCCCATTTGCTCAGGCTTTATTCTCTCGAACA
GAACCAGCTTCTATCCGAGAAAGCGGTGATCTAGCTAGATTATTTACATTACAGCAAGCCCATACTGCCG
TTGTCTCTCCAATAGGAATCAAAGGAGCTTATTCTTCTGATACATGGCCAACACTCTCTTGGGAAATGGA
ACTAGCTTACCAACCCACCCTCTACTGGAAACGTCCTCTACTCAACACACTATTAATCCAAAATAACGGT
TCTTGGGTCACCACAAATACCCCATTAGCTAAACATTCCTTTTATGGGAGAGGTTCTCACTCCCTCAAAT
TTTCTCATCTGAAACTATTTGCTAACTATCAAGCAGAAGTGGCTACTTCCACTGTCTCACACTACATCAA
TGCAGGAGGAGCTCTGGTCTTTTAA
>gi|3329344|gb|AAC68467.1| Putative Outer Membrane Protein E (Chlamydia
trachomatisi]
MKKAFFFFLIGNSLSGLAREVPSRIFLMPNSVPDPTKESLSNKISLTGDTHNLTNCYLDNLRYILAILQK
TPNEGAAVTITDYLSFFDTQKEGIYFAKNLTPESGGAIGYASPNSPTVEIRDTIGPVIFENNTCCRLFTW
RNPYAADKIREGGAIHAQNLYINHNHDVVGFMKNFSYVQGGAISTANTFVVSENQSCFLFMDNICIQTNT
AGKGGAIYAGTSNSFESNNCDLFFINNACCAGGAIFSPICSLTGNRGNIVFYNNRCFKNVETASSEASDG
GAIKVTTRLDVTGNRGRIFFSDNITKNYGGAIYAPVVTLVDNGPTYFINNIANNKGGAIYIDGTSNSKIS
ADRHAIIFNENIVTNVTNANGTSTSANPPRRNAITVASSSGEILLGAGSSQNLIFYDPIEVSNAGVSVSF
NKEADQTGSVVFSGATVNSADFHQRNLQTKTPAPLTLSNGFLCIEDHAQLTVNRFTQTGGVVSLGNGAVL
SCYKNGTGDSASNASITLKHIGLNLSSILKSGAEIPLLWVEPTNNSNNYTADTAATFSLSDVKLSLIDDY
GNSPYESTDLTHALSSQPMLSISEASDNQLQSENIDFSGLNVPHYGWQGLWTWGWAKTQDPEPASSATIT
DPQKANRFHRTLLLTWLPAGYVPSPKHRSPLIANTLWGNMLLATESLKNSAELTPSGHPFWGITGGGLGM
MVYQDPRENHPGFHMRSSGYSAGMIAGQTHTFSLKFSQTYTKLNERYAKNNVSSKNYSCQCEMLFSLQEG
FLLTKLVGLYSYGDHNCHHFYTQGENLTSQGTFRSQTMGGAVFFDLPMKPFGSTHILTAPFLGALGIYSS
LSHFTEVGAYPRSFSTKTPLINVLVPIGVKGSFMNATHRPQAWTVELAYQPVLYRQEPGIAAQLLASKGI
WFGSGSPSSRHAMSYKISQQTQPLSWLTLHFQYHGFYSSSTFCNYLNGEIALRF
>gi|3329342|gb|AE001360.1: c4464-1570,
ATGAAAAAAGCGTTTTTCTTTTTCCTTATCGGAAACTCCCTATCAGGACTAGCTAGAGAGGTTCCTTCTA
GAATCTTTCTTATGCCCAACTCAGTTCCAGATCCTACGAAAGAGTCGCTATCAAATAAAATTAGTTTGAC
AGGAGACACTCACAATCTCACTAACTGCTATCTCGATAACCTACGCTACATACTGGCTATTCTACAAAAA
ACTCCCAATGAAGGAGCTGCTGTCACAATAACAGATTACCTAAGCTTTTTTGATACACAAAAAGAAGGTA
TTTATTTTGCAAAAAATCTCACCCCTGAAAGTGGTGGTGCGATTGGTTATGCGAGTCCCAATTCTCCTAC
CGTGGAGATTCGTGATACAATAGGTCCTGTAATCTTTGAAAATAATACTTGTTGCAGACTATTTACATGG
AGAAATCCTTATGCTGCTGATAAAATAAGAGAAGGCGGAGCCATTCATGCTCAAAATCTTTACATAAATC
ATAATCATGATGTGGTCGGATTTATGAAGAACTTTTCTTATGTCCAAGGAGGAGCCATTAGTACCGCTAA
TACCTTTGTTGTGAGCGAGAATCAGTCTTGTTTTCTCTTTATGGACAACATCTGTATTCAAACTAATACA
GCAGGAAAAGGTGGCGCTATCTATGCTGGAACGAGCAATTCTTTTGAGAGTAATAACTGCGATCTCTTCT
TCATCAATAACGCCTGTTGTGCAGGAGGAGCGATCTTCTCCCCTATCTGTTCTCTAACAGGAAATCGTGG
TAACATCGTTTTCTATAACAATCGCTGCTTTAAAAATGTAGAAACAGCTTCTTCAGAAGCTTCTGATGGA
GGAGCAATTAAAGTAACTACTCGCCTAGATGTTACAGGCAATCGTGGTAGGATCTTTTTTAGTGACAATA
TCACAAAAAATTATGGCGGAGCTATTTACGCTCCTGTAGTTACCCTAGTGGATAATGGCCCTACCTACTT
TATAAACAATATCGCCAATAATAAGGGGGGCGCTATCTATATAGACGGAACCAGTAACTCCAAAATTTCT
GCCGACCGCCATGCTATTATTTTTAATGAAAATATTGTGACTAATGTAACTAATGCAAATGGTACCAGTA
CGTCAGCTAATCCTCCTAGAAGAAATGCAATAACAGTAGCAAGCTCCTCTGGTGAAATTCTATTAGGAGC
AGGGAGTAGCCAAAATTTAATTTTTTATGATCCTATTGAAGTTAGCAATGCAGGGGTCTCTGTGTCCTTC
AATAAGGAAGCTGATCAAACAGGCTCTGTAGTATTTTCAGGAGCTACTGTTAATTCTGCAGATTTTCATC
AACGCAATTTACAAACAAAAACACCTGCACCCCTTACTCTCAGTAATGGTTTTCTATGTATCGAAGATCA
TGCTCAGCTTACAGTGAATCGATTCACACAAACTGGGGGTGTTGTTTCTCTTGGGAATGGAGCAGTTCTG
AGTTGCTATAAAAATGGTACAGGAGATTCTGCTAGCAATGCCTCTATAACACTGAAGCATATTGGATTGA
ATCTTTCTTCCATTCTGAAAAGTGGTGCTGAGATTCCTTTATTGTGGGTAGAGCCTACAAATAACAGCAA
TAACTATACAGCAGATACTGCAGCTACCTTTTCATTAAGTGATGTAAAACTCTCACTCATTGATGACTAC
GGGAACTCTCCTTATGAATCCACAGATCTGACCCATGCTCTGTCATCACAGCCTATGCTATCTATTTCTG
AAGCTAGCGATAACCAGCTACAATCAGAAAATATAGATTTTTCGGGACTAAATGTCCCTCATTATGGATG
GCAAGGACTTTGGACTTGGGGCTGGGCAAAAACTCAAGATCCAGAACCAGCATCTTCAGCAACAATCACT
GATCCACAAAAAGCCAATAGATTTCATAGAACCTTACTACTAACATGGCTTCCTGCCGGGTATGTTCCTA
GCCCAAAACACAGAAGTCCCCTCATAGCTAACACCTTATGGGGGAATATGCTCCTTGCAACAGAAAGCTT
AAAAAATAGTGCAGAGCTGACACCTAGTGGTCATCCTTTCTGGGGAATTACAGGAGGAGGACTAGGCATG
ATGGTTTACCAAGATCCTCGAGAAAATCATCCTGGATTCCATATGCGCTCTTCCGGATACTCTGCGGGGA
TGATAGCAGGGCAGACACACACCTTCTCATTGAAATTCAGTCAGACCTACACCAAACTCAATGAGCGTTA
CGCAAAAAACAACGTATCTTCTAAAAATTACTCATGCCAAGGAGAAATGCTCTTCTCATTGCAAGAAGGT
TTCTTGCTGACTAAATTAGTTGGGCTTTACAGCTATGGAGACCATAACTGTCACCATTTCTATACTCAAG
GAGAAAATCTAACATCTCAAGGGACGTTCCGCAGTCAAACGATGGGAGGTGCTGTCTTTTTTGATCTCCC
TATGAAACCCTTTGCATCAACGCATATACTGACAGCTCCCTTTTTAGGTGCTCTTGGTATTTATTCTAGC
CTGTCTCACTTTACTGAGGTGGGAGCCTATCCGCGAAGCTTTTCTACAAAGACTCCTTTGATCAATGTCC
TAGTCCCTATTGGAGTTAAAGGTAGCTTTATGAATGCTACCCACAGACCTCAAGCCTGGACTGTAGAATT
GGCATACCAACCCGTTCTGTATAGACAAGAACCAGGGATCGCAGCCCAGCTCCTAGCCAGTAAGGGTATT
TGGTTCGGTAGTGGAAGCCCCTCATCGCGTCATGCCATGTCCTATAAAATCTCACAGCAAACACAACCTT
TGAGTTGGTTAACTCTCCATTTCCAGTATCATGGATTCTACTCCTCTTCAACCTTCTGTAATTATCTCAA
TGGGGAAATTGCTCTGCGATTCTAG
>gi|3329279|gb|AAC68408.1| Putative Outer Membrane Protein D [Chlamydia
trachomatis]
MSSEKDIKSTCSKFSLSVVAAILASVSGLASCVDLHAGGQSVNELVYVGPQAVLLLDQIRDLFVGSKDSQ
AEGQYRLIVGDPSSFQEKDADTLPGKVEQSTLFSVTNPVVFQGVDQQDQVSSQGLICSFTSSNLDSPRDG
ESFLGIAFVGDSSKAGITLTDVKASLSGAALYSTEDLIFEKIKGGLEFASCSSLEQGGACAAQSILIHDC
QGLQVKHCTTAVNAEGSSANDHLGFGGGAFFVTGSLSGEKSLYMPAGDMVVANCDGAISFECNSANFANG
GAIAASGKVLFVANDKKTSFIENRALSGGAIAASSDIAFQNCAELVFKGNCAIGTEDKGSLGGGAISSLG
TVLLQGNHGITCDKNESASQGGAIFGKNCQISDNEGPVVFRDSTACLGGGAIAAQEIVSIQNNQAGISFE
GGKASFGGGIACGSFSSAGGASVLGTIDISKNLGAISFSRTLCTTSDLGQMEYQGGGALFGENISLSENA
GVLTFKDNIVKTFASNGKILGGGAILATGKVEITNNSEGISFTGNAPAPQALPTQEEFPLFSKKEGRPLS
SGYSGGGAILGREVAILHNAAVVFEQNRLQCSEEEATLLGCCGGGAVHGMDSTSIVGNSSVRFGNNYAMG
QGVSGGALLSKTVQLAGNGSVDFSRNIASLGGGALQASEGNCELVDNGYVLFRDNRGRVYGGAISCLRGD
VVISGNKGRVEFKDNIATRLYVEETVEKVEEVEPAPEQKDNNELSFLGRAEQSFITAANQALFASEDGDL
SPESSISSEELAKRRECAGGAIFAKRVRIVDNQEAVVFSNNFSDIYGGAIFTGSLREEDKLDGQIPEVLI
SGNAGDVVFSGNSSKRDEHLPHTGGGAICTQNLTISQNTGNVLFYNNVACSGGAVRIEDHGNVLLEAFGG
DIVFKGNSSFRAQGSDAIYFAGKESHITALNATEGHAIVFHDALVFENLEERKSAEVLLINSRENPGYTG
SIRFLEAESKVPQCIHVQQCSLELLMGATLCSYCFKQDAGAKLVLAAGAKLKILDSGTPVQQGHAISKPE
AEIESSSEPEGAHSLWIAKNAQTTVPMVDIHTISVDLASFSSSQQEGTVEAPQVIVPGGSYVRSGELNLE
LVNTTGTGYENHALLKNEAKVPLMSFVASGDEASAEISNLSVSDLQIHVVTPEIEEDTYGHMCDWSEAKI
QDGTLVISWNPTGYRLDPQKAGALVFNALWEEGAVLSALKNARFAHNLTAQRMEFDYSTNVWGFAFGGFR
TLSAENLVAIDGYKGAYGGASAGVDIQLMEDFVLGVSGAAFLGKMDSQKFDAEVSRKGVVGSVYTGFLAG
SWFFKGQYSLGETQNDMKTRYGVLGESSASWTSRGVLADALVEYRSLVGPVRPTFYALHFNPYVEVSYAS
MKFPGFTEQGREARSFEDASLTNITIPLGMKFELAFIKGQFSEVNSLGISYAWEAYRKVEGGAVQLLEAG
FDWEGAPMDLPRQELRVALENNTEWSSYFSTVLGLTAFCGGFTSTDSKLGYEANTGLRLIF
>gi|3329271|gb|AE001353.1:9710-14305,
ATGAGTTCCGAGAAAGATATAAAAAGCACCTCTTCTAAGTTTTCTTTGTCTGTAGTAGCAGCTATCCTTG
CCTCTGTTAGCGGGTTAGCTAGTTGCGTAGATCTTCATGCTGGAGGACAGTCTGTAAATGAGCTGGTATA
TGTAGGCCCTCAAGCGGTTTTATTGTTAGACCAAATTCGAGATCTATTCGTTGGGTCTAAAGATAGTCAG
GCTGAAGGACAGTATAGGTTAATTGTAGGAGATCCAAGTTCTTTCCAAGAGAAAGATGCGGATACTCTTC
CCGGGAAGGTAGAGCAAAGTACTTTGTTCTCAGTAACCAATCCCGTGGTTTTCCAAGGTGTGGACCAACA
GGATCAAGTCTCTTCCCAAGGGTTAATTTGTAGTTTTACGAGCAGCAACCTTGATTCTCCTCGTGACGGA
GAATCTTTTTTAGGTATTGCTTTTGTTGGGGATAGTAGTAAGGCTCGAATCACATTAACTCACGTGAAAG
CTTCTTTGTCTGGAGCGGCTTTATATTCTACAGAAGATCTTATCTTTGAAAAGATTAAGGGTGGATTGGA
ATTTGCATCATGTTCTTCTCTAGAACAGGGGGGAGCTTGTGCAGCTCAAAGTATTTTGATTCATGATTGT
CAAGGATTGCAGGTTAAACACTGTACTACAGCCGTGAATGCTGAGGGGTCTAGTGCGAATGATCATCTTG
GATTTGGAGGAGGCGCTTTCTTTGTTACGGGTTCTCTTTCTGGAGAGAAAAGTCTCTATATGCCTGCAGG
AGATATGGTAGTTGCGAATTGTGATGGGGCTATATCTTTTGAAGGAAACAGCGCGAACTTTGCTAATGGA
GGAGCGATTGCTGCCTCTGGGAAAGTGCTTTTTGTCGCTAATGATAAAAAGACTTCTTTTATAGAGAACC
GAGCTTTGTCTGGAGGAGCGATTGCAGCCTCTTCTGATATTGCCTTTCAAAACTGCGCAGAACTAGTTTT
CAAAGGCAATTGTGCAATTGGAACAGAGGATAAAGGTTCTTTAGGTGGAGGGGCTATATCTTCTCTAGGC
ACCGTTCTTTTGCAAGGGAATCACGGGATAACTTGTGATAAGAATGAGTCTGCTTCGCAAGGAGGCGCCA
TTTTTGGCAAAAATTGTCAGATTTCTGACAACGAGGGGCCAGTGGTTTTCAGAGATAGTACAGCTTGCTT
AGGAGGAGGCGCTATTGCAGCTCAACAAATTGTTTCTATTCAGAACAATCAGGCTGGGATTTCCTTCGAG
GGAGCTAAGGCTAGTTTCGGAGGAGGTATTGCGTGTGGATCTTTTTCTTCCGCAGGTGGTGCTTCTGTTT
TAGGGACCATTGATATTTCGAAGAATTTAGGCGCGATTTCGTTCTCTCGTACTTTATGTACGACCTCAGA
TTTAGGACAAATGGAGTACCAGGGAGGAGGAGCTCTATTTGGTGAAAATATTTCTCTTTCTGAGAATGCT
GGTGTGCTCACCTTTAAAGACAACATTGTGAAGACTTTTGCTTCGAATGGGAAAATTCTGGGAGCAGGAG
CGATTTTAGCTACTGGTAAGGTGGAAATTACTAATAATTCCGAAGGAATTTCTTTTACAGGAAATGCGAG
AGCTCCACAAGCTCTTCCAACTCAAGAGGAGTTTCCTTTATTCAGCAAAAAAGAAGGGCGACCACTCTCT
TCAGGATATTCTGGGGGAGGAGCGATTTTAGGAAGAGAAGTAGCTATTCTCCACAACGCTGCAGTAGTAT
TTGAGCAAAATCGTTTGCAGTGCAGCGAAGAAGAAGCGACATTATTAGGTTGTTGTGGAGGAGGCGCTGT
TCATGGGATGGATAGCACTTCGATTGTTGGCAACTCTTCAGTAAGATTTGGTAATAATTACGCAATGGGA
CAAGGAGTCTCAGGAGGAGCTCTTTTATCTAAAACAGTGCAGTTAGCTGGGAATGGAAGCGTCGATTTTT
CTCGAAATATTGCTAGTTTGGGAGGAGGAGCTCTTCAAGCTTCTGAAGGAAATTGTGAGCTAGTTGATAA
CGGCTATGTGCTATTCAGAGATAATCGAGGGAGGGTTTATGGGGGTGCTATTTCTTGCTTACGTGGAGAT
GTAGTCATTTCTGGAAACAAGGGTAGAGTTGAATTTAAAGACAACATAGCAACACGTCTTTATGTGGAAG
AAACTGTAGAAAAGGTTGAAGAGGTAGAGCCAGCTCCTGAGCAAAAAGACAATAATGAGCTTTCTTTCTT
AGGGAGAGCAGAACAGAGTTTTATTACTGCAGCTAATCAAGCTCTTTTCGCATCTGAAGATGGGGATTTA
TCACCTGAGTCATCCATTTCTTCTGAAGAACTTGCGAAAAGAAGAGAGTGTGCTGGAGGAGCTATTTTTG
CAAAACGGGTTCGTATTGTAGATAACCAAGAGGCCGTTGTATTCTCGAATAACTTCTCTGATATTTATGG
CGGCGCCATTTTTACAGGTTCTCTTCGAGAAGAGGATAAGTTAGATGGGCAAATCCCTGAAGTCTTGATC
TCAGGCAATGCAGGGGATGTTGTTTTTTCCCGAAATTCCTCGAAGCGTGATGAGCATCTTCCTCATACAG
GTGGGGGAGCCATTTGTACTCAAAATTTGACGATTTCTCAGAATACAGGGAATGTTCTGTTTTATAACAA
CGTGGCCTGTTCGGGAGGAGCTGTTCGTATAGAGGATCATGGTAATGTTCTTTTAGAAGCTTTTGGAGGA
GATATTGTTTTTAAAGGAAATTCTTCTTTCAGAGCACAAGGATCCGATGCTATCTATTTTGCAGGTAAAG
AATCGCATATTACAGCCCTGAATGCTACGGAAGGACATGCTATTGTTTTCCACGACGCATTAGTTTTTGA
AAATCTAGAACAAAGGAAATCTGCTGAAGTATTGTTAATCAATAGTCGAGAAAATCCAGGTTACACTGGA
TCTATTCGATTTTTAGAAGCAGAAAGTAAAGTTCCTCAATGTATTCATGTACAACAAGGAAGCCTTGAGT
TGCTAAATGGAGCCACATTATGTAGTTATGGTTTTAAACAAGATGCTGGAGCTAAGTTGGTATTGGCTGC
TGGAGCTAAACTGAAGATTTTAGATTCAGGAACTCCTGTACAACAAGGGCATGCTATCAGTAAACCTGAA
GCAGAAATCGAGTCATCTTCTGAACCAGAGGGTGCACATTCTCTTTGGATTGCGAAGAATGCTCAAACAA
CAGTTCCTATGGTTGATATCCATACTATTTCTGTAGATTTAGCCTCCTTCTCTTCTAGTCAACAGCAGGG
GACAGTAGAAGCTCCTCAGGTTATTGTTCCTGGAGGAAGTTATGTTCGATCTGGAGAGCTTAATTTGGAG
TTAGTTAACACAACAGGTACTGGTTATGAAAATCATGCTTTATTGAAGAATGAGGCTAAAGTTCCATTGA
TCTCTTTCGTTGCTTCTGGTCATGAAGCTTCAGCCGAAATCAGTAACTTGTCGGTTTCTGATTTACACAT
TCATGTAGTAACTCCAGAGATTGAAGAAGACACATACGGCCATATGGGAGATTGGTCTGAGGCTAAAATT
CAAGATGGAACTCTTGTCATTAGTTGGAATCCTACTGGATATCGATTAGATCCTCAAAAAGCAGGGGCTT
TAGTATTTAATGCATTATGGGAAGAAGGGGCTGTCTTGTCTGCTCTGAAAAATGCACGCTTTGCTCATAA
TCTCACTGCTCAGCGTATGGAATTCGATTATTCTACAAATGTGTGGGGATTCGCCTTTGGTGGTTTCCGA
ACTCTATCTGCAGAGAATCTGGTTGCTATTGATGGATACAAAGGAGCTTATGGTGGTGCTTCTGCTGGAG
TCGATATTCAATTGATGGAAGATTTTGTTCTAGGAGTTAGTGGAGCTGCTTTCCTAGGTAAAATGGATAG
TCAGAAGTTTGATGCGGAGGTTTCTCGGAAGGGAGTTGTTGGTTCTGTATATACAGGATTTTTAGCTGGA
TCCTGGTTCTTCAAAGGACAATATAGCCTTGGAGAAACACAGAACGATATGAAAACGCGTTATGGAGTAC
TAGGAGAGTCGAGTGCTTCTTGCACATCTCGAGGAGTACTGGCAGATGCTTTAGTTGAATACCGAAGTTT
AGTTGGTCCTGTGAGACCTACTTTTTATGCTTTGCATTTCAATCCTTATGTCGAAGTATCTTATGCTTCT
ATGAAATTCCCTGGCTTTACAGAACAAGGAAGAGAAGCGCGTTCTTTTGAAGACGCTTCCCTTACCAATA
TCACCATTCCTTTAGGGATGAAGTTTGAATTGGCGTTCATAAAAGGACAGTTTTCAGAGGTGAACTCTTT
GGGAATAAGTTATGCATGGGAAGCTTATCGAAAAGTAGAAGGAGGCGCGGTGCAGCTTTTAGAAGCTGGG
TTTGATTGGGAGGGAGCTCCAATGGATCTTCCTAGACAGGAGCTGCGTGTCGCTCTGGAAAATAATACGG
AATGGAGTTCTTACTTCAGCACAGTCTTAGGATTAACAGCTTTTTGTGGAGGATTTACTTCTACAGATAG
TAAACTAGGATATGAGGCGAATACTGGATTGCGATTGATCTTTTAA
>gi|3329169|gb|AAC68308.1| Outer Membrane Protein Analog [Chlamydia
trachomatis]
MSSKLVNYLRLTFLSFLGIASTSLDAMPAGNPAFPVIPGINIEQKNACSFDLCNSYDVLSALSGNLKLCF
CGDYIFSEEAQVKDVPVVTSVTTAGVGPSPDITSTTKTRNFDLVNCNLNTNCVAVAFSLPDRSLSAIPLF
DVSFEVKVGGLKQYYRLPMNAYRDFTSEPLNSESEVTDGMIEVQSNYGFVWDVSLKKVIWKDGVSFVGVG
ADYRHASCPIDYIIANSQANPEVFIADSDGKLNFKEWSVCVGLTTYVNDYVLPYLAFSIGSVSRQAPDDS
FKKLEDRFTNLKFKVRKITSSHRGNICIGATNYVADNFFYNVEGRWGSQBAVNVSGGFQF
>gi|3329166|gb|AE001342.1:C4638-3616,
ATGAGTAGCAAGCTAGTGAACTATCTCCGTTTGACTTTCCTATCTTTTTTAGGGATCGCATCTACTTCAT
TAGACCCTATGCCTGCGGGGAATCCGGCGTTTCCAGTCATCCCGGGGATTAATATTGAACAGAAAAATGC
CTGTTCTTTCGATTTATGTAATTCTTATGATGTACTATCCGCACTGTCCGGTAACCTGAAGCTCTGCTTC
TGCGGAGATTATATCTTTTCAGAAGAAGCTCAGGTAAAAGATGTCCCTGTCGTTACCTCTGTGACAACAG
CTGGGGTTGGTCCTTCTCCTCATATTACTTCGACAACCAAAACGCGAAATTTCGATCTCGTGAACTGTAA
TCTCAATACAAACTGTGTAGCTGTAGCTTTTTCCCTTCCTGATCGTTCGCTGAGCGCCATTCCTCTGTTT
GATGTGAGTTTCGAAGTGAAAGTAGGAGGACTCAAACAATACTACCGCCTTCCCATGAATGCCTATCGAG
ACTTCACCTCGGAACCTCTCAATTCTGAATCAGAAGTTACGGACGGGATGATTGAAGTACAGTCCAATTA
CGGATTTGTTTGGGATGTTAGCTTGAAAAAAGTCATATGGAAAGATGGCGTTTCCTTTGTAGGCGTCGGT
GCAGACTATCGCCATGCTTCTTGCCCTATTGACTACATCATTGCAAACAGTCAAGCTAATCCAGAAGTAT
TCATCGCTGACTCGGATGGGAAACTGAACTTCAAGGAGTGGAGTGTCTGCGTAGGTCTTACTACCTATGT
GAATGACTACGTTCTTCCTTACTTAGCGTTTTCTATACGGAGTGTTTCTCGCCAAGCTCCGGACGACAGC
TTCAAAAAATTAGAAGATCGCTTCACTAACCTCAAATTTAAAGTTCGTAAAATTACCAGCTCTCATCGTG
GAAACATCTGCATCGGAGCGACAAACTATGTCGCCGATAACTTCTTCTACAACGTAGAAGGAAGATGGGG
AAGCCAGCGCGCTGTGAACGTCTCCGGAGGATTCCAATTCTAA
>gi |3328866|gb|AAC68034.1|Sulfite Reductase [Chlamydia
trachomatis]
MSLFSKFKAQWMFLHSRELCSSTSDIGNTCSDPVFQVLCNPVRSEISYKVGDSLGVFPTNPSILVDSVLD
ALQYGPRSPVVSRHADSVLPLHEFLTSYVDLDKIPKSLRPFFPGDLDDTWSLAEAILVYQPRIPFEEFIR
SAMPLLPRFYSIASSPTCSHGKLELLVRCVSFQGKTQLRYGLCSAFLCKDLQEGESFRGFIQPTRHFTLE
QKNFGKPLIMIGAGTGIAPYKGFLQHRIYHQDVGSNILFFGERFEKSNFYYRDFLQELIVSGKLQLFTAF
SRDSESKLYVQNVIEQQKELIQEVYEQEAFFFVCGKKILGTEVKRALEQILGPKAVRELIAQKRLVSDVY
>gi|3328863|gb|AE001317:c2573-1521,
ATGTCTTTATTTTCTAAATTCAAAGCTCAGTGGATGTTTTTACATTCACGTGAGCTTTGTTCTTCCACAT
CGGATATTGGGAATACTTGTTCGGATCCTGTTTTTCAGGTTTTATGTAATCCGGTTCGTTCTGAGATTTC
CTATAAAGTTGGGGATTCTTTGGGGGTATTCCCAACAAATCCTTCCATATTAGTCGATTCAGTTCTAGAT
GCTTTACAGTATGGCCCCAGGTCTCCTGTCGTATCTCGGCATGCAGATTCTGTTCTCCCTCTTCACGAAT
TTCTTACTAGTTACGTACACTTAGATAAAATTCCAAAATCGTTAAGACCTTTTTTCCCAGGGGATTTAGA
CGATACCTGGTCTTTAGCTGAAGCTATTTTGGTTTACCAGCCGCGTATTCCTTTTGAAGAGTTTATTCGG
AGTGCGATGCCTTTATTGCCTCGATTTTATTCTATAGCTTCTTCTCCAACATGTTCTCATGGGAAGCTAG
AGTTGCTCGTGCGCTGTGTTAGTTTCCAAGGTAAAACGCAGCTGCGCTATGGATTATGTTCGGCTTTTTT
ATGTAAGGACTTACAAGAGGGAGAGTCTTTTCGTGGGTTTATACAACCGACGCGGCATTTTACTTTGGAG
CAGAAAAATTTTGGGAAACCTTTAATTATGATCGGAGCAGGGACAGGTATCGCTCCGTACAAAGGGTTCT
TACAACATCGAATATACCATCAGGACGTAGGCTCCAATATTCTATTCTTTGGAGAGCGTTTTGAGAAAAG
TAACTTCTATTACCGGGATTTTCTCCAGGAGCTGATCGTTTCAGGAAAACTCCAGTTATTCACAGCCTTT
TCCAGAGATTCCGAGTCTAAATTGTATGTTCAGAATGTTATAGAGCAACAAAAAGAACTTATACAAGAAG
TCTACGAACAAGAAGCTTTCTTTTTTGTTTGTGGGAAAAAAATCCTTGGTACGGAAGTTAAACGTGCTTT
AGAGCAGATATTAGGTCCTAAGGCGGTACGAGAGCTGATTGCACAGAAGAGACTAGTTTCAGACGTATAC
TAA
>gi|3328843|gb|AAC68011.1| Putative outer membrane protein C [Chlamydia
trachomatis]
MKFMSATAVFAAALSSVTEASSIQDQIKNTDCNVSKLGYSTSQAFTDMMLADNTEYRAADSVSFYDFSTS
SRLPRKHLSSSSEASPTTEGVSSSSSGETDEKTEEELDNGGIIYAREKLTISESQDSLSNQSIELHDNSI
FFGEGEVIFDHRVALKNGGAIYGEKEVVFENIKSLLVEVNIAVEKGGSVYAKERVSLENVTEATFSSNGG
EQGGGGIYSEQDMLISDCNNVHFQGNAAGATAVKQCLDEEMIVLLAECVDSLSEDTLDSTPETEQTESNG
NQDGSSETEDTQVSESPESTPSPDDVLGKGGGIYTEKSLTITGITGTIDFVSNIATDSGAGVFTKENLSC
TNTNSLQFLKNSAGQHGGGAYVTQTMSVTNTTSESITTPPLIGEVIFSENTAKCHGGGICTNKLSLSNLK
TVTLTKNSAKESGGAIFTDLASIPITDTPESSTPSSSSPASTPEVVASAKINRFFASTAKPAAPSLTEAE
SDQTDQTETSDTNSDIDVSIENILNVAINQNTSAKKGGAIYGKKAKLSRINNLELSGNSSQDVGGGLCLT
ESVEFDAIGSLLSHYNSAAKEGGAIHSKTVTLSNLKSTFTFADNTVKAIVESTPEAPEEIPPVEGEESTA
TEDPNSNTEGSSANTNLEGSQGDTADTGTGDVNNESQDTSDTGNAESEEQLQDSTQSNEENTLPNSNIDQ
SNENTDESSDSHTEEITDESVSSSSESGSSTPQDGGAASSGAPSGDQSISANACLAKSYAASTDSSPVSN
SSGSEEPVTSSSDSDVTASSDNPDSSSSGDSAGDSEEPTEPEAGSTTETLTLIGGGAIYGETVKIENFSG
QGIFSGNKAIDNTTEGSSSKSDVLGGAVYAKTLFNLDSGSSRRTVTFSGNTVSSQSTTGQVAGGAIYSPT
VTIATPVVFSKNSATNNANNTTDTQRKDTFGGAIGATSAVSLSGGAHFLENVADLGSAIGLVPGTQNTET
VKLESGSYYFEKNKALKPATIYAPVVSIKAYTATFNQNRSLEEGSAIYFTKEASIESLGSVLFTGNLVTL
TLSTTTEGTPATTSGDVTKYGAAIFGQIASSNGSQTDNLPLKLIASGGNICFRNNEYRPTSSDTGTSTFC
SIAGDVKLTMQAAKGKTISFFDAIRTSTKKTGTQATAYDTLDINKSEDSETVNSAFTGTILFSSELHENK
SYIPQNVVLHSGSLVLKPNTELHVISFEQKEGSSLVMTPGSVLSNQTVADGALVINNMTIDLSSVEKNGI
AEGNIFTPPELRIIDTTTGGSGGTPSTDSESNQNSDDTEEQNNNDASNQGESANGSSSPAVAAAHTSRTR
NFAAAATATPTTTPTATTTTSNQVILGGEIKLIDPNGTFFQNPALRSDQQISLLVLPTDSSKNQAQKIVL
TGDIAPQKGYTGTLTLDPDQLQNGTISVLWKFDSYRQWAYVPRDNHFYANSILGSQMLMVTVKQGLLNDK
MNLARFEEVSYNNLWISGLGTMLSQVGTPTSEEFTYYSRGASVALDAKPAHDVIVGAAFSKMIGKTKSLK
RENNYTHKGSEYSYQASVYGGKPFHFVINKKTEKSLPLLLQGVISYGYIKHDTVTHYPTIRERNKGEWED
LGWLTALRVSSVLRTPAQGDTKRITVYGELEYSSIRQKQFTETEYDPRYFDNCTYRNLAIPMGLAFEGEL
SGNDILMYNRFSVAYMLSIYRNSPTCKYQVLSSGEGGEIICGVPTRNSARGEYSTQLYLGPLWTLYGSYT
IEADAHTLAHMMNCGARMTF
>gi|3328842|gb|AE001315.1:120-5432,
ATGAAATTTATGTCAGCTACTGCTGTATTTGCTGCAGCACTCTCCTCCGTTACTGAGGCGAGCTCGATCC
AAGATCAAATAAAGAATACCGACTGCAATGTTAGCAAATTAGGATATTCAACTTCTCAAGCATTTACTGA
TATGATGCTAGCAGACAACACAGAGTATCGAGCTGCTGATAGTGTTTCATTCTATGACTTTTCGACATCT
TCCAGATTACCTAGAAAACATCTTAGTAGTAGTAGTGAAGCTTCTCCAACGACAGAAGGAGTGTCTTCAT
CTTCATCTGGAGAAACTGATGAGAAAACAGAAGAAGAACTAGACAATGGCGGAATCATTTATGCTAGAGA
GAAACTAACTATCTCAGAATCTCAGGACTCTCTCTCTAATCAAAGCATAGAACTCCATGACAATAGTATT
TTCTTCGGAGAAGGTGAAGTTATCTTTGATCACAGAGTTGCCCTCAAAAACGGAGGAGCTATTTATGGAG
AGAAAGAGGTAGTCTTTGAAAACATAAAATCTCTACTAGTAGAAGTAAATATCGCGGTCGAGAAAGGGGG
TAGCGTCTATGCAAAAGAACGAGTATCTTTAGAAAATGTTACCGAAGCAACCTTCTCCTCCAATGCTGGG
GAACAAGGTGGTGGTGGAATCTATTCAGAACAGGATATGTTAATCAGTGATTGCAACAATGTACATTTCC
AAGGGAATGCTGCAGGAGCAACAGCAGTAAAACAATGTCTGGATGAAGAAATGATCGTATTGCTCGCAGA
ATGCGTTGATAGCTTATCCGAAGATACACTGCATAGCACTCCAGAAACGGAACAGACTGAGTCAAATGGA
AATCAAGACGGTTCGTCTGAAACAGAAGATACACAAGTATCAGAATCACCAGAATCAACTCCTAGCCCCG
ACGATGTTTTAGGTAAAGGTGGTGGTATCTATACAGAAAAATCTTTGACCATCACTGGAATTACAGGGAC
TATAGATTTTGTCAGTAACATAGCTACCGATTCTGGAGCAGGTGTATTCACTAAAGAAAACTTGTCTTGC
ACCAACACGAATAGCCTACAGTTTTTGAAAAACTCGGCAGGTCAACATGGAGGAGGAGCCTACGTTACTC
AAACCATGTCTGTTACTAATACAACTAGTGAAAGTATAACTACTCCCCCTCTCATAGGAGAAGTGATTTT
CTCTGAAAATACAGCTAAAGGGCACGGTGGTGGTATCTGCACTAACAAACTTTCTTTATCTAATTTAAAA
ACGGTGACTCTCACTAAAAACTCTGCAAAGGAGTCTGGAGGAGCTATTTTTACAGATCTGGCGTCTATAC
CAATAACAGATACCCCACAATCTTCTACCCCCTCTTCCTCCTCGCCTGCAAGCACTCCTGAAGTAGTTGC
TTCTGCTAAAATAAATCGATTCTTTGCCTCTACGGCAAAACCGGCAGCCCCTTCTCTAACAGAGGCTGAG
TCTGATCAAACGGATCAAACAGAAACTTCTGATACTAATAGCGATATAGACGTGTCGATTGAGAACATTT
TGAATGTCGCTATCAATCAAAACACTTCTGCGAAAAAAGGAGGGGCTATTTACGGGAAAAAAGCTAAACT
TTCCCGTATTAACAATCTTGAACTTTCAGGGAATTCATCCCAGGATGTAGGAGGAGCTCTCTGTTTAACT
GAAAGCGTAGAATTTGATGCAATTGGATCGCTCTTATCCCACTATAACTCTGCTGCTAAAGAAGGTGGGG
CTATTCATTCTAAAACGGTTACTCTATCTAACCTCAAGTCTACCTTCACTTTTGCAGATAACACTGTTAA
AGCAATAGTAGAAAGCACTCCTGAAGCTCCAGAAGAGATTCCTCCAGTAGAAGGAGAAGAGTCTACAGCA
ACAGAAGATCCAAATTCTAATACAGAAGGAAGTTCGGCTAACACTAACCTTGAAGGATCTCAAGGGGATA
CTGCTGATACAGGGACTCGTGATGTTAACAATGAGTCTCAAGACACATCAGATACTGGAAACGCTGAATC
TGAAGAACAACTACAAGATTCTACACAATCTAATGAAGAAAATACCCTTCCCAATAGTAATATTGATCAA
TCTAACCAAAACACAGACGAATCATCTGATAGCCACACTGAGGAAATAACTGACGAGAGTGTCTCATCGT
CCTCTGAAAGTGGATCATCTACTCCTCAAGATGGAGGAGCAGCTTCTTCAGGGGCTCCCTCAGGAGATCA
ATCTATCTCTGCAAACGCTTGTTTAGCTAAAAGCTATGCTGCGAGTACTGATAGCTCCCCCGTATCTAAT
TCTTCAGGTTCAGAAGAGCCTGTCACTTCTTCTTCAGATTCAGACGTTACTGCATCTTCTGATAATCCAG
ACTCTTCCTCATCTGGAGATAGCGCTGGAGACTCTGAAGAACCGACTGAGCCAGAAGCTGGTTCTACAAC
AGAAACTCTTACTTTAATAGGAGGAGGTGCTATCTATGGAGAAACTGTTAAGATTGAGAACTTCTCTGGC
CAAGGAATATTTTCTGGAAACAAAGCTATCGATAACACCACAGAAGGCTCCTCTTCCAAATCTGACGTCC
TCGGAGGTGCGGTCTATGCTAAAACATTGTTTAATCTCGATAGCGGGAGCTCTAGACGAACTGTCACCTT
CTCCGGGAATACTGTCTCTTCTCAATCTACAACAGGTCAGGTTGCTGGAGGAGCTATCTACTCTCCTACT
GTAACCATTGCTACTCCTGTAGTATTTTCTAAAAACTCTGCAACAAACAATGCTAATAACACTACAGATA
CTCAGAGAAAAGACACCTTTGGAGGAGCTATCGGAGCTACTTCTGCTGTTTCTCTATCAGGAGGGCCTCA
TTTCTTAGAAAACGTTGCTGACCTCGGATCTGCTATTGGGTTGGTGCCAGGCACACAAAATACAGAAACA
GTGAAATTAGAGTCTGGCTCCTACTACTTTGAAAAAAATAAAGCTTTAAAACGAGCTACTATTTACGCAC
CTGTCGTTTCCATTAAAGCCTATACTGCGACATTTAACCAAAACAGATCTCTAGAAGAAGGAAGCGCGAT
TTACTTTACAAAAGAAGCATCTATTGAGTCTTTAGGCTCTGTTCTCTTCACAGGAAACTTAGTAACCCTA
ACGCTAAGCACAACTACAGAAGGCACACCAGCCACAACCTCAGGAGATGTAACAAAATATGGTGCTGCTA
TCTTTGGACAAATAGCAAGCTCAAACGGATCTCAGACGGATAACCTTCCCCTGAAACTCATTGCTTCAGG
AGGAAATATTTGTTTCCGAAACAATGAATACCGTCCTACTTCTTCTGATACCGCAACCTCTACTTTCTCT
AGTATTGCGGGAGATGTTAAATTAACCATGCAAGCTGCAAAAGGGAAAACGATCAGTTTCTTTGATGCAA
TCCGGACCTCTACTAAGAAAACAGGTACACAGGCAACTGCCTACGATACTCTCGATATTAATAAATCTGA
GGATTCAGAAACTGTAAACTCTGCGTTTACAGGAACGATTCTGTTCTCCTCTGAATTACATGAAAATAAA
TCCTATATTCCACAAAACGTAGTTCTACACAGTGGATCTCTTGTATTGAAGCCAAATACCGAGCTTCATG
TTATTTCTTTTGAGCAGAAAGAAGGCTCTTCTCTCGTTATGACACCTGGATCTGTTCTTTCGAACCAGAC
TGTTGCTGATGGAGCTTTGGTCATAAATAACATGACCATTGATTTATCCAGCGTAGAGAAAAATGGTATT
GCTGAAGGAAATATCTTTACTCCTCCAGAATTGAGAATCATAGACACTACTACAGGTGGAAGCGGTGGAA
CCCCATCTACAGATAGTGAAAGTAACCAGAATAGTGATGATACCGAGGAGCAAAATAATAATGACGCCTC
GAATCAAGGAGAAAGCGCGAATGGATCGTCTTCTCCTGCAGTAGCTGCTGCACACACATCTCGTACAAGA
AACTTTGCCGCTGCAGCTACAGCCACACCTACGACAACACCAACGGCTACAACTACAACAAGCAACCAAG
TAATCCTAGGAGGAGAAATTAAACTCATCGATCCTAATGGGACCTTCTTCCAGAACCCTGCATTAAGATC
CGACCAACAAATCTCCTTGTTAGTGCTCCCTACAGACTCATCAAAAATGCAAGCTCAGAAAATAGTACTG
ACGGGTGATATTGCTCCTCAGAAAGGATATACAGGAACACTCACTCTGGATCCTGATCAACTACAAAATG
GAACGATCTCAGTGCTCTGGAAATTTGACTCTTATAGACAATGGGCTTATGTACCTAGAGACAATCATTT
CTATGCGAACTCGATTCTGGGATCTCAAATGTTAATGGTCACAGTCAAACAAGGCTTGCTCAACGATAAA
ATGAATCTAGCTCGCTTTGAGGAAGTTAGCTATAACAACCTGTGGATATCAGGACTAGGAACGATGCTAT
CGCAAGTAGGAACACCTACTTCTGAAGAATTCACTTATTACAGCAGAGGAGCTTCTGTTGCCTTAGATGC
TAAACCAGCCCATGATGTGATTGTTGGAGCTGCATTTAGTAAGATCATCGGGAAAACAAAATCCTTGAAA
AGAGAGAATAACTACACTCACAAAGGATCCGAATATTCTTACCAAGCATCGGTATACGGAGGCAAACCAT
TCCACTTTGTAATCAATAAAAAAACGGAAAAATCGCTACCGCTATTGTTACAAGGAGTCATCTCTTACGG
ATATATCAAACATGATACAGTGACTCACTATCCAACGATCCGTGAACGAAACAAAGGAGAATGGGAAGAC
TTAGGATGGCTGACAGCTCTCCGTGTCTCCTCTGTCTTAAGAACTCCTGCACAAGGGGATACTAAACGTA
TCACTGTTTACGGAGAATTGGAATACTCCAGTATCCGTCAGAAACAATTCACAGAAACAGAATACGATCC
TCGTTACTTCGACAACTGCACCTATAGAAACTTAGCAATTCCTATGGGGTTAGCATTCGAAGGAGACCTC
TCTGGTAACGATATTTTGATGTACAACAGATTCTCTGTAGCATACATGCTATCAATCTATCGAAATTCTC
CAACATGCAAATACCAAGTGCTCTCTTCAGGAGAAGGCGGAGAAATTATTTGTGGAGTACCGACAAGAAA
CTCAGCTCGCGGAGAATACAGCACGCAGCTGTACCTGGGACCTTTGTGGACTCTGTATGGATCCTACACG
ATAGAAGCAGACGCACATACACTAGCTCATATGATGAACTGCGGTGCTCGTATGACATTCTAA
>gi|3328815|gb|AAC67986.1| hypothetical protein
[Chlamydia trachomatis]
MMKPLRFGYFFCAIYFTLLQAAFAKEPNSCPDCQNNWKEVTHTDQLPENIIHADDACYHSGYVQALIDMH
FLDSCCQVIVENQTAYLFSLPTDDVTRNAIINLIKDLPFIHSVEICQASYQTCHHQGPHGKTSLPEQRSF
CTKVCGKEAIWLPQNTILFSPLVADPRQATNSAGIRFNDEVLGKRVGSATFGGDFIFLRLFDISRFHGDM
DIGLQGAVFSVFDLDHPEACMVNSDFFVAALCNFAVNKWSYRFRLWHLSSHLGDEFILANQLPPKKRYNR
SDEAVDFFASFRYTPQIRVYGGIGYIISRDLTFPEDPLYFEGGIELRPFGLREDNLHAQPVFAMHFRFWE
EHDFSIDQTYIVGMEWSKFQDVGRKVRAVLEYHQGFSHEGQFVREECDYYGFRLSYGF
>gi|3328812|gb|AE001312.1:2790-4016,
ATGATGAAACCTCTACGTTTCGGTTATTTCTTTTGCGCAATCTATTTTACTTTGTTACAGGCAGCGTTTG
CTAAAGAACCGAATTCTTGTCCCGACTGCCAGAATAATTGGAAAGAAGTCACCCACACGGATCAACTCCC
AGAAAACATCATTCATGCTGATGATGCTTGTTATCACTCTGGTTATGTACAGGCTCTCATTGATATGCAT
TTCTTAGATAGCTGCTGCCAGGTCATCGTTGAAAACCAAACTGCTTACTTATTTTCTCTTCCTACAGATG
ATGTTACGCGCAACGCCATTATCAACCTAATTAAAGACCTTCCATTCATTCACTCCGTAGAAATCTGCCA
AGCATCCTATCAAACCTGTCATCATCAAGGCCCTCATGGAAAGACTTCTCTTCCAGAACAACCTTCTTTC
TGTACAAAGGTCTGTGGAAAAGAAGCTATTTGGTTACCACAGAATACCATCCTATTCTCGCCTCTTGTAG
CAGATCCTAGACAAGCAACTAATAGTGCAGGTATCCGTTTTAACGACGAAGTCTTAGCAAAACGTGTTGG
CTCTGCTACCTTCGGTGGAGATTTCATCTTCTTACGATTATTTGATATCTCCCGATTCCATGGAGACATG
GATATTGGTCTCCAAGGAGCTGTATTCTCTGTTTTCGACCTGGATCATCCAGAAGCTTGCATGGTCAACT
CTGACTTTTTTGTCGCCGCTTTGTGCAACTTTGCAGTGAACAAATGGAGCTACCGCTTCAGACTATGGCA
TCTTTCTTCTCATCTTGGCGACGAATTTATTCTTGCCAACCAGTTACCTCCTAAAAAACGTTATAATCGA
AGCGATGAAGCCGTCGATTTCTTTGCTTCTTTTCGTTACACTCCACAGATCCGTGTTTATGGAGGTATTG
GGTATATCATTAGTCGAGATTTAACATTCCCTGAAGATCCTCTTTACTTTGAAGCAGGTATCGAACTACG
TCCTTTCGGATTACGGGAAGACAACCTTCATGCCCAACCCGTCTTTGCTATGCATTTTCGCTTTTGGGAA
GAGCATGACTTTTCTATAGACCAAACTTATATAGTAGGCATGGAGTGGTCCAAATTCCAGGATGTAGGGA
GAAAAGTGCGCGCTGTATTGGAATACCACCAAGGTTTCTCCCACGAAGGACAATTTGTCCGAGAAGAATG
CGATTATTATGGCTTTCGATTAAGTTATGGCTTCTAG
>gi|3328651|gb|AAC67834.1|Omp85 Analog [Chlamydia trachomatis]
MLGIRKKTILQLAVLLLLTFSRSSFCSTSEGRMVVESITITTQGENTQNKRAIPKIKTKQGTLFSQADFD
EDLRTLSKDFDRVEPIVEFRNGQAVISLILTAKPVIREINISGNEAIPTHKILKTLELYKNDLFDRELFF
KNFDALRTLYLKRGYYDSQLSYSHNHNEKEGFIDISIEIKEGRHGRIKKLTISGITRTEASDLGDIVLTK
QYSTTTSWFTCAGVYHPDMVEQDLFAITNYFQNKGYADAKVSKEVSTDAKGNITLLIVVDKGPLYTLGHV
HIEGFTALSKRLLDKQLLVGPNSLYCPDKIWTGAQKIRSAYARYGYVNTNVDVSFSAHPTLPVYDVTYRV
SEGSPYKIGLIKIKGNTHTKHDVILHETSLFPGDTFDRLKLEGTETRLRNTGYFKSVSVYTVRSQLDPLD
SNDLYRDVFIEVKETETGNLGLFLGFSSIDHLFGGAEIAESNFDLFGARNFLKKGFKSLRGGGEYLFLKA
NLGDKVTDYTVKWTKPHFLNTPWILGVELDKSINKALSKDYSVDTYGGNISTTYILNDKLKYGMYYRGSQ
TSLSLRKKTSSSNRPGPDLDSNKGFVSAAGLNVLYDSIDNPRKPTMGIRSSLNFELSGLGGTYQFTKLTA
SGSIYRLLTKKGVLKVRAEAKFIKPFGTTTAQGIPVSERFFLGGETTVRGYKPFIIGPKFSPTEPQGGLS
SLLLTEEFQYPLISQPCINAFVFLDSGFIGIEEYTIRLKDLCSSAGFGLRFDMMNNVPIMLGWGWPFRPT
EILNNEKIDVSQRFFFALGGVF
>gi|3328646|gb|AE001297.1:4000-6378,
ATGCTTGGAATACGCAAAAAAACGATTCTGCAACTCGCTGTTTTACTGTTGCTCACCTTTTCACGAAGTT
CTTTCTGTTCAACTTCAGAAGGACGTATGGTCGTAGAGTCTATCACCATTACGACTCAAGGAGAGAATAC
TCAAAATAAACGAGCTATTCCTAAAATAAAAACAAAGCAGGGGACGTTGTTCTCTCAAGCAGATTTTGAT
GAAGATCTAAGAACACTTTCGAAAGATTTTGATCGAGTAGAGCCTATCGTAGAGTTTCGTAATGGACAAG
CTGTGATCTCTCTGATTCTGACGGCAAAACCTGTTATCAGAGAGATCAATATTTCAGGAAATGAAGCTAT
CCCCACTCATAAAATTCTGAAAACTTTACAGCTTTATAAAAATGATCTTTTTGATCGGGAATTATTCTTT
AAAAATTTTGATGCGCTAAGAACTCTTTATTTGAAACGAGGGTACTACGATTCTCAACTCTCCTATTCTC
ATAATCATAATGAGAAAGAGGGCTTTATCGATATTTCCATCGAGATTAAACAAGGACGTCACGGTCGCAT
AAAAAAATTAACGATTTCGGGAATTACGCGAACAGAAGCATCAGACTTAGGTGACATTGTTTTAACTAAA
CAATACTCCACAACAACGAGCTGGTTCACTGGTGCCGGAGTGTATCATCCGGACATGGTAGAGCAAGACT
TATTTGCTATCACAAATTACTTCCAAAATAAAGGATATGCTGATGCTAAAGTAAGCAAAGAGGTCTCTAC
AGATGCTAAAGGAAACATTACTTTGCTTATCGTTGTAGACAAAGGACCTTTATACACATTAGGTCACGTA
CATATAGAAGGATTCACAGCGTTATCCAAAAGACTGCTCGATAAACAACTATTGGTTGGACCTAACTCCT
TATATTGCCCAGATAAAATTTGGACTGGAGCACAAAAGATTCGTAGCGCATACGCTAGATATGGCTACGT
GAACACTAACGTTGATGTCTCCTTCTCAGCGCACCCCACTCTACCTGTTTACGATGTTACCTATCGAGTG
AGTGAAGGATCTCCCTACAAAATCGGGTTAATTAAAATCAAAGGGAACACTCATACTAAGCATGATGTGA
TTTTGCATGAGACTAGTCTTTTCCCTGGAGACACTTTTGATAGATTAAAACTGGAAGGTACAGAGACTCG
TTTACGCAACACCGGCTACTTTAAAAGTGTAAGTGTCTATACGGTTCGTTCCCAATTAGATCCTCTTGAT
TCTAACGACCTTTATCGAGATGTTTTTATTGAAGTCAAAGAGACTGAAACAGGAAATCTTGGGCTATTCT
TAGGATTCAGCTCCATTGACCATTTATTTGGAGGGGCAGAAATTGCAGAAAGCAACTTTGATTTATTTGG
AGCCCGAAACTTTCTCAAAAAAGGATTCAAATCTTTAAGAGGTGGTGGAGAATACCTCTTCCTAAAAGCT
AATTTAGGAGATAAGGTCACCGATTACACTGTTAAATGGACGAAACCACACTTCTTAAATACCCCTTGGA
TTCTTGCAGTAGAATTAGATAAATCAATTAATAAAGCTTTATCAAAAGACTACTCTGTGGATACCTATGG
AGGGAATATCAGTACCACCTACATTCTTAACGATAAGTTAAAATATGGGATGTATTACCGTGGTAGCCAA
ACAAGCTTAAGTTTGCGCAAAAAAACGTCCAGCTCTAATAGACCTGGACCAGATTTAGATAGTAATAAAG
GATTTGTTTCCGCAGCGGGACTCAATGTTCTCTATGATTCTATTGATAATCCTAGAAAACCTACTATGGG
AATCCGCAGCTCCTTAAACTTTGAATTATCTGGTTTAGGCGGAACTTACCAATTTACTAAACTAACAGCT
AGTGGTTCTATCTATCGCTTATTAACTAAAAAAGGTGTTTTGAAAGTCCGTGCAGAAGCTAAGTTTATCA
AACCTTTCGGAACAACAACTGCACAAGGCATTCCTGTCAGCGAACGGTTCTTCTTAGGAGGTGAAACCAC
TGTTCGCGGTTACAAACCTTTTATTATTGGACCGAAATTTTCTCCTACTGAACCACAAGCAGGCTTGTCT
TCCCTACTATTAACAGAAGAATTTCAATATCCTTTGATTTCTCAACCTTGCATTAATGCCTTTGTATTTC
TAGATTCCGGATTCATTGGGATAGAAGAGTACACTATTCGCCTGAAAGACCTTTGCAGTAGCCCCGGATT
TGGTCTACGCTTTGATATGATGAATAATGTGCCAATTATGCTAGGCTGGGGTTGGCCGTTCCGCCCAACA
GAAATCCTCAATAATGAAAAAATTGATGTATCTCAAAGATTCTTTTTTGCCTTGGGAGGAGTATTCTAG
>gi|3328587|gb|AAC67774.1|CMP-2-keto-3-deoxyoctulosonic acid
synthetase [Chlamydia trachomatis]
MFAFLTSKKVGILPSRWGSSRFPGKPLAKILGKTLVQRSYENALSSQSLDCVVVATDDQRIFDHVVEFGG
LCVMTSTSCANGTERVEEVVSRHFPQAEIVVNIQGDEPCLSPTVIDGLVSTLENNPAADMVTSVTETTDP
EAILTDHKVKCVFDKNGKALYFSRSAIPHNFKHPTPIYLHIGVYAFRKAFLSEYVKIPPSSLSLAEDLEQ
LRVLEIGRSIYVHVVQNATGPSVDYPEDTTKVEQYLLCLSKASF
>gi|3328586|gb|AE001292.1:216-980,
GTGTTTGCGTTTTTAACCAGCAAAAAAGTCGGCATTCTCCCCTCTAGATGGGGAAGCTCCCGCTTCCCCG
GAAAACCTCTAGCAAAAATTCTAGGGAAAACCCTTGTTCAAAGATCCTATGAAAATGCCTTAAGCAGTCA
ATCTCTAGATTGCGTTGTTGTGGCAACAGATGATCAACGAATTTTTGACCATGTCGTTGAATTTGGGGGG
CTCTGTGTCATGACTAGCACATCTTGCGCTAACGGAACTGAGCGAGTAGAAGAGGTTGTGTCTCGACATT
TTCCTCAAGCAGAGATTGTTGTGAACATCCAAGGAGACGAGCCCTGTTTATCTCCTACCGTCATAGATGG
GCTTGTGAGCACGCTAGAGAACAATCCTGCTGCAGATATGGTCACATCTGTTACAGAAACAACAGACCCC
GAAGCGATATTGACAGATCACAAAGTGAAGTGTGTTTTCGATAAGAATGGCAAAGCTCTTTACTTTAGCA
GAAGCGCTATTCCTCACAACTTTAAACACCCAACGCCTATTTATCTGCATATTGGTGTTTATGCTTTTAG
AAAAGCTTTTCTAAGTGAATATGTTAAAATTCCTCCTTCCTCGTTAAGCCTAGCCGAAGATCTTGAACAA
TTACGAGTATTAGAAATAGGTCGTTCTATCTACGTTCATGTCGTTCAGAATGCAACGGGCCCTTCTGTTG
ATTATCCCGAAGATATAACCAAAGTGCAGCAGTATTTATTATGTCTTTCAAAAGCATCTTTTTGA
>gi|3329039|gb|AAC68197.1|Thio:disulfide Interchange Protein
[Chlamydia trachomatis]
MIRQWYGFFLCLLFSYTSCFGVEENSGRATPTVELVSESEQAVEGEVLRIGVLIAIPEGEHIYWKNPGKL
GMPLRISWDLPSGCRLLEEHWPTPEIFEEDGVVYFGYKHSTMVVADIRVSKEIETRPLEIKAQVEWLSCG
ASCLPGSSSRVLVIPIDQGPLIPNSKETFTFSRALAAQPRPLDAAIKISYQPDGLDVFVPAGKADRATQA
WFIAENTRDFAYAQEVPLEQATTYIWKLKHPEGNMPKGIGLSCILIFKDDAGKVVASYQVEENQVEQLSA
LSWRFLSILLMAFIGCILLNIMPCVLPLITLKVFSLIKSAADHHSSSVIGGIGFTLGAIVSFWGLAFCAF
LLKVLGQNIGWGFQLQEPMFVAVLIIVFFLFALSSLGVFEMGIICLSLGKKLQEEGGASVRKNQIWGAFF
NGMLTTLVTTPCTGPFLGSVFGLVMAVSFVKQLAIFTAIGLGMASPYLLFASFPKNLAILPKPGPWMSTF
KQLTGFMLLATATWLIWIFGVETSATAVTILLVGLWLAAVGAWILGRWGTLVSPRNQRLLASVVFIFCIL
SSLVITSIGVRYFDENVPPAHSSDWQSFSPEKLADLREKGIPVFVNFTAKWCLTCQLNKPLLHANMQAFA
AKGVVTLEADWTKKDPKITEELARLGRASVPSYVYYPAGNKAPLILPERLSQSALEEMVFSQ
>gi|3329034|gb|AE001330.1:c6695-4617,
ATGATTCGGCAATGGTATGGATTTTTTCTTTGCTTGCTGTTCAGCTATACGTCTTGTTTTGGTGTAGAAG
AAAATAGTGGAAGAGCTACGCCTACAGTAGAACTTGTTAGTGAAAGCGAACAAGCTGTTGAAGGAGAAGT
GCTTCGTATCGGAGTATTGATTGCTATTCCAGAAGGAGAGCATATCTACTGGAAAAATCCAGCGAAGCTT
GGAATGCCTTTGCGCATTTCTTGGGATTTGCCATCAGCATGTAGGTTGCTGGAGGAACATTGGCCGACTC
CACAGATTTTCCAAGAGCATGGCGTTGTTTATTTTGGTTATAAACATTCTACAATGGTGGTTGCGGATAT
TCGCGTTTCTAAAGAGATAGAAACGCGTCCATTGGAGATAAAAGCGCAAGTTGAATGGTTGTCTTGCGGT
GCATCTTGTCTCCCAGGTTCTTCGTCAAGGGTTCTTGTGATTCCTATAGATCAGGGGCCGTTAATTCCTA
ATAGTAAAGAGACATTCACTTTTTCCCGTGCGTTAGCGGCTCAACCTCGACCTTTGGATGCTGCCATAAA
GATTTCTTATCAGCCTGATGGCTTAGATGTTTTTGTGCCAGCAGGGAAAGCGGATCGGGCAACCCAGGCA
TGGTTCATTGCTGAAAACACGCCAGATTTTGCTTATGCTCAAGAGGTTCCTCTTGAGCAAGCGACTACGT
ACATATGGAAGTTGAAACATCCTGAAGGAAATATGCCTAAGGGTATTGGGTTGTCGGGGATTCTTATATT
CAAGGATGATGCAGGGAAAGTAGTCGCTTCGTATCAAGTAGAAGAGAATCAAGTCGAACACCTTTCGGCA
TTGAGCTGGAGGTTTCTCTCTATTCTTCTTATGGCTTTCATTGGTGGAATCTTATTAAACATCATGCCCT
GTGTATTGCCTCTGATTACTTTGAAAGTATTTAGTTTAATTAAATCGGCGGCAGATCACCATTCTTCCTC
TGTGATTGGAGGGATTGGGTTTACTTTACGGGCTATTGTAAGCTTTTGGGGACTCGCTTTTTGTGCGTTT
TTGTTAAAGGTTTTAGGGCAAAATATTGGATGGGGATTCCAGCTTCAACAACCCATGTTTGTTGCCGTTT
TAATTATTGTCTTCTTCTTATTTGCTCTGAGTTCGTTAGGCGTTTTTGAGATGGGAATAATTTGTCTGAG
CCTAGGGAAAAAATTGCAAGAAGAGGCAGGGGCATCGGTAAGGAAGAATCAGATCTGGGGAGCTTTTTTC
AATGGCATGTTGACTACCCTGGTTACAACTCCTTGCACTGGGCCTTTTCTTGGCTCTGTATTTGGATTAG
TTATGGCAGTGTCTTTTGTTAAGCAGCTGGCAATTTTTACTGCTATAGGATTAGGAATGGCAAGTCCCTA
TCTATTATTTGCTTCTTTTCCGAAGATGCTAGCCATTTTACCTAAACCTGGTCCTTGGATGAGTACGTTT
AAACAGTTGACTGGGTTTATGTTGCTTGCTACTGCAACTTGGCTTATCTCGATTTTTGGGGTAGAGACGA
GTGCAACCGCTGTAACTATTCTTCTTGTAGGATTGTGGTTGGCTGCTGTAGGTGCATGGATTCTAGGGAG
ATGGGGAACCCTTGTATCTCCGCGTAATCAGCGGCTTCTTGCTTCCGTTGTATTCATTTTCTGTATTTTA
AGTTCCTTAGTGATTACCTCTATAGGTGTCCGTTATTTTGATGAGAACGTCCCTCCTGCACATAGCTCTG
ATTGGCAATCTTTTTCTCCCGAAAAGCTAGCTGATTTACGCGAAAAAGGGATTCCAGTTTTTGTAAATTT
CACTGCAAAGTGGTGTTTAACGTGTCAACTCAATAAGCCTCTTCTTCATGCCAATATGCAAGCTTTTGCT
GCTAAGGGCGTAGTTACTTTAGAAGCAGATTGGACGAAAAAACATCCAAAAATTACAGAAGAACTCGCTC
GTTTAGGCCGAGCCAGTGTACCTTCTTATGTGTATTACCCTGCGGGGAACAAAGCTCCCCTTATTCTTCC
ACAAAGATTATCGCAATCTGCTTTGGAAGAGATGGTTTTTTCTCAGTAG
>gi|3329000|gb|AAC68161.1|Yop proteins translocation lipoprotein J
[Chlamydia trachomatis]
MFRYTLSRSLFFILALFFCSACDSRSMITHGLSGRDANEIVVLLVSKGVAAQKVPQAASSTGGSGEQLWD
ISVPAAQITEALAILNQAGLPRMKGTSLLDLFAKQGLVPSEMQEKIRYQEGLSEQMATTIRKMDGIVDAS
VQISFSPEEEDQRPLTASVYIKHRGVLDNPNSIMVSKIKRLVASAVPGLCPENVSVVSDRASYSDITING
PWGLSDEMNYVSVWGIILAKHSLTKFRLVFYFLILLLFILSCGLLWVIWKTHTLISALGGTKGFFDPAPY
SQLSFTQNKPAPKETPGAAEGAEAQTASEQPSKENAEKQEENNEDA
>gi|3328999|gb|AE001327.1:84-1064,
ATGTTTCGTTATACTCTTTCTCGATCCTTATTTTTCATTTTGGCTCTTTTCTTCTGCTCGGCTTGTGATA
GTCGTTCCATGATTACACACGGCTTGTCAGGACGTGATGCTAATGAAATCGTAGTGCTTCTAGTCAGTAA
AGGGGTCCCTGCACAGAAAGTTCCCCAAGCAGCGTCCTCAACAGGAGGATCTGGAGAACAACTCTGGGAT
ATTTCGGTTCCTGCAGCACAAATTACAGAGGCTCTAGCTATTCTGAACCAAGCTGGGCTTCCAAGAATGA
AAGGAACCAGCCTTCTTGATCTATTCGCTAAACAAGGGCTGGTCCCTTCTGAAATGCAAGAAAAAATCCG
CTACCAAGAAGGTCTTTCAGAACAAATGGCTACGACCATTAGAAAGATGGACGGTATCGTCGATGCGAGC
GTACAGATTTCCTTTTCTCCTGAAGAAGAACATCAACGGCCGCTAACAGCCTCTGTATATATCAAACACA
GAGGGGTATTAGACAACCCTAACAGTATTATGGTGTCTAAGATTAAACGTTTAGTTGCGAGTGCTGTCCC
AGGACTATGTCCCGAGAACGTTTCCGTAGTCAGTGACCGAGCTTCTTATAGTGACATTACTATTAATGGC
CCTTGGGGACTCTCCGATGAAATGAATTATGTTTCTGTATGGGGGATCATTCTAGCTAAGCATTCCCTTA
CTAAATTCCGCCTTGTTTTCTATTTCTTAATTCTCCTTCTCTTCATTCTTTCCTGTGGGCTACTCTGGGT
CATTTGGAAAACACACACACTGATTTCTGCTCTGGGTGGAACAAAAGGATTCTTTGATCCTGCTCCTTAC
TCACAGCTCTCTTTCACTCAGAATAAGCCAGCTCCAAAAGAAACTCCTGGAGCAGCAGAAGGTGCAGAAG
CGCAAACCGCTTCCGAACAACCCTCTAAAGAAAACGCAGAAAAACAAGAAGAGAATAACGAGGACGCTTA
A
>gi|3328905|gb|AAC68071.1| hypothetical protein [Chlamydia trachomatis]
MEKRGVIVHILVCLLTIFGTFSLPAFGAHFLAEEEQFYMDRFVFSGQYPDMETMEIHAERKKRVQFDVTG
SFPKLESVVYKGSFGLLRSKIKGECPELSSVNLSCTSCRMDLDFRCEWKKNASIYIRNEQEPITIMLPKD
IGVVVYTQVDNNSKVVAEGSLIKRGRGFWKKTFRNSLVGESPVTLTFHVETRNGGVIFLR
>gi|332889|gb|AE001320.1:c11104-10502,
ATGGAGAAGAGAGGCGTTATTGTGCATATACTAGTTTGTTTGTTGACAATCTTCGGAACGTTCAGTTTAC
CCGCTTTCGGCGCGCATTTTCTCGCGGAAGAAGAGCAGTTTTATATGGATCGGTTTGTTTTCTCTGGGCA
GTATCCAGATATGGAAACTATGGAAATCCATGCAGAAACAAAAAAACGTGTACAATTTGATGTGACGGGA
AGCTTCCCTAAGTTGGAGAGCGTGGTTTATAAGGGATCTTTTGGATTGCTGCGTTCGAAAATAAAGGGAG
AGTGTCCAGAACTGTCTTCTGTAAATCTTTCTTGTACCTCCTGCAGAATGGATTTAGATTTTCGAGGGGA
GTGGAAAAAGAATGCGTCTATTTATATTCGTAATGAGCAAGAGCCAATTACAATTATGTTGCCTAAAGAC
ATTGGTGTAGTTGTCTATACGCAGGTTGATATGAATAGTAAAGTAGTTGCAGAGGGATCACTAATCAAGA
GAGGAAGAGGTTTTTGGAAGAAAACTTTTCGGAATTCTTTGGTAGGAGAATCCCCTGTGACGCTAACTTT
TCATGTAGAGACTCGTAATGGAGGAGTTATTTTTCTCCGTTAG
>gi|3328884|gb|AAC68051.1| Phosphatidate Cytidylytransferase
[Chlamydia trachomatis]
MFDSDHNSIFQSDLCQRLVVHSILLTFLVILLCTSLYPSSAFIVGLLSSACAALGTYEMGAMVRIKFPFS
FTRYSALGSAIFIALTCLTARCKMCFPEHIDLLPWFFLFFWTIRLVFKSRHYKLGPIGSTGLALFCMLYV
SVPIRLFLHILYGFVHTDTPFVGIWWAIFLIATTKSSDIFGYFFGKAFGKKRIAPVISPNKTVVGFIAGC
CGSILVSLLFYSHLPKAFADQIAVPWILIALGTVLGVSGFFGDIIESTFKRDAQIKNSSDLESIGGMLDV
LDSLLLSTPIVYAILLITQNRTFLG
>gi|3328881|gb|AE001319.1:1804-2721,
ATGTTCGATTCGGATCATAATTCCATTTTTCAAAGCGATTTGTGTCAGCGTCTGGTCGTCCACTCGATTC
TTCTTACTTTCCTTGTCATTCTTCTCTGTACATCTTTATATCCCAGCTCAGCCTTTATTGTAGGGCTTCT
TTCCTCCGCTTGCGCAGCTCTAGGAACATATGAGATGGGGGCTATGGTTAGAATCAAGTTTCCATTTTCT
TTCACACGCTATAGTGCATTAGGATCCGCTATTTTCATTGCTCTGACCTGTCTTACAGCTCGTTGTAAAA
TGTGTTTTCCAGAGCATATAGACCTACTTCCTTGGTTCTTTCTCTTCTTTTGGACGATTCGCTTAGTATT
TAAAAGTCGCCATTATAAACTTGGTCCCATAGGCTCAACTGGGCTCGCGTTGTTTTGTATGCTTTATGTA
TCAGTCCCTATCCGCTTGTTCCTCCACATTTTGTATGGGTTTGTGCATACCGATACTCCATTTGTAGGAA
TTTGGTGGGCGATTTTTCTTATCGCTACAACAAAAAGCTCTGATATTTTTGGTTACTTCTTTGGAAAAGC
TTTTGGGAAAAAACGCATTGCACCAGTCATTAGCCCGAACAAAACAGTAGTAGGCTTCATTGCTGGTTGC
TGTGGATCTATCTTGGTTAGCCTTCTTTTCTACTCCCATCTTCCTAAAGCCTTTGCTGATCAGATTGCGG
TGCCTTGGATCCTCATTGCTTTAGGTACTGTGTTGGGCGTTAGTGGATTCTTTGGAGACATTATCGAATC
TACGTTCAAACGGGATGCACAGATCAAGAACAGCAGTGATCTGGAGTCTATCGGAGGAATGCTAGATGTG
CTAGACTCCTTGCTTCTTTCGACTCCTATCGTTTACGCTATCCTCCTTATCACTCAAAATAGGACATTTT
TAGGATCA
>gi|3328855|gb|AAC68022.1|hypothetical protein [Chlamydia trachomatis]
MRRSVCYVTPSVARAGQISTWRFEYSSANFLPEGTLLKFDLGIDGRPIDWEIPSTDLSQPCNTIYLETPS
EDIVAAKAVYAPGGYIPTFEFTLPCDVEAGDTFSIILGSSPNFPQEDSSGNGAQLFTQRRKPFSLYVDPS
GKGSFEDPDIFTMDIRGNVLKNIRIFAPSYVIKNKRFDITVRFEDEFGNLTNFSPEETHIELSYEHLREN
LNWQLFIPETGFVILPNLYFNEPGIYRIQLRNQATKEVFTSAPIKCFAETSSHLLWGLLHGESERVDSEG
NIESCLRYFRDDCALNFFATSSFEIQDGLTPETIKTINQTVADFNEEDRFIALSGAQYLSEEPGEGIREV
LLMKEPKSPGKHKECKLFPLSKLYKQSTSHELISIPSFTASKKFGYNFNNFHPEFERVVEIYNAWGCSER
TEAEGNPFPIKGSIDSENPEGTVLSALKRNLRFGFVAGGLDDRNLYNHFFDSDQQQYSPGLTAVICNKYS
RDSLLEALYQRQCYATTGQRIIVNFQITSAPMGSELSTAIKPGLVINRHISGYVAGTAKIASIEIIRNED
ILHTFHPDGNNFEYEYDDLSPFAQVTLKDPQNGAPFAFYYLRVTQENGAMAWSSPIWIDLN
>gi|3328850|gb|AE001316.1:4105-5970,
ATGCGCAGATCTGTTTGTTACGTTACTCCTTCAGTTGCTAGGGCTGGTCAAATTTCTACCTGGCGTTTCG
AATATTCTTCAGCTAATTTCCTTCCCGAAGGCACATTGCTAAAATTTGACCTGGGAATAGACGGACGCCC
TATAGACTGGGAGATTCCTTCTACAGATCTTTCTCAACCATGTAATACAATTTATTTAGAAACGCCTTCC
GAGGATATTGTGGCTGCAAAAGCTGTGTATGCTCCCGGAGGCTATATCCCTACTTTCGAATTTACTCTCC
CTTGTGATGTGGAAGCTGGGGACACTTTCTCTATTATTCTTGGCTCCTCTCCCAACTTCCCTCAAGAGGA
CTCTTCAGGTAATGGTGCTCAATTATTTACTCAACGCCGTAAACCTTTCTCTCTTTATGTTGACCCATCA
GGGAAAGGATCTTTTCAAGATCCCGATATCTTCACAATGGATATCAGAGGAAATGTATTAAAAAATATCC
GGATTTTTGCTCCTTCTTATGTGATCAAAAACAAACGCTTTGATATTACAGTGCGCTTCGAAGATGAATT
TGGGAACTTAACCAATTTCTCCCCAGAAGAGACCCATATCGAGCTTTCGTACGAACATCTTCGCGAAAAC
CTCAATTGGCAATTGTTCATCCCTGAAACAGGCTTTGTGATCCTTCCAAACCTGTATTTCAATGAACCAG
GTATTTATCGTATTCAACTACGCAATCAAGCAACAAAAGAGGTCTTTACATCAGCGCCTATCAAATGTTT
TGCAGAAACCTCCTCTCATCTTTTGTGGGGGCTTCTACATGGAGAATCTGAACGTGTCGACTCTGAAGGT
AATATCGAGTCTTGCTTGCGTTATTTTCGTGATGACTGCGCGTTAAACTTTTTTGCAACATCCTCTTTCG
AAATTCAAGATGGCCTGACCCCAGAAACCATTAAAACCATTAACCAAACCGTTGCTGATTTTAATGAAGA
AGATCGTTTCATTGCCTTATCCGGAGCACAGTACCTTTCTGAAGAGCCTGGCGAGGGAATTCGTGAAGTA
TTGCTGATGAAGGAACCCAAATCCCCAGGGAAACATAAAGAATGCAAACTATTTCCTTTATCTAAGCTAT
ATAAGCAATCAACTAGTCATGAGTTAATCTCAATCCCCAGCTTCACTGCTTCAAAGAAATTTGGATACAA
TTTTAATAATTTCCATCCTGAATTTGAAAGAGTTGTTGAAATTTATAATGCCTGGGGATGCTCTGAAAGA
ACTGAAGCTGAAGGAAACCCTTTCCCTATTAAAGGTTCTATCGACTCAGAAAATCCAGAGGGAACTGTTC
TATCTGCTTTAAAGAGAAACCTGCGTTTTGGATTCGTAGCCGGTGGTCTTGATGATAGAAATCTATACAA
TCACTTTTTTGATTCCGATCAACAGCAATACTCCCCTGGATTAACAGCTGTGATCTGCAATAAATATTCT
CGGGATTCCTTACTCGAGGCATTATACCAACGACAATGCTATGCTACAACCGGCCAAAGAATTATCGTGA
ATTTCCAGATTACATCTGCTCCTATGGGCTCCGAACTCTCCACAGCCATTAAACCAGGGCTCGTGATCAA
TAGACATATTTCGGGATATGTAGCAGGAACTGCCAAGATTGCGTCGATCGAAATCATCCGCAATGAGGAT
ATTCTCCATACCTTCCACCCAGATGGAAATAACTTTGAGTATGAGTACGACGATCTCTCTCCTTTTGCAC
AAGTCACTCTAAAAGATCCTCAAAATGGAGCTCCTTTTGCTTTTTACTACTTACGAGTCACTCAAGAGAA
TGGAGCTATGGCTTGGAGCTCTCCTATTTGGATAGATCTTAACTAA
>gi|3328772|gb|AAC67946.1| hypothetical protein [Chlamydia
trachomatis]
MKRFFPLFIGVLLAHTLPSEGLSHQQAVQKKISYLSHFKGITGIMDVEDGVLHIHDDLRLQANKAYVENR
TDCGIKIVAHGNVMVNYRGKILICDYLEYYEDTDSCLLTNGRCSLYPWFIGGSTITISPSSIIIHKGYIS
TSEGPQKHICLSGDYLKYSSDSVLSMGPSRLSICNTPVLLLPQISIMPMEIPKPPITFRGGSGGFLGSYL
GVSYSPISKKHCSTTLFLDGFFKHGIGLGYNMRFSSQENPSNAINIKSYYAHRLAIDSSGAKDRYRLHGD
FTFSKERAHLAGEFHLSDSWETVVDIFPNNFSLKNTGPTEVSLSWRDNNLFGKMTSSVKVNSFQNVKQEL
PQAILHHRPVRIRRSRIFLENRLEAGFLDFHFSSNIPGSNFSSWRFSSAHKVYRGLVLPIGTLTPSLSGT
AIYYTRMLSPNAAHCQLSGSLSFDYRVALQKEYRHARHIVEPFCSFLKTTRPVLSSDEPHIFSIKDAFHS
INLLHVGLESKVLNKHSTPSHLKLWTTYIFDEPHAKDTFPKTACWFSLPLTLQNTLSLDAEWIWKKSRWD
HLNVIWEWILNDNLGLTLEFLHRSKYGFIKCAKDNYTLDVSRSLDTLLASPLSDRRNLITGKLFVRPHPH
WNYNLNLRYGWHRPDSPSYLEYQMILGHKIFEHWQLFSVYEKREADKRCFFYLKLDKRKQKHRHPFG
>gi|3328766|gb|AE001308.1:6085-8178,
GTGAAACGATTTTTCCCACTTTTTATTGGAGTGCTGCTCGCGCACACTTTGCCGTCAGAAGGTCTTTCTC
ATCAACAAGCTGTCCAAAAAAAAATTTCTTATCTGAGCCATTTTAAAGGCATTACACGAATTATGGATGT
TGAGGATGGGGTATTACATATCCATGATGATCTACGTCTTCAAGCCAATAAAGCCTATGTTGAAAATCGC
ACGGATTGTGGGATCAAAATCGTTGCTCATGGCAACGTTATGGTCAATTATCGCGGGAAAATTTTAATCT
GTGATTATCTTGAGTACTATGAAGATACAGATTCTTGTTTACTCACCAATGGCCGCTGTTCGTTATACCC
ATGGTTCATTGGAGGATCCACTATAACGATCTCACCATCTTCTATTATCATTCATAAAGGCTATATCTCG
ACTTCTGAAGGTCCTCAGAAACATATTTGTTTATCCGGAGATTATTTAAAATACTCTTCAGACAGCGTAT
TATCTATGGGACCCTCACGTCTTTCTATCTGTAATACGCCTCTGTTATTGCTTCCTCAAATCTCCATTAT
GCCTATGGAGATTCCTAAGCCTCCGATTACTTTTCGAGGTGGGAGTGGAGGATTTCTGGGATCCTACTTA
GGTGTTAGTTATTCCCCTATATCTAAAAAGCATTGTTCTACGACTTTGTTCTTGGATGGTTTTTTTAAAC
ATGGAATAGGTCTCGGCTATAACATGCGCTTTTCCTCTCAGGAAAATCCAAGCAATGCCATAAATATTAA
AAGCTATTACGCACATCGATTAGCTATTGATTCATCAGGAGCAAAAGATCGCTATCGATTACATGGAGAC
TTCACTTTTTCCAAAGAACGAGCCCATCTTGCTGGTGAATTCCATTTAAGTGATAGCTGGGAAACAGTTG
TGGATATCTTCCCAAATAACTTCTCTTTAAAAAATACAGGCCCTACAGAAGTTAGCCTATCATGGCGCGA
TAACAATTTATTTGGGAAAATGACTTCCTCTGTCAAAGTCAACTCCTTTCAAAATCTTAAACAAGAATTG
CCTCAAGCAATTCTTCATCACCGACCAGTACGTATCAGGCGCTCTCGCATTTTCCTAGAGAATCGCTTAG
AAGCTGGTTTTTTAGATTTTCATTTCAGTAGTAATATTCCAGGCTCTAACTTCTCATCATGGAGGTTCTC
ATCCGCTCACAAAGTCTACCGTGGGCTTGTTCTTCCTATAGGAACGTTAACCCCTTCGCTATCTGGAACT
GCTATCTACTATACCCGCATGCTCTCCCCAAATGCAGCCCATTGTCAATTATCTGGATCCCTATCTTTTG
ATTATCGCGTTGCTTTACAAAAAGAATATCGGCATGCAAGACATATTGTAGAGCCTTTTTGCTCCTTTTT
AAAAACCACTCGTCCTGTATTATCCTCTGATGAGCCTCATATTTTCTCGATTAAAGATGCTTTTCACTCT
ATCAACCTTCTACATGTAGGATTGGAGTCAAAAGTCTTAAACAAACATTCCACTCCTTCGCATCTGAAAC
TATGGACGACCTATATCTTTGATGAACCTCACGCTAAGGACACTTTCCCTAAAACTGCTTGCTGGTTCTC
TCTTCCTCTTACACTCCAAAATACTTTATCCTTAGATGCGGAATGGATTTGGAAAAAAAGCCGATGGGAT
CATCTCAATGTAATCTGGGAATGGATTTTGAATGATAATCTCGGTCTTACTTTACAATTTTTACATAGAA
GTAAGTATGGCTTTATTAAGTGCGCTAAAGATAACTACACACTCGATGTAAGCCGATCTTTAGACACATT
ACTAGCCTCTCCTCTTTCCGATCGAAGAAATTTGATTACTGGCAAACTTTTTGTTCGTCCACATCCTCAT
TGGAATTATAATCTTAATCTTCGTTATGGATGGCATCGTCCAGACTCTCCATCCTATTTAGAATACCAGA
TGATTCTGGGTCATAAAATCTTTGAGCACTGGCAGCTATTCTCTGTCTACGAAAAACGTGAAGCTGATAA
GCGCTGCTTCTTTTATCTAAAATTAGATAAACGAAAACAGAAACACCGCCATCCTTTTGGATAA
>gi|3329347|gb|AAC68470.1|Putative Outer Membrane Protein H
[Chlamydia trachomatis]
MPFSLRSTSFCFLACLCSYSYGFASSPQVLTPNVTTPFKGDDVYLNGDCAFVNVYAGAENGSIISANGDN
LTITGQNHTLSFTDSQGPVLQNYAFISAGETLTLKDFSSLMFSKNVSCGEKGMISGKTVSISGAGEVIFW
DNSVGYSPLSIVPASTPTPPAPAPAPAASSSLSPTVSDARKGSIFSVETSLEISGVKKGVMFDNNAGNFG
TVFRGNSNNNAGSGGSGSATTPSFTVKNCKGKVSFTDNVASCGGGVVYKGTVLFKDNEGGIFFRGNTAYD
DLGILAATSRDQNTETGGGGGVICSPDDSVKFEGNKGSIVFDYNFAKCRGGSILTKEFSLVADDSVVFSN
NTAEKGGGAIYAPTIDISTNGGSILFERNRAAEGGAICVSEASSGSTGNLTLSASDGDIVFSGNMTSDRP
GERSAARILSDGTTVSLNASGLSKLTFYDPVVQNNSAAGASTPSPSSSSMPGAVTINQSGNGSVIFTAES
LTPSEKLQVLNSTSNFPGALTVSGGELVVTEGATLTTGTITATSGRVTLGSGASLSAVAGAANNNYTCTV
SKLGIDLESFLTPNYKTAILGADGTVTVNSGSTLDLVMESEAEVYDNPLFVGSLTIPFVTLSSSSASNGV
TKNSVTINDADAAHYGYQGSWSADWTKPPLAPDAKGMVPPNTNNTLYLTWRPASNYGEYRLDPQRKGELV
PNSLWVAGSALRTFTNGLKEHYVSRDVGFVASLHALGDYILNYTQDDRDCFLARYGGFQATAASHYENGS
IFGVAFCQLYGQTKSRMYYSKDAGNMTMLSCFGRSYVDIKGTETVMYWETAYGYSVHRMHTQYFNDKTQK
FDHSKCHWHNNNYYAFVGAEHNFLEYCIPTRQFARDYELTGFMRFEMAGGWSSSTRETGSLTRYFARGSG
HNMSLPIGIVAHAVSHVRRSPPSKLTLNMGYRPDIWRVTPHCNMEIIANGVKTPIQGSPLARHAFFLEVH
DTLYIHHFGRAYMNYSLDARRRQTAHFVSMGLNRIF
>gi|3329342|gb|AE001360.1:10808-13858,
ATGCCTTTTTCTTTGAGATCTACATCATTTTGTTTTTTAGCTTGTTTGTGTTCCTATTCGTATGGATTCG
CGAGCTCTCCTCAAGTGTTAACACCTAATGTAACCACTCCTTTTAAGGGGGACGATGTTTACTTGAATGG
AGACTGCGCTTTTGTCAATGTCTATGCAGGGGCAGAGAACGGCTCAATTATCTCAGCTAATGGCGACAAT
TTAACGATTACCGGACAAAACCATACATTATCATTTACAGATTCTCAAGGGCCAGTTCTTCAAAATTATG
CCTTCATTTCAGCAGGAGAGACACTTACTCTGAAAGATTTTTCGAGTTTGATGTTCTCGAAAAATGTTTC
TTGCGGAGAAAAGGGAATGATCTCAGGGAAAACCGTGAGTATTTCCGGAGCAGGCGAAGTGATTTTTTGG
GATAACTCTGTGGGGTATTCTCCTTTGTCTATTGTGCCAGCATCGACTCCAACTCCTCCAGCACCAGCAC
CAGCTCCTGCTGCTTCAACCTCTTTATCTCCAACAGTTAGTGATGCTCGGAAAGGGTCTATTTTTTCTGT
AGAGACTAGTTTGGAGATCTCAGGCGTCAAAAAAGGGGTCATGTTCGATAATAATGCCGGGAATTTTGGA
ACAGTTTTTCGAGGTAATAGTAATAATAATGCTGGTAGTGGGGGTAGTGGGTCTCCTACAACACCAAGTT
TTACAGTTAAAAACTGTAAAGGGAAAGTTTCTTTCACAGATAACGTAGCCTCCTGTGGAGGCCGAGTAGT
CTACAAAGGAACTGTGCTTTTCAAAGACAATGAAGGAGGCATATTCTTCCGAGGGAACACAGCATACGAT
GATTTAGGGATTCTTGCTGCTACTAGTCGGGATCAGAATACGGAGACAGGAGGCGGTGGAGGAGTTATTT
GCTCTCCAGATGATTCTGTAAAGTTTGAAGGCAATAAAGGTTCTATTGTTTTTGATTACAACTTTGCAAA
ACGCAGAGGCGGAAGCATCCTAACCAAAGAATTCTCTCTTGTAGCAGATGATTCGGTTGTCTTTAGTAAC
AATACAGCAGAAAAAGGCGGTGGAGCTATTTATGCTCCTACTATCGATATAAGCACGAATGGAGGATCGA
TTCTATTTGAAAGAAACCGAGCTGCAGAAGGAGGCGCCATCTGCGTGAGTGAAGCAAGCTCTGGTTCAAC
TGGAAATCTTACTTTAAGCGCTTCTGATGGGGATATTGTTTTTTCTGGGAATATGACGAGTGATCGTCCT
GGAGAGCGCACCGCAGCAAGAATCTTAAGTGATGGAACGACTGTTTCTTTAAATGCTTCCGCACTATCGA
AGCTGATCTTTTATGATCCTGTAGTACAAAATAATTCAGCAGCGGGTGCATCGACACCATCACCATCTTC
TTCTTCTATGCCTGGTGCTGTCACGATTAATCAGTCCGGTAATGGATCTGTGATTTTTACCGCCGAGTCA
TTGACTCCTTCAGAAAAACTTCAAGTTCTTAACTCTACTTCTAACTTCCCAGGAGCTCTGACTGTGTCAG
GAGGGGAGTTGGTTGTGACGCAAGGAGCTACCTTAACTACTGGGACCATTACAGCCACCTCTGGACGAGT
GACTTTAGGATCCGGAGCTTCGTTGTCTGCCGTTGCAGGTGCTGCAAATAATAATTATACTTGTACAGTA
TCTAAGTTGCGGATTGATTTACAATCCTTTTTAACTCCTAACTATAAGACGGCCATACTGGGTGCGGATG
GAACAGTTACTGTTAACAGCGGCTCTACTTTAGACCTAGTGATGGAGAGTGACGCAGAGGTATATGATAA
TCCGCTTTTTGTGGGATCGCTGACAATTCCTTTTGTTACTCTATCTTCTAGTAGTGCTAGTAACGGAGTT
ACAAAAAATTCTGTCACTATTAATGATGCAGACGCTGCGCACTATGGGTATCAAGGCTCTTGGTCTGCAG
ATTGGACGAAACCGCCTCTGGCTCCTGATGCTAAGGGGATGGTACCTCCTAATACCAATAACACTCTGTA
TCTGACATGGAGACCTGCTTCGAATTACGGTGAATATCGACTGGATCCTCAGAGAAAGGGAGAACTAGTA
CCCAACTCTCTTTGGCTAGCGGGATCTGCATTAAGAACCTTTACTAATGGTTTGAAAGAACACTATGTTT
CTAGAGATGTTGGATTTGTAGCATCTCTGCATGCTCTCGGGGATTATATTTTGAATTATACGCAAGATGA
TCGGGATGGCTTTTTAGCTAGATATGGGGGATTCCAGGCGACCGCAGCCTCCCATTATGAAAATGGGTCA
ATATTTGGAGTGGCTTTTGGACAACTCTATGGTCAGACAAAGAGCAGAATGTATTACTCTAAAGATGCTG
GGAACATGACGATGTTGTCCTGTTTCGGAAGAAGTTACGTAGATATTAAAGGAACAGAAACTGTTATGTA
TTGGGAGACGGCTTATGGCTATTCTGTGCACAGAATGCATACGCAGTATTTTAATGACAAAACGCAGAAG
TTCGATCATTCGAAATGTCATTGGCACAACAATAACTATTATGCGTTTGTGGGTGCCGAGCATAATTTCT
TAGAGTACTGCATTCCTACTCGTCAGTTCGCTAGAGATTATGAGCTTACAGGGTTTATGCGTTTTGAAAT
GGCCGGAGGATGGTCCAGTTCTACACGAGAAACTGGCTCCCTAACTAGATATTTCGCTCGCGGGTCAGGG
CATAATATGTCGCTTCCAATAGGAATTGTAGCTCATGCAGTTTCTCATGTGCGAAGATCTCCTCCTTCTA
AACTGACACTAAATATGGGATATAGACCAGACATTTGGCGTGTCACTCCACATTCCAATATGGAAATTAT
TGCTAACGGAGTGAAGACACCTATACAAGGATCTCCGCTGGCACGGCATGCCTTCTTCTTAGAAGTGCAT
GATACTTTGTATATTCATCATTTTGGAAGAGCCTATATGAACTATTCGCTGGATGCTCGTCGTCGACAAA
CGGCACATTTTGTATCCATGGGCTTGAATAGAATCTTTTAA
>gi|3328874|gb|AAC68042.1| 60kDa Cysteine-Rich OMP
[Chlamydia trachomatis]
MRIGDPNNKLIRRAVTIFAVTSVASLFASGVLETSMAESLSTNVISLADTKAKDNTSHKSKKARKNHSKE
TPVDRKEVAPVHESKATGPKQDSCFGRMYTVKVNDDRNVEITQAVPEYATVGSPYPIEITATGKRDCVDV
IITQQLPCEAEFVRSDPATTPTADGKLVWKIDRLGQGEKSKITVWVKPLKEGCCFTAATVCACPEIRSVT
KCGQPAICVKQEGPENACLRCPVVYKINIVNQGTATARNVVVENPVPDGYAHSSGQRVLTFTLGDMQPGE
HRTITVEFCPLKRGRATNIATVSYCGGHKNTASVTTVINEPCVQVSIAGADWSYVCKPVEYVISVSNPGD
LVLRDVVVEDTLSPGVTVLEAAGAQISCNKVVWTVKELNPGESLQYKVLVRAQTPGQFTNNVVVKSCSDC
GTCTSCAEATTYWKGVAATHMCVVDTCDPVCVGENTVYRICVTNRGSAEDTNVSLMLKFSKELQPVSFSG
PTKGTITGNTVVFDSLPRLGSKETVEFSVTLKAVSAGDARGEAILSSDTLTVPVSDTENTHIY
>gi|3328863|gb|AE001317.1:c11039-9378,
ATGCCAATAGGAGATCCTATGAACAAACTCATCAGACGAGCAGTGACGATCTTCGCGGTGACTAGTGTGG
CGAGTTTATTTGCTAGCGGGGTGTTAGAGACCTCTATGGCAGAGTCTCTCTCTACAAACGTTATTAGCTT
AGCTGACACCAAAGCGAAAGACAACACTTCTCATAAAAGCAAAAAAGCAAGAAAAAACCACAGCAAAGAG
ACTCCCGTAGACCGTAAAGAGGTTGCTCCGGTTCATGAGTCTAAAGCTACAGGACCTAAACAGGATTCTT
GCTTTGGCAGAATGTATACAGTCAAAGTTAATGATGATCGCAATGTTGAAATCACACAAGCTGTTCCTGA
ATATGCTACCGTAGGATCTCCCTATCCTATTGAAATTACTGCTACAGGTAAAAGGGATTGTGTTGATGTT
ATCATTACTCAGCAATTACCATGTGAAGCAGAGTTCGTACGCAGTGATCCAGCGACAACTCCTACTGCTG
ATGGTAAGCTAGTTTGGAAAATTGACCGCTTAGGACAAGGCGAAAAGAGTAAAATTACTGTATGGGTAAA
ACCTCTTAAAGAAGGTTGCTGCTTTACAGCTGCAACAGTATGCGCTTGTCCAGACATCCGTTCGCTTACA
AAATGTGGACAACCTGCTATCTGTGTTAAACAAGAAGGCCCAGAGAATGCTTGTTTGCGTTGCCCAGTAG
TTTACAAAATTAATATAGTGAACCAAGGAACAGCAACAGCTCGTAACGTTGTTGTTGAAAATCCTGTTCC
AGATGGTTACGCTCATTCTTCTGGACAGCGTGTACTGACGTTTACTCTTGGAGATATGCAACCTGGAGAG
CACAGAACAATTACTGTAGAGTTTTGTCCGCTTAAACGTGGTCGTGCTACCAATATAGCAACGGTTTCTT
ACTGTGGAGGACATAAAAATACAGCAAGCGTAACAACTGTGATCAACGAGCCTTGCGTACAAGTAAGTAT
TGCAGGAGCAGATTGGTCTTATGTTTGTAAGCCTGTAGAATATGTGATCTCCGTTTCCAATCCTGCAGAT
CTTGTGTTGCGAGATGTCGTCGTTGAAGACACTCTTTCTCCCGGAGTCACAGTTCTTGAAGCTGCAGGAG
CTCAAATTTCTTGTAATAAAGTAGTTTGGACTGTGAAAGAACTGAATCCTGGAGAGTCTCTACAGTATAA
AGTTCTAGTAAGAGCACAAACTCCTGGACAATTCACAAATAATGTTGTTGTGAAGAGCTGCTCTGACTGT
GGTACTTGTACTTCTTGCGCAGAAGCGACAACTTACTGGAAAGGAGTTGCTGCTACTCATATGTGCGTAG
TAGATACTTGTGACCCTGTTTGTGTAGGAGAAAATACTGTTTACCGTATTTGTGTCACCAACAGAGGTTC
TCCAGAAGATACAAATGTTTCTTTAATGCTTAAATTCTCTAAAGAACTGCAACCTGTATCCTTCTCTGGA
CCAACTAAAGGAACGATTACAGGCAATACAGTAGTATTCGATTCGTTACCTAGATTAGGTTCTAAAGAAA
CTGTAGAGTTTTCTGTAACATTGAAAGCAGTATCAGCTGGAGATGCTCGTGGGGAAGCGATTCTTTCTTC
CGATACATTGACTGTTCCAGTTTCTGATACAGAGAATACACACATCTATTAA
>gi|3328841|gb|AAC68010.1| Putative outer membrane protein B
[Chlamydia trachomatis]
MKWLSATAVFAAVLPSVSGFCFPEPKELNFSRVGTSSSTTFTETVGEAGAEYIVSGNASFTKFTNIPTTD
TTTPTNSNSSSSNGETASVSEDSDSTTTTPDPKGGGAFYNAHSGVLSFMTRSGTEGSLTLSEIKITGEGG
AIFSQGELLFTDLTGLTIQNNLSQLSGGAIFGESTISLSGITKATFSSNSAEVPAPVKKPTEPKAQTASE
TSGSSSSSGNDSVSSPSSSRAEPAAANLQSHFICATATPAAQTDTETSTPSHKPGSGGAIYAKGDLTIAD
SQEVLFSINKATKDGGAIFAEKDVSFENITSLKVQTNGAEEKGGAIYAKGDLSIQSSKQSLFNSNYSKQG
GGALYVEGDINFQDLEEIRIKYNKAGTFETKKITLPKAQASAGNADAWASSSPQSGSGATTVSNSGDSSS
GSDSDTSETVPATAKGGGLYTDKNLSITNITGIIEIANNKATDVGGGAYVKGTLTCENSHRLQFLKNSSD
KQGGGIYGEDNITLSNLTGKTLFQENTAKEEGGGLFIKGTDKALTMTGLDSFCLINNTSEKHGGGAFVTK
EISQTYTSDVETIPGITPVHGETVITGNKSTGGNGGGVCTKRLALSNLQSISISGNSAAENGGCAHTCPD
SFPTADTAEQPAAASAATSTPESAPVVSTALSTPSSSTVSSLTLLAASSQASPATSNKETQDPNADTDLL
IDYVVDTTISKNTAKKGGGIYAKKAKMSRIDQLNISENSATEIGGGICCKESLELDALVSLSVTENLVGK
EGGGLHAKTVNISNLKSGFSFSNNKANSSSTGVATTASAPAAAAASLQAAAAAVPSSPATPTYSGVVGGA
IYGEKVTFSQCSGTCQFSGNQAIDNNPSQSSLNVQGGAIYAKTSLSIGSSDAGTSYIFSGNSVSTGKSQT
TGQIAGGAIYSPTVTLNCPATFSNNTASMATPKTSSEDGSSGNSIKDTIGGAIAGTAITLSGVSRFSGNT
ADLGAAIGTLANANTPSATSGSQNSITEKITLENGSFIFERNQANKRGAIYSPSVSIKGNNITFNQNTST
HDGSAIYFTKDATIESLGSVLFTGNNVTATQASSATSGQNTNTANYGAAIFGDPGTTQSSQTDAILTLLA
SSGNITFSNNSLQNNQGDTPASKFCSIAGYVKLSLQAAKGKTISFFDCVHTSTKKIGSTQNVYETLDINK
EENSNPYTGTIVFSSELHENKSYIPQNAILHNGTLVLKEKTELHVVSFEQKEGSKLIMKPGAVLSNQNIA
NGALVINGLTIDLSSMGTPQAGEIFSPPELRIVATTSSASGGSGVSSSIPTNPKRISAAAPSGSAATTPT
MSENKVFLTGDLTLIDPNGNFYQNPMLGSDLDVPLIKLPTNTSDVQVYDLTLSGDLFPQKGYMGTWTLDS
NPQTGKLQARWTFDTYRRWVYIPRDNHFYANSILGSQNSMIVVKQGLINNMLNNARFDDIAYNNFWVSGV
GTFLAQQGTPLSEEFSYYSRGTSVAIDAKPRQDFILGAAFSKMVGKTKAIKKMHNYFHKGSEYSYQASVY
GGKFLYFLLNKQHGWALPFLIQGVVSYGHIKHDTTTLYPSIHERNKGDWEDLGWLADLRISMDLKEPSKD
SSKRITVYGELEYSSIRQKQFTEIDYDPRHFDDCAYRNLSLPVGCAVEGAIMNCNILMYNKLALAYMPSI
YRNNPVCKYRVLSSNEAGQVICGVPTRTSARAEYSTQLYLGPFWTLYGNYTIDVGMYTLSQMTSCGARMI
F
>gi|3328833|gb|AE001314.1:9601-14856,
ATGAAATGGCTGTCAGCTACTGCGGTGTTTGCTGCTGTTCTCCCCTCAGTTTCAGGGTTTTGCTTCCCAG
AACCTAAAGAATTAAATTTCTCTCGCGTAGGAACTTCTTCCTCTACCACTTTTACTGAAACAGTTGGAGA
AGCTGGGGCAGAATATATCGTCTCTGGTAACGCATCTTTCACAAAATTTACCAACATTCCTACTACCGAT
ACAACAACTCCCACGAACTCAAACTCCTCTAGCTCTAACGGAGAGACTGCTTCCGTTTCTGAGGATAGTG
ACTCTACAACAACGACTCCTGATCCTAAAGGTGGCGGCGCCTTTTATAACGCGCACTCCGGAGTTTTATC
CTTTATGACACGATCAGGAACAGAAGGTTCCTTAACTCTGTCTGAGATAAAAATAACTGCTGAAGGCGGT
GCTATCTTCTCTCAAGGAGAGCTGCTATTTACAGATCTGACAGGTCTAACCATCCAAAATAACTTATCCC
AGCTATCCGGAGGAGCGATTTTTGGAGAATCTACAATCTCCCTATCAGGGATTACTAAAGCGACTTTCTC
CTCCAACTCTGCAGAAGTTCCTGCTCCTGTTAAGAAACCTACAGAACCTAAACCTCAAACAGCAAGCGAA
ACGTCGGGTTCTAGTAGTTCTAGCGGAAATGATTCGGTGTCTTCCCCCAGTTCCAGTAGAGCTGAACCCG
CAGCAGCTAATCTTCAAAGTCACTTTATTTGTGCTACAGCTACTCCTGCTGCTCAAACCGATACAGAAAC
ATCAACTCCCTCTCATAAGCCAGGATCTGGGGGAGCTATCTATGCTAAAGGCGACCTTACTATCGCAGAC
TCTCAAGAGGTACTATTCTCAATAAATAAAGCTACTAAAGATGGAGGAGCGATCTTTGCTGAGAAAGATG
TTTCTTTCGAGAATATTACATCATTAAAAGTACAAACTAACGGTGCTGAAGAAAAGGGAGGAGCTATCTA
TGCTAAAGGTGACCTCTCAATTCAATCTTCTAAACAGAGTCTTTTTAATTCTAACTACAGTAAACAAGGT
GGTGGGGCTCTATATGTTGAAGGAGATATAAACTTCCAAGATCTTGAAGAAATTCGCATTAAGTACAATA
AAGCTGGAACGTTCGAAACAAAAAAAATCACTTTACCAAAAGCTCAAGCATCTGCAGGAAATGCACATGC
TTGGGCCTCTTCCTCTCCTCAATCTGGTTCTGGAGCAACTACAGTCTCCAACTCAGGAGACTCTAGCTCT
GGCTCAGACTCGGATACCTCAGAAACAGTTCCAGCCACAGCTAAAGGCGGTGGGCTTTATACTGATAAGA
ATCTTTCGATTACTAACATCACAGGAATTATCGAAATTGCAAATAACAAAGCGACAGATGTTGGAGGTGG
TGCTTACGTAAAAGGAACCCTTACTTGTGAAAACTCTCACCGTCTACAATTTTTGAAAAACTCTTCCGAT
AAACAAGGTGGAGGAATCTACGGAGAAGACAACATCACCCTATCTAATTTGACAGGGAAGACTCTATTCC
AAGAGAATACTGCCAAAGAAGAGGGCGGTGGACTCTTCATAAAAGGTACAGATAAAGCTCTTACAATGAC
AGGACTGGATAGTTTCTGTTTAATTAATAACACATCAGAAAAACATGGTGGTGGAGCCTTTGTTACCAAA
GAAATCTCTCAGACTTACACCTCTGATGTGGAAACAATTCCAGGAATCACGCCTGTACATGGTGAAACAG
TCATTACTGGCAATAAATCTACAGGAGGTAATGGTGGAGGCGTGTGTACAAAACGTCTTGCCTTATCTAA
CCTTCAAAGCATTTCTATATCCGGGAATTCTGCAGCTGAAAATGGTGGTGGAGCCCACACATGCCCAGAT
AGCTTCCCAACGGCGGATACTGCAGAACAGCCCGCAGCAGCTTCTGCCGCGACGTCTACTCCCGAGTCTG
CCCCAGTGGTCTCAACTGCTCTAAGCACACCTTCATCTTCTACCGTCTCTTCATTAACCTTACTAGCAGC
CTCTTCACAAGCCTCTCCTGCAACCTCTAATAAGGAAACTCAAGATCCTAATGCTGATACAGACTTATTG
ATCGATTATGTAGTTGATACGACTATCAGCAAAAACACTGCTAAGAAAGGCGGTGGAATCTATGCTAAAA
AAGCCAAGATGTCCCGCATAGACCAACTGAATATCTCTGAGAACTCCGCTACAGAGATAGGTGGAGGTAT
CTGCTGTAAAGAATCTTTAGAACTAGATGCCCTAGTCTCCTTATCTGTAACAGAGAACCTTGTTGGGAAA
GAAGGTGGAGGCTTACATGCTAAAACTGTAAATATTTCTAATCTGAAATCAGGCTTCTCTTTCTCGAACA
ACAAAGCAAACTCCTCATCCACAGGAGTCGCAACAACACCTTCAGCACCTGCTGCAGCTGCTGCTTCCCT
ACAAGCAGCCGCAGCAGCCGTACCATCATCTCCAGCAACACCAACTTATTCAGGTGTAGTAGGAGGAGCT
ATCTATGGAGAAAAGGTTACATTCTCTCAATGTAGCGGGACTTGTCAGTTCTCTGGGAACCAAGCTATCG
ATAACAATCCCTCCCAATCATCGTTGAACGTACAAGGAGCAGCCATCTATGCCAAAACCTCTTTGTCTAT
TGGATCTTCCGATGCTGGAACCTCCTATATTTTCTCGGGGAACAGTGTCTCCACTGGGAAATCTCAAACA
ACAGGGCAAATAGCGGGAGGAGCGATCTACTCCCCTACTGTTACATTGAATTGTCCTGCGACATTCTCTA
ACAATACAGCCTCTATGGCTACACCAAAGACTTCTTCTGAAGATGGATCCTCAGGAAATTCTATTAAAGA
TACCATTGGAGGAGCCATTGCAGGGACAGCCATTACCCTATCTGGAGTCTCTCGATTTTCAGGGAATACG
GCTGATTTAGGAGCTGCAATAGGAACTCTAGCTAATGCAAATACACCCAGTGCAACTAGCGGATCTCAAA
ATAGCATTACAGAAAAAATTACTTTAGAAAACGGTTCTTTTATTTTTGAAAGAAACCAAGCTAATAAACG
TGGAGCGATTTACTCTCCTAGCGTTTCCATTAAAGGGAATAATATTACCTTCAATCAAAATACATCCACT
CATGATGGAAGTGCTATCTACTTTACAAAAGATGCTACGATTGAGTCTTTAGGATCTGTTCTTTTTACAG
GAAATAACGTTACAGCTACACAAGCTAGTTCTGCAACATCTGGACAAAATACAAATACTGCCAACTATGG
GGCAGCCATCTTTGGAGATCCAGGAACCACTCAATCGTCTCAAACAGATGCCATTTTAACCCTTCTTGCT
TCTTCTGGAAACATTACTTTTAGCAACAACAGTTTACAGAATAACCAAGGTGATACTCCCGCTAGCAAGT
TTTGTAGTATTGCAGGATACGTCAAACTCTCTCTACAAGCCGCTAAAGGGAAGACTATTAGCTTTTTCGA
TTGTGTGCACACCTCTACCAAAAAAATAGGTTCAACACAAAACGTTTATGAAACTTTAGATATTAATAAA
GAAGAGAACAGTAATCCATATACAGGAACTATTGTGTTCTCTTCTGAATTACATGAAAACAAATCTTACA
TCCCACAGAATGCAATCCTTCACAACGGAACTTTAGTTCTTAAAGAGAAAACAGAACTCCACGTAGTCTC
TTTTGAGCAGAAAGAAGGGTCTAAATTAATTATGAAACCCGGAGCTGTGTTATCTAACCAAAACATAGCT
AACGGAGCTCTAGTTATCAATGGGTTAACGATTGATCTTTCCAGTATGGGGACTCCTCAAGCAGGGGAAA
TCTTCTCTCCTCCAGAATTACGTATCGTTGCCACGACCTCTAGTGCATCCGGAGGAAGCGGGGTCAGCAG
TAGTATACCAACAAATCCTAAAAGGATTTCTGCAGCAGCGCCTTCAGGTTCTGCCGCAACTACTCCAACT
ATGAGCGAGAACAAAGTTTTCCTAACAGGAGACCTTACTTTAATAGATCCTAATGGAAACTTTTACCAAA
ACCCTATGTTAGGAAGCGATCTAGATGTACCACTAATTAAGCTTCCGACTAACACAAGTGACGTCCAAGT
CTATGATTTAACTTTATCTGGGGATCTTTTCCCTCAGAAAGGGTACATGGGAACCTGGACATTAGATTCT
AATCCACAAACAGGGAAACTTCAAGCCAGATGGACATTCGATACCTATCGTCGCTGGGTATACATACCTA
GGGATAATCATTTTTATGCGAACTCTATCTTAGGCTCCCAAAACTCAATGATTGTTGTGAAGCAAGGGCT
TATCAACAACATGTTGAATAATGCCCGCTTCGATGATATCGCTTACAATAACTTCTGGGTTTCAGGAGTA
GGAACTTTCTTAGCTCAACAAGGAACTCCTCTTTCCGAAGAATTCAGTTACTACAGCCGCGGAACTTCAG
TTGCCATCGATGCCAAACCTAGACAAGATTTTATCCTAGGAGCTGCATTTAGTAAGATGGTGGGGAAAAC
CAAAGCCATCAAAAAAATGCATAATTACTTCCATAAGGGCTCTGAGTACTCTTACCAAGCTTCTGTCTAT
GGAGGTAAATTCCTGTATTTCTTGCTCAATAAGCAACATGGTTGGGCACTTCCTTTCCTAATACAAGGAG
TCGTGTCCTATGGACATATTAAACATGATACAACAACACTTTACCCTTCTATCCATGAAAGAAATAAAGG
AGATTGGGAAGATTTAGGATGGTTAGCGGATCTTCGTATCTCTATGGATCTTAAAGAACCTTCTAAAGAT
TCTTCTAAACGGATCACTGTCTATGGGGAACTTGAGTATTCCAGCATTCGCCAGAAACAGTTCACAGAAA
TCGATTACGATCCAAGACACTTCGATGATTGTGCTTACAGAAATCTGTCGCTTCCTGTGGGATGCGCTGT
CGAAGGAGCTATCATGAACTGTAATATTCTTATGTATAATAAGCTTGCATTAGCCTACATGCCTTCTATC
TACAGAAATAATCCTGTCTGTAAATATCGGGTATTGTCTTCGAATGAAGCTGGTCAAGTTATCTGCGGAG
TGCCAACTAGAACCTCTGCTAGAGCAGAATACAGTACTCAACTATATCTTGGTCCCTTCTGGACTCTCTA
CGGAAACTATACTATCGATGTAGGCATGTATACGCTATCGCAAATGACTAGCTGCGGTGCTCGCATGATC
TTCTAA
>gi|3328840|gb|AAC68009.1| Putative outer membrane protein A
[Chlamydia trachomatis]
MNRVIEIHAHYDQRQLSQSPNTNFLVHHPYLTLIPKFLLGALIVYAPYSFAEMELAISGHKQGKDRDTFT
MISSCPEGTNYIINRKLILSDFSLLNKVSSGGAFRNLAGKISFLGKNSSASIHFKHININGFGAGVFSES
SIEFTDLRKLVAFGSESTGGIFTAKEDISFKNNHHIAFRNNITKGNGGVIQLQGDMKGSVSFVDQRGAII
FTNNQAVTSSSMKHSGRGGAISGDFAGSRILFLNNQQITFEGNSAVHGGAIYNKNGLVEFLGNAGPLAFK
ENTTIANGGAIYTSNFKANQQTSPILFSQNHANKKGGAIYAQYVNLEQNQDTIRFEKNTAKEGGGAITSS
QCSITAHNTIIFSDNAAGDLGGGAILLEGKKPSLTLIAHSGNIAFSGNTMLHITKKASLDRHNSILIKEA
PYKIQLAANKNHSIHFFDPVMALSASSSPIQINAPEYETPFFSPKGMIVFSGANLLDDAREDVANRTSIF
NQPVHLYNGTLSIENGAHLIVQSFKQTGGRISLSPGSSLALYTMNSFFHGNISSKEPLEINGLSFGVDIS
PSNLQAEIRAGNAPLRLSGSPSIHDPEGLFYENRDTAASPYQMEILLTSDKIVDISKFTTDSLVTNKQSG
FQGAWHFSWQPNTINNTKQKILRASWLPTGEYVLESNRVGRAVPNSLWSTFLLLQTASHNLGDHLCNNRS
LIPTSYFGVLIGGTGAEMSTHSSEEESFISRLGATGTSIIRLTPSLTLSGGGSHMFGDSFVADLPEHITS
EGIVQNVGLTHVWGPLTVNSTLCAALDHNAMVRICSKKDHTYGKWDTFGMRGTLGASYTFLEYDQTMRVF
SFANIEATNILQRAFTETGYNPRSFSKTKLLNIAIPIGIGYEFCLCNSSFALLGKGSIGYSRDIKRENPS
TLAHLAMNDFAWTTNGCSVPTSAHTLANQLILRYKACSLYITAYTINREGKNLSNSLSCGGYVGF
>giP51 3328833|gb|AE001314.1:6535-9462,
ATGAATCGAGTTATAGAAATCCATGCTCACTACGATCAAAGACAACTTTCTCAATCTCCAAATACAAACT
TCTTAGTACATCATCCTTATCTTACTCTTATTCCCAAGTTTCTACTAGGAGCTCTAATCGTCTATGCTCC
TTATTCGTTTGCAGAAATGGAATTAGCTATTTCTGGACATAAACAAGGTAAAGATCGAGATACCTTTACC
ATGATCTCTTCCTGTCCTGAAGGCACTAATTACATCATCAATCGCAAACTCATACTCAGTGATTTCTCGT
TACTAAATAAAGTTTCATCAGGGGGAGCCTTTCGGAATCTAGCAGGGAAAATTTCCTTCTTAGGAAAAAA
TTCTTCTGCGTCCATTCATTTTAAACACATTAATATCAATGGTTTTGGAGCCGGAGTCTTTTCTGAATCC
TCTATTGAATTTACTGATTTACCAAAACTTGTTGCTTTTGGATCTGAAAGCACAGGAGGAATTTTTACTG
CGAAAGAGGACATCTCTTTTAAAAACAACCACCACATTGCCTTCCGCAATAATATCACCAAAGGGAATGG
TGGCGTTATCCAGCTCCAAGGAGATATGAAAGGAAGCGTATCCTTTGTAGATCAACGTGGAGCTATCATC
TTTACCAATAACCAAGCTGTAACTTCTTCATCAATGAAACATAGTGGTCGTGGAGGAGCAATTAGCCGTG
ACTTCGCAGGATCCAGAATTCTTTTTCTTAATAACCAACAAATTACTTTCGAAGGCAATAGCGCTGTGCA
TGGAGGTGCTATCTACAATAAGAATGGCCTTGTCGAGTTCTTAGGAAATGCAGGACCTCTTGCCTTTAAA
GAGAACACAACAATAGCTAACGGGGGAGCTATATACACAAGTAATTTCAAAGCGAATCAACAAACATCCC
CCATTCTATTCTCTCAAATCATGCGAATAAGAAAGGCGGAGCGATTTACGCGCAATATGTGAAACTTAGA
ACAGAATCAAGATACTATTCGCTTTGAAAAAAATACCGCTAAAGAAGGCGGTGGAGCCATCACCTCTTCT
CAATGCTCAATTACTGCTCATAATACCATCATTTTTTCCGATAATGCTGCCGGAGATCTTGGAGGAGGAG
CAATTCTTCTAGAAGGGAAAAAACCTTCTCTAACCTTGATTGCTCATAGTGGTAATATTGCATTTAGCGG
CAATACCATGCTTCATATCACCAAAAAAGCTTCCCTAGATCGACACAATTCTATCTTAATCAAAGAAGCT
CCCTATAAAATCCAACTTGCAGCGAACAAAAACCATTCTATTCATTTCTTTGATCCTGTCATGGCATTGT
CAGCATCATCTTCCCCTATACAAATCAATGCTCCTGAGTATGAAACTCCCTTCTTCTCACCTAAGGGTAT
GATCGTTTTCTCGGGTGCGAATCTTTTAGATGATGCTAGGGAAGATGTTGCAAATAGAACATCGATTTTT
AACCAACCCGTTCATCTATATAATGGCACCCTATCTATCGAAAATGGAGCCCATCTGATTGTCCAAAGCT
TCAAACAGACCGCAGGACGTATCAGTTTATCTCCAGGATCCTCCTTGGCTCTATACACGATGAACTCGTT
CTTCCATGGCAACATATCCAGCAAAGAACCCCTAGAAATTAATGGTTTAAGCTTTGGAGTAGATATCTCT
CCTTCTAATCTTCAAGCAGAGATCCGTGCCGGCAACGCTCCTTTACGATTATCCGGATCCCCATCTATCC
ATGATCCTGAAGGATTATTCTACGAAAATCGCGATACTGCAGCATCACCATACCAAATGGAAATCTTGCT
CACCTCTGATAAAATTGTAGATATCTCCAAATTTACTACTGATTCTCTAGTTACGAACAAACAATCAGGA
TTCCAAGGAGCCTGGCATTTTAGCTGGCAGCCAAATACTATAAACAATACTAAACAAAAAATATTAAGAG
CTTCTTGGCTCCCAACAGGAGAATATGTCCTTGAATCCAATCGAGTGGGGCGTGCCGTTCCTAATTCCTT
ATGGAGCACATTTTTACTTTTACAGACAGCCTCTCATAACTTAGGCGATCATCTATGTAATAATCGATCT
CTTATTCCTACTTCATACTTCGGAGTTTTAATTGGAGGAACTGGAGCAGAAATGTCTACCCACTCCTCAG
AAGAAGAAAGCTTTATATCTCGTTTAGGAGCTACAGGAACCTCTATCATACGCTTAACTCCCTCCCTGAC
ACTCTCTGGAGGAGGCTCACATATGTTCGGAGATTCGTTCGTTGCAGACTTACCAGAACACATCACTTCA
GAAGGAATTGTTCAGAATGTCGGTTTAACCCATGTCTGGGGACCCCTTACTGTCAATTCTACATTATGTG
CAGCCTTAGATCACAACGCGATGGTCCGCATATGCTCCAAAAAAGATCACACCTATGGGAAATGGGATAC
ATTCGGTATGCGAGGAACATTAGGAGCCTCTTATACATTCCTAGAATATGATCAAACTATGCGCGTATTC
TCATTCGCCAACATCGAAGCCACAAATATCTTGCAAAGAGCTTTTACTGAAACAGGCTATAACCCAAGAA
GTTTTTCCAAGACAAAACTTCTAAACATCGCCATCCCCATAGGGATTGGTTATGAATTCTGCTTAGGGAA
TAGCTCTTTTGCTCTACTAGGTAAGGGATCCATCGGTTACTCTCGAGATATTAAACGAGAAAACCCATCC
ACTCTTGCTCACCTGGCTATGAATGATTTTGCTTGGACTACCAATGGCTGTTCAGTTCCAACCTCTGCAC
ACACATTGGCAAATCAATTGATTCTTCGCTATAAAGCATGTTCCTTATACATCACGGCATATACTATCAA
CCGTGAAGGGAAGAACCTCTCCAATAGCTTATCCTGCGGAGGCTATGTTGGCTTCTAA
>gi|3328763|gb|AAC67938.1| O-Sialoglycoprotein Endopeptidase family
[Chlamydia trachomatis]
MYKYFIVDTSGSQPFLAYVDCRDVLEVWSLPTGPDQGVVLNFIFNSLDLPFQGIGVSVGPGGFSATRVGV
AFAQGLSLAKNVPLVGYSSLEGYLSLGQEEEALLLPLGKKGGIVALNSELSLDGFLLTDTTSTPGILLSY
SEALEYCLDKGCCHVISPDPTYFVELFSSRISVRKVVPCIDRIRKYVVSQFVLSQNLPLCLDYRSISSFF
>gi|3328757|gb|AE001307.1:c6730-6098,
GTGTACAAATATTTTATTGTAGACACTTCCGGTTCTCAGCCGTTTTTGGCCTATGTCGATTGTCGAGATG
TTTTAGAAGTATGGTCTTTGCCTACAGGGCCAGATCAAGGAGTTGTGTTAAATTTCATTTTCAACAGCCT
GGATTTGCCTTTCCAAGGTATTGGAGTCTCTGTTGGTCCTGGGGGATTTTCTGCAACTAGAGTGGGAGTT
GCTTTTGCTCAAGGGCTCTCTCTGGCTAAAAATGTCCCTTTAGTTGGCTATAGCTCTTTAGAAGGATATC
TTTCTTTGCGTCAAGAAGAGGAGGCTTTGCTTTTGCCTCTAGGGAAAAAGGGTGGGATTGTAGCTTTAAA
CTCAGAGCTTTCTCTTGATGGTTTTCTGCTTACAGATACTACTTCTACTCCGGGAATTTTATTGTCTTAT
TCTGAAGCTCTAGAGTATTGTTTAGATAAGGGATGTTGTCATGTGATCTCTCCGGATCCAACGTATTTCG
TAGAACTATTTTCTTCGCGTATTTCGGTAAGGAAGGTGGTTCCTTGTATCGATCGAATCCGTAAGTACGT
TGTTTCGCAGTTTGTTCTGTCTCAAAATCTTCCGTTGTGTTTAGATTACCGGAGCATCTCTTCCTTTTTT
TAG
>gi|6578102|gb|AAC67897.2|ATP Synthase Subunit K [Chlamydia
trachomatis]
MIDVSVVGPVLAMALAMIGSAVGCGMAGVASHAVMSRIDEGHGKIIGLSAMPSSQSIYGLIFMLLLNDAI
KDGKVSAVSGIVMGIAVGSALLLSAFMQGKCCVSAIQAYARSSAIYGKSFASIGIVESFALFAFVFALLL
F
>gi|3328718|gb|AE001303.1:C956-531,
ATGATAGATGTATCAGTAGTGGGGCCTGTATTGGCTATGGCTTTGGCAATGATTGGTAGCGCTGTTGGAT
GTGGAATGGCTGGAGTCGCTTCTCACGCAGTGATGTCTCGAATCGATGAAGGACACGGGAAGATTATTGG
TCTGTCTGCTATGCCCTCATCACAATCCATTTACGGGTTGATTTTCATGTTACTGCTGAATGATGCAATT
AAGGATGGAAAAGTCTCTGCAGTCAGTGGTATCGTAATGGGTATAGCTGTAGGATCTGCGTTATTGCTTT
CTGCTTTTATGCAAGGGAAGTGCTGTGTGAGTGCTATTCAAGCCTATGCGCGTTCCTCTGCAATATATGG
TAAATCATTTCCTTCGATTGGGATTGTTGAGTCTTTTGCGTTATTTGCTTTCGTTTTTGCGCTATTGTTA
TTCTAA
>gi|3329252|gb|AAC68382.1|S14 Ribosomal Protein [Chlamydia
trachomatis]
MAKKSAVAREVKRRKLVEANFQKHAELRKLAKSLSVSEEERERAREALNKMRRDTSPSRLHNRCLLTGRP
RGYLRKFAISRICFRQMASMGDIPGVVKASW
>gi|3522908|gb|AE001351.1:2436-2741,
ATGGCGAAGAAGTCAGCAGTAGCTAGAGAAGTTAAACGTCGAAAGTTAGTAGAAGCTAATTTTCAGAAGA
GAGCAGAGCTTCGAAAACTTGCGAAGAGTTTATCTGTTAGCGAGGAAGAAAGAGAAAGAGCTCGGGAAGC
TCTCAATAAAATGAGAAGAGATACTTCTCCTTCTCGTTTACATAATAGATGCCTATTAACAGGCCGTCCT
CGTGGATACCTTAGAAAGTTTGCTATCTCAAGAATTTGTTTTAGACAAATGGCTTCTATGGGAGATATCC
CAGGCGTTGTGAAAGCAAGTTGGTAG
>gi|3329133|gb|AAC68276.1| Major Outer Membrane Protein [Chlamydia
trachomatis]
MKKLLKSVLVFAALSSASSLQALPVGNPAEPSLMIDGILWEGFGGDPCDPCATWCDAISMRVGYYGDFVF
DRVLKTDVNKEFQMGAKPTTDTGNSAAPSTLTARENPAYGRHMQDAEMFTNAACMALNIWDRFDVFCTLG
ATSGYLKGNSASFNLVGLFGDNENQKTVKAESVPNMSFDQSVVELYTDTTFAWSVGARAALWECGCATLG
ASFQYAQSKPKVEELNVLCNAAEFTINKPKGYVGKEFPLDLTAGTDAATGTKDASIDYHEWQASLALSYR
LNMFTPYIGVKWSRASFDADTIRIAQPKSATAIFDTTTLNPTIAGAGDVKTGAEGQLGDTMQIVSLQLNK
MKSRKSCGIAVGTTIVDADKYAVTVETRLIDERAAHVNAQFRF
>gi|3329126|gb|AE001338.1:c6759-5578,
ATGAAAAAACTCTTGAAATCGGTATTAGTATTTGCCGCTTTGAGTTCTGCTTCCTCCTTGCAAGCTCTGC
CTGTGGGGAATCCTGCTGAACCAAGCCTTATGATCGACGGAATTCTGTGGGAAGGTTTCGGCGCAGATCC
TTGCGATCCTTCCGCCACTTGGTGTGACGCTATCAGCATGCGTGTTGGTTACTACGGAGACTTTGTTTTC
GACCGTGTTTTGAAAACTGATGTGAATAAAGAATTTCAGATGGGTGCCAACCCTACAACTGATACAGGCA
ATAGTCCAGCTCCATCCACTCTTACAGCAAGACAGAATCCTGCTTACGGCCGACATATGCAGGATGCTGA
GATGTTTACAAATGCCGCTTGCATGGCATTGAATATTTGGGATCGTTTTGATGTATTCTGTACATTAGGA
GCCACCAGTGGATATCTTAAAGGAAACTCTGCTTCTTTCAATTTAGTTGGATTGTTTGGAGATAATGAAA
ATCAAAAAACGGTCAAAGCGGAGTCTGTACCAAATATGAGCTTTGATCAATCTGTTGTTGAGTTGTATAC
AGATACTACTTTTGCGTGGAGCGTCGGCGCTCGCGCAGCTTTGTGGGAATGTGGATGTGCAACTTTAGGA
GCTTCATTCCAATATGCTCAATCTAAACCTAAAGTAGAAGAATTAAACGTTCTCTGCAATGCAGCAGAGT
TTACTATTAATAAACCTAAAGGGTATGTAGGTAAGGAGTTTCCTCTTGATCTTACAGCAGGAACAGATGC
TGCGACAGGAACTAAGGATGCCTCTATTGATTACCATGAATGGCAAGCAAGTTTAGCTCTCTCTTACAGA
CTGAATATGTTCACTCCCTACATTGGAGTTAAATGGTCTCGAGCAAGCTTTGATGCCGATACGATTCGTA
TAGCCCAGCCAAAATCAGCTACAGCTATTTTTGATACTACCACGCTTAACCCAACTATTGCTGGAGCTGG
CGATGTGAAAACTGGCGCAGAGGGTCAGCTCGGAGACACAATGCAAATCGTTTCCTTGCAATTGAACAAG
ATGAAATCTAGAAAATCTTGCGGTATTGCAGTAGGAACAACTATTGTGGATGCAGACAAATACGCAGTTA
CAGTTGAGACTCGCTTGATCGATCAGAGAGCAGCTCACGTAAATGCACAATTCCGCTTCTAA
>gi|3328987|gb|AAC68150.1| hypothetical protein
[Chlamydia trachomatis]
MLKMFWLNSLVFFSLLLSACGYTVLSPHYVEKKFSLSEGIYVCPIEGDSLGDLVSSLSYELEKRGLHTRS
QGTSSGYVLKVSLFNETDENIGFAYTPQKPDEKPVKHFIVSNEGRLALSAKVQLIKNRTQEILVEKCLRK
SVTFDFQPDLGTANAHQLALGQFEMHNEAIKSASRILYSQLAETIVQQVYYDLF
>gi|3328980|gb|AE001325.1:10880-11464,
ATGCTGAAAATGTTTTGGTTGAATAGCCTCGTTTTCTTCTCGTTACTACTATCAGCCTGCGGCTATACAG
TGCTCTCCCCCCACTATGTAGAAAAGAAATTCTCGCTTTCCGAAGGCATCTATGTCTGCCCTATCGAAGG
AGATTCATTAGGAGATCTCGTATCCTCTCTTTCTTACGAATTAGAAAAGCGAGGACTCCACACACGATCT
CAAGGAACCTCTTCTGGTTATGTACTCAAAGTCTCTCTTTTCAATGAGACTGATGAAAATATTGGATTCG
CATACACTCCCCAAAAACCTGATGAAAAACCTGTAAAACACTTCATTGTCTCTAATGAAGGGCGCTTAGC
GTTATCAGCAAAAGTCCAACTAATCAAAAACCGCACACAAGAAATATTAGTGGAGAAATGCCTGAGAAAA
TCGGTTACTTTTGATTTTCAACCTGACCTCGGAACCGCGAATGCTCATCAGCTAGCTCTCGGACAATTTG
AAATGCATAATGAAGCAATAAAAAGCGCTTCTCGTATATTGTATTCGCAATTAGCAGAGACTATTGTACA
ACAGGTATACTATGACCTTTTCTGA
>gi|3328972|gb|AAC68136.1| Apolipoprotein N-Acetyltransferase
[Chlamydia trachomatis]
MFKLVSYIILSWVLVCLAQPDVSVVASVVSCICGYSLLWAGLFALVEQLSWKKVWCIAFIWTWTVEGAHF
SWMLEDLYVGTSIYFVWGILLSYLATLFASFSCLVVWCCRKQYRGALVWLPGVWVAIEAIRYYGLLSGVS
FDFIGWPLTATAYGRQFGSFFGWAGQSFLVIAANICCFAVCLLKHSFSKGLWLTLCAFPYLLGGAHYEYL
KKHFSDSEVLRVAIVQPGYSPHMHAGRTASAIWRGLVSLCQTIQTPVDVIVFPEVSVPFGLHRQAYTLHE
NQPVLESLLPNKSWGEFFTNLDWIQAIAERYQCTVIMGMERWENKGGILHLYNAAECVSREGEITSYDKR
ILVPGGEYIPGGKIGFSLCQTFFPEFALPFQRLPGEFSGVVNITERIKAGISICYEETFGYAIRPYKRQQ
ADILVNLTNDCWYPRSRLPLVHFYHGMLRNQELGIPCIHACRTGVSAAVDSLGRIVGILPWESRTCPVST
GVLQVSVPLYSYHTVYARLGDAPLLLIAVCSVIGAIAYFYRKKKETPPQTFF
>gi|3328966|gb|AE001324.1:c6152-4524,
GTGTTTAAACTTGTGTCATACATCATCCTTTCTTCGGTGCTGGTCTGTTTGGCTCAGCCGGATGTAAGTG
TTGTAGCTTCTGTTGTTAGTTGTATTTGCGGTTACAGCTTACTTTGGGCTGGGCTTTTTGCTTTAGTAGA
GCAATTATCTTGGAAGAAAGTTTGGTGCATCGCTTTTATTTGGACTTGGACTGTCGAAGGCGCTCATTTC
TCTTGGATGCTTGAAGATCTTTATGTAGGGACAAGCATCTATTTTGTTTGGGGTATACTGCTTTCTTATC
TCGCCACCCTATTTGCTAGTTTTTCTTGTTTGGTTGTGTGGTGTTGTCGCAAGCAATATAGGGGAGCTCT
TGTTTGGCTTCCAGGGGTTTGGGTGGCGATAGAAGCAATACGCTATTATGGGTTGCTTTCAGGAGTTTCT
TTTGATTTTATTGGCTGGCCTCTTACAGCGACAGCCTATGGCCGGCAATTCGGCAGCTTTTTTGGATGGG
CTGCACAAAGCTTTCTAGTTATTGCTGCCAATATATGCTGTTTTGCAGTATGTTTATTAAAACACTCTTT
TTCCAAAGGTTTGTGGTTGACGTTGTGCGCGTTCCCTTATCTGTTAGGCGGAGCGCATTACGAATACCTA
AAGAAGCATTTTTCCGACTCTGAAGTGCTTCGAGTTGCCATCGTGCAGCCTGGATATAGTCCTCATATGC
ATGCAGGGAGGACGGCTAGTGCTATTTGGAGAGGTTTGGTTTCTTTGTGCCAGACTATTCAAACTCCTGT
AGATGTGATCGTTTTCCCAGAAGTAAGTGTTCCTTTTGGCTTACATAGACAAGCCTATACTCTTCATGAA
AATCAGCCTGTATTAGAAAGTTTGCTTCCTAACAAATCTTGGGGCGAGTTTTTCACAAATTTGGATTGGA
TCCAAGCGATAGCTGAACGTTATCAATGCACCGTTATCATGGGAATGGAACGATGGGAAAATAAAGGGGG
AATACTGCATTTGTATAATGCTGCTGAATGCGTATCGCGAGAAGGGGAAATAACTAGCTATGATAAGCGG
ATTCTTGTTCCTGGAGGTGAGTACATCCCTGGAGGGAAAATAGGTTTTTCCTTGTGTCAAACCTTTTTCC
CAGAATTTGCTCTTCCCTTTCAACGTTTGCCAGGAGAGTTTTCTGGAGTTGTGAATATAACAGAGCGAAT
AAAAGCTGGGATCTCTATTTGTTATGAGGAGACATTTGGGTATGCAATTCGCCCTTACAAAAGGCAACAA
GCCGATATTTTAGTAAATCTTACTAATGACGGTTGGTATCCGCGTTCAAGGCTGCCTCTAGTACATTTTT
ATCATGGCATGTTACGTAATCAAGAGTTGGGTATACCTTGTATTCGCGCCTGTCGCACAGGAGTTTCTGC
TGCAGTGGATTCTTTGGGTACAATTGTCGGCATACTTCCCTGGGAATCGAGAACTTGCCCAGTTTCTACA
GGAGTACTCCAAGTTTCCGTCCCTCTTTACAGTTATCATACTGTATATGCAAGGCTGGGTGATGCTCCTC
TGTTACTGATTGCAGTTTGTTCGGTTATCGGAGCGATTGCCTATTTTTATAGGAAAAAGAAAGACACCCC
ACCACAAACATTTTTTTGA
>gi|3328612|gb|AAC67797.1| Fructose-6-P Phosphotransferase
[Chlamydia trachomatis]
MSSNKHASLCQKTPSLCRELQKAPALLLTEDIRFKALLNERIDSVAELFPCTYNSPYYKFISKSDLSAET
SPLKVGVMLSGGPAPGGHNVILGLLHSIKKLHPNSQLLGFIRNGEGLLNNNTVEITDEFIEEFRNSGGFN
CIGTGRTNIITEENKARCLQTANELDLDGLVIIGGDGSNTATAILAEYFAKHQAKTVLVGVPKTIDGDLQ
HLFLDLTFGFDTATKFYSSIISNISRDALSCKGHYHFIKLMGRSSSHITLECALQTHPNIALIGEEIAEK
SISLETLIHDICETIADRAAMGKYHGVILIPEGVIEFIPEIQSLVKEIESIPEQENLYQALSLSSQQLLC
QFPEDICHQLLYNRDAHGNVYVSKISVDKLLIHLVRQHLETHFRQVPFNAISHFLGYEGRSGTPTHFDNV
YSYNLGYGAGVLVFNRCNGYLSTIEGLTSPIEKWRLRALPIVRMLTTKQGKDSKHYPLIKKRLVDIASPV
FNKFSLYRKIWALEDSYRFVGPLQIHSPEDAHSDDFPPLILFLNHEWQKRCSICLEIPDQDY
>gi|3328609|gb|AE001294.1:2452-4113,
ATGTCGTCGAATAAACATGCTTCTCTTTGTCAAAAGACGCCTTCTTTGTGTCGGGAGCTTCAAAAAGCTC
CTGCTCTTCTATTAACAGAAGACATAAGGTTTAAAGCTCTTCTTAATGAACGCATTGACTCTGTTGCAGA
ACTATTTCCATGCACTTATAACTCTCCCTACTACAAATTTATTTCGAAGTCCGATCTTTCCGCTGAGACC
TCTCCCCTTAPAGTGGGCGTTATGCTTTCTGGAGGCCCAGCTCCTGGTGGGCACAATGTCATCTTAGGAT
TGCTACACAGTATTAAAAAGCTCCATCCGAATAGTCAGCTTTTAGGATTTATTCGCAATGGAGAAGGACT
TCTCAATAATAATACTGTAGAAATCACAGATGAATTCATTGAAGAGTTTCGTAACTCTGGAGGCTTTAAT
TGCATAGGAACAGGTCGCACTAATATCATAACCGAAGAAAATAAAGCGCGCTGTTTACAAACAGCAAATG
AACTCGATTTAGATGGATTAGTGATTATTGGAGGCGATGGTTCGAATACAGCCACGGCGATTCTTGCTGA
ATATTTTGCTAAGCATCAAGCAAAAACGGTATTAGTTGGTGTTCCCAAAACTATTGATGGAGATTTGCAG
CACCTATTTTTAGACCTCACATTTGGGTTTGATACTGCTACTAAATTTTATTCATCCATCATCAGCAACA
TTTCTAGAGACGCATTATCGTGTAAAGGCCACTATCATTTTATTAAACTAATGGGCCGGTCTTCTTCTCA
TATCACGCTAGAATGCGCACTACAGACTCACCCAAATATTGCTCTTATAGGCGAAGAGATTGCAGAAAAA
AGCATCTCCTTAGAAACATTAATCCATGATATTTGTGAAACAATAGCAGATCGAGCTGCTATGGGGAAAT
ACCATGGCGTTATTCTCATCCCTGAAGGAGTCATTGAGTTTATTCCTGAAATACAGTCTCTGGTTAAAGA
AATTGAATCCATTCCAGAGCAGGAGAATCTTTACCAAGCTTTATCCTTATCTTCTCAGCAACTTTTATGC
CAATTTCCGGAAGATATTTGCCATCAGCTCTTGTATAATAGAGATGCTCATGGCAACGTCTATGTATCAA
AAATTAGTGTTGATAAACTTCTGATTCATCTAGTTCGTCAACATTTAGAAACACATTTTAGACAAGTTCC
CTTCAATGCAATCTCCCATTTTTTAGGTTATGAAGGGCGTTCAGGAACTCCTACACATTTTGATAATGTG
TATAGCTATAACTTAGGATATGGTGCTGGGGTTCTCGTTTTTAACCGCTGTAATGGGTATTTATCCACGA
TCGAAGCTCTAACTAGCCCTATTCAAAAATGGCGATTGCGCGCTTTACCCATTGTTCGAATGTTGACGAC
CAAGCAGGGGAAAGACAGTAAACATTATCCTCTGATAAAAAAAAGATTGGTAGATATTGCTAGTCCTGTT
TTTAATAAGTTCTCACTGTATCGGAAAATCTGGGCTTTAGAAGACTCCTATCGCTTTGTAGGGCCATTAC
AAATACATTCTCCGGAGGATGCTCATTCTGATGATTTTCCTCCTTTAATTTTGTTTTTGAATCATAATGA
ATGGCAAAAACGCTGTTCTATTTGTTTAGAAATCCCCGATCAGGATTATTAA
>gi|3328517|gb|AAC67709.1| hypothetical protein
[Chlamydia trachomatis]
MICCDKVLSSVQSMPVIDKCSVTKCLQTAKQAAVLALSLFAVFASGSLSILSAAVLFSGTAAVLPYLLIL
TTALLGFVCAVIVLLRNLSAVVQSCKKRSPEEIEGAARPSDQQESGGRLSEESASPQASPTSSTFGLESA
LRSIGDSVSGAFDDINKDNSRSRSHSF
>gi|3328516|gb|AE001286.1:75-578,
ATGATCTGCTGTGACAAAGTCTTGTCGAGCGTACAATCAATGCCTGTTATAGATAAATGCTCTGTAACGA
AATGCTTACAAACGGCTAAGCAAGCAGCTGTTCTTGCGTTGTCTTTGTTTGCGGTGTTTGCTTCAGGAAG
TTTATCCATATTATCAGCGGCGGTACTGTTTAGTGGCACTGCTGCTGTTCTTCCATATCTGCTGATATTA
ACAACAGCTCTTCTAGGATTTGTTTGTGCTGTTATTGTGCTTTTAAGAAATTTATCAGCAGTTGTTCAGA
GTTGTAAAAAGAGATCACCTGAAGAAATTCAAGGGGCTGCTCGTCCCTCTGATCAGCAGGAATCAGGAGG
ACGTTTGTCCGAGGAGAGCGCTTCACCACAAGCATCTCCTACTTCGTCTACTTTTGGTCTTGAATCCGCT
TTGCGCTCAATAGGAGATA
>gi|3328482|gb|AAC67677.1| L28 Ribosomal Protein [Chlamydia
trachomatis]
MSKKCALTGRKPRRGYSYAIRGISKKKKGIGLKVTGRTKRRFFPNNMTKRLWSTEENRFLKLKISAAALR
LVDKLGLDQVVAHAKSKGF
>gi|3328480|gb|AE001283.1:c2251-1982,
ATGTCGAAAAAATGTGCGCTTACAGGAAGAAAGCCTCGTCGCGGTTATAGCTATGCTATCCGAGGGATTT
CTAAAAAGAAAAAAGGGATCGGTTTGAAAGTTACAGGAAGAACAAAACGTCGATTCTTCCCTAATATGAT
GACTAAGAGACTATGGTCTACTGAGGAAAATCGCTTCCTCAAACTCAAAATTTCTGCAGCAGCTTTACGC
CTTGTTGATAAACTAGGGTTAGATCAGGTTGTTGCTAGAGCTAAAAGCAAGGCTTTTTAG
>gi|3328436|gb|AAC67635.1|SS DNA Binding Protein [Chlamydia
trachomatis]
MLFGYLVGFLAADPEERMTSGGKRVVVLRLGVKSRVGSKDETVWCRCNIWNNRYDKMLPYLKKGSSVIVA
GELSLESYVGRDGSPQASISVSVDTLKFNSGSSRPDARGSDEGRQBANDNVSIGFDGESLDTDSALDKEV
YAGFGEDQQYASEDVPF
>gi|3328434|gb|AE001279.1:1060-1533,
ATGTTGTTCGGATATTTGGTAGGATTTCTAGCTGCCGATCCTGAAGAAAGAATGACATCCGGAGGTAAAC
GGGTTGTTGTTTTACGTTTGGGTGTAAAATCTCGTGTAGGATCTAAAGATGAAACAGTGTGGTGCAGATG
CAATATCTGGAACAACCGTTATGATAAGATGCTTCCTTATTTGAAGAAAGGTTCTTCAGTCATTGTTGCT
GGAGAGCTTTCTTTAGAAAGCTATGTAGGTAGAGACGGTTCTCCACAAGCTTCTATTTCTGTAAGCGTAG
ATACATTAAAATTTAATTCCGGATCTTCTCGTCCTGATGCTAGAGGTTCAGATGAAGGTCGTCAGAGAGC
TAATGATAATGTCTCTATTGGATTTGATGCAGAAAGTTTAGATACAGACTCTGCGCTTGATAAGGAAGTC
TATGCAGGGTTTGGAGAAGACCAACAGTATGCTAGTGAGGATGTTCCTTTTTAG
>gi|3328411|gb|AAC67611.1|hypothetical protein
[Chlamydia trachomatis]
MKKQEKMHPQNLLKVFIFFLAFFISYPSAEAHSPLQSSIQEKILTARPGDYAVLSRGSQKFFFLIRQSSS
EATWVEMSEFASLTQQEKKLVEQSSWKNAFHQLQSSKKVYLLRISKNPLMIFVLKNAQWMPLSEKDPLPF
FVKILRLPLSPAPSHLIKYKGKERTPWSPRTSLNGELITLPSSAWISVWPKDSSPLSEKNILIYFSNNER
LAFPLWTSIDTPTGTVIIKTIEMGHQAASSYPALPNF
>gi|3522886|gb|AE001277.1:c6191-5448,
ATGAAAAAGCAAGAAAAAATGCACCCTCAAAACCTTCTTAAAGTTTTTATTTTTTTCTTGGCATTTTTCA
TATCCTATCCCTCGGCTGAAGCCCATTCTCCTCTCCAATCATCAATCCAAGAAAAAATTCTAACTGCCCG
CCCCGGAGACTATGCCGTCTTAAGCCGAGGATCTCAAAAATTTTTCTTTTTAATTCGCCAAAGTTCTTCG
GAAGCGACTTGGGTCGAAATGTCTGAATTTGCCTCCCTAACACAGCAAGAAAAAAAATTAGTAGAACAGT
CTTCCTGGAAGAATGCCTTCCATCAACTCCAATCTTCAAAAAAAGTGTACTTGTTACGAATTTCCAAAAA
TCCTCTTATGATTTTTGTTCTCAAAAATGCGCAATGGATGCCTCTCTCAGAAAAAGATCCTTTGCCTTTC
TTTGTAAAAATCCTTCGACTCCCTTTATCTCCAGCCCCCTCTCACTTAATTAAATACAAAGGGAAAGAAC
GCACCCCCTGGTCTCCGCGAACATCTTTGAATGGAGAACTCATAACCCTTCCTTCCAGTGCTTGGATTTC
TGTTTGGCCAAAAGATTCTTCTCCTCTATCAGAAAAAAATATTCTCATATATTTTTCTAACAATGAACGT
TTAGCGTTTCCTCTATGGACTAGTATTGATACTCCTACAGGGACAGTGATTATTAAGACTATTGAAATGG
GGCACCAAGCCGCCTCCTCCTATCCAGCTCTTCCCAATTTCTAG

crpA, CHLTR 15 kD Cysteine-Rich Protein (Chlamydia trachomatis Serovar D (D/UV-3/Cx)

DNA Sequence

AATATGAGCACTGTACCCGTTGTTCAAGGAGCGGATCTTCCAATTCGGCA
CAGGATATTTCCATAGACCATTAACACTGAAAGAGCGTATATCGAATCTT
CTATCTTCCACTGCATTTAAGGTGGGATTAGTGGTGATAGGACTACTTTT
AGTGATTGCTACTTTGATATTCCTAGTTTCGGCAGCTTCGTTTGTAAATG
CCATCTATGTAGTAGCTATTCCTGCTTTTGGGATGCGTGAATATCTGCGT
AGGAATTTTATCCATGGAAGGACACTGTTCTCCGGAGAGATGGATCTTAT
GTAAGAAGGTATAAAGACTTCAGAAGATATCATCGATGATGGGCAGATAA
ACAACTCTAATAAAGTGTITACTGATGAGAGGTTGAATGCCATAGGTGGG
GTAGTGGAATCTCTATCTAGAAGAAATAGTCTGGTGGATCAGACCCAAT
GA

Translated Amino Acid Sequence

NMS TVPVVQGAGS SNSAQDISTR PLTLKERISN LLSSTAFKVG
LVVIGLLLVI ATLIFLVSAA SFVNAIYLVA IPAILGCVNI CVGILSMEGH
CSPERWILCK KVLKTSEDII DDGQINNSNK VFTDERLNAI GGVVESLSRR
NSLVDQTQ*

OmcA, CHLTR 9 kD Cystein-Rich Outer Membrane Complex Lipoprotein (Chlamydia trachomatis Serovar D (D/UW-3/Cx)

DNA Sequence

GGGCTAGTTTCTTTTATTGTTAAAAGAATTGCTTTTATCGATAAAAGAAA
CTTCAAGAGCCCTTTTCTAGAAAGGAGTCTGGAAGTTATGAAAAAAACTG
CTTTACTCGCTGCTTTATGTAGTGTTGTTTAAGTAGTTGTTGTCGTATCG
TTGACTGTTGCTTCGAAGATCCATGCGCACCTATCCAATGTTCACCTTGT
GAATCTAAGAAGAAAGACGTAGACGGTGGTTGCAAC
TCTTGTAACGGGTATGTCCCAGCTTGCAAACCTTGCGGAGGGGATACGCA
CCAAGATGCTAAACATGGCCCTCAAGCTA
G
AGGAATTCCAGTTGACGGCAAATGCAGACAATAG

Translated Amino Acid Sequence

GL VSFIVKRIAF IDKRNFKSPF
LERSLEVMKK TALLAALCSV VSLSSCCRIV DCCFEDPCAP IQCSPCESKK
KDVDGGCNSC NGYVPACKPC GGDTHQDAKH GPQARGIPVD GKCRQ*

cutE, Apolipoprotein N-acyltransferase (Chlamydia trachomatis Serovar D (D/UW-3/Cx)

DNA Sequence

GCTAGTAAGGGAGCCCCTTTAGTGTTTAAACTTGTGTCATACATCATCCT
TTCTTGGGTGCTGGTCTGTTTGGCTCAGCCGGATGTAAGTGTTGTAGCTT
CTGTTGTTAGTTGTATTTGCGGTTACAGCTTACTTTGGGCTGGGCTTTTT
GCTTTAGTAGAGCAATTATCTTGGAAGAAAGTTTGGTGCATCGCTTTTAT
TTGGACTTGGACTGTCGAAGGCGCTCATTTCTCTTGGATGCTTGAAGATC
TTTATGTAGGGACAAGCATCTTATTTTGTTTGGGGTATACTGCTTTCTTA
TCTCGCCACCCTATTTGCTAGTTTTTCTTGTTTGGTTGTGTGGTGTTGTC
GCAAGCAATATAGGGGAGCTCTTGTTTGGCTTCCAGGGGTTTGGGTGGCG
ATAGAAGCAATACGCTATTATGGGTTGCTTTCAGGAGTTTCTTTTGATTT
TATTGGCTGGCCTCTTACAGCGACAGCCTATGGCCGGCAATTCGGCAGCT
TTTTGGATGGGCTGGACAAAGCTTTCTAGTTATTGGTGCCAATATATGCT
GTTTTGCAGTATGTTTATTAAAACACTCTTTTCCAAAGGTTTGTGGTTGA
CGTTGTGCGCGTTCCCTTATCTGTTAGGCGGAGCGCATTACGAATACCTA
AAGAAGCATTTTTTCCGACTCTGAAGTGCTTCGAGTTGCCATCGTGCAGC
CTGGATATAGTCCTCATATGCATGCAGGGAGGACGGCTAGTGCTATTTGG
AGAGGTGGTTTCTTTGTGCCAGACTATTCAAACTCCTGTAGATGTGATCG
TTTTCCCAGAAGTAAGTGTTCCTGGCTTACATAGACAAGCCTATACTCTT
CATGAAAATCAGCCTGTATTAGAAAGTTTGCTTCCTAACAAATCTTGGGG
CGAGTTTTTCACAAATTTGGATTGGATCCAAGCGATAGCTGAACGTTATC
AATGCACCGTTATCATGGGAATGGAACGATGGGAAAATAAAGGGGGAATA
CTGCATTTGTATAATGCTGGTGAATGCGTATCGCGAGAAGGGGAAATAAC
TAGCTATGATAAGCGGATTCTTGTTCCTGGAGGTGAGTACATCCCTGGAG
GGAAAATAGGTTTTTTCCTTGTGTCAAACCTTTTTCCCAGAATTTGCTCT
TCCCTTTCAACGTTTGCCAGGAGAGTTTTCTGGAGTTGTGAATATAACAG
AGCGAATAAAAGCTGGGATCTCTATTTGTTATGAGGAGACATTTGGGTAT
GCAATTTCGCCCTTACAAAAGGCAACAAGCCGATATTTTAGTAAATCTTA
CTAATGACGGTTGGTATCCGCGTTCAAGGCTGCCTCTAGTACATTTTTAT
CATGGCATGTTACGTAATCAAGAGTTGGGTATACCTTGTATTCGCGCCTG
TCGCACAGGAGTTTCTGCTGCAGTGGATTCTTTGGGTAGAATTGTCGGCA
TACTCCGTGGGAATCGAGAACTTGCCCAGTTTCTACAGGAGTACTCCAAG
TTTCCGTCCCTCTTTACAGTTATCATACTGTATATGCAAGGCTGGGTGAT
GCTCCTCTGTTAGTGATTGCAGTTTGTTCGGTTATCGGAGCGATTGCCTA
TTTTTATAGGAAAAAGAAAGAGACCCCACCACAAACATTTTTTTGA

Translated Amino Acid Sequence

ASKG
APLVFKLVSY IILSWVLVCL AQPDVSVVAS VVSCICGYSL LWAGLFALVE
QLSWKKVWCI AFIWTWTVEG AHFSWMLEDL YVGTSIYFVW GILLSYLATL
FASFSCLVVW CCRKQYRGAL VWLPGVWVAI EAIRYYGLLS GVSFDFIGWP
LTATAYGRQF GSFFGWAGQS FLVIAANICC FAVCLLKHSF SKGLWLTLCA
FPYLLGGAHY EYLKKHFSDS EVLRVAIVQP GYSPHMHAGR TASAIWRGLV
SLCQTIQTPV DVIVFPEVSV PFGLHRQAYT LHENQPVLES LLPNKSWGEF
FTNLDWIQAI AERYQCTVIM GMERWENKGG ILHLYNAAEC VSREGEITSY
DKRLLVPGGE YIPGGKIGFS LCQTFFPEFA LPFQRLPGEF SGVVNITERI
KAGISICYEE TFGYAIRPYK RQQADILVNL TNDGWYPRSR LPLVHFYHGM
LRNQELGLPC IRACRTGVSA AVDSLGRIVG ILPWESRTCP VSTGVLQVSV
PLYSYHTVYA RLGDAPLLLI AVCSVIGAIA YFYRKKKETP PQTFF*

pal, Peptidoglycan-Associated Lipoprotein (Chlamydia trachomatis Serovar D (D/UW-3/Cx)

DNA Sequence

G AAAATTGTTA TAGGATCAGG
AGAGAAACGT TTCCCATGCT GGGGAGCATT TCCCTTACAA CATATAAAGA
AAACCTCATG AGAAAGACTA TTTTTAAAGC GTTTAATTTA TTATTCTCCC
TTCTTTTTCT TTCTTCATGC TCTTATCCTT GCAGAGATTG GGAATGCCAT
GGTTGCGACT CCGCAAGACC TCGTAAATCC TCTTTTGGAT TCGTACCTTT
CTACTCCGAT GAAGAAATTC AACAAGCTTT TGTTGAAGAT TTTGATTCCA
AAGAAGAGCA GCTGTACAAA ACGAGCGCAC AGAGTACCTC TTTCCGAAAT
ATCACTTTCG CTACAGATAG TTATTCTATT AAAGGAGAGG ATAACCTCAC
GATTCTTGCA AGCTTAGTTC GTCATTTGCA TAAATCTCCT AAAGCTACGC
TATATATAGA GGGCCATACA GATGAACGTG GAGCTGCAGC TTATAACCTA
GCTTTAGGAG TTCGTCGTGC GAATGCTGTA AAACAATACC TCATCAAACA
GGGAATCGCT GCAGACCGCT TATTCACTAT TTCTTACGGA AAAGAACATC
CTGTTCATCC AGGCCATAAT GAATTAGCTT GGCAACAAAA TCGTCGTACT
GAATTTAAGA TCCATGCTCG CTAA

Translated Amino Acid Sequence

| ENCYRIR | |
|---|---|
| | RETFPMLGSI SFTTYKENLM RKTIFKAFNL LFSLLFLSSC SYPCRDWECH |
| | GCDSARPRKS SFGFVPFYSD EEIQQAFVED FDSKEEQLYK TSAQSTSFRN |
| | ITFATDSYSI KGEDNLTILA SLVRHLHKSP KATLYIEGHT DERGAAAYNL |
| | ALGARRANAV KQYLIKQGIA ADRLFTISYG KEHPVHPGHN ELAWQQNRRT |
| | EFKIHAR* |

The following Chlamydia trachomatis outer membrane proteins (full sequences above) are disclosed for the first time as being useful in a C. trachomatis vaccine. A vaccine comprising one or more of these proteins (or native or functional analogues thereof) is a further aspect of this invention (particularly in the context of being presented on the surface of a bleb).

Amino Acid Sequences:

>gi|6578118|gb|AAC68456.2| predicted Protease containing IRBP and DHR domains [Chlamydia trachomatis]
>gi|6578109|gb|AAC68227.2| CHLPN 76 kDa Homolog [Chlamydia trachomatis]
>gi|3328866|gb|AAC68034.1| Sulfite Reductase [Chlamydia trachomatis]
>gi|3328815|gb|AAC67986.1| hypothetical protein [Chlamydia trachomatis]
>gi|3328587|gb|AAC67774.1| CMP-2-keto-3-deoxyoctulosonic acid synthetase [Chlamydia trachomatis]
>gi|3329039|gb|AAC68197.1| Thio:disulfide Interchange Protein [Chlamydia trachomatis]
>gi|3329000|gb|AAC68161.1| Yop proteins translocation lipoprotein J [Chlamydia trachomatis]
>gi|3328905|gb|AAC68071.1| hypothetical protein [Chlamydia trachomatis]
>gi|3328884|gb|AAC68051.1| Phosphatidate Cytidylytransferase [Chlamydia trachomatis]
>gi|3328855|gb|AAC68022.1| hypothetical protein [Chlamydia trachomatis]
>gi|3328772|gb|AAC67946.1| hypothetical protein [Chlamydia trachomatis]
>gi|3328763|gb|AAC67938.1| O-Sialoglycoprotein Endopeptidase family [Chlamydia trachomatis]
>gi|6578102|gb|AAC67897.2| ATP Synthase Subunit K [Chlamydia trachomatis]
>gi|3329252|gb|AAC68382.1| S14 Ribosomal Protein [Chlamydia trachomatis]
>gi|3328987|gb|AAC68150.1| hypothetical protein [Chlamydia trachomatis]
>gi|3328972|gb|AAC68136.1| Apolipoprotein N-Acetyltransferase [Chlamydia trachomatis]
>gi|3328612|gb|AAC67797.1| Fructose-6-P Phosphotransferase [Chlamydia trachomatis]
>gi|3328517|gb|AAC67709.1| hypothetical protein [Chlamydia trachomatis]
>gi|3328482|gb|AAC67677.1| L28 Ribosomal Protein [Chlamydia trachomatis]
>gi|3328436|gb|AAC67635.1| SS DNA Binding Protein [Chlamydia trachomatis]
>gi|3328411|gb|AAC67611.1| hypothetical protein [Chlamydia trachomatis]

Again, when such blebs are present in a vaccine formulation they may be more protective against Chlamydia trachomatis infection than the use of the protein in isolation.

Particularly beneficial pairs of Chlamydia trachomatis antigens are further preferred embodiments of this invention. Thus in a further aspect a Gram-negative bleb (preferably from gonococcus) is provided presenting on its surface both the PorB and PmpG outer membrane proteins from Chlamydia trachomatis. Furthermore, a Gram-negative bleb (preferably from gonococcus) is provided presenting on its surface both the PorB and MOMP (from one or more serovars) outer membrane proteins from Chlamydia trachomatis. Lastly, a Gram-negative bleb (preferably from gonococcus) is provided presenting on its surface both the PmpG and MOMP (from one or more serovars) outer membrane proteins from Chlamydia trachomatis.

By MOMP (or OMP1 or OMP I) from one or more (1, 2, 3, 4, 5, 6, 7, 8, 9, 10 or more) serovars it is preferred that one or more should be selected from a list serovars consisting of: B, Ba, D, Da, E, L1, L2, L2a, F, G, K, L3, A, C, H, I, Ia, & J; more preferably from a list consisting of D, E, F, G, K, H, I, & J. Most preferably one or more MOMPs should at least comprise MOMP from serovar D or E (most preferably D). A further preferred strategy is the selection of one or more MOMP from each of the following 3 serogroups: B-serogroup (consisting of serovars B, Ba, D, Da, E, L1, L2 and L2a, and preferably selected from serovars D, Da, & E); F-G-serogroup (consisting of serovars F and G); and C-serogroup (consisting of servars A, C, H, I, Ia, J, K, and L3, and preferably selected from serovars H, I, Ia, J and K).

Most preferably the genes for the Chlamydia trachomatis antigens should be inserted at the PorA locus of Neisseria (preferably gonococcus).

Such a prepartion formulated as a vaccine may give enhanced protection to a host against Chlamydia trachomatis than when a single antigen is administered.

Preferably the bleb has been derived from a strain (preferably gonococcus) which has been modified to upregulate one or more protective outer membrane antigens (as described below).

Preferably the bleb has been derived from a strain (preferably gonococcus) which has been modified to downregulate one or more immunodominant variable or non-protective outer membrane antigens (as described below).

Preferably the blebs are derived from a strain (preferably gonococcus) which has a detoxified lipid A portion of bacterial LPS, due to the strain having been engineered to reduce or switch off expression of one or more genes which cause LPS to be toxic (preferably selected from the following genes, or homologues thereof htrB, msbB and lpxK; see section below).

Preferably the blebs are derived from a strain (preferably gonococcus) which has a detoxified lipid A portion of bacterial LPS, due to the strain having been engineered to express at a higher level of one or more genes producing a gene product that is capable of detoxifying LPS (preferably selected from the following genes, or homologues thereof: pmrA, pmrB, pmrE and pmrF; see section below).

Vaccine compositions comprising the bleb of the invention and a pharmaceutically suitable excipient or carrier is also envisaged. Preferably the vaccine additionally comprises a mucosal adjuvant. Mucosal adjuvants are well known in the art (see Vaccine Design “The subunit and adjuvant approach” (eds Powell M. F. & Newman M. J.) (1995) Plenum Press New York). A preferred mucosal adjuvant is LT2 (or LTII, which can be split into LTIIa and LTIIb—see Martin et al. Infection and Immunity, 2000, 68:281-287). Preferably such vaccines should be formulated and administered as described below in “vaccine formulations”.

The content of blebs per dose in the vaccine will typically be in the range 1-100 μg, preferably 5-50 μg, most typically in the range 5-25 μg.

Optimal amounts of components for a particular vaccine can be ascertained by standard studies involving observation of appropriate immune responses in subjects. Following an initial vaccination, subjects may receive one or several booster immunisations adequately spaced.

A method of preventing Chlamydia trachomatis infection in a host is also provided comprising the steps of administering an effective amount of the above vaccine to a host in need thereof. Preferably the vaccine is mucosally administered via either a intranasal, oral, intradermal or intravaginal route.

Chlamydia pneumoniae Antigens Integrated into a Gram Negative Bacterial Bleb

In a further aspect, the invention provides a Gram-negative bleb presenting on its surface a protective antigen from Chlamydia pneumoniae. Neisseria meningitidis, Moraxella catharralis, and Haemophilus influenzae are preferred species for the production of said bleb. A bacterial strain capable of producing such a bleb is a further aspect of the invention. Such protective antigens are preferably one or more of those listed below:

| | |
|---|---|
| Gene: | Protein Function: |

1) Cell Envelope: Membrane Proteins, Lipoproteins and Porins

| | |
|---|---|
| yaeT | OMP85 homolog |
| 60IM | 60 kD inner membrane protein |
| lgt | prolipoprotein diacylglyceryl transferase |
| crpA | CHLTR 15 kD cysteine-rich protein |
| omcB | 60 kD cysteine-rich outer membrane complex protein |
| omcA | 9 kD cysteine-rich outer membrane complex lipoprotein |
| cutE | apolipoprotein N-acetyltransferase |
| ompA | major outer membrane protein |
| pal | peptidoglycan-associated lipoprotein |
| porB | outer membrane protein analog |

2) Coding Genes (Not in C. trachomatis)

| | |
|---|---|
| yqfF | conserved hypothetical inner membrane protein |
| yxjG | hypothetical protein |
| guaA | GMP synthase |
| guaB | inosine 5′-monophosphate dehydrogenase |
| argR | similarity to arginine repressor |
| CPn0232 | similarity to 5′-methylthioadenosine nucleosidase |
| CPn0251 | conserved hypothetical protein |
| CPn0278 | conserved outer membrane lipoprotein protein/a> |
| CPn0279 | possible ABC transporter permease |
| yxjG | hypothetical protein |
| yqeV | hypothetical protein |
| CPn0486 | hypothetical proline permease |
| CPn0505 | 3-methyladenine DNA glycosylase |
| CPn0562 | CHLPS 43 kDa protein |
| CPn0585 | similarity to CHLPS IncA |
| yvyD | conserved hypothetical protein |
| CPn0608 | uridine 5′-monophosphate synthase |
| CPn0735 | uridine kinase |
| CPn0907 | CutA-like periplasmic divalent cation tolerance protein |
| CPn0927 | CHLPS 43 kDa protein |
| CPn0928 | CHLPS 43 kDa protein |
| CPn0929 | CHLPS 43 kDa protein |
| CPn0980 | similar to S. cerevisiae 52.9 kDa protein |
| bioA | adenosylmethionine-8-amino-7-oxononanoate |
| | aminotransferase |
| bioD | dethiobiotin synthetase |
| bioB | biotin synthase |
| CPn1045 | conserved hypothetical membrane protein |
| CPn1046 | tryptophan hydroxylase |

3) Chlamydia-Specific Proteins

| | |
|---|---|
| pmp_1 | polymorphic outer membrane protein |
| pmp_2 | polymorphic outer membrane protein |
| pmp_3 | polymorphic outer membrane protein |
| pmp_3 | polymorphic outer membrane protein |
| pmp_4 | polymorphic outer membrane protein |
| pmp_4 | polymorphic outer membrane protein |
| pmp_5 | polymorphic outer membrane protein |
| pmp_5 | polymorphic outer membrane protein |
| CPn0133 | CHLPS hypothetical protein |
| CPn0186 | similarity to IncA |
| incB | inclusion membrane protein B |
| incC | inclusion membrane protein C |
| CPn0332 | CHLTR T2 protein |
| ltuB | LtuB protein |
| pmp_6 | polymorphic outer membrane protein |
| pmp_7 | polymorphic outer membrane protein |
| pmp_8 | polymorphic outer membrane protein |
| pmp_9 | polymorphic outer membrane protein |
| pmp_10 | polymorphic outer membrane protein |
| pmp_10 | polymorphic outer membrane protein |
| pmp_11 | polymorphic outer membrane protein |
| pmp_12 | polymorphic outer membrane protein |
| pmp_13 | polymorphic outer membrane protein |
| pmp_14 | polymorphic outer membrane protein |
| pmp_15 | polymorphic outer membrane protein |
| pmp_16 | polymorphic outer membrane protein |
| pmp_17 | polymorphic outer membrane protein |
| pmp_17 | polymorphic outer membrane protein |
| pmp_17 | polymorphic outer membrane protein |
| pmp_18 | polymorphic outer membrane protein |
| pmp_19 | polymorphic outer membrane protein |
| pmp_20 | polymorphic outer membrane protein |
| euo | CHLPS Euo protein |
| CPn0562 | CHLPS 43 kDa protein homolog |
| CPn0585 | similar to CHLPS inclusion membrane protein A |
| CPn0728 | CHLPN 76 kDa protein homolog |
| CPn0729 | CHLPN 76 kDa protein homolog |
| gp6D | CHLTR plasmid protein |
| CPn0927 | CHLPS 43 kDa protein homolog |
| CPn0928 | CHLPS 43 kDa protein homolog |
| CPn0929 | CHLPS 43 kDa protein homolog |
| pmp_21 | polymorphic outer membrane protein |
| ltuA | LtuA protein |

(Full sequence information has been published at the Chlamydia Genome Project web site: http://chlamydia-www.berkeley.edu:4231/index.html).

Additional Chlamydia Genes, and Encoded Proteins, Suitable for Expression in a Gram-Negative Bacteria for OMV Vaccine Preparation:

| | |
|---|---|
| Chlamydia pneumoniae 98 kD putative outer membrane protein gene. | WO200026237-A2 |
| Patent Inventors | |
| DUNN PL | |
| OOMEN RP | |
| MURDIN AD | |
| Chlamydia POMP91B precursor gene. | WO200026239-A2 |
| Patent Inventors | |
| DUNN PL | |
| OOMEN RP | |
| MURDIN AD | |
| Chlamydia antigen CPN100634 full length coding sequence. | WO200032794-A2 |
| Patent Inventors | |
| OOMEN RP | |
| WANG J | |
| MURDIN AD | |
| Chlamydia antigen CPN100634 gene open reading frame. | WO200032794-A2 |
| Patent Inventors | |
| OOMEN RP | |
| WANG J | |
| MURDIN AD | |
| Chlamydia antigen CPN100635 full length coding sequence. | WO200032794-A2 |
| Patent Inventors | |
| OOMEN RP | |
| WANG J | |
| MURDIN AD | |
| Chlamydia antigen CPN100635 gene open reading frame. | WO200032794-A2 |
| Patent Inventors | |
| OOMEN RP | |
| WANG J | |
| MURDIN AD | |
| Chlamydia antigen CPN100638 full length coding sequence. | WO200032794-A2 |
| Patent Inventors | |
| OOMEN RP | |
| WANG J | |
| MURDIN AD | |
| Chlamydia antigen CPN100638 gene open reading frame. | WO200032794-A2 |
| Patent Inventors | |
| OOMEN RP | |
| WANG J | |
| MURDIN AD | |
| Chlamydia antigen CPN100639 full length coding sequence. | WO200032794-A2 |
| Patent Inventors | |
| OOMEN RP | |
| WANG J | |
| MURDIN AD | |
| Chlamydia antigen CPN100639 gene open reading frame. | WO200032794-A2 |
| Patent Inventors | |
| OOMEN RP | |
| WANG J | |
| MURDIN AD | |
| Chlamydia antigen CPN100708 full length coding sequence. | WO200032794-A2 |
| Patent Inventors | |
| OOMEN RP | |
| WANG J | |
| MURDIN AD | |
| Chlamydia antigen CPN100708 gene open reading frame. | WO200032794-A2 |
| Patent Inventors | |
| OOMEN RP | |
| WANG J | |
| MURDIN AD | |
| C. pneumoniae ATP/ADP translocase coding sequence. | WO200039157-A1 |
| Patent Inventors | |
| DUNN P | |
| OOMEN RP | |
| WANG J | |
| MURDIN AD | |
| Chlamydia pneumoniae 98 kDa outer membrane protein CPN100640 gene. | WO200032784-A1 |
| Patent Inventors | |
| DUNN P | |
| OOMEN RP | |
| WANG J | |
| MURDIN AD | |
| Chlamydia pneumoniae 98 kDa outer membrane protein coding region. | WO200032784-A1 |
| Patent Inventors | |
| DUNN P | |
| OOMEN RP | |
| WANG J | |
| MURDIN AD | |
| DNA encoding a 9 kDa cysteine-rich membrane protein. | WO200053764-A1 |
| Patent Inventors | |
| DUNN P | |
| OOMEN RP | |
| WANG J | |
| MURDIN AD | |
| DNA encoding a 60 kDa cysteine-rich membrane protein. | WO200055326-A1 |
| Patent Inventors | |
| DUNN P | |
| OOMEN RP | |
| WANG J | |
| MURDIN AD | |
| A 9 kDa cysteine-rich membrane protein. | WO200053764-A1 |
| Patent Inventors | |
| DUNN P | |
| OOMEN RP | |
| WANG J | |
| MURDIN AD | |
| A 60 kDa cysteine-rich membrane protein of Chlamydia pneumoniae. | WO200055326-A1 |
| Patent Inventors | |
| DUNN P | |
| OOMEN RP | |
| WANG J | |
| MURDIN AD | |
| C. pneumoniae mip (outer membrane protein). | WO200006741-A1 |
| Patent Inventors | |
| DUNN PL | |
| OOMEN RP | |
| MURDIN AD | |
| C. pneumoniae mip (outer membrane protein) truncated protein. | WO200006741-A1 |
| Patent Inventors | |
| DUNN PL | |
| OOMEN RP | |
| MURDIN AD | |
| C. pneumoniae omp protein sequence. | WO200006743-A2 |
| Patent Inventors | |
| DUNN PL | |
| OOMEN RP | |
| MURDIN AD | |
| C. pneumoniae omp protein truncated sequence. | WO200006743-A2 |
| Patent Inventors | |
| DUNN PL | |
| OOMEN RP | |
| MURDIN AD | |
| Amino acid sequence of the CPN100111 polypeptide. | WO200011183-A2 |
| Patent Inventors | |
| OOMEN RP | |
| MURDIN AD | |
| Amino acid sequence of the CPN100224 polypeptide. | WO200011183-A2 |
| Patent Inventors | |
| OOMEN RP | |
| MURDIN AD | |
| Amino acid sequence of the CPN100230 polypeptide. | WO200011183-A2 |
| Patent Inventors | |
| OOMEN RP | |
| MURDIN AD | |
| Amino acid sequence of the CPN100231 polypeptide. | WO200011183-A2 |
| Patent Inventors | |
| OOMEN RP | |
| MURDIN AD | |
| Amino acid sequence of the CPN100232 polypeptide. | WO200011183-A2 |
| Patent Inventors | |
| OOMEN RP | |
| MURDIN AD | |
| Amino acid sequence of the CPN100233 polypeptide. | WO200011183-A2 |
| Patent Inventors | |
| OOMEN RP | |
| MURDIN AD | |
| Amino acid sequence of the CPN100394 polypeptide. | WO200011183-A2 |
| Patent Inventors | |
| OOMEN RP | |
| MURDIN AD | |
| Amino acid sequence of the CPN100395 polypeptide. | WO200011183-A2 |
| Patent Inventors | |
| OOMEN RP | |
| MURDIN AD | |
| Amino acid sequence of the POMP91A protein of Chlamydia pneumoniae. | WO200011180-A1 |
| Patent Inventors | |
| DUNN PL | |
| OOMEN RP | |
| MURDIN AD | |
| Chlamydia pneumoniae antigen CPN100202 protein sequence. | WO200006739-A2 |
| Patent Inventors | |
| OOMEN RP | |
| MURDIN AD | |
| Chlamydia pneumoniae antigen CPN100149 protein SEQ ID NO: 2. | WO200006740-A1 |
| Patent Inventors | |
| OOMEN RP | |
| MURDIN AD | |
| Chlamydia pneumoniae antigen CPN100605 protein SEQ ID NO: 2. | WO200006742-A2 |
| Patent Inventors | |
| OOMEN RP | |
| MURDIN AD | |
| Chlamydia antigen CPN100634. | WO200032794-A2 |
| Patent Inventors | |
| OOMEN RP | |
| WANG J | |
| MURDIN AD | |
| Chlamydia antigen CPN100635. | WO200032794-A2 |
| Patent Inventors | |
| OOMEN RP | |
| WANG J | |
| MURDIN AD | |
| Mature Chlamydia antigen CPN100635. | WO200032794-A2 |
| Patent Inventors | |
| OOMEN RP | |
| WANG J | |
| MURDIN AD | |
| Chlamydia antigen CPN100638. | WO200032794-A2 |
| Patent Inventors | |
| OOMEN RP | |
| WANG J | |
| MURDIN AD | |
| Chlamydia antigen CPN100639. | WO200032794-A2 |
| Patent Inventors | |
| OOMEN RP | |
| WANG J | |
| MURDIN AD | |
| Chlamydia antigen CPN100708. | WO200032794-A2 |
| Patent Inventors | |
| OOMEN RP | |
| WANG J | |
| MURDIN AD | |
| C. pneumoniae ATP/ADP translocase protein sequence. | WO200039157-A1 |
| Patent Inventors | |
| DUNN P | |
| OOMEN RP | |
| WANG J | |
| MURDIN AD | |
| Chlamydia pneumoniae 98 kD putative outer membrane protein. | WO200026237-A2 |
| Patent Inventors | |
| DUNN PL | |
| OOMEN RP | |
| MURDIN AD | |
| Chlamydia POMP91B precursor protein. | WO200026239-A2 |
| Patent Inventors | |
| DUNN PL | |
| OOMEN RP | |
| MURDIN AD | |
| Chlamydia pneumoniae 98 kDa outer membrane protein CPN100640. | WO200032784-A1 |
| Patent Inventors | |
| DUNN P | |
| OOMEN RP | |
| WANG J | |
| MURDIN AD | |
| Chlamydia pneumoniae processed 98 kDa outer membrane protein | WO200032784-A1 |
| CPN100640. | |
| Patent Inventors | |
| DUNN P | |
| OOMEN RP | |
| WANG J | |
| MURDIN AD | |
| C. pneumoniae mip (outer membrane protein) encoding DNA. | WO200006741-A1 |
| Patent Inventors | |
| DUNN PL | |
| OOMEN RP | |
| MURDIN AD | |
| C. pneumoniae omp protein encoding DNA. | WO200006743-A2 |
| Patent Inventors | |
| DUNN PL | |
| OOMEN RP | |
| MURDIN AD | |
| DNA encoding the CPN100111 polypeptide. | WO200011183-A2 |
| Patent Inventors | |
| OOMEN RP | |
| MURDIN AD | |
| DNA encoding the CPN100224 polypeptide. | WO200011183-A2 |
| Patent Inventors | |
| OOMEN RP | |
| MURDIN AD | |
| DNA encoding the CPN100230 polypeptide. | WO200011183-A2 |
| Patent Inventors | |
| OOMEN RP | |
| MURDIN AD | |
| DNA encoding the CPN100231 polypeptide. | WO200011183-A2 |
| Patent Inventors | |
| OOMEN RP | |
| MURDIN AD | |
| DNA encoding the CPN100232 polypeptide. | WO200011183-A2 |
| Patent Inventors | |
| OOMEN RP | |
| MURDIN AD | |
| DNA encoding the CPN100233 polypeptide. | WO200011183-A2 |
| Patent Inventors | |
| OOMEN RP | |
| MURDIN AD | |
| DNA encoding the CPN100394 polypeptide. | WO200011183-A2 |
| Patent Inventors | |
| OOMEN RP | |
| MURDIN AD | |
| DNA encoding the CPN100395 polypeptide. | WO200011183-A2 |
| Patent Inventors | |
| OOMEN RP | |
| MURDIN AD | |
| Nucleotide sequence of the POMP91A gene of Chlamydia pneumoniae. | WO200011180-A1 |
| Patent Inventors | |
| DUNN PL | |
| OOMEN RP | |
| MURDIN AD | |
| Chlamydia pneumoniae antigen CPN100202 nucleotide sequence. | WO200006739-A2 |
| Patent Inventors | |
| OOMEN RP | |
| MURDIN AD | |
| Chlamydia pneumoniae antigen CPN100149 protein encoding DNA SEQ ID | WO200006740-A1 |
| NO: 1. | |
| Patent Inventors | |
| OOMEN RP | |
| MURDIN AD | |
| Chlamydia pneumoniae antigen CPN100605 protein encoding DNA SEQ ID | WO200006742-A2 |
| NO: 1. | |
| Patent Inventors | |
| OOMEN RP | |
| MURDIN AD | |

When such blebs are present in a vaccine formulation they may be more protective against Chlamydia pneumoniae infection than the use of the protein/antigen in isolation.

Particularly beneficial pairs of Chlamydia pneumoniae antigens have also been found. Thus in a further aspect a Gram-negative bleb (preferably from meningococcus) is provided presenting on its surface both the PorB and MOMP outer membrane proteins from Chlamydia pneumoniae. Furthermore, a Gram-negative bleb (preferably from meningococcus) is provided presenting on its surface both MOMP and one or more Pmp outer membrane proteins from Chlamydia pneumoniae. A Gram-negative bleb (preferably from meningococcus) is additionally provided presenting on its surface both the PorB and one or more Pmp outer membrane proteins from Chlamydia pneumoniae. A Gram-negative bleb (preferably from meningococcus) is also provided presenting on its surface both the PorB and Nptl proteins from Chlamydia pneumoniae. A Gram-negative bleb (preferably from meningococcus) is additionally provided presenting on its surface both the Nptl and one or more Pmp proteins from Chlamydia pneumoniae. Lastly, a Gram-negative bleb (preferably from meningococcus) is provided presenting on its surface both the Nptl and MOMP proteins from Chlamydia pneumoniae. Bacterial strains from which these blebs are derived are further aspects of this invention.

Such prepartions formulated as a vaccine can give enhanced protection to a host against Chlamydia than when a single antigen is administered.

Preferably the bleb has been derived from a strain which has been modified to upregulate one or more protective outer membrane antigens (see below; for instance for meningocococcal protective outer membrane antigens see section “Neisserial bleb preparations” for those antigens that should preferably be upregulated).

Preferably the bleb has been derived from a strain which has been modified to downregulate one or more immunodominant variable or non-protective outer membrane antigens (as described below; for instance for meningocococcal variable/non-protective outer membrane antigens see section “Neisserial bleb preparations” for those antigens that should preferably be downregulated).

Preferably the blebs are derived from a strain which has a detoxified lipid A portion of bacterial LPS, due to the strain having been engineered to reduce or switch off expression of one or more genes which cause LPS to be toxic (preferably selected from the following genes, or homologues thereof htrB, msbB and lpxK; see section below).

Preferably the blebs are derived from a strain which has a detoxified lipid A portion of bacterial LPS, due to the strain having been engineered to express at a higher level of one or more genes producing a gene product that is capable of detoxifying LPS (preferably selected from the following genes, or homologues thereof: pmrA, pmrB, pmrE and pmrF; see section below).

Vaccine compositions comprising the bleb of the invention and a pharmaceutically suitable excipient or carrier are also envisaged. Preferably the vaccine additionally comprising a mucosal adjuvant. Mucosal adjuvants are well known in the art (see Vaccine Design “The subunit and adjuvant approach” (eds Powell M. F. & Newman M. J.) (1995) Plenum Press New York). A preferred mucosal adjuvant is LT2 (or LTII, which can be split into LTIIa and LTIIb—see Martin et al. Infection and Immunity, 2000, 68:281-287). Preferably such vaccines should be formulated and administered as described below in “Vaccine formulations”.

The content of blebs per dose in the vaccine will typically be in the range 1-100 μg, preferably 5-50 μg, most typically in the range 5-25 μg.

Optimal amounts of components for a particular vaccine can be ascertained by standard studies involving observation of appropriate immune responses in subjects. Following an initial vaccination, subjects may receive one or several booster immunisations adequately spaced.

The efficacy of a C. pneumoniae vaccine can be evaluated in a mouse model of infection such as the one described by Murdin et al., 2000, J. Infect. Dis. 181 (suppl 3):S5444-51. The protection elicited by a vaccine formulation can be assessed by reduction of the bacterial load in the lung after a challenge infection with C. pneumoniae.

A method of preventing Chlamydia pneumoniae infection in a host is also provided comprising the steps of administering an effective amount of the above vaccine to a host in need thereof. Preferably the vaccine is mucosally administered via either an intranasal, intradermal or oral route.

Further Improvements in the Bacteria and Blebs of the Invention

The Gram-negative bacterium of the invention may be further genetically engineered by one or more processes selected from the following group: (a) a process of down-regulating expression of immunodominant variable or non-protective antigens, (b) a process of upregulating expression of protective OMP antigens, (c) a process of down-regulating a gene involved in rendering the lipid A portion of LPS toxic, (d) a process of upregulating a gene involved in rendering the lipid A portion of LPS less toxic, and (e) a process of down-regulating synthesis of an antigen which shares a structural similarity with a human structure and may be capable of inducing an auto-immune response in humans. These processes are described in detail in WO 01/09350 (incorporated by reference herein).

Such bleb vaccines of the invention are designed to focus the immune response on a few protective (preferably conserved) antigens or epitopes—formulated in a multiple component vaccine. Where such antigens are integral OMPs, the outer membrane vesicles of bleb vaccines will ensure their proper folding. This invention provides methods to optimize the OMP and LPS composition of OMV (bleb) vaccines by deleting immunodominant variable as well as non protective OMPs, by creating conserved OMPs by deletion of variable regions, by upregulating expression of protective OMPs, and by eliminating control mechanisms for expression (such as iron restriction) of protective OMPs. In addition the invention provides for the reduction in toxicity of lipid A by modification of the lipid portion or by changing the phosphoryl composition whilst retaining its adjuvant activity or by masking it. Each of these new methods of improvement individually improve the bleb vaccine, however a combination of one or more of these methods work in conjunction so as to produce an optimised engineered bleb vaccine which is immuno-protective and non-toxic—particularly suitable for paediatric use.

(a) A Process of Down-Regulating Expression of Immunodominant Variable or Non-Protective Antigens

Many surface antigens are variable among bacterial strains and as a consequence are protective only against a limited set of closely related strains. An aspect of this invention covers the reduction in expression, or, preferably, the deletion of the gene(s) encoding variable surface protein(s) which results in a bacterial strain producing blebs which, when administered in a vaccine, have a stronger potential for cross-reactivity against various strains due to a higher influence exerted by conserved proteins (retained on the outer membranes) on the vaccinee's immune system. Examples of such variable antigens include: for Neisseria—pili (PilC) which undergoes antigenic variations, PorA, Opa, TbpB, FrpB; for H. influenzae —P2, P5, pilin, IgA1-protease; and for Moraxella—CopB, OMP106.

Other types of gene that could be down-regulated or switched off are genes which, in vivo, can easily be switched on (expressed) or off by the bacterium. As outer membrane proteins encoded by such genes are not always present on the bacteria, the presence of such proteins in the bleb preparations can also be detrimental to the effectiveness of the vaccine for the reasons stated above. A preferred example to down-regulate or delete is Neisseria Opc protein. Anti-Opc immunity induced by an Opc containing bleb vaccine would only have limited protective capacity as the infecting organism could easily become Opc⁻. H. influenzae HgpA and HgpB are other examples of such proteins.

In process a), these variable or non-protective genes are down-regulated in expression, or terminally switched off. This has the surprising advantage of concentrating the immune system on better antigens that are present in low amounts on the outer surface of blebs.

The strain can be engineered in this way by a number of strategies including transposon insertion to disrupt the coding region or promoter region of the gene, or point mutations or deletions to achieve a similar result. Homologous recombination may also be used to delete a gene from a chromosome (where sequence X comprises part (preferably all) of the coding sequence of the gene of interest). It may additionally be used to change its strong promoter for a weaker (or no) promoter. All these techniques are described in WO 01/09350 (published by WIPO on Aug. 2, 2001 and incorporated by reference herein).

(b) A Process of Upregulating Expression of Protective OMP Antigens

This may be done by inserting a copy of such a protective OMP into the genome (preferably by homologous recombination), or by upregulating expression of the native gene by replacing the native promoter for a stronger promoter, or inserting a strong promoter upstream of the gene in question (also by homologous recombination). Such methods can be accomplished using the techniques described in WO 01/09350 (published by WIPO on Aug. 2, 2001 and incorporated by reference herein).

Such methods are particularly useful for enhancing the production of immunologically relevant Bleb components such as outer-membrane proteins and lipoproteins (preferably conserved OMPs, usually present in blebs at low concentrations).

(c) A Process of Down-Regulating a Gene Involved in Rendering the Lipid A Portion of LPS Toxic

The toxicity of bleb vaccines presents one of the largest problems in the use of blebs in vaccines. A further aspect of the invention relates to methods of genetically detoxifying the LPS present in Blebs. Lipid A is the primary component of LPS responsible for cell activation. Many mutations in genes involved in this pathway lead to essential phenotypes. However, mutations in the genes responsible for the terminal modifications steps lead to temperature-sensitive (htrB) or permissive (msbB) phenotypes. Mutations resulting in a decreased (or no) expression of these genes result in altered toxic activity of lipid A. Indeed, the non-lauroylated (htrB mutant) [also defined by the resulting LPS lacking both secondary acyl chains] or non-myristoylated (msbB mutant) [also defined by the resulting LPS lacking only a single secondary acyl chain] lipid A are less toxic than the wild-type lipid A. Mutations in the lipid A 4′-kinase encoding gene (lpxK) also decreases the toxic activity of lipid A.

Process c) thus involves either the deletion of part (or preferably all) of one or more of the above open reading frames or promoters. Alternatively, the promoters could be replaced with weaker promoters. Preferably the homologous recombination techniques are used to carry out the process. Preferably the methods described in WO 01/09350 (published by WIPO on Aug. 2, 2001 and incorporated by reference herein) are used. The sequences of the htrB and msbB genes from Neisseria meningitidis B, Moraxella catarrhalis, and Haemophilus influenzae are provided in WO 01/09350 for this purpose.

(d) A Process of Upregulating a Gene Involved in Rendering the Lipid a Portion of LPS Less Toxic

LPS toxic activity could also be altered by introducing mutations in genes/loci involved in polymyxin B resistance (such resistance has been correlated with addition of aminoarabinose on the 4′ phosphate of lipid A). These genes/loci could be pmrE that encodes a UDP-glucose dehydrogenase, or a region of antimicrobial peptide-resistance genes common to many enterobacteriaciae which could be involved in aminoarabinose synthesis and transfer. The gene pmrF that is present in this region encodes a dolicol-phosphate manosyl transferase (Gunn J. S., Kheng, B. L., Krueger J., Kim K., Guo L., Hackett M., Miller S. I. 1998. Mol. Microbiol. 27: 1171-1182).

Mutations in the PhoP-PhoQ regulatory system, which is a phospho-relay two component regulatory system (f. i. PhoP constitutive phenotype, PhoP^{c}), or low Mg⁺⁺ environmental or culture conditions (that activate the PhoP-PhoQ regulatory system) lead to the addition of aminoarabinose on the 4′-phosphate and 2-hydroxymyristate replacing myristate (hydroxylation of myristate). This modified lipid A displays reduced ability to stimulate E-selectin expression by human endothelial cells and TNF-α secretion from human monocytes.

Process d) involves the upregulation of these genes using a strategy as described in WO 01/09350 (published by WIPO on Aug. 2, 2001 and incorporated by reference herein).

(e) A Process of Down-Regulating Synthesis of an Antigen which Shares a Structural Similarity with a Human Structure and May be Capable of Inducing an Auto-Immune Response in Humans

The isolation of bacterial outer-membrane blebs from encapsulated Gram-negative bacteria often results in the co-purification of capsular polysaccharide. In some cases, this “contaminant” material may prove useful since polysaccharide may enhance the immune response conferred by other bleb components. In other cases however, the presence of contaminating polysaccharide material in bacterial bleb preparations may prove detrimental to the use of the blebs in a vaccine. For instance, it has been shown at least in the case of N. meningitidis that the serogroup B capsular polysaccharide does not confer protective immunity and is susceptible to induce an adverse auto-immune response in humans. Consequently, process e) of the invention is the engineering of the bacterial strain for bleb production such that it is free of capsular polysaccharide. The blebs will then be suitable for use in humans. A particularly preferred example of such a bleb preparation is one from N. meningitidis serogroup B devoid of capsular polysaccharide.

This may be achieved by using modified bleb production strains in which the genes necessary for capsular biosynthesis and/or export have been impaired as described in WO 01/09350 (published by WIPO on Aug. 2, 2001 and incorporated by reference herein). A preferred method is the deletion of some or all of the Neisseria meningitidis cps genes required for polysaccharide biosynthesis and export. For this purpose, the replacement plasmid pMF121 (described in Frosh et al.1990, Mol. Microbiol. 4:1215-1218) can be used to deliver a mutation deleting the cpsCAD (+galE) gene cluster. Alternatively the siaD gene could be deleted, or down-regulated in expression (the meningococcal siaD gene encodes alpha-2,3-sialyltransferase, an enzyme required for capsular polysaccharide and LOS synthesis). Such mutations may also remove host-similar structures on the saccharide portion of the LPS of the bacteria.

Combinations of Methods a)-e)

It may be appreciated that one or more of the above processes may be used to produce a modified strain from which to make improved bleb preparations of the invention. Preferably one such process is used, more preferably two or more (2, 3, 4, or 5) of the processes are used in order to manufacture the bleb vaccine. As each additional method is used in the manufacture of the bleb vaccine, each improvement works in conjunction with the other methods used in order to make an optimised engineered bleb preparation.

A preferred meningococcal (particularly N. meningitidis B) bleb preparation comprises the use of processes b), c) and e) (optionally combined with process a)). Such bleb preparations are safe (no structures similar to host structures), non-toxic, and structured such that the host immune response will be focused on high levels of protective (and preferably conserved) antigens. All the above elements work together in order to provide an optimised bleb vaccine.

Similarly for M. catarrhalis, non-typeable H. influenzae, gonococcus, and non serotype B meningococcal strains (e.g. serotype A, C, Y or W), preferred bleb preparations comprise the use of processes b) and c), optionally combined with process a).

Preferred Neisserial Bleb Preparations

One or more of the following genes (encoding protective antigens) are preferred for upregulation via process b) when carried out on a Neisserial strain, including gonococcus, and meningococcus (particularly N. meningitidis B): NspA (WO 96/29412), Hsf-like (WO 99/31132), Hap (PCT/EP99/02766), PorA, PorB, OMP85 (WO 00/23595), PilQ (PCT/EP99/03603), PldA (PCT/EP99/06718), FrpB (WO 96/31618), TbpA (U.S. Pat. No. 5,912,336), TbpB, FrpA/FrpC (WO 92/01460), LbpA/LbpB (PCT/EP98/05117), FhaB (WO 98/02547), HasR (PCT/EP99/05989), lipo02 (PCT/EP99/08315), Tbp2 (WO 99/57280), MItA (WO 99/57280), and ctrA (PCT/EP00/00135). They are also preferred as genes which may be heterologously introduced into other Gram-negative bacteria.

One or more of the following genes are preferred for downregulation via process a): PorA, PorB, PilC, TbpA, TbpB, LbpA, LbpB, Opa, and Opc (most preferably PorA).

One or more of the following genes are preferred for downregulation via process c): htrB, msbB and lpxK (most preferably msbB which removes only a single secondary acyl chain from the LPS molecule).

One or more of the following genes are preferred for upregulation via process d): pmrA, pmrB, pmrE, and pmrF.

One or more of the following genes are preferred for downregulation via process e): galE, siaA, siaB, siaC, siaD, ctrA, ctrB, ctrc, and ctrD (the genes are described in described in WO 01/09350—published by WIPO on Aug. 2, 2001 and incorporated by reference herein).

Many of the above open reading frames and upstream regions are described in WO 01/09350 (incorporated by reference herein).

Preferred gonococcal genes to upregulate via process b) include one or more of the following:

| Neisseria gonorrheae lactoferrin receptor precursor (lbpA) gene, | | |
|---|---|---|
| complete cds. | | |
| ACCESSION U16260 | | |
| VERSION U16260.1 GI:915277 | | |
| Source: Neisseria gonorrhoeae/strain = “FA19” | | |
| gene = “lbpA” nucleotides: 278..3109 | | |
| protein_id = “AAC13780.1”/db_xref = “GI:915278” | | |
| /translation = “MNKKHGFPLTLTALAIATAFPAYAAQAGAAALDAAQSQSLKEVT | | |
| VRAAKVGRRSKEATGLGKIVKTSETLNKEQVLGIRDLTRYDPGVAVVEQGNGASGGYS | | |
| IRGVDKNRVAVSVDGVAQIQAFTVQGSLSGYGGRGGSGAINEIEYENISTVEIDKGAG | | |
| SSDHGSGALGGAVAFRTKEAADLISDGKSWGIQAKTAYGSKNRQFMKSLGAGFSKDGW | | |
| EGLLIRTERQGRETRPHGDIADGVEYGIDRLDAFRQTYDIKRKTTEPFFLVEGENTLK | | |
| PVAKLAGYGIYLNRQLNRWVKERIEQNQPLSAEEEAQVREAQARHENLSAQAYTGGGR | | |
| ILPDPMDYRSGSWLAKLGYRFGGRHYVGGVFEDTKQRYDIRDMTEKQYYGTDEAEKFR | | |
| DKSGVYDGDDFRDGLYFVPNIEEWKGDKNLVKGIGLKYSRTKFIDEHHRRRRMGLLYR | | |
| YENEKYSDNWADKAVLSFDKQGVATDNNTLKLNCAVYPAVDKSCRASADKPYSYDSSD | | |
| RFHYREQHNVLNASFEKSLKNKWTKHHLTLGFGYDASKAVSRPEQLSHNAARISESTG | | |
| FDEKNQDKYRLGKPEVVEGSVCGYIETLRSRKCVPRKINGSNIHISLNDRFSIGKYFD | | |
| FSLGGRYDRKNFTTSEELVRSGRYADRSWNSGIVFKPNRHFSVSYRASSGFRTPSFQE | | |
| LFGIDIYHDYPKGWQRPALKSEKAANREIGLQWKGDFGFLEISSFRNRYTDMIAVADQ | | |
| KTKLPDSAGRLTEIDIRDYYNAQNMSLQGINILGKIDWNGVYGKLPEGLYTTLAYNRI | | |
| KPKSVSNRPDLSLRSYALDAVQPSRYVLGFGYDQPEGKWGANIMLTYSKGKNPDELAY | | |
| LAGDQKRYSAGRVTSSWKTADVSAYLNLKKRLTLRAAIYNIGNYRYVTWESLRQTAES | | |
| TANRHGGDSNYGRYAAPGRNFSLALEMKF” | | |
| | 1 | ctcgggataa cggcatcaat ctttcgggaa atggttcgac taatcctcaa |
| | | agtttcaaag |
| | 61 | ccgacaatct tcttgtaacg ggcggctttt acggcccgca ggcggcggaa |
| | | ttgggcggca |
| | 121 | ctattttcaa taaggatggg aaatctcttg gtataactga agatattgaa |
| | | aatgaagttg |
| | 181 | aaaatgaagc tgatgttggc gaacagttag aacctgaagt taaaccccaa |
| | | ttcggcgtgg |
| | 241 | tattcggtgc gaagaaagat aataaagagg tggaaaaatg aataagaaac |
| | | acggttttcc |
| | 301 | gctgactttg acggcgttgg ccattgcaac cgcttttccg gcttatgctg |
| | | cccaagcggg |
| | 361 | ggcggcggca cttgatgcgg cgcaaagtca atcattgaaa gaggttaccg |
| | | tccgtgccgc |
| | 421 | caaagtggga cggcgatcga aagaggcgac aggtttgggc aaaatcgtca |
| | | aaacgtcgga |
| | 481 | aacgttgaac aaagaacagg tactcggtat ccgcgacctg acgcgctacg |
| | | atccgggcgt |
| | 541 | ggcggttgtc gaacagggca acggcgcgag cggcggctac tcgatacgcg |
| | | gcgtagataa |
| | 601 | aaaccgtgtg gcggtttcgg ttgacggcgt tgcccaaata caggcgttta |
| | | ccgtgcaggg |
| | 661 | atcgttgagc ggatacggcg gacgcggcgg cagcggcgca atcaacgaaa |
| | | tcgaatatga |
| | 721 | aaacatcagc acggtggaaa tcgacaaagg cgccggttcg tccgatcacg |
| | | gcagcggcgc |
| | 781 | actcggcggc gcggtcgcct tccgcaccaa agaggcggca gacctgattt |
| | | cagacggcaa |
| | 841 | aagctggggg atacaggcaa aaaccgctta cggcagtaaa aaccgccaat |
| | | ttatgaagtc |
| | 901 | gctcggcgcg gggttcagca aagacggttg ggaagggctg ctaatccgaa |
| | | ccgaacgcca |
| | 961 | agggcgggaa acgcgcccgc acggcgatat tgcggacggg gtggaatacg |
| | | gcatagaccg |
| | 1021 | tttggacgcg ttccgccaga catacgatat taaacgcaag acaacagagc |
| | | catttttctt |
| | 1081 | agtagagggc gagaatacac tcaagcccgt ggcaaaattg gcgggctacg |
| | | ggatatattt |
| | 1141 | gaaccgccag ctcaaccgct gggtaaaaga acgtattgaa caaaatcagc |
| | | ctttaagtgc |
| | 1201 | tgaagaagag gcgcaggtgc gggaggcgca ggcgcgccac gaaaacctgt |
| | | ccgcccaagc |
| | 1261 | ctacacgggc ggcggcagga tattgcccga tccgatggat taccgcagcg |
| | | gctcttggct |
| | 1321 | tgccaagctg ggctaccgct tcggcggcag gcattatgtc ggcggcgtgt |
| | | ttgaggatac |
| | 1381 | caaacagcgt tacgacatcc gcgatatgac ggaaaaacag tattacggta |
| | | cggacgaggc |
| | 1441 | ggaaaagttt agagacaaga gcggggtgta cgacggcgac gatttccgcg |
| | | acggcttgta |
| | 1501 | ttttgtgccg aatatagaag agtggaaggg cgataaaaat ttggtcaagg |
| | | gcataggttt |
| | 1561 | gaaatattcc cgcaccaaat ttattgacga acatcaccgc cgccgccgta |
| | | tgggtttgct |
| | 1621 | gtaccgttat gaaaatgaga aatactcgga caactgggcg gataaggcgg |
| | | tgttgtcgtt |
| | 1681 | tgacaaacag ggcgtggcaa ccgacaacaa cacgctgaag ctgaattgcg |
| | | ccgtgtatcc |
| | 1741 | tgccgtggac aaatcctgcc gcgcgtcggc ggacaaaccg tattcctacg |
| | | acagcagcga |
| | 1801 | ccgtttccac taccgcgaac agcacaatgt tttgaatgcc tcgtttgaga |
| | | agtcgctgaa |
| | 1861 | aaacaaatgg acgaaacacc atctgacttt gggcttcggt tacgatgctt |
| | | ccaaagcagt |
| | 1921 | atcccgccca gaacagcttt cccacaatgc ggcaaggatt tcggaatcca |
| | | cgggattcga |
| | 1981 | tgaaaagaat caagataagt accgtttggg taagcccgaa gtcgtcgaag |
| | | ggtcggtctg |
| | 2041 | cggctatatc gaaaccctgc gttcccgcaa atgcgtgcca agaaaaatca |
| | | acggcagcaa |
| | 2101 | tatccacatt tctttgaacg accgtttttc aatcggcaaa tattttgatt |
| | | tcagcttggg |
| | 2161 | cggcaggtac gaccggaaaa acttcaccac gtcggaagaa ctcgtccgca |
| | | gcgggcggta |
| | 2221 | tgccgaccgt tcgtggaaca gcggcatcgt gttcaaaccg aaccggcatt |
| | | tttccgtgtc |
| | 2281 | ttaccgcgcc tccagcggct tcagaacgcc ttccttccaa gaacttttcg |
| | | ggatagacat |
| | 2341 | ttatcacgat tatccgaaag gctggcagcg tcccgccctg aaatcggaaa |
| | | aggcagccaa |
| | 2401 | ccgggaaatc ggtttgcagt ggaagggcga tttcggcttt ttggaaatca |
| | | gcagtttccg |
| | 2461 | caaccgttat accgatatga ttgccgttgc cgatcaaaaa accaaattgc |
| | | cggattcagc |
| | 2521 | aggacgattg acagagattg atatacgcga ttattacaat gcccaaaata |
| | | tgtcgcttca |
| | 2581 | aggcatcaac atcttgggga aaatcgactg gaacggcgta tacggcaaac |
| | | tgcccgaagg |
| | 2641 | cctgtacacc acattggcgt acaaccgtat caaaccgaaa tcggtatcca |
| | | accggccgga |
| | 2701 | cttgtccctc cgcagctatg ctttggatgc ggtacagccg tcgcgttatg |
| | | ttttggggtt |
| | 2761 | cggatacgac cagcccgagg ggaaatgggg cgcaaacatt atgctgacct |
| | | attccaaagg |
| | 2821 | gaaaaaccct gacgagcttg cttatctggc aggcgatcaa aaacgatatt |
| | | cggcaggaag |
| | 2881 | ggttacgtct tcttggaaaa cggcagatgt ttccgcttat ctgaatctga |
| | | aaaaacggct |
| | 2941 | gaccttgagg gcggctatct acaatatcgg caactaccgc tacgttactt |
| | | gggaatcctt |
| | 3001 | gcgccagact gcggaaagca cggcaaaccg gcacggcggc gacagcaact |
| | | atggaaggta |
| | 3061 | tgccgcaccg ggcaggaact tcagcctcgc gctcgaaatg aagttttaaa |
| | | ggaaatgccg |
| | 3121 | tctggaagct tgatctgcac cccaaaagtc ggactaaacc gccaactgat |
| | | taaggtgcag |
| | 3181 | gtttttttga ttcaatataa acaagatttc cgccgtcatt cccgcgcagg |
| | | cgggaatccg |
| | 3241 | gacattcaat gctaaggcaa tttatcggaa atgactgaaa ctcaaaaaac |
| | | cggattccca |
| | // | |

| Neisseria gonorrhoeae lactoferrin binding protein B precursor. | | |
|---|---|---|
| Source: Neisseria gonorrhoeae “/strain = “FA19” | | |
| ACCESSION AAD08809 | | |
| PID g4106393 | | |
| VERSION AAD08809.1 GI:4106393 | | |
| /gene = “lbpB”coding sequence: 1..728, “AF072890.1:310..2496” | | |
| | 1 | mrklnyygia llplmlascg gnfgvqpvve stptaypvtf kskdvptspp |
| | | paepsvettp |
| | 61 | vnrpavgaam rllrrntafh redgtaipds kqaeeklsfk egdvlflygs |
| | | kgnklqqlks |
| | 121 | eihkrdsdve irtsekenkk ygyefvdagy vytkngkdei eqnsggkrft |
| | | hrfgydgfvy |
| | 181 | ysgerpsqsl psagtvkyfg nwqymtdakr hrtgkavasd dlgyitfygn |
| | | digatsyaak |
| | 241 | daddrekhpa eytvdfdkki lkgelikncw vqkkndpkkp ltiynitadl |
| | | ngnrftgsak |
| | 301 | vntevktrha dkeylffhtd adgrleggff gdngeelagr fisndngvfg |
| | | vfagkqktna |
| | 361 | sgtnpampfg khtkildslk isvdeatden prpfevstmp dfghpdkllv |
| | | egreiplvsk |
| | 421 | ektidladgr kmtvsaccdf ltyvklgrik terpavkpka qdeedsginn |
| | | geesedeeei |
| | 481 | aeesedevse ddngededei veeeadeaee ieeeaeeeep eeespeegng |
| | | vsdgippape |
| | 541 | alkgrdidlf lkgirtaead ipktgtahyt gtwearigep iqwdnkadka |
| | | akaefdvdfg |
| | 601 | nksisgtlte qngvepafri engviegngf hptartrdng inlsgngstn |
| | | pqsfkadnll |
| | 661 | vtggfygpga aelggtifnk dgkslgited ienevenead vgeqlepevk |
| | | pqfgvvfgak |
| | 721 | kdnkevek |

| Neisseria gonorrhoeae transferrin-binding protein A (tbpA) gene, | | |
|---|---|---|
| complete cds. | | |
| ACCESSION AF241227 | | |
| VERSION AF241227.1 G1:9719361 | | |
| source :Neisseria gonorrhoeae/strain = “Pgh3-2” | | |
| gene “tbpA”coding sequence: 223..2946 | | |
| /proteinid_id = “AAF97766.1” | | |
| /db_xref = “GI:9719362” | | |
| /translation = “MQQQHLFRFNILCLSLMTALPAYAENVQAGQAQEKQLDTIQVKA | | |
| KKQKTRRDNEVTGLGKLVKTADTLSKEQVLDIRDLTRYDPGIAVVEQGRGASSGYSIR | | |
| GMDKNRVSLTVDGLAQIQSYTAQAALGGTRTAGSSGAINEIEYENVKAVEISKGSNSV | | |
| EQGSGALAGSVAFQTKTADDVIGEGRQWGIQSKTAYSGKNRGLTQSIALAGRIGGAEA | | |
| LLIRTGRHAGEIRAHEAAGRGVQSFNRLAPVEDGSDYAYFVVEGECPDGYAACKDKPK | | |
| KDVVGEDKRQTVSTRDYTGPNRFLADPLSYESRSWLFRPGFRFENKRHYIGGILERTQ | | |
| QTFDTRDMTVPAFLTKAVFDANSKQAGSLRGNGKYAGNHKYGGLFTNGENNAPVGAEY | | |
| GTGVFYDETHTKSRYGLEYVYTNADKDTWADYARLSYDRQGIGLDNHFQQTHCSADGS | | |
| DKYCRPSADKPFSYYKSDRVIYGESHRLLQAAFKKSFDTAKIRHNLSVNLGYDRFGSN | | |
| LRHQDYYYQSANRAYSLKTPPQNNGKKTSPYWVSIGRGNVVTGQICRSCNNTYTDCTP | | |
| RSINGKSYYAAVRDNVRLGRWADVGAGLRYDYRSTHSDDGSVSTGTHRTLSWNTGIVL | | |
| KPADWLDLTYRTSTGFRLPSFAEMYGWRSGDKIKAVKIDPEKSFNKEAGIVFKGDFGN | | |
| LEASWFDNAYRDLIVRGYEAEIKNGKEQAKGAPAYLNAQSARITGINILGKIDWNGVW | | |
| DKLPEGWYSTFAYNRVRVRDIKKRADRTDIQSHLFDAIQPSRYVVGSGYDQPEGKWGV | | |
| NGMLTYSKAKEITELLGSRALLNGNSRNTKATARRTRPWYIVDVSGYYTVKKHFTLRA | | |
| GVYNLLNHRYVTWENVRQTAAGAVNQHKNVGVYNRYAAPGRNYTFSLEMKF” | | |
| | 1 | cgaagagttg ggcggatggt ttgcctatcc gggcaatgaa caaacgaaaa atgcgcaagc |
| | 61 | ttcatccggc aatggaaatt cagcaggcag cgcgaccgtg gtattcggtg |
| | | cgaaacgcca |
| | 121 | aaagcttgtg caataagcac ggctgccgaa caatcgagaa taaggcttca |
| | | gacggcatcg |
| | 181 | ttcctgccga ttccgtctga aagcgaagat tagggaaaca ctatgcaaca |
| | | gcaacatttg |
| | 241 | ttccgattca atattttatg cctgtcttta atgactgcgc tgcccgctta |
| | | tgcagaaaat |
| | 301 | gtgcaagccg gacaagcaca ggaaaaacag ttggacacca tacaggtaaa |
| | | agccaaaaaa |
| | 361 | cagaaaaccc gccgcgataa cgaagtaacc ggtttgggca aattggtcaa |
| | | aaccgccgac |
| | 421 | acactcagca aagaacaggt actcgacatc cgcgacctga cgcgttacga |
| | | ccccggcatc |
| | 481 | gccgtcgtcg aacaggggcg cggcgcaagc tcgggctact cgatacgcgg |
| | | tatggacaaa |
| | 541 | aaccgcgtct ccttgacggt ggacggcttg gcgcaaatac agtcctacac |
| | | cgcgcaggcg |
| | 601 | gcattgggcg ggacgaggac ggcgggcagc agcggcgcaa tcaatgaaat |
| | | cgagtatgaa |
| | 661 | aacgttaagg ctgtcgaaat cagcaaaggc tcaaactcgg tcgaacaagg |
| | | cagcggcgca |
| | 721 | ttggcgggtt cggtcgcatt tcaaaccaaa accgcagacg atgttatcgg |
| | | ggaaggcagg |
| | 781 | cagtggggca ttcagagtaa aaccgcctat tccggcaaaa accgggggct |
| | | tacccaatcc |
| | 841 | atcgcgctgg cggggcgcat cggcggtgcg gaggctttgc tgatccgcac |
| | | cggccggcac |
| | 901 | gcgggggaaa tccgcgccca cgaagccgcc ggacgcggcg ttcagagctt |
| | | taacaggctg |
| | 961 | gcgccggttg aagacggcag tgactatgcc tattttgtgg tcgaaggaga |
| | | atgccctgat |
| | 1021 | ggatatgcgg cttgtaaaga caaaccgaaa aaagatgttg tcggcgaaga |
| | | caaacgtcaa |
| | 1081 | acggtttcca cccgagacta cacgggcccc aaccgcttcc ttgccgatcc |
| | | gctttcatac |
| | 1141 | gaaagccggt cgtggctgtt ccgcccgggt tttcgttttg agaataagcg |
| | | gcactacatc |
| | 1201 | ggcggcatac tcgaacgcac gcaacaaact ttcgacacgc gcgatatgac |
| | | ggttccggca |
| | 1261 | ttcctgacca aggcggtttt tgatgcaaat tcaaaacagg cgggttcttt |
| | | gcgcggcaac |
| | 1321 | ggcaaatacg cgggcaacca caaatacggc gggctgttta ccaacggcga |
| | | aaacaatgcg |
| | 1381 | ccggtgggcg cggaatacgg tacgggcgtg ttttacgacg agacgcacac |
| | | caaaagccgc |
| | 1441 | tacggtttgg aatatgtcta taccaatgcc gataaagaca cttgggcgga |
| | | ttatgcccgc |
| | 1501 | ctctcttacg accggcaggg catcggtttg gacaaccatt ttcagcagac |
| | | gcactgttct |
| | 1561 | gccgacggtt cggacaaata ttgccgcccg agtgccgaca agccgttttc |
| | | ctattacaaa |
| | 1621 | tccgaccgcg tgatttacgg ggaaagccac aggctcttgc aggcggcatt |
| | | caaaaaatcc |
| | 1681 | ttcgataccg ccaaaatccg ccacaacctg agcgtgaatc tcggttacga |
| | | ccgcttcggc |
| | 1741 | tctaatctgc gccatcagga ttattattat caaagtgcca accgcgccta |
| | | ttcgttgaaa |
| | 1801 | acgccccctc aaaacaacgg caaaaaaacc agcccctatt gggtcagcat |
| | | aggcagggga |
| | 1861 | aatgtcgtta cggggcaaat ctgccgctcg ggcaacaata cttatacgga |
| | | ctgcacgccg |
| | 1921 | cgcagcatca acggcaaaag ctattacgcg gcggtccggg acaatgtccg |
| | | tttgggcagg |
| | 1981 | tgggcggatg tcggcgcggg cttgcgctac gactaccgca gcacgcattc |
| | | ggacgacggc |
| | 2041 | agcgtttcca ccggcacgca ccgcaccctg tcctggaaca ccggcatcgt |
| | | cctcaaacct |
| | 2101 | gccgactggc tggatttgac ttaccgcact tcaaccggct tccgcctgcc |
| | | ctcgtttgcg |
| | 2161 | gaaatgtacg gctggcggtc gggcgataaa ataaaagccg tcaaaatcga |
| | | tccggaaaaa |
| | 2221 | tcgttcaaca aagaagccgg catcgtgttt aaaggcgatt tcggcaactt |
| | | ggaggcaagt |
| | 2281 | tggttcgaca atgcctaccg cgatttgatt gtccggggtt atgaagcgga |
| | | aattaaaaac |
| | 2341 | ggcaaagaac aagccaaagg cgccccggct tacctcaatg cccaaagcgc |
| | | gcggattacc |
| | 2401 | ggcatcaata ttttgggcaa aatcgattgg aacggcgtat gggataaatt |
| | | gcccgaaggt |
| | 2461 | tggtattcta catttgccta taatcgtgtc cgtgtccgcg acatcaaaaa |
| | | acgcgcagac |
| | 2521 | cgcaccgata ttcaatcaca cctgtttgat gccatccaac cctcgcgcta |
| | | tgtcgtcggc |
| | 2581 | tcgggctatg accaaccgga aggcaaatgg ggcgtgaacg gtatgctgac |
| | | ttattccaaa |
| | 2641 | gccaaggaaa tcacagagtt gttgggcagc cgggctttgc tcaacggcaa |
| | | cagccgcaat |
| | 2701 | acaaaagcca ccgcgcgccg tacccgccct tggtatattg tggatgtgtc |
| | | cggttattac |
| | 2761 | acggttaaaa aacacttcac cctccgtgcg ggcgtgtaca acctcctcaa |
| | | ccaccgctat |
| | 2821 | gttacttggg aaaatgtgcg gcaaactgcc gccggcgcag tcaaccaaca |
| | | caaaaatgtc |
| | 2881 | ggcgtttaca accgatatgc cgcccccggc cgcaactaca catttagctt |
| | | ggaaatgaag |
| | 2941 | ttctaaacgt ccgaacgccg caaatgccgt ctgaaaggct tcagacggcg |
| | | ttttttacac |
| | 3001 | aatccccacc gtttcccatc cttcccgata caccg |

| Neisseria gonorrhoeae strain UU1008 transferrin-binding protein 2 | | |
|---|---|---|
| (tbpB) gene, complete cds. | | |
| ACCESSION U65222 | | |
| VERSION U65222.1 GI:2286066 | | |
| Source: Neisseria gonorrhoeae/strain = “UU1008” | | |
| gene = “tbpB”coding sequence: 1..2052 | | |
| /protein_id = “AAB64243.1” | | |
| /db_xref = “GI:2286O67” | | |
| /translation = “MNNPLVNQAAMVLPVFLLSACLGGGGSFDLDSVDTEAPPAAPKY | | |
| QDVPSKKPEARKDQGGYGFAMRFKRRNWYRAANENEVKLKESDWEQTDDDEIKNPFKQ | | |
| KNIINALPGNEGELLQDSSQQGKGTSKVRDHHDFKYVWSGFFYKRIKITTKKDESHKI | | |
| IEARSGPDGYIFYKGRNPSRKLPVSGEVTYKGTWDFLTDVKANQKFTDLGNASTKSGD | | |
| QYSAFSGELDYIVKKEEDKKEKHKGLGLTTEITVDFEKKTLIGKLIKNNMLINNNTKP | | |
| TTQYYSLEAQVTGNRFSGKANATEKGENKQHPFVSDSSSLSGGFFGPQGEELGFRFLS | | |
| DDGKVAVVGSAKTKDETASSGGTSGGASVSTSNGAAGTSSENKLTTVLDAVELTPNGK | | |
| KIKDLDNFSNAAQLVVDGIMIPLLPKDSESGGSHTDKGENGKTAFIYETTYTPESDKE | | |
| DAQTGMATNGVQTVSNTAGGTSGKTKTHYEVQACCSNLNYLKYGLLTRKNSSQADAKM | | |
| GQVEQSMFLQGERTDEKEIPQEQNVVYSGTWYGHIATNGTSWTREASDQENGNRANFD | | |
| VNFKDKRITGTLTAENRSEATFTIEAMIEGNGFKGTAKTGNGGFAPDQNSSTGTHKVH | | |
| ITNAAVQGGFYGPNAEELGGWFAYPGNGQTKNAQTSSGNGNSAGSATVVFGAKRQQLV | | |
| K” | | |
| | 1 | atgaacaatc cattggtgaa tcaggctgct atggtgctgc ccgtgttttt gttgagcgct |
| | 61 | tgtctgggcg gaggcggcag tttcgatctt gattctgtcg ataccgaagc |
| | | cccgcgtgcc |
| | 121 | gcgccaaagt atcaagatgt tccttccaaa aaaccggaag cccgaaaaga |
| | | ccaaggcgga |
| | 181 | tacggtttcg cgatgcgctt caagcggcgg aattggtatc gggcggcaaa |
| | | cgaaaacgag |
| | 241 | gttaaactga aagagagtga ttgggaacaa acggatgatg atgagatcaa |
| | | aaaccctttc |
| | 301 | aaacaaaaaa atattattaa tgccttacct ggaaatgagg gggaattatt |
| | | gcaagattcc |
| | 361 | agtcaacaag gtaagggtac atctaaggtt agggaccatc acgattttaa |
| | | atacgtatgg |
| | 421 | tcgggttttt tttataaacg gattaagatt acaactaaaa aagacgaatc |
| | | tcataaaata |
| | 481 | atcgaagcca gaagcggtcc tgacggttat attttttata aaggcagaaa |
| | | tccctcgaga |
| | 541 | aaacttcctg tttcagggga ggttacgtac aaaggtactt gggatttttt |
| | | aactgatgtg |
| | 601 | aaagcaaatc agaaatttac agatttagga aatgcttcta cgaaatccgg |
| | | agaccaatat |
| | 661 | agtgcttttt ccggggagtt ggattatata gtcaaaaaag aggaggataa |
| | | aaaagaaaag |
| | 721 | cacaaaggtt tgggattaac aacggaaata acggttgatt ttgagaaaaa |
| | | aaccctgatc |
| | 781 | ggaaaattaa ttaaaaacaa catgttaatc aataataaca ctaaacccac |
| | | cacccaatat |
| | 841 | tacagccttg aggctcaagt aacaggcaac cgcttcagcg gcaaggcgat |
| | | ggcaaccgaa |
| | 901 | aaaggcgaaa acaaacaaca tccctttgtt tccgactcgt cttctctgag |
| | | cggcggcttt |
| | 961 | ttcggcccgc agggtgagga attgggtttc cgctttttga gcgacgatgg |
| | | aaaagttgcc |
| | 1021 | gttgtcggca gcgcgaaaac caaagacgaa accgcaagca gtggcggcac |
| | | ttcgggcggt |
| | 1081 | gcaagcgttt ccacatcaaa cggtgcggca ggcacgtcgt ctgaaaacaa |
| | | gctgaccacg |
| | 1141 | gttttggatg cggttgaatt gacaccaaac ggcaagaaaa tcaaagatct |
| | | cgacaacttc |
| | 1201 | agcaacgccg cccaactggt tgtcgacggc attatgattc cgctcctgcc |
| | | caaggattcc |
| | 1261 | gaaagcgggg gcagtcatac agataaaggt gaaaacggca aaacagcctt |
| | | tatctacgaa |
| | 1321 | acaacctaca cgccggaaag tgataaagaa gacgctcaaa caggtatggc |
| | | gaccaatggc |
| | 1381 | gtgcaaaccg tttcaaatac ggcaggcggc acaagtggca aaacaaaaac |
| | | ccattatgaa |
| | 1441 | gtccaagcct gctgttccaa cctcaattat ctgaaatacg ggttgctgac |
| | | gcgtaaaaac |
| | 1501 | agtagtcaag ctgacgctaa aatgggacaa gttgaacaaa gtatgttcct |
| | | ccaaggcgag |
| | 1561 | cgcaccgatg aaaaagaaat tccacaagaa caaaatgtcg tttattcagg |
| | | cacttggtac |
| | 1621 | gggcatattg ccaccaacgg cacaagttgg acccgcgaag cctccgatca |
| | | ggaaaatggt |
| | 1681 | aatcgggcaa attttgacgt gaatttcaaa gacaaaagaa ttaccggcac |
| | | gttaaccgct |
| | 1741 | gaaaacaggt cggaggcaac ctttaccatt gaagccatga ttgagggcaa |
| | | cggctttaaa |
| | 1801 | ggtacggcga aaaccggtaa tggcggcttt gcgccggatc aaaacagcag |
| | | caccggtaca |
| | 1861 | cataaagtgc acatcacaaa tgccgcggtg cagggcggtt tttacgggcc |
| | | taacgccgaa |
| | 1921 | gagttgggcg gttggtttgc ctatccgggc aatggacaaa cgaaaaatgc |
| | | gcaaacttca |
| | 1981 | tccggcaatg gaaattcagc aggcagcgcg accgtggtat tcggtgcgaa |
| | | acgccaacag |
| | 2041 | cttgtgaaat aa |

| Neisseria gonorrhoeae pilus biogenesis gene cluster, pilO, pilP and | | |
|---|---|---|
| pilQ genes, complete cds. | | |
| ACCESSION U40596 | | |
| VERSION U40596.1 GI:1173872 | | |
| source: Neisseria gonorrhoeae/strain = “MS11” | | |
| gene = “pilO”coding sequence 22..669 | | |
| /protein_id = “AAC43601.1” | | |
| /db_xref = “GI:1173873” | | |
| /translation = “MASKSSKTNLDLNNLHLLNLPARLFIALLVVAAVLGLGYAGLFK | | |
| SQMESLEEYEAKETELKNTYKQKSIDAASLNNLRDELASIRSAFDIMLKQLPTDAEIP | | |
| NLVQELHQAGSSNGLRLDSVMPQPPVDDGPIKKLPYSISITGNYEQISQFTRDVGSLS | | |
| RIITLESLKIAQSPENGGNPDGKSSILNLSAIATTYQAKSIEELAAEAAQNAEQK” | | |
| gene= “pilP”coding sequence 687..1229 | | |
| /protein_id = “AAC43602.1” | | |
| /db_xref = “GI:1173874” | | |
| /translation= “MKHYALLISFLALSACSQSSEDLNEWMAQTRREAKAEIIPFQAP | | |
| TLPVAPVYSPPQLTGPNAFDFRRMETAKKGENAPDTKRIKETLEKFSLENMRYVGILK | | |
| SGQKVSGFIEAEGYVYTVGVGNYLGQNYGRIESITDDSIILNELIEDSTGNWVSRKAE | | |
| LLLNSSDKNTEQAAQPEEQN | | |
| gene= “pilQ”coding sequence 1248..3410 | | |
| /protein_id= “AAC43603.1” | | |
| /db_xref= “GI:1173875” | | |
| /translation= “MNTKLTKIISGLFVATAAFQTASAGNITDIKVSSLPNKQKIVKV | | |
| SFDKEIVNPTGFVTSSPARIALDFEQTGISMDQQVLEYADPLLSKISAAQNSSRARLV | | |
| LNLNKPGQYNTEVRGNKVWIFINESDDTVSAPARPAVKAAPAAPAKQQAAAPFTESVV | | |
| SVSAPFSPAKQQAAASAKQQAATPAKQTNIDFRKDGKNAGIIELAALGFAGQPDISQQ | | |
| HDHIIVTLKNHTLPTALQRSLDVADFKTPVQKVTLKRLNNDTQLIITTTGNWELVNKS | | |
| AAPGYFTFQVLPKKQNLESGGVNNAPKTFTGRKISLDFQDVEIRTILQILAKESGMNI | | |
| VASDSVSGKMTLSLKDVPWDQALDLVMQARNLDMRQQGNIVNMAPRRAACQRQSLLTS | | |
| GKRHCRSGRAVFPKLPIEIQKCGRIPQHPALDNADTTGNRNTLVSGRGSVLIDPATNT | | |
| LIVTDTRSVIEKFRKLIDELDVPAQQVMIEARIVEAADGFSRDLGVKFGATGRKKLKN | | |
| ETSAFGWGVNSGFGGCDKWEAKPKSTCRLPCRKQHFAGARDFSGALNLELSASESLSK | | |
| TKTLANPRVLTQNRKEAKIESGYEIPFTVTTRSGGGNSTNTELKKAVLGLTVTANITP | | |
| DOQIIMTVKINKDSPRQCASGNNTILCTSTKSLNTQAMVENGGTLIVGGIYEENNGNT | | |
| LTKVPLLATSPLSATSLKHSGKNRPPRTADFQLPPREL” | | |
| | 1 | aaacgcacag gaggaaactg aatggcttct aaatcatcta aaaccaactt |
| | | ggatctcaac |
| | 61 | aaccttcacc tgctcaacct tcctgccagg ctttttatcg ccctgctggt |
| | | cgttgccgcc |
| | 121 | gtgctggggc tcggttatgc cggattgttc aaaagccaga tggaatccct |
| | | tgaggaatat |
| | 181 | gaagcaaaag aaaccgaact gaaaaacacc tacaaacaga aaagtatcga |
| | | cgcggccagc |
| | 241 | ctgaacaacc ttagggacga acttgcctca atccgctctg ccttcgatat |
| | | catgttgaaa |
| | 301 | cagctgccga cagatgcaga aattcccaat ttggttcaag agcttcatca |
| | | ggcgggttcg |
| | 361 | agcaacggtc tgcgcttgga cagcgttatg ccccaacctc ccgtagatga |
| | | cggtcccatc |
| | 421 | aaaaaattac cctattccat ttccattacc ggaaattacg aacagatcag |
| | | ccaatttacc |
| | 481 | cgcgatgtcg gcagtctctc ccgaatcatt acccttgagt cgctgaaaat |
| | | cgcccaatct |
| | 541 | ccggaaaacg gcggcaatcc tgacggcaag agcagtatcc tgaacctcag |
| | | cgccattgcc |
| | 601 | accacctacc aagcaaaatc catagaagag cttgccgcag aagcggcaca |
| | | aaatgccgag |
| | 661 | caaaaataac ttacgttagg gaaaccatga aacactatgc cttactcatc |
| | | agctttctgg |
| | 721 | ctctctccgc gtgttcccaa agttctgaag acctaaacga atggatggca |
| | | caaacgcgac |
| | 781 | gcgaagccaa agcagaaatc atacctttcc aagcacctac cctgccggtt |
| | | gcgccggtat |
| | 841 | acagcccgcc gcagcttaca gggccgaacg cattcgactt ccgccgcatg |
| | | gaaaccgcca |
| | 901 | aaaaagggga aaatgccccc gacaccaagc gtattaaaga aacgctggaa |
| | | aaattcagtt |
| | 961 | tggaaaatat gcgttatgtc ggcattttga agtccggaca gaaagtctcc |
| | | ggcttcatcg |
| | 1021 | aggctgaagg ttatgtctac actgtcggtg tcggcaacta tttgggacaa |
| | | aactacggta |
| | 1081 | gaatcgaaag cattaccgac gacagcatca tcctgaacga gctgatagaa |
| | | gacagcacgg |
| | 1141 | gcaactgggt ttcccgtaaa gcagaactgc tgttgaattc ttccgacaaa |
| | | aacaccgaac |
| | 1201 | aagcggcaca gcctgaggaa caaaattaag aagaggatta ctccattatg |
| | | aataccaaac |
| | 1261 | tgacaaaaat catttccggt ctctttgtcg caaccgccgc ctttcagacg |
| | | gcatcggcag |
| | 1321 | gaaacattac agacatcaaa gtttcctccc tgcccaacaa acagaaaatc |
| | | gtcaaagtca |
| | 1381 | gctttgacaa agagattgtc aacccgaccg gcttcgtaac ctcctcaccg |
| | | gcccgcatcg |
| | 1441 | ccttggactt tgaacaaacc ggcatttcca tggatcaaca ggtactcgaa |
| | | tatgccgatc |
| | 1501 | ctctgttgag caaaatcagt gccgcacaaa acagcagccg tgcgcgtctg |
| | | gttctgaatt |
| | 1561 | tgaacaaacc gggccaatac aataccgaag tacgcgggaa caaagtttgg |
| | | atattcatta |
| | 1621 | acgaatcgga cgataccgtg tccgcccccg cccgcccagc cgtaaaagcc |
| | | gcgcctgccg |
| | 1681 | caccggcaaa acaacaggct gccgcaccgt ttaccgagtc cgtagtatcc |
| | | gtatccgcac |
| | 1741 | cgttcagccc ggcaaaacaa caggcagcgg catcggcaaa acaacaggcg |
| | | gcgacaccgg |
| | 1801 | caaaacaaac caatatcgat ttccgcaaag acggcaaaaa tgccggcatt |
| | | atcgaattgg |
| | 1861 | cggcattggg ctttgccggg cagcccgaca tcagccaaca gcacgaccac |
| | | atcatcgtta |
| | 1921 | cgctgaaaaa ccataccctg ccgaccgcgc tccaacgcag tttggatgtg |
| | | gcagacttca |
| | 1981 | aaacaccggt tcaaaaggtt acgctgaaac gcctcaataa cgacacccag |
| | | ctgattatca |
| | 2041 | caacaaccgg caactgggaa ctcgtcaaca aatccgccgc gcccggatac |
| | | tttaccttcc |
| | 2101 | aagtcctgcc gaaaaaacaa aacctcgagt caggcggcgt gaacaatgcg |
| | | cccaaaacct |
| | 2161 | tcacaggccg gaaaatctcc cttgacttcc aagatgtcga aatccgcacc |
| | | atcctgcaga |
| | 2221 | ttttggcaaa agaatccggg atgaacattg ttgccagcga ctccgtcagc |
| | | ggcaaaatga |
| | 2281 | ccctctccct caaagacgta ccttgggatc aggctttgga tttggttatg |
| | | caggcgcgca |
| | 2341 | acctcgatat gcgccagcaa gggaacatcg tcaacatggc cccgcgacga |
| | | gctgcttgcc |
| | 2401 | aaagacaaag ccttcttaca agcggaaaaa gacattgccg atctgggcgc |
| | | gctgtattcc |
| | 2461 | caaaacttcc aattgaaata caaaaatgtg gaagaattcc gcagcatcct |
| | | gctttggaca |
| | 2521 | atgccgacac gaccggaaac cgcaacacgc ttgtcagcgg caggggcagc |
| | | gtgctgatcg |
| | 2581 | atcccgccac caacaccctg attgttaccg atacccgcag cgtcatcgaa |
| | | aaattccgca |
| | 2641 | aactgattga cgaattggac gtacccgcgc aacaagtgat gattgaggcg |
| | | cgtatcgtcg |
| | 2701 | aagcggcaga cggcttctcg cgcgatttgg gcgttaagtt cggcgcgaca |
| | | ggcaggaaaa |
| | 2761 | aactgaaaaa tgagacgagc gcattcggct ggggcgtgaa ctccggcttc |
| | | gggggcggcg |
| | 2821 | ataaatggga ggccaaacca aaatcaacct gccggttgcc gtgccgcaaa |
| | | cagcatttcg |
| | 2881 | ctggtgcgcg cgatttctcc ggcgcgttga atttggaatt gtccgcatcc |
| | | gagtcgcttt |
| | 2941 | caaaaaccaa aacgcttgcc aatccgcgcg tgctgaccca aaaccgcaaa |
| | | gaggccaaaa |
| | 3001 | tcgaatccgg ttacgaaatt ccttttaccg taactacacg ctcgggcggc |
| | | ggcaactcta |
| | 3061 | ccaacacgga actcaaaaaa gccgtcttgg ggctgaccgt tacggcgaac |
| | | atcacgcccg |
| | 3121 | acggacaaat catcatgacc gtcaaaatca acaaagactc gcctcgacaa |
| | | tgtgcttcag |
| | 3181 | gcaacaacac aatcctatgt atttcgacca aaagcctgaa tacgcaggct |
| | | atggttgaaa |
| | 3241 | acggcggcac tttgattgtc ggcggtattt atgaagaaaa caacggcaat |
| | | acgctgacca |
| | 3301 | aagtccccct gttggctaca tccccgttat cggcaacctc tttaaaacac |
| | | tcgggaaaaa |
| | 3361 | accgaccgcc gcgaactgct gattttcaat tacccccgag ggaattatag |
| | | atacggcgca |
| | 3421 | acagcctgcg ctattgatgc gtcaaaataa gggcatatgt tttacagcat |
| | | atgccctttc |
| | 3481 | tttatgcttt ttgccgcgac cgaaatgccg tcattcccgc gagcgaatcc |
| | | aacttgtccg |
| | 3541 | gtttcggttg tttttcgtct cgtaactttt gagccgtcat tcccgcgaaa |
| | | tcggaaatcc |
| | 3601 | agtccgttca gtttcggtca tttccgataa attcctgttg cttttcattt |
| | | ctagattccc |
| | 3661 | actttcgtgg aataacggcg gaagggataa atcctcgcaa tccaaagcct |
| | | gctcatttcc |
| | 3721 | acaaaaaaca gcaacccgaa acaccccgtc attcccgagc aggcggaatc |
| | | tagaaccgca |
| | 3781 | acgccagqaa tctgtcggat acggctgaaa ccgaacgact ggattcccg |

| NspA | | |
|---|---|---|
| Neisseria gonorrhoeae outer membrane protein gene, complete cds | | |
| ACCESSION U52069 | | |
| VERSION U52069.1 GI:1808968 | | |
| source Neisseria gonorrhoeae/strain = “B2” | | |
| Gene “NspA”coding sequence : 141..665 | | |
| /protein_id = “AAB41581.1” | | |
| /db_xref = “GI:1808969” | | |
| /translation = “MKKALAALIALALPAAALAEGASGFYVQADAAHAKASSSLGSAK | | |
| GFSPRISAGYRINDLRFAVDYTRYKNYKAPSTDFKLYSIGASVIYDFDTQSPVKPYFG | | |
| ARLSLNRASAHLGGSDSFSKTSAGLGVLAGVSYAVTPNVDLDAGYRYNYVGKVNTVKN | | |
| VRSGELSAGVRVKF” | | |
| | 1 | cggcaaagca gccggatgcc gccgcgtatc ttgaggcatt gaaaatatta |
| | | cgatgcaaaa |
| | 61 | agaaaatttc agtataatac ggcaggattc tttaacggat tattaacaat |
| | | ttttctccct |
| | 121 | gaccataaag gaaccaaaat atgaaaaaag cacttgccgc actgattgcc |
| | | ctcgcactcc |
| | 181 | cggccgccgc actggcggaa ggcgcatccg gcttttacgt ccaagccgat |
| | | gccgcacacg |
| | 241 | ccaaagcctc aagctcttta ggttctgcca aaggcttcag cccgcgcatc |
| | | tccgcaggct |
| | 301 | accgcatcaa cgacctccgc ttcgccgtcg attacacgcg ctacaaaaac |
| | | tataaagccc |
| | 361 | catccaccga tttcaaactt tacagcatcg gcgcgtccgt catttacgac |
| | | ttcgacaccc |
| | 421 | aatcgcccgt caaaccgtat ttcggcgcgc gcttgagcct caaccgcgct |
| | | tccgcccact |
| | 481 | tgggcggcag cgacagcttc agcaaaacct ccgccggcct cggcgtattg |
| | | gcgggcgtaa |
| | 541 | gctatgccgt taccccgaat gtcgatttgg atgccggcta ccgctacaac |
| | | tacgtcggca |
| | 601 | aagtcaacac tgtcaaaaac gtccgttccg gcgaactgtc cgccggcgtg |
| | | cgcgtcaaat |
| | 661 | tctgatatac gcgttattcc gcaaaccgcc gagccttcgg cggttttttg |

| Neisseria gonorrhoeae outer membrane protein (omp85) gene, complete | | |
|---|---|---|
| cds. | | |
| ACCESSION U81959 | | |
| VERSION U81959.1 GI:1766041 | | |
| Source: Neisseria gonorrhoeae/strain = “FA19” | | |
| gene = “omp85”coding sequence 1..2379 | | |
| /protein_id = “AAC17600.1” | | |
| /db_xref = “GI:1766042” | | |
| /translation = “MKLKQIASALMMLGISPLAFADFTIQDIRVEGLQRTEPSTVFNY | | |
| LPVKVGDTYNDTHGSAIIKSLYATGFFDDVRVETADGLLLLTVIVCPTIGSLNITGAK | | |
| MLQNDAIKKNLESFGLAQSQYFNQATLNQAVAGLKEEYLGRGKLNIQITPKVTKLARN | | |
| RVDIDITIDEGKSAKITDIEFEGNQVYSDRKLMRQMSLTEGGIWTWLTRSDRFDRQKF | | |
| AQDMEKVTDFYQNNGYFDFRILDTDIQTNEDKTRQTIKITVHEGGRFRWGKVSIEGDT | | |
| NEVPKAELEKLLTMKPGKWYERQQMTAVLGEIQNRMGSAGYAYSEISVQPLPNAGTKT | | |
| VDFVLHIEPGRKIYVNEIHITGNNKTRDEVVRRELRQMESAPYDTSKLQRSKERVELL | | |
| GYFDNVQFDAVPLAGTPDKVDLNMSLTERSTGSLDLSAGWVQDTGLVMSAGVSQDNLF | | |
| GTGKSAALRASRSKTTLNGSLSFTDPYFTADGVSLGYDIYGKAFDPRKASTSVKQYKT | | |
| TTAGGGVRMGIPVTEYDRVNFGLAAEHLTVNTYNKAPKRYADFIKQYGKTDGADGSFK | | |
| GLLYKGTVGWGRNKTDSALWPTRGYLTGVNAEIALPGSKLQYYSATHNQTWFFPLSKT | | |
| FTLMLGGEVGIAGGYGRTKEIPFFENFYGGGLGSVRGYESGTLGPKVYDEYGEKISYG | | |
| GNKKANVSAELLFPMPGAKDARTVRLSLFADAGSVWDGRTYTAAENGNNKSVYSENAH | | |
| KSTFTNELRYSAGGAVTWLSPLGPMKFIYAYPLKKKPEDEIQRFQFQLGTTF” | | |
| | 1 | atgaaactga aacagattgc ctccgcactg atgatgttgg gcatatcgcc |
| | | tttggcattt |
| | 61 | gccgacttca ccatccaaga catccgtgtc gaaggcttgc agcgtaccga |
| | | gccgagcacc |
| | 121 | gtattcaact acctgcccgt caaagtcggc gacacctaca acgacacaca |
| | | cggcagtgcc |
| | 181 | atcatcaaaa gcctgtacgc caccggtttc tttgacgacg tacgagtcga |
| | | aactgcggac |
| | 241 | gggctgcttc tgctgaccgt tatcgtatgc cctaccatcg gctcgctcaa |
| | | catcaccggc |
| | 301 | gccaaaatgc tgcagaacga cgccatcaag aaaaacctcg aatcgttcgg |
| | | gctggcgcag |
| | 361 | tcgcaatact ttaatcaggc gacactcaac caggcagtcg ccggcctgaa |
| | | agaagaatat |
| | 421 | ctcgggcgcg gcaaactcaa tatccaaatc acgcccaaag taaccaaact |
| | | cgcccgcaac |
| | 481 | cgcgtcgaca tcgacatcac gattgacgag ggcaaatccg ccaaaatcac |
| | | cgacatcgaa |
| | 541 | tttgaaggca accaagtcta ttccgaccgc aaactgatgc ggcagatgtc |
| | | gctgaccgaa |
| | 601 | ggcggcattt ggacatggct gacacgaagc gaccggttcg accgccagaa |
| | | attcgcccaa |
| | 661 | gacatggaaa aagtaaccga cttctaccag aacaacggct acttcgattt |
| | | ccgtatcctc |
| | 721 | gataccgaca tccaaaccaa cgaagacaaa accaggcaga ccatcaaaat |
| | | caccgtccac |
| | 781 | gaaggcggac gtttccgctg gggcaaagtg tcgattgaag gcgacaccaa |
| | | cgaagtcccc |
| | 841 | aaggccgaac tggaaaaact gctgaccatg aagcccggca aatggtacga |
| | | acgccagcag |
| | 901 | atgaccgccg ttttgggtga gattcagaac cgcatgggct cggcaggcta |
| | | cgcatacagc |
| | 961 | gaaatcagcg tacagccgct gccgaacgcc ggaaccaaaa ccgtcgattt |
| | | cgtcctgcac |
| | 1021 | atcgaaccgg gcagaaaaat ctacgtcaac gaaatccaca tcaccggcaa |
| | | caacaaaacc |
| | 1081 | cgcgacgaag tcgtgcgccg cgaattgcgc caaatggaat ccgcgcctta |
| | | cgacacctcc |
| | 1141 | aagctgcaac gctccaaaga gcgcgtcgag cttttgggct acttcgacaa |
| | | cgtacagttt |
| | 1201 | gatgccgtcc cgcttgccgg tacgcccgac aaagtcgatt tgaacatgag |
| | | cctgaccgaa |
| | 1261 | cgttccaccg gctcgctcga cttgagcgcg ggctgggttc aggataccgg |
| | | cttggtcatg |
| | 1321 | tccgccggcg tatcgcagga caacctgttc ggtacgggca agtcggccgc |
| | | cctgcgcgcc |
| | 1381 | tcgcgaagca aaaccacgct caacggctcg ctgtcgttta ccgacccgta |
| | | cttcacggca |
| | 1441 | gacggggtca gcctgggcta cgatatttac ggaaaagcct tcgacccgcg |
| | | caaagcatcg |
| | 1501 | accagcgtca aacaatataa aaccaccacc gccggcggcg gcgtaaggat |
| | | gggtatcccc |
| | 1561 | gttaccgaat acgaccgcgt caatttcggg ctggcggcgg aacacctgac |
| | | cgtcaacacc |
| | 1621 | tacaacaaag cacccaaacg ctatgccgac tttatcaaac aatacggcaa |
| | | aaccgacggc |
| | 1681 | gcagacggca gcttcaaagg cctgctgtac aaaggcactg tcggctgggg |
| | | gcgcaacaag |
| | 1741 | accgacagcg ccttatggcc gacgcgcggc tacctgaccg gcgtaaatgc |
| | | cgaaatcgcc |
| | 1801 | ctgcccggca gcaaactgca atactactcc gccacccaca accaaacctg |
| | | gttcttcccc |
| | 1861 | ttaagcaaaa ccttcacgct gatgctcggc ggcgaagtcg gcattgcggg |
| | | cggctacggc |
| | 1921 | agaaccaaag aaatcccctt ctttgaaaac ttctacggcg gcggcctggg |
| | | ttcggtgcgc |
| | 1981 | ggctacgaaa gcggcacgct cggcccgaaa gtgtatgacg aatacggcga |
| | | aaaaatcagc |
| | 2041 | tacggcggca acaaaaaagc caacgtctcc gccgagctgc tcttcccgat |
| | | gcccggtgcg |
| | 2101 | aaagacgcac gcaccgtccg cctgagcctg tttgccgacg caggcagcgt |
| | | gtgggacggc |
| | 2161 | agaacctata ccgccgccga aaacggtaac aacaaatcgg tttactcgga |
| | | aaacgcgcat |
| | 2221 | aaatccacct ttaccaacga attgcgctat tccgccggcg gcgcggttac |
| | | ctggctctcg |
| | 2281 | cctttgggcc cgatgaaatt catctacgcc tacccgctga agaaaaaacc |
| | | ggaagacgaa |
| | 2341 | atccaacgct tccaattcca gctcggcacg acgttctaa |

PldA1 homolog in Neisseria gonorrhoeae
Source: U. of Oklahoma sequencing project
PldA1-like coding sequence:
>GONOCTG01_15 Continuation (15 of 22) of gonoctg01 from base 1400001
ATGAATACACGAAATATGCGCTATATTCTTTTGACAGGACTGTTGCCGACGGCATCCGCT
TTTGGAGAGACCGCGCTGCAATGCGCCGCTTTGACGGACAATGTTACGCGTTTGGCGTGT
TACGACAGGATTTTTGCGGCACAGCTTCCGTCTTCGGCAGGGCAGGAAGGGCAGGAGTCG
AAAGCCGTACTCAATCTGACGGAAACCGTCCGCAGCAGCTTGGATAAGGGCGAGGCGGTC
ATTGTTGTTGAAAAAGGCGGGGATGCGCTTCCTGCCGACAGTGCGGGCGAAACCGCCGAT
ATCTATACGCCTTTGAGCCTGATGTACGACTTGGACAAAAACGATTTGCGCGGGCTGTTG
GGCGTACGCGAACACAATCCGATGTACCTTATGCCGTTTTGGTATAACAATTCGCCCAAC
TATGCCCCGAGTTCGCCGACGCGCGGTACGACTGTACAGGAAAAATTCGGACAGCAGAAA
CGTGCGGAAACCAAATTGCAGGTTTCGTTCAAAAGCAAAATTGCCGAAAATTTGTTTAAA
ACCCGCGCGGATCTGTGGTTCGGCTACACCCAAAGATCCGATTGGCAGATTTACAACCAA
GGCAGGAAATCCGCGCCGTTCCGCAATACGGATTACAAACCTGAAATTTTCCTGACCCAG
CCTGTGAAGGCGGATTTGCCGTTCGGCGGCAGGCTGCGTATGCTCGGTGCGGGTTTTGTC
CACCAGTCCAACGGACAGAGCCGTCCCGAATCGCGTTCGTGGAACAGGATTTATGCCATG
GCAGGCATGGAATGGGGCAAATTGACGGTGATTCCGCGCGTGTGGGTGCGTGCGTTCGAT
CAGAGCGGCGATAAAAACGACAATCCCGATATTGCCGACTATATGGGGTATGGCGACGTG
AAGCTGCAGTACCGCCTGAACGACAGGCAGAATGTGTATTCCGTATTGCGCTACAACCCC
AAAACGGGCTACGGCGCGATTGAAGCCGCCTACACGTTTCCGATTAAGGGCAAACTCAAA
GGCGTGGTACGCGGATTCCACGGTTACGGCGAGAGCCTGATCGACTACAACCACAAGCAG
AACGGTATCGGTATCGGGTTGATGTTCAACGACTGGGACGGCATCTGA
PldA1-like amino acid sequence
MNTRNMRYILLTGLLPTASAFGETALQCAALTDNVTRLACYDRIFAAQLPSSAGQEGQESKAVLNLTE
TVRSSLDKGEAV
IVVEKGGDALPADSAGETADIYTPLSLMYDLDKNDLRGLLGVREHNPMYLMPFWYNNSPNYAPSSPT
RGTTVQEKFGQQK
RAETKLQVSFKSKIAENLFKTRADLWFGYTQRSDWQIYNQGRKSAPFRNTDYKPEIFLTQPVKADLPFG
GRLRMLGAGFV
HQSNGQSRPESRSWNRIYAMAGMEWGKLTVIPRVWVRAFDQSGDKNDNPDIADYMGYGDVKLQYR
LNDRQNVYSVLRYNP
KTGYGAIEAAYTFPIKGKLKGVVRGFHGYGESLIDYNHKQNGIGIGLMFNDWDGI.
1000 base pairs upstream PldA1-like sequence (usuable for replacing
the promoter for a stronger sequence)
>GONOCTG01_15 Continuation (15 of 22) of gonoctg01 from base 1400001
TTTTGGCTTCCAGCGTTTCGTTGTTTTCGTACAAGTCGTAAGTCAGCTTCAGATTGTTGG
CTTTTTTAAAGTCTTCGACCGTACTCTCGTCAACATAATTCGACCAGTTGTAGATGTTCA
GAGTATCGGTGGCAGCGGCTTCGGCATTGGCAGCAGGTGCGCTGCCTGCTTGAGGCTGCA
CGGCGTTTTTTTCGCTGCCGCCGCAGGCTGCCAGAGACAGCGCGGCCAAAACGGCTAATA
CGGATTTTTTCATACGGGCAGATTCCTGATGAAAGAGGTTGGAAAAAAAGAAAACCCCGC
GCCCCATAAACACCCCGGCGCAAGGTTTGGGTATTGTAAAGTAAATTTGTGCAAACTCAA
AGCGATATTGGCCTGATTTTCCTAAAAAATTACCCTGTTTCCAAAAAAGGGGGGGAAACG
GCCGCCCGATTTTGCCGTTTTTTTGCGCCGTCAGGGTGTCCGACGGGCGGATAGAGAAAA
AAGGCTTGCATATAATGTAAACCCCCTTTAAAATTGCGCGTTTACAGAATTTATTTTTCT
TTCAGGAGATTCCAATATGGCAAACAGCGCACAAGCACGCAAACGTGCCCGCCAGTCCGT
CAAACAACGCGCCCACAACGCTAGCCTGCGTACCGCATTCCGCACCGCAGTGAAAAAAGT
ATTGAAAGCAGTCGAAGCAGGCGATAAAGCTGCCGCACAAGCGGTTTACCAAGAGTCCGT
CAAAGTCATCGACCGCATCGCCGACAAAGGCGTGTTTCATAAAAACAAAGCGGCTCGCCA
CAAAAGCCGCCTGTCTGCAAAAGTAAAAGCACTGGCTTGATTTTTGCAAAACCGCCAAGG
CGGTTGATACGCGATAAGCGGAAAACCCTGAAGCCCGACGGTTTCGGGGTTTTCTGTATT
TCGGGGGTAAAGTTCGAAATGGCGGAAAGGGTGCGGTTTTTTATCCGAATCCGCTATAAA
ATGCCGTTTGAAAACCAATATGCCGACAATGGGGGCGGAG

Preferred gonococcal genes to downregulate via process a) include one or more of the following:

| Neisseria gonorrhoeae iron-regulated outer membrane protein preFrpB | | |
|---|---|---|
| (frpB) gene, complete cds. | | |
| ACCESSION U13980 | | |
| VERSION U13980.1 GI:833694 | | |
| Source: Neisseria gonorrhoeae/strain = “FA19” | | |
| gene = “frpB”coding sequence: 318..2459 | | |
| /protein_id = “AAC43332.1” | | |
| /db_xref = “GI:833695” | | |
| /translation= “MNAPFFRLSLLSLTLAAGFAHAAENNANVALDTVTVKGDRQGSK | | |
| IRTNIVTLQQKDESTATDMRELLKEEPSIDFGGGNGTSQFLTLRGMGQNSVDIKVDNA | | |
| YSDSQILYHQGRFIVDPALVKVVSVQKGAGSASAGIGATNGAIIAKTVDAQDLLKGLD | | |
| KNWGVRLNSGFAGNNGASYGASVFGKEGNFDGLFSYNRNDEKDYEAGKGFRNDNGGKT | | |
| VPYSALDKRSYLAKIGTTFGDGDHRIVLSHMKDQHRGIRTVREEFAVSEKNSRITIKR | | |
| QAPSYRETTQSNTNLAYTGKDLGFVEKLDANAYVLEKKRYSADDKDNGYAGNVKGPNH | | |
| TRIATRSMNFNFDSRLAEQTLLKYGINYRHQEIKPQAFLNSEFEIKDKEKATNEEKKK | | |
| NRENEKIAKAYRLTNPTKTDTGAYIEAIHEIDGFTLTGGLRYDRFKVKTHDGKTVSSS | | |
| SLNPSFGVIWQPREHWSFSASHNYAGRSPRLYDALQTHGKRGIISIADGTKAERARNT | | |
| EIGFNYNDGTFAANGSYFRQTIKDALANPQNRHDSVAVREAVNAGYIKNHGYELGASY | | |
| RTGGLTAKVGVSHSKPRFYDTHKDKLLSANPEFGAQVGRTWTASLAYRFKNPNLEIGW | | |
| RGRYVQKAVGSILAAGQKDRDGKLENVVRQGFGVNDVFANWKPLGKDTLNVNLSVNNV | | |
| FDKFYYPHSQRWTNTLPGVGRDVRLGVNYKF” | | |
| | 1 | aaaccggtac ggcgttgccc cgccttagct caaagagaac gattccctaa ggtgctgaag |
| | 61 | caccgagtga atcggttccg tactatttgt actgtctgcg gcttcgccgc |
| | | cttgtcctga |
| | 121 | tttttgttag tccacatata catttccgac aaaacctgtc aacaaaaaac |
| | | aacgcttcgc |
| | 181 | aaataaaaac gataatcagc tttacacaac ccccccccgc taatataaac |
| | | aaaaataatt |
| | 241 | attattattt tttcttatcc tgccaaacct taacggtttg gcttaacttc |
| | | ccttcataca |
| | 301 | ctcaaaagga cgaacaaatg aacgccccgt ttttccgcct cagcctgctc |
| | | tcgctcacac |
| | 361 | ttgccgccgg ctttgcccac gcggcagaaa ataatgccaa tgtcgcattg |
| | | gataccgtta |
| | 421 | ccgtaaaagg cgaccgccaa ggcagcaaaa tccgtaccaa catcgttacg |
| | | cttcaacaaa |
| | 481 | aagacgaaag caccgcaacc gatatgcgcg aactcttaaa agaagagccc |
| | | tccatcgatt |
| | 541 | tcggcggcgg caacggcacg tcccaattcc tgacgctgcg cggtatgggt |
| | | cagaactctg |
| | 601 | tcgacatcaa ggtggacaac gcctattccg acagccaaat cctttaccac |
| | | caaggcagat |
| | 661 | ttattgtcga tcccgctttg gttaaagtcg tttccgtaca gaaaggcgcg |
| | | ggttccgcct |
| | 721 | ctgccggtat cggcgcgacc aacggcgcga tcatcgccaa aaccgtcgat |
| | | gcccaagacc |
| | 781 | tgctcaaagg cttggataaa aactggggcg tgcgcctcaa cagcggcttt |
| | | gccggcaaca |
| | 841 | acggcgcaag ctacggcgca agcgtattcg gaaaagaggg caacttcgac |
| | | ggtttgttct |
| | 901 | cttacaaccg caacgatgaa aaagattacg aagccggcaa aggtttccgc |
| | | aatgacaacg |
| | 961 | gcggcaaaac cgtaccgtac agcgcgctgg acaaacgcag ctacctcgcc |
| | | aaaatcggaa |
| | 1021 | caaccttcgg cgacggcgac caccgcatcg tgttgagcca tatgaaagac |
| | | caacaccggg |
| | 1081 | gcatccgcac tgtgcgtgaa gagtttgccg tcagcgaaaa aaattcacgg |
| | | ataactatta |
| | 1141 | aacgccaagc cccatcctac cgcgaaacca ctcaatccaa caccaacttg |
| | | gcgtacaccg |
| | 1201 | gcaaagattt gggctttgtc gaaaaactgg atgccaacgc ctatgtgttg |
| | | gaaaagaaac |
| | 1261 | gctattccgc cgatgacaaa gataacggct acgcaggcaa tgtaaaaggc |
| | | cccaaccata |
| | 1321 | cccgaatcgc cactcggagt atgaacttca acttcgacag ccgccttgcc |
| | | gaacaaaccc |
| | 1381 | tgttgaaata cggcatcaac taccgccatc aggaaatcaa accgcaagcg |
| | | tttttgaact |
| | 1441 | cggaatttga aataaaagat aaagaaaaag caactaatga agagaaaaag |
| | | aagaaccgtg |
| | 1501 | aaaatgaaaa aattgccaaa gcctaccgcc tgaccaaccc gaccaaaacc |
| | | gataccggcg |
| | 1561 | cgtatatcga agccattcac gagattgacg gctttaccct gaccggcggg |
| | | ctgcgttacg |
| | 1621 | accgcttcaa ggtgaaaacc cacgacggca aaaccgtttc aagcagcagc |
| | | ctcaacccga |
| | 1681 | gtttcggcgt gatttggcag ccgcgcgaac actggagctt cagcgcgagc |
| | | cacaactacg |
| | 1741 | ccggccgcag cccgcgcctg tatgacgctc tgcaaaccca cggcaagcgc |
| | | ggcatcatct |
| | 1801 | cgattgccga cggcacgaaa gccgaacgcg cgcgcaatac cgaaatcggc |
| | | ttcaactaca |
| | 1861 | acgacggcac gtttgccgca aacggcagct acttccggca gaccatcaaa |
| | | gacgcgcttg |
| | 1921 | ccaatccgca aaaccgccac gactccgtcg ccgtccgcga agccgtcaac |
| | | gccggctaca |
| | 1981 | tcaaaaacca cggttacgaa ttgggcgcgt cctaccgcac cggcggcctg |
| | | accgccaaag |
| | 2041 | tcggcgtaag ccacagcaaa ccgcgctttt acgatacgca caaagacaag |
| | | ctgttgagcg |
| | 2101 | cgaaccctga atttggcgca caagtcggcc gcacttggac ggcctccctt |
| | | gcctaccgct |
| | 2161 | tcaaaaaccc gaatctggaa atcggctggc gcggtcgtta tgttcaaaaa |
| | | gccgtgggtt |
| | 2221 | cgatattggc ggcaggtcaa aaagaccgcg acggcaaatt ggaaaacgtt |
| | | gtacgccaag |
| | 2281 | gtttcggtgt gaacgatgtc ttcgccaact ggaaaccgct gggcaaagac |
| | | acgctcaatg |
| | 2341 | ttaatctttc ggttaacaac gtgttcgaca agttctacta tccgcacagc |
| | | caacgctgga |
| | 2401 | ccaataccct gccgggcgtg ggacgtgatg tacgcctggg cgtgaactac |
| | | aagttctaaa |
| | 2461 | acgcacatcc cgaaaaaatg ccgtctgaaa gcctttcaga cggcatctgt |
| | | cctgataatt |
| | 2521 | tgatatatag tggattaaca aaaaccggta cggcgttgcc ccgccttagc |
| | | tcaaagggaa |
| | 2581 | cgattcccta aggtgctgaa |

| N. gonorrhoeae structural gene for gonococcal protein III (PIII). | | |
|---|---|---|
| ACCESSION X05105 | | |
| VERSION X05105.1 GI:44889 | | |
| source:Neisseria gonorrhoeae/db_xref = “taxon: 485” | | |
| Gene PIII coding sequence: 103..813 | | |
| /protein_id = “CAA28752.1” | | |
| /db_xref = “GI:44890” | | |
| /db_xref = “SWISS-PROT:P07050” | | |
| /translation = “MTKQLKLSALFVALLASGTAVAGEASVQGYTVSGQSNEIVRNNY | | |
| GECWKNAYFDKASQGRVECGDAVAVPEPEPAPVAVVEQAPQYVDETISLSAKTLFGFD | | |
| KDSLRAEAQDNLKVLAQRLSRTNVQSVRVEGHTDFMGSEKYNQALSERRAYVVANNLV | | |
| SNGVPASRISAVGLGESQAQMTQVCQAEVAKLGAKASKAKKREALIACIEPDRRVDVK | | |
| IRSIVTRQVVPARNHQHH” | | |
| | 1 | gaattcctat ccgatttgcc gccatgtttc tacagcggcc tgtatgttgg caattcagca |
| | 61 | gttgcttctg tatctgctgt acaaatctaa tgagggaata aaatgaccaa |
| | | acagctgaaa |
| | 121 | ttaagcgcat tattcgttgc attgctcgct tccggcactg ctgttgcggg |
| | | cgaggcgtcc |
| | 181 | gttcagggtt acaccgtaag cggccagtcg aacgaaatcg tacgcaacaa |
| | | ctatggagaa |
| | 241 | tgctggaaaa acgcctactt tgataaagca agccaaggtc gcgtagaatg |
| | | cggcgatgcg |
| | 301 | gttgccgtcc ccgagcccga acccgcgcct gtcgccgttg tggagcaggc |
| | | tcctcaatat |
| | 361 | gttgatgaaa ccatttccct gtctgccaaa accctgttcg gtttcgataa |
| | | ggattcattg |
| | 421 | cgcgccgaag ctcaagacaa cctgaaagta ttggcgcaac gcctgagtcg |
| | | aaccaatgtc |
| | 481 | caatctgtcc gcgtcgaagg ccataccgac tttatgggtt ctgaaaaata |
| | | caatcaggct |
| | 541 | ctgtccgaac gccgcgcata cgtagtggca aacaacctgg tcagcaacgg |
| | | cgtacctgct |
| | 601 | tctagaattt ctgctgtcgg cttgggcgaa tctcaagcgc aaatgactca |
| | | agtttgtcaa |
| | 661 | gccgaagttg ccaaactggg tgcgaaagcc tctaaagcca aaaaacgtga |
| | | ggctctgatt |
| | 721 | gcatgtatcg aacctgaccg ccgcgtagat gtgaaaatcc gcagcatcgt |
| | | aacccgtcag |
| | 781 | gttgtgccgg cacgcaatca tcaccaacac taaggctagg taatatcttg |
| | | ccgatgcatg |
| | 841 | aggttagcgg attttgtacc gggtactgtt gcaatattcg tgaaacgtcg |
| | | gccggtatcg |
| | 901 | atgatgtgaa acaaaccccg cttttgcggg gtttgttttt ttgggtggtt |
| | | ttctgaaacg |
| | 961 | gctatcgtca gaatcggggt gcaggttcgg attcggattc agattcatgt |
| | | ttgtgtccca |
| | 1021 | ttgccgcgct ttatagtgga ttaacaaaaa tcaggacaag gcgacgaagc |
| | | cgcagacagt |
| | 1081 | acaatagtac ggcaaggcga ggcaacgccg taccggttta aatttaatcc |
| | | actatatcgg |
| | 1141 | ttgaaactct gattttaagg cggtaggatg tgggtttgcc catagcaagg |
| | | gaatcctttc |
| | 1201 | tgtatcaagc cccgaaaggg ataattcata caaattcacg cctttccccc |
| | | tcattgggaa |
| | 1261 | atggatggaa tcgtgcccga tgtgtgcggc actgtatgcc ggatatggtt |
| | | ttatcatcat |
| | 1321 | cccttttcgg ttgaaacccc gcggaattc |

Preferred Pseudomonas aeruginosa Bleb Preparations

One or more of the following genes (encoding protective antigens) are preferred for upregulation via process b): PcrV, OprF, OprI. They are also preferred as genes which may be heterologously introduced into other Gram-negative bacteria.

Preferred Moraxella catarrhalis Bleb Preparations

One or more of the following genes (encoding protective antigens) are preferred for upregulation via process b): OMP106 (WO 97/41731 & WO 96/34960), HasR (PCT/EP99/03824), PilQ (PCT/EP99/03823), OMP85 (PCT/EP00/01468), lipo06 (GB 9917977.2), lipo10 (GB 9918208.1), lipo11 (GB 9918302.2), lipo18 (GB 9918038.2), P6 (PCT/EP99/03038), ompCD, CopB (Helminen M E, et al (1993) Infect. Immun. 61:2003-2010), D15 (PCT/EP99/03822), OmplA1 (PCT/EP99/06781), Hly3 (PCT/EP99/03257), LbpA and LbpB (WO 98/55606), TbpA and TbpB (WO 97/13785 & WO 97/32980), OmpE, UspA1 and UspA2 (WO 93/03761), FhaB (WO 99/58685) and Omp21. They are also preferred as genes which may be heterologously introduced into other Gram-negative bacteria.

One or more of the following genes are preferred for downregulation via process a): CopB, OMP106, OmpB1, TbpA, TbpB, LbpA, and LbpB.

One or more of the following genes are preferred for downregulation via process c): htrB, msbB and lpxK (most preferably msbB).

One or more of the following genes are preferred for upregulation via process d): pmrA, pmrB, pmrE, and pmrF.

Many of the above open reading frames and upstream regions are described in WO 01/09350 (incorporated by reference herein).

Preferred Haemophilus influenzae Bleb Preparations

One or more of the following genes (encoding protective antigens) are preferred for upregulation via process b): D15 (WO 94/12641), P6 (EP 281673), TbpA, TbpB, P2, P5 (WO 94/26304), OMP26 (WO 97/01638), HMW1, HMW2, HMW3, HMW4, Hia, Hsf, Hap, Hin47, Iomp1457 (GB 0025493.8), YtfN (GB 0025488.8), VirG (GB 0026002.6), Iomp1681 (GB 0025998.6), OstA (GB 0025486.2) and Hif (all genes in this operon should be upregulated in order to upregulate pilin). They are also preferred as genes which may be heterologously introduced into other Gram-negative bacteria.

One or more of the following genes are preferred for downregulation via process a): P2, P5, Hif, IgA1-protease, HgpA, HgpB, HMW1, HMW2, Hxu, ThpA, and TbpB.

One or more of the following genes are preferred for downregulation via process c): htrB, msbB and lpxK (most preferably msbB).

One or more of the following genes are preferred for upregulation via process d): pmrA, pmrB, pmrE, and pmrF.

Many of the above open reading frames and upstream regions are described in WO 01/09350 (incorporated by reference herein).

Preparations of Membrane Vesicles (Blebs) of the Invention

The manufacture of bleb preparations from any of the aforementioned modified strains may be achieved by harvesting blebs naturally shed by the bacteria, or by any of the methods well known to a skilled person (e.g. as disclosed in EP 301992, U.S. Pat. No. 5,597,572, EP 11243 or U.S. Pat. No. 4,271,147). For Neisseria, the method described in the Example below is preferably used

A preparation of membrane vesicles obtained from the bacterium of the invention is a further aspect of this invention. Preferably, the preparation of membrane vesicles is capable of being filtered through a 0.22 μm membrane.

A sterile (preferably homogeneous) preparation of membrane vesicles obtainable by passing the membrane vesicles from the bacterium of the invention through a 0.22 μm membrane is also envisaged.

Vaccine Formulations

A preferred embodiment of the invention is the formulation of the bleb preparations of the invention in a vaccine which may also comprise a pharmaceutically acceptable excipient.

Vaccine preparation is generally described in Vaccine Design (“The subunit and adjuvant approach” (eds Powell M. F. & Newman M. J.) (1995) Plenum Press New York).

The bleb preparations of the present invention may be adjuvanted in the vaccine formulation of the invention. Suitable adjuvants include an aluminium salt such as aluminum hydroxide gel (alum) or aluminium phosphate, but may also be a salt of calcium (particularly calcium carbonate), iron or zinc, or may be an insoluble suspension of acylated tyrosine, or acylated sugars, cationically or anionically derivatised polysaccharides, or polyphosphazenes.

Suitable Th1 adjuvant systems that may be used include, Monophosphoryl lipid A, particularly 3-de-O-acylated monophosphoryl lipid A, and a combination of monophosphoryl lipid A, preferably 3-de-O-acylated monophosphoryl lipid A (3D-MPL) together with an aluminium salt. An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO96/33739. A particularly potent adjuvant formulation involving QS21 3D-MPL and tocopherol in an oil in water emulsion is described in WO95/17210 and is a preferred formulation.

The vaccine may comprise a saponin, more preferably QS21. It may also comprise an oil in water emulsion and tocopherol. Unmethylated CpG containing oligo nucleotides (WO 96/02555) are also preferential inducers of a TH1 response and are suitable for use in the present invention.

The vaccine preparation of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Thus one aspect of the present invention is a method of immunizing a human host against a disease caused by infection of a gram-negative bacteria, which method comprises administering to the host an immunoprotective dose of the bleb preparation of the present invention.

The amount of antigen in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-100 μg of protein antigen, preferably 5-50 μg, and most typically in the range 5-25 μg.

An optimal amount for a particular vaccine can be ascertained by standard studies involving observation of appropriate immune responses in subjects. Following an initial vaccination, subjects may receive one or several booster immunisations adequately spaced.

Ghost or Killed Whole Cell Vaccines

The inventors envisage that the above modified bacterial strains may not only be useful in generating bleb preparations useful in vaccines—they may also be easily used to make ghost or killed whole cell preparations and vaccines (with identical advantages). Methods of making ghost preparations (empty cells with intact envelopes) from Gram-negative strains are well known in the art (see for example WO 92/01791). Methods of killing whole cells to make inactivated cell preparations for use in vaccines are also well known. The terms ‘bleb preparations’ and ‘bleb vaccines’ as well as the processes described throughout this document are therefore applicable to the terms ‘ghost preparation’ and ‘ghost vaccine’, and ‘killed whole cell preparation’ and ‘killed whole cell vaccine’, respectively, for the purposes of this invention.

### EXAMPLES

The examples below are carried out using standard techniques, which are well known and routine to those of skill in the art, except where otherwise described in detail. The examples are illustrative, but do not limit the invention.

### Example 1

### Previously Reported Examples

Examples describing: Construction of a Neisseiria meningitidis serogroup B strain lacking capsular polysaccharides; Construction of versatile gene delivery vectors (the pCMK series) targeting integration in the porA locus of Neisseiria meningitidis; Construction of a Neisseiria meningitidis serogroup B strain lacking both capsular polysaccharides and the major immunodominant antigen PorA; Up-regulation of the NspA outer membrane protein production in blebs derived from a recombinant Neisseiria meningitidis serogroup B strain lacking functional porA and cps genes; Up-regulation of the D15/Omp85 outer membrane protein antigen in blebs derived from a recombinant Neisseiria meningitidis serogroup B strain lacking functional cps genes but expressing PorA; Construction of versatile promoter delivery vectors; Fermentation processes for producing recombinant blebs; Identification of bacterial promoters suitable for up-regulation antigens-coding genes; Up-regulation of the N. meningitidis serogroup B Omp85 gene by promoter replacement; Up-regulation of the Hsf protein antigen in a recombinant Neisseiria meningitidis serogroup B strain lacking functional cps genes but expressing PorA; Expression of the Green Fluorescent Protein in a recombinant Neisseria meningitidis serogroup B strain lacking functional cps genes but expressing PorA; Up-regulation of the N. meningitidis serogroup B NspA gene by promoter replacement; Up-regulation of the N. meningitidis serogroup B pldA (omplA) gene by promoter replacement; Up-regulation of the N. meningitidis serogroup B tbpA gene by promoter replacement; Up-regulation of the N. meningitidis serogroup B pilQ gene by promoter replacement; Construction of a kanR/sacB cassette for introducing “clean”, unmarked mutations in the N. meningitidis chromosome; Use of small recombinogenic sequences (43 bp) to allow homologous recombination in the chromosome of Neisseria meningitidis; Active protection of mice immunized with WT and recombinant Neisseria meningitidis blebs; and Immunogenicity of recombinant blebs measured by whole cell & specific ELISA methods have been described in WO 01/09350 (incorporated by reference herein).

### Example 2

### Gonococcal Blebs Expressing Chlamydia trachomatis Proteins on its Surface for use in a Vaccine Composition

Both Chlamydia trachomatis and N. gonorrhoeae cause sexually transmitted diseases, including urethritis, cervicitis, salpingitis and pelvic inflammatory disease. Mixed infection with both CT and GC does occur. Therefore, in the design of a vaccine targeting one, or more of these diseases, the possibility to afford protection against both organisms with one single formulation creates a technical advantage.

Protection Against N. gono.

A N. gonorrhoeae OMV vaccine can be obtained from bleb producing strain(s) in which the expression of one or several genes have been up and/or down regulated. A list of genes encoding N. gonorrhoeae proteins for which it is particularly useful to up/down regulate expression is provided above.

A successful vaccine for the prevention of infection by N. gono may require more than one of the following elements: generation of serum and/or mucosal antibodies to facilitate complement mediated killing of the gonococcus, and/or to enhance phagocytosis and microbial killing by leukocytes such as polymorphonuclear leukocytes, and/or to prevent attachment of the gonococci to the host tissues; induction of a cell mediated immune response may also participate to protection.

The potential of a bleb gono vaccine preparation can be evaluated by analyzing the induced immune response for serum and/or mucosal antibodies that have antiadherence, and/or opsonizing properties, and/or bactericidal activity, as described by others (McChesney D et al, Infect. Immun. 36: 1006, 1982; Boslego J et al: Efficacy trialof a purified gonococcl pilus vaccine, in Program and Abstracts of the 24^{th} Interscience Conference on Antimicrobial Agents and Chemotherapy, Whashington, American Society for Microbiology, 1984; Siegel M et al, J. Infect. Dis 145: 300, 1982; de la Pas, Microbiology, 141 (Pt4): 913-20, 1995).

A mouse model of genital infection by N. gono has recently been described (Plante M, J. Infect. Dis., 182: 848-55, 2000). The efficiency of a bleb gono vaccine could also be evaluated by its ability to prevent or to reduce colonization by N. gono in this mouse model of infection.

Protection Against CT

A GC/CT bleb vaccine can be obtained from a strain expressing one or several Chlamydia genes, preferably selected from the above list of genes encoding predicted outer membrane proteins.

Other genes of interest for overexpression in Neisseria are C. trachomatis genes for which no homolog has been found in C. pneumoniae. Such a set of genes has been described in Richard S.; p:9-27, Stephens Stephens Ed. ASM Press, Washington D.C., Chlamydia: Intracellular Biology, Pathogenesis, and Immunity ISBN: 1-55581-155-8 pages: 380.

Most preferred combinations of Chlamydia trachomatis genes are as follows: Major outer membrane protein MOMP (from one or several different serovars) and the Outer membrane Protein Analog (also known as PorB), MOMP (from one or several different serovars) and the Putative Outer Membrane Protein G (pmpG); & PorB and pmpG.

Although the immunity to CT is not fully understood, there is evidence that Ab play a role in protection. Ab to CT in genital fluids have been associated with immunity to CT (Brunham R C, Infect Immun. 1983 March;39(3):1491-4.). A protective role of serum antibody in immunity to chlamydial genital infection has also been shown (Rank R G, Infect Immun. 1989 January;57(1):299-301.). Antibodies, e.g. MOMP specific antibodies, have been shown to be capable to neutralize CT infection in vitro and in vivo (Caldwell et al. 1982 Infect. Immun. 38: 745-54, Lucero et al, 1985, Infect. Immun. 50: 595-97, Zhang et al. 1987 J. Immunol. 138: 575-581). The MOMP surface antigen of CT has been shown to bear non linear surface epitopes which are target of neutralizing antibodies (Fan J, J. Infect Dis 1997, 176(3):713-21).

Thus, an important objective in the design of a protective chlamydia vaccine includes the identification of formulation(s) of the CT antigens able to optimize the induction of a chlamydia specific antibody responses. Optimization of the Ab response includes targeting to the genital mucosa, and/or presentation of properly folded Chlamydia antigens, and/or combination of several antibody targets.

Mucosal targeting of the immune response to Chlamydia antigen can be achieved by mucosal administration of the vaccine. Intranasal administration of a outer membrane vesicle vaccine can induce persistent local mucosal antibodies and serum antibodies with strong bactericidal activity in humans.

For certain B cell epitopes, such as non linear epitopes, the presentation of the antigen to the immune system in a properly folded manner is critical. A bleb vaccine prepared from a strain expressing Chlamydia antigen(s) offers to chlamydia OMP an outer membrane environment which can be critical to maintain these antigens in a properly folded structure.

Combination of several antibody targets can create an increased efficacy by tackling the infection at different steps of the life cycle of the bacteria, such as adhesion to the host cell, internalization by the host cell and/or interference with further steps of the intracellular development.

The induction and recruitement of Th1 cells into the local genital mucosae are important for immunity against Chlamydia. Thus, an important objective in designing a protective anti-chlamydia vaccine includes the identification of formulation(s) of CT antigen(s) able to optimize the induction of chlamydia speicific Th1 cells, and preferably recruitment of these cells into the genital mucosae. A bleb vaccine prepared from a strain expressing chlamydia antigen(s) can induce a chlamydia specific CMI response. Antigen-specific T-cell responses can be induced in humans after intranasal immunization with an outer membrane vesicle vaccine.

A particular advantage of a GC/CT bleb vaccine is its capability to induce both Ab and CMI responses.

The efficacy of the GC/CT bleb vaccine can be evaluated by its ability to elicit Ag or Chlamydia-specific Ab and/or CMI responses. Ab responses can be evaluated by classical techniques such as ELISA or western blot. Preferably, the induced antibodies can neutralize the infectivity of Chlamydia in an in vitro assay (Byrne G. et al. (J Infect Dis. 1993 August;168(2):415-20). Preferably, the CMI response is biased toward the Th1 phenotype. A Th1 biased immune response can be assessed by elevated antigen-specific IgG2a/IgG1 ratios in mice (Snapper et al. 1987, Science 236:944-47). Elevated ratio of Th1/Th2 cytokine, e.g. elevated IFN-gamma/IL-5, ratio upon in vitro restimulation of immune T cells with the antigen(s) can also indicate such a biased Th1 response.

The ability of the formulation to elicit Ag specific mucosal Ab is of particular interest, and can be demonstrated by detection of antibodies, such as IgG and/or IgA in mucosal fluids, such as genital tract secretions, vaginal lavages. To this end, certain route of administration of the vaccine may be particularly desired such as intranasal, oral, intravaginal, intradermal, deliveries.

The efficacy of the GC/CT bleb vaccine can be evaluated by its ability to induce protection against a Chlamydia challenge in animal model(s). Examples of such animal models have been described in the literature: genital infection with MoPn in mice (Barron et al. J. Infect. Dis. 1143:63-66), genital infection with human strains in mice (Igietseme et al.2000, Infect. Immun. 68:6798-806, Tuffrey et al. 1992 J. Gen. Microbiol. 138: 1707-1715), Tuffrey), genital infection with GPIC strain in guinea pigs (Rank et al. 1992 Am. J. Pathol. 140:927-936). Protection against infection can be assessed by reduction of shed Chlamydia from the infected site and/or reduction of the histopathological reactions after a challenge infection in immunized animals.

The advantage of combining two or more Chlamydia antigens (as described above) can be evaluated by one or more of the following techniques:
Ability to elicit a multi-target Ab and/or T cell protective response
Ability to elicit Ab titers in an in vitro neutralizing assay, and/or neutralizing Ab against multiple strains (antigenically distinct)
Ability to elicit a protective immune response against Chlamydia in a mouse model of genital infection as assessed by reduced shedding of bacteria and/or pathology after challenge.

### Example 3

### Expression of Heterologous Antigens (Clamydia trachomatis MOMP and PorB) in blebs Derived from a Recombinant Neisseiria meningitidis Serogroup B Strain Lacking Functional porA and cps Genes

Other genes of interest for over-expression in Neisseria are Chlamydia trachomatis genes for which no homologue has been found in Chlamydia pneumoniae. Among those, the major outer membrane protein (MOMP) and the outer membrane protein analog (PorB) have been shown to play a protective role against chlamydial genital infection. Optimization of the Ab response could be achieved by presentation of properly folded proteins.

MenB bleb vesicles may be used as delivery vectors to express heterologous membrane protein antigens under the control of the engineered porA-lacO promoter described in WO 01/09350. Expressed in the bleb context, recombinant MOMP and PorB from Chlamydia trachomatis serovar D and K can be correctly folded in the membrane and exposed at the surface. Neisseiria meningitidis strains lacking functional cps genes are advantageously used as recipient strains to express the heterologous antigens (WO 01/09350).

PCR Amplifications of the Genes Coding for MOMP (Chlamydia trachomatis).

Murine McCoy cells (ATCC) infected either, with Chlamydia trachomatis Serovar K (UW31-CX-serK), or Serovar D (UW31-CX-serD), were lysed in 400 μl of lysis buffer: 50 mM KCl, 10 mM Tris-HCl pH 8.3, 2.5 mM MgCl2, 0.45% Nonidet P40, 0.45% Tween 20 containing 60 μg/ml proteinase K, 3 hours at 56° C. Ten μl of the lysate were used as template to amplify the corresponding genes. The gene coding for MOMP (Serovar K) (SEQ ID No 1 below) was PCR amplified using the CYK/OMP/5/NDE and CYKD/OMP/3/BG oligonucleotide primers (see table 1). The gene coding for MOMP (Serovar D) (SEQ ID N° 2 below) was PCR amplified using the CYD/OMP/5/NRU and CYKD/OMP/3/BG oligonucleotide primers (see table 1). The conditions used for PCR amplification were those described by the supplier (HiFi DNA polymerase, Boehringer Mannheim, GmbH). Thermal cycling was the following: 25 times (94° C. Imin., 52° C. lmin., 72° C. 3 min.) and 1 time (72° C. 10 min., 4° C. up to recovery). The corresponding amplicons (1194 bp) were digested with either NdeI/BglII or NruI/BglII restriction enzymes and can be cloned in the corresponding restriction sites of pCMK (+) delivery vector (as described in WO 01/09350).

PCR Amplifications of the Genes Coding for PorB (Chlamydia trachomatis).

Murine McCoy cells (ATCC) infected either, with Chlamydia trachomatis Serovar K (UW31-CX-serK), or Serovar D (UW31-CX-serD), were lysed in 400 μl of lysis buffer: 50 mM KCl, 10 mM Tris-HCl pH 8.3, 2.5 mM MgCl2, 0.45% Nonidet P40, 0.45% Tween 20 containing 60 μg/ml proteinase K, 3 hours at 56° C. Ten μl of the lysate were used as template to amplified the corresponding genes.

PorB sequences are highly conserved amongst serovar D and K (SEQ ID No 3 below). The same primers were used to amplify the corresponding genes in both serovars: CYD/PORB/5/NRU and CYD/PORB/3/BG (see table 1). The conditions used for PCR amplification were those described by the supplier (HiFi DNA polymerase, Boehringer Mannheim, GmbH). Thermal cycling was the following: 25 times (94° C. 1 min., 52° C. 1 min., 72° C. 3 min.) and 1 time (72° C. 10 min., 4° C. up to recovery). The corresponding amplicons (1035 bp) were digested with NruI/BglII restriction enzymes and can be cloned in the corresponding restriction sites of pCMK (+) delivery vector (as described in WO 01/09350).

Transformation

Linearized recombinant pCMK plasmids can be transformed within a Neisseria meningitidis serogroup B strain lacking functional cps genes (described in WO 01/09350). Integration resulting from a double crossing-over between the pCMK vectors and the chromosomal porA locus can be selected by a combination of PCR and Western Blot screening as described in WO 01/09350.

TABLE 1
Oligonucleotides used in this work

| | | |
|---|---|---|
| Oligonucleotides | Sequence | Remark(s) |

| | | |
|---|---|---|
| CYK/OMP/5/NDE | 5′-GGG AAT CCA TAT GAA | NdeI |
| | AAA ACT CTT GAA | cloning site |
| | ATC GG-3′ | |
| CYKD/OMP/3/BG | 5′-GGA AGA TCT TTA GAA | Bgl II |
| | GCG GAA TTG TGC AT-3′ | cloning site |
| CYD/OMP/5/NRU | 5′-CTG CAG AAT CGC GAA | Nru I |
| | TGA AAA AAC TCT | cloning site |
| | TGA AAT CGG-3′ | |
| CYD/POR/5/NRU | 5′-CTG CAG AAT CGC GAA | Nru I |
| | TGA GTA GCA AGC | cloning site |
| | TAG TGA AC-3′ | |
| CYD/POR/3/BG | 5′-AGG AGA TCT TTA GAA | Bgl II |
| | TTG GAA TCC TCC GG-3′ | cloning site |

SEQID N°1:
Nucleotide sequence of DNA coding for Chlamydia trachomatis MOMP
serovar K protein.
atgaaaaaactcttgaaatcggtattagtatttgccgctttgagttctgcttcctccttgcaagctctgcctgtggggaa
tcctgctgaaccaagccttatgatcgacggaattctgtgggaaggtttcggcggagatccttgcgatccttgcaccactt
ggtgtgacgctatcagcatgcgcgttggttactacggagactttgttttcgaccgtgttttgaaaactgatgtgaataaa
gaatttcagatgggagcggcgcctactaccagcgatgtagaaggcttacaaaacgatccaacaacaaatgttgctcgtcc
aaatcccgcttatggcaaacacatgcaagatgctgaaatgtttacgaacgctgcttacatggcattaaatatctgggatc
gttttgatgtattttgtacattgggagcaactaccggttatttaagaggaaactccgcttccttcaacttagttggatta
ttcggaacaaaaacacaatattctaagtttaatacagcgaatcttgttcctaacactgctttggatcgagctgtggttga
gctttatacagacaccacctttgcttggagcgtaggtgctcgtgcagctctctgggaatgtgggtgtgcaacgttaggag
cttctttccaatatgctcaatctaaacctaaagtagaagagttaaatgttctttgtaatgcatccgaatttactattaat
aagccgaaaggatatgttggggtggaatttccacttgatattaccgcaggaacagaagctgcgacagggactaaggatgc
ctctattgactaccatgagtggcaagcaagtttagccctttcttacagattaaatatgttcactccttacattggagtta
aatggtctagagtaagttttgatgccgacacgatccgtatcgctcagcctaaattggctgaagcaatcttggatgtcact
actctaaacccgaccatcgctggtaaaggagctgtggtctcttccggaagcgataacgaactggctgatacaatgcaaat
cgtttccttgcagttgaacaagctgaaatctagaaaatcttgcggtattgcagtaggaacgactattgtagatgcagata
aatacgcagttacagttgagactcgcttgatcgatgagagagcagctcacgtaaatgcacaattccgcttctaa
SEQID N°2:
Nucleotide sequence of DNA coding for Chlamydia trachomatis MOMP serovar
D protein.
atgaaaaaactcttgaaatcggtattagtatttgccgctttgagttctgcttcctccttgcaagctctgcctgtggggaa
tcctgctgaaccaagccttatgatcgacggaattctgtgggaaggtttcggcggagatccttgcgatccttgcgccactt
ggtgtgacgctatcagcatgcgtgttggttactacggagactttgttttcgaccgtgttttgaaaactgatgtgaataaa
gaatttcagatgggtgccaagcctacaactgatacaggcaatagtgcagctccatccactcttacagcaagagagaatcc
tgcttacggccgacatatgcaggatgctgagatgtttacaaatgccgcttgcatggcattgaatatttgggatcgttttg
atgtattctgtacattaggagccaccagtggatatcttaaaggaaactctgcttctttcaatttagttggattgtttgga
gataatgaaaatcaaaaaacggtcaaagcggagtctgtaccaaatatgagctttgatcaatctgttgttgagttgtatac
agatactacttttgcgtggagcgtcggcgctcgcgcagctttgtgggaatgtggatgtgcaactttaggagcttcattcc
aatatgctcaatctaaacctaaagtagaagaattaaacgttctctgcaatgcagcagagtttactattaataaacctaaa
gggtatgtaggtaaggagtttcctcttgatcttacagcaggaacagatgctgcgacaggaactaaggatgcctctattga
ttaccatgaatggcaagcaagtttagctctctcttacagactgaatatgttcactccctacattggagttaaatggtctc
gagcaagctttgatgccgatacgattcgtatagcccagccaaaatcagctacagctatttttgatactaccacgcttaac
ccaactattgctggagctggcgatgtgaaaactggcgcagagggtcagctcggagacacaatgcaaatcgtttccttgca
attgaacaagatgaaatctagaaaatcttgcggtattgcagtaggaacaactattgtggatgcagacaaatacgcagtta
cagttgagactcgcttgatcgatgagagagcagctcacgtaaatgcacaattccgcttctaa
SEQID N°3:
Nucleotide sequence of DNA coding for Chlamydia trachomatis PorB serovar D
protein.
atgagtagcaagctagtgaactatctccgtttgactttcctatcttttttagggatcgcatctacttcattagacgctat
gcctgcggggaatccggcgtttccagtcatcccggggattaatattgaacagaaaaatgcctgttctttcgatttatgta
attcttatgatgtactatccgcactgtccggtaacctgaagctctgcttctgcggagattatatcttttcagaagaagct
caggtaaaagatgtccctgtcgttacctctgtgacaacagctggggttggtccttctcctgatattacttcgacaaccaa
aacgcgaaatttcgatctcgtgaactgtaatctcaatacaaactgtgtagctgtagctttttcccttcctgatcgttcgc
tgagcgcgattcctctgtttgatgtgagtttcgaagtgaaagtaggaggactgaaacaatactaccgccttcccatgaat
gcctatcgagacttcacctcggaacctctcaattctgaatcagaagttacggacgggatgattgaagtacagtccaatta
cggatttgtttgggatgttagcttgaaaaaagtcatatggaaagatggcgtttcctttgtaggcgtcggtgcagactatc
gccatgcttcttgccctattgactacatcattgcaaacagtcaagctaatccagaagtattcatcgctgactcggatggg
aaactgaacttcaaggagtggagtgtctgcgtaggtcttactacctatgtgaatgactacgttcttccttacttagcgtt
ttctatagggagtgtttctcgccaagctccggacgacagcttcaaaaaattagaagatcgcttcactaacctcaaattta
aagttcgtaaaattaccagctctcatcgtggaaacatctgcatcggagcgacaaactatgtcgccgataacttcttctac
aacgtagaaggaagatggggaagccagcgcgctgtgaacgtctccggaggattccaattctaa

### Example 4

### Isolation and Purification of Blebs from Meningococci Devoid of Capsular Polysaccharide

Recombinant blebs can be purified as described below. The cell paste (42gr) is suspended in 211 ml of 0.1M Tris-Cl buffer pH 8.6 containing 10 mM EDTA and 0.5% Sodium Deoxycholate (DOC). The ratio of buffer to biomass should be 5/1 (V/W). The biomass is extracted by magnetic stirring for 30 minutes at room temperature. Total extract is then centrifuged at 20,000 g for 30 minutes at 4° C. (13,000 rpm in a JA-20 rotor, Beckman J2-HS centrifuge). The pellet should be discarded. The supernatant is ultracentrifuged at 125,000 g for 2 hours at 4° C. (40,000 rpm in a 50.2Ti rotor, Beckman L8-70M ultracentrifuge) in order to concentrate vesicles. The supernatant should be discarded. The pellet is gently suspended in 25 ml of 50 mM Tris-Cl buffer pH 8.6 containing 2 mM EDTA, 1.2% DOC and 20% sucrose. After a second ultracentrifugation step at 125,000 g for 2 hours at 4° C., vesicles are gently suspended in 44 ml of 3% sucrose and stored at 4° C. All solutions used for bleb extraction and purification contained 0.01% thiomersalate. As illustrated in WO 01/019350, this procedure yields protein preparations highly enriched in outer-membrane proteins.

### Example 5

### Models for testing protection against Gonococcal and C. trachomatis Infection

This can be done as described above in Example 2. In addition Whittum-Hudson et al. (Vaccine 2001 Jul. 16;19(28-29):4061-71) “The anti-idiotypic antibody to chlamydial glycolipid exoantigen (GLXA) protects mice against genital infection with a human biovar of Chlamydia trachomatis” is a vaginal inoculation model for C. trachomatis (incorporated by reference herein) which can also be used to test vaccine efficacy.

1. A Gram-negative bacterial bleb presenting on its surface the PorB outer membrane protein from Chlamydia trachomatis.

2. The Gram-negative bleb of claim 1 further presenting on its surface the PmpG outer membrane proteins from Chlamydia trachomatis.

3. The Gram-negative bleb of claim 1 further presenting on its surface MOMP from one or more serovars from Chlamydia trachomatis.

4. A Gram-negative bleb presenting on its surface both the PmpG and MOMP (from one or more serovars) outer membrane proteins from Chlamydia trachomatis.

5. The bleb of claim 1, wherein the bleb is a gonococcal bleb.

6. (canceled)

7. (canceled)

8. The bleb of claim 5 wherein the bleb is derived from a strain that has a detoxified lipid A portion of bacterial LPS, due to the strain having been engineered to reduce or switch off expression of at least one gene selected from the group consisting of: htrB, msbB and lpxK.

9. The bleb of claim 5 wherein the bleb is derived from a strain that has a detoxified lipid A portion of bacterial LPS, due to the strain having been engineered to express at a higher level at least one gene selected from the group consisting of: pmrA, pmrB, pmrE and pmrF.

10. A vaccine composition comprising the bleb of claim 1 and a pharmaceutically suitable excipient or carrier.

11. The vaccine of claim 10, additionally comprising a mucosal adjuvant.

12. A method of preventing Chlamydia trachomatis infection in a host comprising the step of administering an effective amount of the vaccine of claim 10 to a host in need thereof.

13. The method of claim 12 wherein the vaccine is mucosally administered via a route chosen from the group of: intranasal, oral, and intravaginal.

14. A Gram-negative bleb presenting on its surface a protective antigen from Chlamydia pneumoniae.

15-20. (canceled)

21. The bleb of claim 14, wherein the bleb is a meningococcal bleb.

22. The bleb of claim 21, wherein the bleb is derived from a meningococcal strain that has been modified to upregulate at least one protective meningococcal outer membrane antigen.

23. The bleb of claim 21, wherein the bleb is derived from a meningococcal strain that has been modified to downregulate at least one immunodominant variable or non-protective meningococcal outer membrane antigen.

24. The bleb of claim 21, wherein the bleb is derived from a strain that has a detoxified lipid A portion of bacterial LPS, due to the strain having been engineered to reduce or switch off expression of at least one gene selected from the group consisting of: htrB, msbB and lpxK.

25. The bleb of claim 21, wherein the bleb is derived from a strain that has a detoxified lipid A portion of bacterial LPS, due to the strain having been engineered to express at a higher level at least one gene selected from the group consisting of: pmrA, pmrB, pmrE and pmrF.

26. A vaccine composition comprising the bleb of claim 14 and a pharmaceutically suitable excipient or carrier.

27. (canceled)

28. A method of preventing Chlamydia pneumoniae infection in a host comprising the step of administering an effective amount of the vaccine of claim 26 to a host in need thereof.

29. The method of claim 28 where in the vaccine is mucosally administered via a route chosen from the group of: intranasal and oral.
